SEQUENCE LISTING (482 sequences)

SEQ ID NO: 1 - protein, 277 aa, Escherichia coli
MQRLFLLVAVMLLSGCLTAPPKEAARPTLMPRAQSYKDLTHLPAPTGKIFVSVYNIQDETGQFKPYPASNFSTAVPQSATAMLVTALKDSRWFIPLERQGLQNLLNERKIIRAAQENGTVAINNRIPLQSLTAANIMVEGSIIGYESNVKSGGVGARYFGIGADTQYQLDQIAVNLRVVNVSTGEILSSVNTSKTILSYEVQAGVFRFIDYQRLLEGEVGYTSNEPVMLCLMSAIETGVIFLINDGIDRGLWDLQNKAERQNDILVKYRHMSVPPES

SEQ ID NO: 2 - DNA, 834 bp, Escherichia coli
  source 1..834: mol_type = genomic DNA
atgcagcgcttatttcttttggttgccgtcatgttactgagcggatgcttaaccgccccgcctaaagaagccgccagaccgacattaatgcctcgtgctcagagctacaaagatttgacccatctgccagcgccgacgggtaaaatctttgtttcggtatacaacattcaggacgaaaccgggcaatttaaaccctacccggcaagtaacttctccactgctgttccgcaaagcgccacggcaatgctggtcacggcactgaaagattctcgctggtttataccgctggagcgccagggcttacaaaacctgcttaacgagcgcaagattattcgtgcggcacaagaaaacggcacggttgccattaataaccgaatcccgctgcaatctttaacggcggcaaatatcatggttgaaggttcgattatcggttatgaaagcaacgtcaaatctggcggggttggggcaagatattttggcatcggtgccgacacgcaataccagctcgatcagattgccgtgaacctgcgcgtcgtcaatgtgagtaccggcgagatcctttcttcggtgaacaccagtaagacgatactttcctatgaagttcaggccggggttttccgctttattgactaccagcgcttgcttgaaggggaagtgggttacacctcgaacgaacctgttatgctgtgcctgatgtcggctatcgaaacaggggtcattttcctgattaatgatggtatcgaccgtggtctgtgggatttgcaaaataaagcagaacggcagaatgacattctggtgaaataccgccatatgtcggttccaccggaatcctga

SEQ ID NO: 3 - protein, 25 aa, Escherichia coli
VYNIQDETGQFKPYPASNFSTAVPQ

SEQ ID NO: 4 - protein, 25 aa, Escherichia coli
VYNIQDETGQFKPYPASNFSTAVPQ

SEQ ID NO: 5 - protein, 25 aa, Escherichia coli
VYNIQDETGQFKPYPASNASTAVPQ

SEQ ID NO: 6 - protein, 25 aa, Escherichia coli
VYNIQDETGQFKPYPASNGSTAVPQ

SEQ ID NO: 7 - protein, 25 aa, Escherichia coli
VYNIQDETGQFKPYPASNVSTAVPQ

SEQ ID NO: 8 - protein, 24 aa, Escherichia coli
VYNIQDETGQFKPYPASNSTAVPQ

SEQ ID NO: 9 - protein, 25 aa, Escherichia coli
VYNIQDETGQFKPYPASAFSTAVPQ

SEQ ID NO: 10 - protein, 25 aa, Escherichia coli
VYNIQDETGQFKPYPASAASTAVPQ

SEQ ID NO: 11 - protein, 25 aa, Escherichia coli
VYNIQDETGQFKPYPASAGSTAVPQ

SEQ ID NO: 12 - protein, 25 aa, Escherichia coli
VYNIQDETGQFKPYPASAVSTAVPQ

SEQ ID NO: 13 - protein, 24 aa, Escherichia coli
VYNIQDETGQFKPYPASASTAVPQ

SEQ ID NO: 14 - protein, 25 aa, Escherichia coli
VYNIQDETGQFKPYPASGFSTAVPQ

SEQ ID NO: 15 - protein, 25 aa, Escherichia coli
VYNIQDETGQFKPYPASGASTAVPQ

SEQ ID NO: 16 - protein, 25 aa, Escherichia coli
VYNIQDETGQFKPYPASGGSTAVPQ

SEQ ID NO: 17 - protein, 25 aa, Escherichia coli
VYNIQDETGQFKPYPASGVSTAVPQ

SEQ ID NO: 18 - protein, 24 aa, Escherichia coli
VYNIQDETGQFKPYPASGSTAVPQ

SEQ ID NO: 19 - protein, 25 aa, Escherichia coli
VYNIQDETGQFKPYPASVFSTAVPQ

SEQ ID NO: 20 - protein, 25 aa, Escherichia coli
VYNIQDETGQFKPYPASVASTAVPQ

SEQ ID NO: 21 - protein, 25 aa, Escherichia coli
VYNIQDETGQFKPYPASVGSTAVPQ

SEQ ID NO: 22 - protein, 25 aa, Escherichia coli
VYNIQDETGQFKPYPASVVSTAVPQ

SEQ ID NO: 23 - protein, 24 aa, Escherichia coli
VYNIQDETGQFKPYPASVSTAVPQ

SEQ ID NO: 24 - protein, 25 aa, Escherichia coli
VYNIQDETGQFKPYPASSFSTAVPQ

SEQ ID NO: 25 - protein, 25 aa, Escherichia coli
VYNIQDETGQFKPYPASSASTAVPQ

SEQ ID NO: 26 - protein, 25 aa, Escherichia coli
VYNIQDETGQFKPYPASSGSTAVPQ

SEQ ID NO: 27 - protein, 25 aa, Escherichia coli
VYNIQDETGQFKPYPASSVSTAVPQ

SEQ ID NO: 28 - protein, 24 aa, Escherichia coli
VYNIQDETGQFKPYPASSSTAVPQ

SEQ ID NO: 29 - protein, 25 aa, Escherichia coli
VYNIQDETGQFKPYPASTFSTAVPQ

SEQ ID NO: 30 - protein, 25 aa, Escherichia coli
VYNIQDETGQFKPYPASTASTAVPQ

SEQ ID NO: 31 - protein, 25 aa, Escherichia coli
VYNIQDETGQFKPYPASTGSTAVPQ

SEQ ID NO: 32 - protein, 25 aa, Escherichia coli
VYNIQDETGQFKPYPASTVSTAVPQ

SEQ ID NO: 33 - protein, 24 aa, Escherichia coli
VYNIQDETGQFKPYPASTSTAVPQ

SEQ ID NO: 34 - protein, 24 aa, Escherichia coli
VYNIQDETGQFKPYPASFSTAVPQ

SEQ ID NO: 35 - protein, 24 aa, Escherichia coli
VYNIQDETGQFKPYPASASTAVPQ

SEQ ID NO: 36 - protein, 24 aa, Escherichia coli
VYNIQDETGQFKPYPASGSTAVPQ

SEQ ID NO: 37 - protein, 24 aa, Escherichia coli
VYNIQDETGQFKPYPASVSTAVPQ

SEQ ID NO: 38 - protein, 23 aa, Escherichia coli
VYNIQDETGQFKPYPASSTAVPQ

SEQ ID NO: 39 - protein, 25 aa, Escherichia coli
VYNIQDETGQFKPAPASNFSTAVPQ

SEQ ID NO: 40 - protein, 25 aa, Escherichia coli
VYNIQDETGQFKPAPASNASTAVPQ

SEQ ID NO: 41 - protein, 25 aa, Escherichia coli
VYNIQDETGQFKPAPASNGSTAVPQ

SEQ ID NO: 42 - protein, 25 aa, Escherichia coli
VYNIQDETGQFKPAPASNVSTAVPQ

SEQ ID NO: 43 - protein, 24 aa, Escherichia coli
VYNIQDETGQFKPAPASNSTAVPQ

SEQ ID NO: 44 - protein, 25 aa, Escherichia coli
VYNIQDETGQFKPAPASAFSTAVPQ

SEQ ID NO: 45 - protein, 25 aa, Escherichia coli
VYNIQDETGQFKPAPASAASTAVPQ

SEQ ID NO: 46 - protein, 25 aa, Escherichia coli
VYNIQDETGQFKPAPASAGSTAVPQ

SEQ ID NO: 47 - protein, 25 aa, Escherichia coli
VYNIQDETGQFKPAPASAVSTAVPQ

SEQ ID NO: 48 - protein, 24 aa, Escherichia coli
VYNIQDETGQFKPAPASASTAVPQ

SEQ ID NO: 49 - protein, 25 aa, Escherichia coli
VYNIQDETGQFKPAPASGFSTAVPQ

SEQ ID NO: 50 - protein, 25 aa, Escherichia coli
VYNIQDETGQFKPAPASGASTAVPQ

SEQ ID NO: 51 - protein, 25 aa, Escherichia coli
VYNIQDETGQFKPAPASGGSTAVPQ

SEQ ID NO: 52 - protein, 25 aa, Escherichia coli
VYNIQDETGQFKPAPASGVSTAVPQ

SEQ ID NO: 53 - protein, 24 aa, Escherichia coli
VYNIQDETGQFKPAPASGSTAVPQ

SEQ ID NO: 54 - protein, 25 aa, Escherichia coli
VYNIQDETGQFKPAPASVFSTAVPQ

SEQ ID NO: 55 - protein, 25 aa, Escherichia coli
VYNIQDETGQFKPAPASVASTAVPQ

SEQ ID NO: 56 - protein, 25 aa, Escherichia coli
VYNIQDETGQFKPAPASVGSTAVPQ

SEQ ID NO: 57 - protein, 25 aa, Escherichia coli
VYNIQDETGQFKPAPASVVSTAVPQ

SEQ ID NO: 58 - protein, 24 aa, Escherichia coli
VYNIQDETGQFKPAPASVSTAVPQ

SEQ ID NO: 59 - protein, 25 aa, Escherichia coli
VYNIQDETGQFKPAPASSFSTAVPQ

SEQ ID NO: 60 - protein, 25 aa, Escherichia coli
VYNIQDETGQFKPAPASSASTAVPQ

SEQ ID NO: 61 - protein, 25 aa, Escherichia coli
VYNIQDETGQFKPAPASSGSTAVPQ

SEQ ID NO: 62 - protein, 25 aa, Escherichia coli
VYNIQDETGQFKPAPASSVSTAVPQ

SEQ ID NO: 63 - protein, 24 aa, Escherichia coli
VYNIQDETGQFKPAPASSSTAVPQ

SEQ ID NO: 64 - protein, 25 aa, Escherichia coli
VYNIQDETGQFKPAPASTFSTAVPQ

SEQ ID NO: 65 - protein, 25 aa, Escherichia coli
VYNIQDETGQFKPAPASTASTAVPQ

SEQ ID NO: 66 - protein, 25 aa, Escherichia coli
VYNIQDETGQFKPAPASTGSTAVPQ

SEQ ID NO: 67 - protein, 25 aa, Escherichia coli
VYNIQDETGQFKPAPASTVSTAVPQ

SEQ ID NO: 68 - protein, 24 aa, Escherichia coli
VYNIQDETGQFKPAPASTSTAVPQ

SEQ ID NO: 69 - protein, 24 aa, Escherichia coli
VYNIQDETGQFKPAPASFSTAVPQ

SEQ ID NO: 70 - protein, 24 aa, Escherichia coli
VYNIQDETGQFKPAPASASTAVPQ

SEQ ID NO: 71 - protein, 24 aa, Escherichia coli
VYNIQDETGQFKPAPASGSTAVPQ

SEQ ID NO: 72 - protein, 24 aa, Escherichia coli
VYNIQDETGQFKPAPASVSTAVPQ

SEQ ID NO: 73 - protein, 23 aa, Escherichia coli
VYNIQDETGQFKPAPASSTAVPQ

SEQ ID NO: 74 - protein, 25 aa, Escherichia coli
VYNIQDETGQFKPGPASNFSTAVPQ

SEQ ID NO: 75 - protein, 25 aa, Escherichia coli
VYNIQDETGQFKPGPASNASTAVPQ

SEQ ID NO: 76 - protein, 25 aa, Escherichia coli
VYNIQDETGQFKPGPASNGSTAVPQ

SEQ ID NO: 77 - protein, 25 aa, Escherichia coli
VYNIQDETGQFKPGPASNVSTAVPQ

SEQ ID NO: 78 - protein, 24 aa, Escherichia coli
VYNIQDETGQFKPGPASNSTAVPQ

SEQ ID NO: 79 - protein, 25 aa, Escherichia coli
VYNIQDETGQFKPGPASAFSTAVPQ

SEQ ID NO: 80 - protein, 25 aa, Escherichia coli
VYNIQDETGQFKPGPASAASTAVPQ

SEQ ID NO: 81 - protein, 25 aa, Escherichia coli
VYNIQDETGQFKPGPASAGSTAVPQ

SEQ ID NO: 82 - protein, 25 aa, Escherichia coli
VYNIQDETGQFKPGPASAVSTAVPQ

SEQ ID NO: 83 - protein, 24 aa, Escherichia coli
VYNIQDETGQFKPGPASASTAVPQ

SEQ ID NO: 84 - protein, 25 aa, Escherichia coli
VYNIQDETGQFKPGPASGFSTAVPQ

SEQ ID NO: 85 - protein, 25 aa, Escherichia coli
VYNIQDETGQFKPGPASGASTAVPQ

SEQ ID NO: 86 - protein, 25 aa, Escherichia coli
VYNIQDETGQFKPGPASGGSTAVPQ

SEQ ID NO: 87 - protein, 25 aa, Escherichia coli
VYNIQDETGQFKPGPASGVSTAVPQ

SEQ ID NO: 88 - protein, 24 aa, Escherichia coli
VYNIQDETGQFKPGPASGSTAVPQ

SEQ ID NO: 89 - protein, 25 aa, Escherichia coli
VYNIQDETGQFKPGPASVFSTAVPQ

SEQ ID NO: 90 - protein, 25 aa, Escherichia coli
VYNIQDETGQFKPGPASVASTAVPQ

SEQ ID NO: 91 - protein, 25 aa, Escherichia coli
VYNIQDETGQFKPGPASVGSTAVPQ

SEQ ID NO: 92 - protein, 25 aa, Escherichia coli
VYNIQDETGQFKPGPASVVSTAVPQ

SEQ ID NO: 93 - protein, 24 aa, Escherichia coli
VYNIQDETGQFKPGPASVSTAVPQ

SEQ ID NO: 94 - protein, 25 aa, Escherichia coli
VYNIQDETGQFKPGPASSFSTAVPQ

SEQ ID NO: 95 - protein, 25 aa, Escherichia coli
VYNIQDETGQFKPGPASSASTAVPQ

SEQ ID NO: 96 - protein, 25 aa, Escherichia coli
VYNIQDETGQFKPGPASSGSTAVPQ

SEQ ID NO: 97 - protein, 25 aa, Escherichia coli
VYNIQDETGQFKPGPASSVSTAVPQ

SEQ ID NO: 98 - protein, 24 aa, Escherichia coli
VYNIQDETGQFKPGPASSSTAVPQ

SEQ ID NO: 99 - protein, 25 aa, Escherichia coli
VYNIQDETGQFKPGPASTFSTAVPQ

SEQ ID NO: 100 - protein, 25 aa, Escherichia coli
VYNIQDETGQFKPGPASTASTAVPQ

SEQ ID NO: 101 - protein, 25 aa, Escherichia coli
VYNIQDETGQFKPGPASTGSTAVPQ

SEQ ID NO: 102 - protein, 25 aa, Escherichia coli
VYNIQDETGQFKPGPASTVSTAVPQ

SEQ ID NO: 103 - protein, 24 aa, Escherichia coli
VYNIQDETGQFKPGPASTSTAVPQ

SEQ ID NO: 104 - protein, 24 aa, Escherichia coli
VYNIQDETGQFKPGPASFSTAVPQ

SEQ ID NO: 105 - protein, 24 aa, Escherichia coli
VYNIQDETGQFKPGPASASTAVPQ

SEQ ID NO: 106 - protein, 24 aa, Escherichia coli
VYNIQDETGQFKPGPASGSTAVPQ

SEQ ID NO: 107 - protein, 24 aa, Escherichia coli
VYNIQDETGQFKPGPASVSTAVPQ

SEQ ID NO: 108 - protein, 23 aa, Escherichia coli
VYNIQDETGQFKPGPASSTAVPQ

SEQ ID NO: 109 - protein, 25 aa, Escherichia coli
VYNIQDETGQFKPVPASNFSTAVPQ

SEQ ID NO: 110 - protein, 25 aa, Escherichia coli
VYNIQDETGQFKPVPASNASTAVPQ

SEQ ID NO: 111 - protein, 25 aa, Escherichia coli
VYNIQDETGQFKPVPASNGSTAVPQ

SEQ ID NO: 112 - protein, 25 aa, Escherichia coli
VYNIQDETGQFKPVPASNVSTAVPQ

SEQ ID NO: 113 - protein, 24 aa, Escherichia coli
VYNIQDETGQFKPVPASNSTAVPQ

SEQ ID NO: 114 - protein, 25 aa, Escherichia coli
VYNIQDETGQFKPVPASAFSTAVPQ

SEQ ID NO: 115 - protein, 25 aa, Escherichia coli
VYNIQDETGQFKPVPASAASTAVPQ

SEQ ID NO: 116 - protein, 25 aa, Escherichia coli
VYNIQDETGQFKPVPASAGSTAVPQ

SEQ ID NO: 117 - protein, 25 aa, Escherichia coli
VYNIQDETGQFKPVPASAVSTAVPQ

SEQ ID NO: 118 - protein, 24 aa, Escherichia coli
VYNIQDETGQFKPVPASASTAVPQ

SEQ ID NO: 119 - protein, 25 aa, Escherichia coli
VYNIQDETGQFKPVPASGFSTAVPQ

SEQ ID NO: 120 - protein, 25 aa, Escherichia coli
VYNIQDETGQFKPVPASGASTAVPQ

SEQ ID NO: 121 - protein, 25 aa, Escherichia coli
VYNIQDETGQFKPVPASGGSTAVPQ

SEQ ID NO: 122 - protein, 25 aa, Escherichia coli
VYNIQDETGQFKPVPASGVSTAVPQ

SEQ ID NO: 123 - protein, 24 aa, Escherichia coli
VYNIQDETGQFKPVPASGSTAVPQ

SEQ ID NO: 124 - protein, 25 aa, Escherichia coli
VYNIQDETGQFKPVPASVFSTAVPQ

SEQ ID NO: 125 - protein, 25 aa, Escherichia coli
VYNIQDETGQFKPVPASVASTAVPQ

SEQ ID NO: 126 - protein, 25 aa, Escherichia coli
VYNIQDETGQFKPVPASVGSTAVPQ

SEQ ID NO: 127 - protein, 25 aa, Escherichia coli
VYNIQDETGQFKPVPASVVSTAVPQ

SEQ ID NO: 128 - protein, 24 aa, Escherichia coli
VYNIQDETGQFKPVPASVSTAVPQ

SEQ ID NO: 129 - protein, 25 aa, Escherichia coli
VYNIQDETGQFKPVPASSFSTAVPQ

SEQ ID NO: 130 - protein, 25 aa, Escherichia coli
VYNIQDETGQFKPVPASSASTAVPQ

SEQ ID NO: 131 - protein, 25 aa, Escherichia coli
VYNIQDETGQFKPVPASSGSTAVPQ

SEQ ID NO: 132 - protein, 25 aa, Escherichia coli
VYNIQDETGQFKPVPASSVSTAVPQ

SEQ ID NO: 133 - protein, 24 aa, Escherichia coli
VYNIQDETGQFKPVPASSSTAVPQ

SEQ ID NO: 134 - protein, 25 aa, Escherichia coli
VYNIQDETGQFKPVPASTFSTAVPQ

SEQ ID NO: 135 - protein, 25 aa, Escherichia coli
VYNIQDETGQFKPVPASTASTAVPQ

SEQ ID NO: 136 - protein, 25 aa, Escherichia coli
VYNIQDETGQFKPVPASTGSTAVPQ

SEQ ID NO: 137 - protein, 25 aa, Escherichia coli
VYNIQDETGQFKPVPASTVSTAVPQ

SEQ ID NO: 138 - protein, 24 aa, Escherichia coli
VYNIQDETGQFKPVPASTSTAVPQ

SEQ ID NO: 139 - protein, 24 aa, Escherichia coli
VYNIQDETGQFKPVPASFSTAVPQ

SEQ ID NO: 140 - protein, 24 aa, Escherichia coli
VYNIQDETGQFKPVPASASTAVPQ

SEQ ID NO: 141 - protein, 24 aa, Escherichia coli
VYNIQDETGQFKPVPASGSTAVPQ

SEQ ID NO: 142 - protein, 24 aa, Escherichia coli
VYNIQDETGQFKPVPASVSTAVPQ

SEQ ID NO: 143 - protein, 23 aa, Escherichia coli
VYNIQDETGQFKPVPASSTAVPQ

SEQ ID NO: 144 - protein, 25 aa, Escherichia coli
VYNIQDETGQFKPLPASNFSTAVPQ

SEQ ID NO: 145 - protein, 25 aa, Escherichia coli
VYNIQDETGQFKPLPASNASTAVPQ

SEQ ID NO: 146 - protein, 25 aa, Escherichia coli
VYNIQDETGQFKPLPASNGSTAVPQ

SEQ ID NO: 147 - protein, 25 aa, Escherichia coli
VYNIQDETGQFKPLPASNVSTAVPQ

SEQ ID NO: 148 - protein, 24 aa, Escherichia coli
VYNIQDETGQFKPLPASNSTAVPQ

SEQ ID NO: 149 - protein, 25 aa, Escherichia coli
VYNIQDETGQFKPLPASAFSTAVPQ

SEQ ID NO: 150 - protein, 25 aa, Escherichia coli
VYNIQDETGQFKPLPASAASTAVPQ

SEQ ID NO: 151 - protein, 25 aa, Escherichia coli
VYNIQDETGQFKPLPASAGSTAVPQ

SEQ ID NO: 152 - protein, 25 aa, Escherichia coli
VYNIQDETGQFKPLPASAVSTAVPQ

SEQ ID NO: 153 - protein, 24 aa, Escherichia coli
VYNIQDETGQFKPLPASASTAVPQ

SEQ ID NO: 154 - protein, 25 aa, Escherichia coli
VYNIQDETGQFKPLPASGFSTAVPQ

SEQ ID NO: 155 - protein, 25 aa, Escherichia coli
VYNIQDETGQFKPLPASGASTAVPQ

SEQ ID NO: 156 - protein, 25 aa, Escherichia coli
VYNIQDETGQFKPLPASGGSTAVPQ

SEQ ID NO: 157 - protein, 25 aa, Escherichia coli
VYNIQDETGQFKPLPASGVSTAVPQ

SEQ ID NO: 158 - protein, 24 aa, Escherichia coli
VYNIQDETGQFKPLPASGSTAVPQ

SEQ ID NO: 159 - protein, 25 aa, Escherichia coli
VYNIQDETGQFKPLPASVFSTAVPQ

SEQ ID NO: 160 - protein, 25 aa, Escherichia coli
VYNIQDETGQFKPLPASVASTAVPQ

SEQ ID NO: 161 - protein, 25 aa, Escherichia coli
VYNIQDETGQFKPLPASVGSTAVPQ

SEQ ID NO: 162 - protein, 25 aa, Escherichia coli
VYNIQDETGQFKPLPASVVSTAVPQ

SEQ ID NO: 163 - protein, 24 aa, Escherichia coli
VYNIQDETGQFKPLPASVSTAVPQ

SEQ ID NO: 164 - protein, 25 aa, Escherichia coli
VYNIQDETGQFKPLPASSFSTAVPQ

SEQ ID NO: 165 - protein, 25 aa, Escherichia coli
VYNIQDETGQFKPLPASSASTAVPQ

SEQ ID NO: 166 - protein, 25 aa, Escherichia coli
VYNIQDETGQFKPLPASSGSTAVPQ

SEQ ID NO: 167 - protein, 25 aa, Escherichia coli
VYNIQDETGQFKPLPASSVSTAVPQ

SEQ ID NO: 168 - protein, 24 aa, Escherichia coli
VYNIQDETGQFKPLPASSSTAVPQ

SEQ ID NO: 169 - protein, 25 aa, Escherichia coli
VYNIQDETGQFKPLPASTFSTAVPQ

SEQ ID NO: 170 - protein, 25 aa, Escherichia coli
VYNIQDETGQFKPLPASTASTAVPQ

SEQ ID NO: 171 - protein, 25 aa, Escherichia coli
VYNIQDETGQFKPLPASTGSTAVPQ

SEQ ID NO: 172 - protein, 25 aa, Escherichia coli
VYNIQDETGQFKPLPASTVSTAVPQ

SEQ ID NO: 173 - protein, 24 aa, Escherichia coli
VYNIQDETGQFKPLPASTSTAVPQ

SEQ ID NO: 174 - protein, 24 aa, Escherichia coli
VYNIQDETGQFKPLPASFSTAVPQ

SEQ ID NO: 175 - protein, 24 aa, Escherichia coli
VYNIQDETGQFKPLPASASTAVPQ

SEQ ID NO: 176 - protein, 24 aa, Escherichia coli
VYNIQDETGQFKPLPASGSTAVPQ

SEQ ID NO: 177 - protein, 24 aa, Escherichia coli
VYNIQDETGQFKPLPASVSTAVPQ

SEQ ID NO: 178 - protein, 23 aa, Escherichia coli
VYNIQDETGQFKPLPASSTAVPQ

SEQ ID NO: 179 - protein, 25 aa, Escherichia coli
VYNIQDETGQFKPIPASNFSTAVPQ

SEQ ID NO: 180 - protein, 25 aa, Escherichia coli
VYNIQDETGQFKPIPASNASTAVPQ

SEQ ID NO: 181 - protein, 25 aa, Escherichia coli
VYNIQDETGQFKPIPASNGSTAVPQ

SEQ ID NO: 182 - protein, 25 aa, Escherichia coli
VYNIQDETGQFKPIPASNVSTAVPQ

SEQ ID NO: 183 - protein, 24 aa, Escherichia coli
VYNIQDETGQFKPIPASNSTAVPQ

SEQ ID NO: 184 - protein, 25 aa, Escherichia coli
VYNIQDETGQFKPIPASAFSTAVPQ

SEQ ID NO: 185 - protein, 25 aa, Escherichia coli
VYNIQDETGQFKPIPASAASTAVPQ

SEQ ID NO: 186 - protein, 25 aa, Escherichia coli
VYNIQDETGQFKPIPASAGSTAVPQ

SEQ ID NO: 187 - protein, 25 aa, Escherichia coli
VYNIQDETGQFKPIPASAVSTAVPQ

SEQ ID NO: 188 - protein, 24 aa, Escherichia coli
VYNIQDETGQFKPIPASASTAVPQ

SEQ ID NO: 189 - protein, 25 aa, Escherichia coli
VYNIQDETGQFKPIPASGFSTAVPQ

SEQ ID NO: 190 - protein, 25 aa, Escherichia coli
VYNIQDETGQFKPIPASGASTAVPQ

SEQ ID NO: 191 - protein, 25 aa, Escherichia coli
VYNIQDETGQFKPIPASGGSTAVPQ

SEQ ID NO: 192 - protein, 25 aa, Escherichia coli
VYNIQDETGQFKPIPASGVSTAVPQ

SEQ ID NO: 193 - protein, 24 aa, Escherichia coli
VYNIQDETGQFKPIPASGSTAVPQ

SEQ ID NO: 194 - protein, 25 aa, Escherichia coli
VYNIQDETGQFKPIPASVFSTAVPQ

SEQ ID NO: 195 - protein, 25 aa, Escherichia coli
VYNIQDETGQFKPIPASVASTAVPQ

SEQ ID NO: 196 - protein, 25 aa, Escherichia coli
VYNIQDETGQFKPIPASVGSTAVPQ

SEQ ID NO: 197 - protein, 25 aa, Escherichia coli
VYNIQDETGQFKPIPASVVSTAVPQ

SEQ ID NO: 198 - protein, 24 aa, Escherichia coli
VYNIQDETGQFKPIPASVSTAVPQ

SEQ ID NO: 199 - protein, 25 aa, Escherichia coli
VYNIQDETGQFKPIPASSFSTAVPQ

SEQ ID NO: 200 - protein, 25 aa, Escherichia coli
VYNIQDETGQFKPIPASSASTAVPQ

SEQ ID NO: 201 - protein, 25 aa, Escherichia coli
VYNIQDETGQFKPIPASSGSTAVPQ

SEQ ID NO: 202 - protein, 25 aa, Escherichia coli
VYNIQDETGQFKPIPASSVSTAVPQ

SEQ ID NO: 203 - protein, 24 aa, Escherichia coli
VYNIQDETGQFKPIPASSSTAVPQ

SEQ ID NO: 204 - protein, 25 aa, Escherichia coli
VYNIQDETGQFKPIPASTFSTAVPQ

SEQ ID NO: 205 - protein, 25 aa, Escherichia coli
VYNIQDETGQFKPIPASTASTAVPQ

SEQ ID NO: 206 - protein, 25 aa, Escherichia coli
VYNIQDETGQFKPIPASTGSTAVPQ

SEQ ID NO: 207 - protein, 25 aa, Escherichia coli
VYNIQDETGQFKPIPASTVSTAVPQ

SEQ ID NO: 208 - protein, 24 aa, Escherichia coli
VYNIQDETGQFKPIPASTSTAVPQ

SEQ ID NO: 209 - protein, 24 aa, Escherichia coli
VYNIQDETGQFKPIPASFSTAVPQ

SEQ ID NO: 210 - protein, 24 aa, Escherichia coli
VYNIQDETGQFKPIPASASTAVPQ

SEQ ID NO: 211 - protein, 24 aa, Escherichia coli
VYNIQDETGQFKPIPASGSTAVPQ

SEQ ID NO: 212 - protein, 24 aa, Escherichia coli
VYNIQDETGQFKPIPASVSTAVPQ

SEQ ID NO: 213 - protein, 23 aa, Escherichia coli
VYNIQDETGQFKPIPASSTAVPQ

SEQ ID NO: 214 - protein, 25 aa, Escherichia coli
VYNIQDETGQFKPNPASNFSTAVPQ

SEQ ID NO: 215 - protein, 25 aa, Escherichia coli
VYNIQDETGQFKPNPASNASTAVPQ

SEQ ID NO: 216 - protein, 25 aa, Escherichia coli
VYNIQDETGQFKPNPASNGSTAVPQ

SEQ ID NO: 217 - protein, 25 aa, Escherichia coli
VYNIQDETGQFKPNPASNVSTAVPQ

SEQ ID NO: 218 - protein, 24 aa, Escherichia coli
VYNIQDETGQFKPNPASNSTAVPQ

SEQ ID NO: 219 - protein, 25 aa, Escherichia coli
VYNIQDETGQFKPNPASAFSTAVPQ

SEQ ID NO: 220 - protein, 25 aa, Escherichia coli
VYNIQDETGQFKPNPASAASTAVPQ

SEQ ID NO: 221 - protein, 25 aa, Escherichia coli
VYNIQDETGQFKPNPASAGSTAVPQ

SEQ ID NO: 222 - protein, 25 aa, Escherichia coli
VYNIQDETGQFKPNPASAVSTAVPQ

SEQ ID NO: 223 - protein, 24 aa, Escherichia coli
VYNIQDETGQFKPNPASASTAVPQ

SEQ ID NO: 224 - protein, 25 aa, Escherichia coli
VYNIQDETGQFKPNPASGFSTAVPQ

SEQ ID NO: 225 - protein, 25 aa, Escherichia coli
VYNIQDETGQFKPNPASGASTAVPQ

SEQ ID NO: 226 - protein, 25 aa, Escherichia coli
VYNIQDETGQFKPNPASGGSTAVPQ

SEQ ID NO: 227 - protein, 25 aa, Escherichia coli
VYNIQDETGQFKPNPASGVSTAVPQ

SEQ ID NO: 228 - protein, 24 aa, Escherichia coli
VYNIQDETGQFKPNPASGSTAVPQ

SEQ ID NO: 229 - protein, 25 aa, Escherichia coli
VYNIQDETGQFKPNPASVFSTAVPQ

SEQ ID NO: 230 - protein, 25 aa, Escherichia coli
VYNIQDETGQFKPNPASVASTAVPQ

SEQ ID NO: 231 - protein, 25 aa, Escherichia coli
VYNIQDETGQFKPNPASVGSTAVPQ

SEQ ID NO: 232 - protein, 25 aa, Escherichia coli
VYNIQDETGQFKPNPASVVSTAVPQ

SEQ ID NO: 233 - protein, 24 aa, Escherichia coli
VYNIQDETGQFKPNPASVSTAVPQ

SEQ ID NO: 234 - protein, 25 aa, Escherichia coli
VYNIQDETGQFKPNPASSFSTAVPQ

SEQ ID NO: 235 - protein, 25 aa, Escherichia coli
VYNIQDETGQFKPNPASSASTAVPQ

SEQ ID NO: 236 - protein, 25 aa, Escherichia coli
VYNIQDETGQFKPNPASSGSTAVPQ

SEQ ID NO: 237 - protein, 25 aa, Escherichia coli
VYNIQDETGQFKPNPASSVSTAVPQ

SEQ ID NO: 238 - protein, 24 aa, Escherichia coli
VYNIQDETGQFKPNPASSSTAVPQ

SEQ ID NO: 239 - protein, 25 aa, Escherichia coli
VYNIQDETGQFKPNPASTFSTAVPQ

SEQ ID NO: 240 - protein, 25 aa, Escherichia coli
VYNIQDETGQFKPNPASTASTAVPQ

SEQ ID NO: 241 - protein, 25 aa, Escherichia coli
VYNIQDETGQFKPNPASTGSTAVPQ

SEQ ID NO: 242 - protein, 25 aa, Escherichia coli
VYNIQDETGQFKPNPASTVSTAVPQ

SEQ ID NO: 243 - protein, 24 aa, Escherichia coli
VYNIQDETGQFKPNPASTSTAVPQ

SEQ ID NO: 244 - protein, 24 aa, Escherichia coli
VYNIQDETGQFKPNPASFSTAVPQ

SEQ ID NO: 245 - protein, 24 aa, Escherichia coli
VYNIQDETGQFKPNPASASTAVPQ

SEQ ID NO: 246 - protein, 24 aa, Escherichia coli
VYNIQDETGQFKPNPASGSTAVPQ

SEQ ID NO: 247 - protein, 24 aa, Escherichia coli
VYNIQDETGQFKPNPASVSTAVPQ

SEQ ID NO: 248 - protein, 23 aa, Escherichia coli
VYNIQDETGQFKPNPASSTAVPQ

SEQ ID NO: 249 - protein, 25 aa, Escherichia coli
VYNIQDETGQFKPQPASNFSTAVPQ

SEQ ID NO: 250 - protein, 25 aa, Escherichia coli
VYNIQDETGQFKPQPASNASTAVPQ

SEQ ID NO: 251 - protein, 25 aa, Escherichia coli
VYNIQDETGQFKPQPASNGSTAVPQ

SEQ ID NO: 252 - protein, 25 aa, Escherichia coli
VYNIQDETGQFKPQPASNVSTAVPQ

SEQ ID NO: 253 - protein, 24 aa, Escherichia coli
VYNIQDETGQFKPQPASNSTAVPQ

SEQ ID NO: 254 - protein, 25 aa, Escherichia coli
VYNIQDETGQFKPQPASAFSTAVPQ

SEQ ID NO: 255 - protein, 25 aa, Escherichia coli
VYNIQDETGQFKPQPASAASTAVPQ

SEQ ID NO: 256 - protein, 25 aa, Escherichia coli
VYNIQDETGQFKPQPASAGSTAVPQ

SEQ ID NO: 257 - protein, 25 aa, Escherichia coli
VYNIQDETGQFKPQPASAVSTAVPQ

SEQ ID NO: 258 - protein, 24 aa, Escherichia coli
VYNIQDETGQFKPQPASASTAVPQ

SEQ ID NO: 259 - protein, 25 aa, Escherichia coli
VYNIQDETGQFKPQPASGFSTAVPQ

SEQ ID NO: 260 - protein, 25 aa, Escherichia coli
VYNIQDETGQFKPQPASGASTAVPQ

SEQ ID NO: 261 - protein, 25 aa, Escherichia coli
VYNIQDETGQFKPQPASGGSTAVPQ

SEQ ID NO: 262 - protein, 25 aa, Escherichia coli
VYNIQDETGQFKPQPASGVSTAVPQ

SEQ ID NO: 263 - protein, 24 aa, Escherichia coli
VYNIQDETGQFKPQPASGSTAVPQ

SEQ ID NO: 264 - protein, 25 aa, Escherichia coli
VYNIQDETGQFKPQPASVFSTAVPQ

SEQ ID NO: 265 - protein, 25 aa, Escherichia coli
VYNIQDETGQFKPQPASVASTAVPQ

SEQ ID NO: 266 - protein, 25 aa, Escherichia coli
VYNIQDETGQFKPQPASVGSTAVPQ

SEQ ID NO: 267 - protein, 25 aa, Escherichia coli
VYNIQDETGQFKPQPASVVSTAVPQ

SEQ ID NO: 268 - protein, 24 aa, Escherichia coli
VYNIQDETGQFKPQPASVSTAVPQ

SEQ ID NO: 269 - protein, 25 aa, Escherichia coli
VYNIQDETGQFKPQPASSFSTAVPQ

SEQ ID NO: 270 - protein, 25 aa, Escherichia coli
VYNIQDETGQFKPQPASSASTAVPQ

SEQ ID NO: 271 - protein, 25 aa, Escherichia coli
VYNIQDETGQFKPQPASSGSTAVPQ

SEQ ID NO: 272 - protein, 25 aa, Escherichia coli
VYNIQDETGQFKPQPASSVSTAVPQ

SEQ ID NO: 273 - protein, 24 aa, Escherichia coli
VYNIQDETGQFKPQPASSSTAVPQ

SEQ ID NO: 274 - protein, 25 aa, Escherichia coli
VYNIQDETGQFKPQPASTFSTAVPQ

SEQ ID NO: 275 - protein, 25 aa, Escherichia coli
VYNIQDETGQFKPQPASTASTAVPQ

SEQ ID NO: 276 - protein, 25 aa, Escherichia coli
VYNIQDETGQFKPQPASTGSTAVPQ

SEQ ID NO: 277 - protein, 25 aa, Escherichia coli
VYNIQDETGQFKPQPASTVSTAVPQ

SEQ ID NO: 278 - protein, 24 aa, Escherichia coli
VYNIQDETGQFKPQPASTSTAVPQ

SEQ ID NO: 279 - protein, 24 aa, Escherichia coli
VYNIQDETGQFKPQPASFSTAVPQ

SEQ ID NO: 280 - protein, 24 aa, Escherichia coli
VYNIQDETGQFKPQPASASTAVPQ

SEQ ID NO: 281 - protein, 24 aa, Escherichia coli
VYNIQDETGQFKPQPASGSTAVPQ

SEQ ID NO: 282 - protein, 24 aa, Escherichia coli
VYNIQDETGQFKPQPASVSTAVPQ

SEQ ID NO: 283 - protein, 23 aa, Escherichia coli
VYNIQDETGQFKPQPASSTAVPQ

SEQ ID NO: 284 - protein, 25 aa, Escherichia coli
VYNIQDETGQFKPFPASNFSTAVPQ

SEQ ID NO: 285 - protein, 25 aa, Escherichia coli
VYNIQDETGQFKPFPASNASTAVPQ

SEQ ID NO: 286 - protein, 25 aa, Escherichia coli
VYNIQDETGQFKPFPASNGSTAVPQ

SEQ ID NO: 287 - protein, 25 aa, Escherichia coli
VYNIQDETGQFKPFPASNVSTAVPQ

SEQ ID NO: 288 - protein, 24 aa, Escherichia coli
VYNIQDETGQFKPFPASNSTAVPQ

SEQ ID NO: 289 - protein, 25 aa, Escherichia coli
VYNIQDETGQFKPFPASAFSTAVPQ

SEQ ID NO: 290 - protein, 25 aa, Escherichia coli
VYNIQDETGQFKPFPASAASTAVPQ

SEQ ID NO: 291 - protein, 25 aa, Escherichia coli
VYNIQDETGQFKPFPASAGSTAVPQ

SEQ ID NO: 292 - protein, 25 aa, Escherichia coli
VYNIQDETGQFKPFPASAVSTAVPQ

SEQ ID NO: 293 - protein, 24 aa, Escherichia coli
VYNIQDETGQFKPFPASASTAVPQ

SEQ ID NO: 294 - protein, 25 aa, Escherichia coli
VYNIQDETGQFKPFPASGFSTAVPQ

SEQ ID NO: 295 - protein, 25 aa, Escherichia coli
VYNIQDETGQFKPFPASGASTAVPQ

SEQ ID NO: 296 - protein, 25 aa, Escherichia coli
VYNIQDETGQFKPFPASGGSTAVPQ

SEQ ID NO: 297 - protein, 25 aa, Escherichia coli
VYNIQDETGQFKPFPASGVSTAVPQ

SEQ ID NO: 298 - protein, 24 aa, Escherichia coli
VYNIQDETGQFKPFPASGSTAVPQ

SEQ ID NO: 299 - protein, 25 aa, Escherichia coli
VYNIQDETGQFKPFPASVFSTAVPQ

SEQ ID NO: 300 - protein, 25 aa, Escherichia coli
VYNIQDETGQFKPFPASVASTAVPQ

SEQ ID NO: 301 - protein, 25 aa, Escherichia coli
VYNIQDETGQFKPFPASVGSTAVPQ

SEQ ID NO: 302 - protein, 25 aa, Escherichia coli
VYNIQDETGQFKPFPASVVSTAVPQ

SEQ ID NO: 303 - protein, 24 aa, Escherichia coli
VYNIQDETGQFKPFPASVSTAVPQ

SEQ ID NO: 304 - protein, 25 aa, Escherichia coli
VYNIQDETGQFKPFPASSFSTAVPQ

SEQ ID NO: 305 - protein, 25 aa, Escherichia coli
VYNIQDETGQFKPFPASSASTAVPQ

SEQ ID NO: 306 - protein, 25 aa, Escherichia coli
VYNIQDETGQFKPFPASSGSTAVPQ

SEQ ID NO: 307 - protein, 25 aa, Escherichia coli
VYNIQDETGQFKPFPASSVSTAVPQ

SEQ ID NO: 308 - protein, 24 aa, Escherichia coli
VYNIQDETGQFKPFPASSSTAVPQ

SEQ ID NO: 309 - protein, 25 aa, Escherichia coli
VYNIQDETGQFKPFPASTFSTAVPQ

SEQ ID NO: 310 - protein, 25 aa, Escherichia coli
VYNIQDETGQFKPFPASTASTAVPQ

SEQ ID NO: 311 - protein, 25 aa, Escherichia coli
VYNIQDETGQFKPFPASTGSTAVPQ

SEQ ID NO: 312 - protein, 25 aa, Escherichia coli
VYNIQDETGQFKPFPASTVSTAVPQ

SEQ ID NO: 313 - protein, 24 aa, Escherichia coli
VYNIQDETGQFKPFPASTSTAVPQ

SEQ ID NO: 314 - protein, 24 aa, Escherichia coli
VYNIQDETGQFKPFPASFSTAVPQ

SEQ ID NO: 315 - protein, 24 aa, Escherichia coli
VYNIQDETGQFKPFPASASTAVPQ

SEQ ID NO: 316 - protein, 24 aa, Escherichia coli
VYNIQDETGQFKPFPASGSTAVPQ

SEQ ID NO: 317 - protein, 24 aa, Escherichia coli
VYNIQDETGQFKPFPASVSTAVPQ

SEQ ID NO: 318 - protein, 23 aa, Escherichia coli
VYNIQDETGQFKPFPASSTAVPQ

SEQ ID NO: 319 - protein, 24 aa, Escherichia coli
VYNIQDETGQFKPPASNFSTAVPQ

SEQ ID NO: 320 - protein, 24 aa, Escherichia coli
VYNIQDETGQFKPPASNASTAVPQ

SEQ ID NO: 321 - protein, 24 aa, Escherichia coli
VYNIQDETGQFKPPASNGSTAVPQ

SEQ ID NO: 322 - protein, 24 aa, Escherichia coli
VYNIQDETGQFKPPASNVSTAVPQ

SEQ ID NO: 323 - protein, 23 aa, Escherichia coli
VYNIQDETGQFKPPASNSTAVPQ

SEQ ID NO: 324 - protein, 24 aa, Escherichia coli
VYNIQDETGQFKPPASAFSTAVPQ

SEQ ID NO: 325 - protein, 24 aa, Escherichia coli
VYNIQDETGQFKPPASAASTAVPQ

SEQ ID NO: 326 - protein, 24 aa, Escherichia coli
VYNIQDETGQFKPPASAGSTAVPQ

SEQ ID NO: 327 - protein, 24 aa, Escherichia coli
VYNIQDETGQFKPPASAVSTAVPQ

SEQ ID NO: 328 - protein, 23 aa, Escherichia coli
VYNIQDETGQFKPPASASTAVPQ

SEQ ID NO: 329 - protein, 24 aa, Escherichia coli
VYNIQDETGQFKPPASGFSTAVPQ

SEQ ID NO: 330 - protein, 24 aa, Escherichia coli
VYNIQDETGQFKPPASGASTAVPQ

SEQ ID NO: 331 - protein, 24 aa, Escherichia coli
VYNIQDETGQFKPPASGGSTAVPQ

SEQ ID NO: 332 - protein, 24 aa, Escherichia coli
VYNIQDETGQFKPPASGVSTAVPQ

SEQ ID NO: 333 - protein, 23 aa, Escherichia coli
VYNIQDETGQFKPPASGSTAVPQ

SEQ ID NO: 334 - protein, 24 aa, Escherichia coli
VYNIQDETGQFKPPASVFSTAVPQ

SEQ ID NO: 335 - protein, 24 aa, Escherichia coli
VYNIQDETGQFKPPASVASTAVPQ

SEQ ID NO: 336 - protein, 24 aa, Escherichia coli
VYNIQDETGQFKPPASVGSTAVPQ

SEQ ID NO: 337 - protein, 24 aa, Escherichia coli
VYNIQDETGQFKPPASVVSTAVPQ

SEQ ID NO: 338 - protein, 23 aa, Escherichia coli
VYNIQDETGQFKPPASVSTAVPQ

SEQ ID NO: 339 - protein, 24 aa, Escherichia coli
VYNIQDETGQFKPPASSFSTAVPQ

SEQ ID NO: 340 - protein, 24 aa, Escherichia coli
VYNIQDETGQFKPPASSASTAVPQ

SEQ ID NO: 341 - protein, 24 aa, Escherichia coli
VYNIQDETGQFKPPASSGSTAVPQ

SEQ ID NO: 342 - protein, 24 aa, Escherichia coli
VYNIQDETGQFKPPASSVSTAVPQ

SEQ ID NO: 343 - protein, 23 aa, Escherichia coli
VYNIQDETGQFKPPASSSTAVPQ

SEQ ID NO: 344 - protein, 24 aa, Escherichia coli
VYNIQDETGQFKPPASTFSTAVPQ

SEQ ID NO: 345 - protein, 24 aa, Escherichia coli
VYNIQDETGQFKPPASTASTAVPQ

SEQ ID NO: 346 - protein, 24 aa, Escherichia coli
VYNIQDETGQFKPPASTGSTAVPQ

SEQ ID NO: 347 - protein, 24 aa, Escherichia coli
VYNIQDETGQFKPPASTVSTAVPQ

SEQ ID NO: 348 - protein, 23 aa, Escherichia coli
VYNIQDETGQFKPPASTSTAVPQ

SEQ ID NO: 349 - protein, 23 aa, Escherichia coli
VYNIQDETGQFKPPASFSTAVPQ

SEQ ID NO: 350 - protein, 23 aa, Escherichia coli
VYNIQDETGQFKPPASASTAVPQ

SEQ ID NO: 351 - protein, 23 aa, Escherichia coli
VYNIQDETGQFKPPASGSTAVPQ

SEQ ID NO: 352 - protein, 23 aa, Escherichia coli
VYNIQDETGQFKPPASVSTAVPQ

SEQ ID NO: 353 - protein, 22 aa, Escherichia coli
VYNIQDETGQFKPPASSTAVPQ

SEQ ID NO: 354 - protein, 23 aa, Escherichia coli
VYNIQDETGQFKGGSNFSTAVPQ

SEQ ID NO: 355 - protein, 23 aa, Escherichia coli
VYNIQDETGQFKGGSNASTAVPQ

SEQ ID NO: 356 - protein, 23 aa, Escherichia coli
VYNIQDETGQFKGGSNGSTAVPQ

SEQ ID NO: 357 - protein, 23 aa, Escherichia coli
VYNIQDETGQFKGGSNVSTAVPQ

SEQ ID NO: 358 - protein, 22 aa, Escherichia coli
VYNIQDETGQFKGGSNSTAVPQ

SEQ ID NO: 359 - protein, 23 aa, Escherichia coli
VYNIQDETGQFKGGSAFSTAVPQ

SEQ ID NO: 360 - protein, 23 aa, Escherichia coli
VYNIQDETGQFKGGSAASTAVPQ

SEQ ID NO: 361 - protein, 23 aa, Escherichia coli
VYNIQDETGQFKGGSAGSTAVPQ

SEQ ID NO: 362 - protein, 23 aa, Escherichia coli
VYNIQDETGQFKGGSAVSTAVPQ

SEQ ID NO: 363 - protein, 22 aa, Escherichia coli
VYNIQDETGQFKGGSASTAVPQ

SEQ ID NO: 364 - protein, 23 aa, Escherichia coli
VYNIQDETGQFKGGSGFSTAVPQ

SEQ ID NO: 365 - protein, 23 aa, Escherichia coli
VYNIQDETGQFKGGSGASTAVPQ

SEQ ID NO: 366 - protein, 23 aa, Escherichia coli
VYNIQDETGQFKGGSGGSTAVPQ

SEQ ID NO: 367 - protein, 23 aa, Escherichia coli
VYNIQDETGQFKGGSGVSTAVPQ

SEQ ID NO: 368 - protein, 22 aa, Escherichia coli
VYNIQDETGQFKGGSGSTAVPQ

SEQ ID NO: 369 - protein, 23 aa, Escherichia coli
VYNIQDETGQFKGGSVFSTAVPQ

SEQ ID NO: 370 - protein, 23 aa, Escherichia coli
VYNIQDETGQFKGGSVASTAVPQ

SEQ ID NO: 371 - protein, 23 aa, Escherichia coli
VYNIQDETGQFKGGSVGSTAVPQ

SEQ ID NO: 372 - protein, 23 aa, Escherichia coli
VYNIQDETGQFKGGSVVSTAVPQ

SEQ ID NO: 373 - protein, 22 aa, Escherichia coli
VYNIQDETGQFKGGSVSTAVPQ

SEQ ID NO: 374 - protein, 23 aa, Escherichia coli
VYNIQDETGQFKGGSSFSTAVPQ

SEQ ID NO: 375 - protein, 23 aa, Escherichia coli
VYNIQDETGQFKGGSSASTAVPQ

SEQ ID NO: 376 - protein, 23 aa, Escherichia coli
VYNIQDETGQFKGGSSGSTAVPQ

SEQ ID NO: 377 - protein, 23 aa, Escherichia coli
VYNIQDETGQFKGGSSVSTAVPQ

SEQ ID NO: 378 - protein, 22 aa, Escherichia coli
VYNIQDETGQFKGGSSSTAVPQ

SEQ ID NO: 379 - protein, 23 aa, Escherichia coli
VYNIQDETGQFKGGSTFSTAVPQ

SEQ ID NO: 380 - protein, 23 aa, Escherichia coli
VYNIQDETGQFKGGSTASTAVPQ

SEQ ID NO: 381 - protein, 23 aa, Escherichia coli
VYNIQDETGQFKGGSTGSTAVPQ

SEQ ID NO: 382 - protein, 23 aa, Escherichia coli
VYNIQDETGQFKGGSTVSTAVPQ

SEQ ID NO: 383 - protein, 22 aa, Escherichia coli
VYNIQDETGQFKGGSTSTAVPQ

SEQ ID NO: 384 - protein, 22 aa, Escherichia coli
VYNIQDETGQFKGGSFSTAVPQ

SEQ ID NO: 385 - protein, 22 aa, Escherichia coli
VYNIQDETGQFKGGSASTAVPQ

SEQ ID NO: 386 - protein, 22 aa, Escherichia coli
VYNIQDETGQFKGGSGSTAVPQ

SEQ ID NO: 387 - protein, 22 aa, Escherichia coli
VYNIQDETGQFKGGSVSTAVPQ

SEQ ID NO: 388 - protein, 21 aa, Escherichia coli
VYNIQDETGQFKGGSSTAVPQ

SEQ ID NO: 389 - DNA, 786 bp, Escherichia coli
  source 1..786: mol_type = genomic DNA
tgtctgaccgcaccgccgaaagaagcggcacgtccgaccctgatgccgcgtgcacagtcttataaagatctgacccatctgccggctccgacgggcaaaatttttgttagcgtctataacatccaggacgaaaccggtcaatttaaaccgtacccggcgagtaatttctccacggccgttccgcagagtgcaaccgctatgctggtcacggcactgaaagattcccgttggttcattccgctggaacgccagggcctgcaaaacctgctgaatgaacgtaaaattatccgcgcagctcaggaaaacggtaccgtggccattaacaatcgtattccgctgcaaagcctgaccgccgcaaacatcatggttgaaggctctatcatcggttacgaatcaaacgtcaaatcgggcggtgtgggcgcacgttattttggcattggtgctgatacccagtaccaactggaccagatcgcagttaacctgcgcgtggttaatgtcagcaccggcgaaattctgagctctgtgaataccagcaaaacgatcctgtcttacgaagtgcaggctggtgtttttcgtttcattgattatcaacgcctgctggaaggcgaagtcggttacacctcaaacgaaccggtgatgctgtgtctgatgtcggcgattgaaacgggtgttattttcctgatcaatgatggcatcgaccgtggtctgtgggatctgcagaacaaagccgaacgtcaaaatgacattctggtgaaataccgccacatgagtgttccgccggaatcc

SEQ ID NO: 390 - protein, 262 aa, Escherichia coli
CLTAPPKEAARPTLMPRAQSYKDLTHLPAPTGKIFVSVYNIQDETGQFKPYPASNFSTAVPQSATAMLVTALKDSRWFIPLERQGLQNLLNERKIIRAAQENGTVAINNRIPLQSLTAANIMVEGSIIGYESNVKSGGVGARYFGIGADTQYQLDQIAVNLRVVNVSTGEILSSVNTSKTILSYEVQAGVFRFIDYQRLLEGEVGYTSNEPVMLCLMSAIETGVIFLINDGIDRGLWDLQNKAERQNDILVKYRHMSVPPES

SEQ ID NO: 391 - protein, 248 aa, Citrobacter koseri
MPRAQSYKDLTHLPMPTGKIFVSVYNIQDETGQFKPYPASNFSTAVPQSATAMLVTALKDSRWFIPLERQGLQNLLNERKIIRAAQENGTVAINNRIPLQSLTAANIMVEGSIIGYESNVKSGGVGARYFGIGADTQYQLDQIAVNLRVVNVSTGEILSSVNTSKTILSYEVQAGVFRFIDYQRLLEGEIGYTSNEPVMLCLMSAIETGVIFLINDGIDRGLWDLQNKAERQNDILVKYRHMSVPPES

SEQ ID NO: 392 - protein, 223 aa, Salmonella enterica
CLTAPPKQAAKPTLMPRAQSYKDLTHLPAPTGKIFVSVYNIQDETGQFKPYPASNFSTAVPQSATAMLVTALKDSRWFIPLERQGLQNLLNERKIIRAAQENGTVAMNNRIPLQSLTAANIMVEGSIIGYESNVKSGGVGARYFGIGADTQYQLDQIAVNLRVVNVSTGEILSSVNTSKTILSYEVQAGVFRFIDYQRLLEGEIGYTSNEPVMLCLMSAIETG

SEQ ID NO: 393 - protein, 262 aa, Citrobacter amalonaticus
CLTAPPKEAAKPTLMPRAQSYKDLTHLPIPTGKIFVSVYNIQDETGQFKPYPASNFSTAVPQSATAMLVTALKDSRWFVPLERQGLQNLLNERKIIRAAQENGTVAINNRIPLQSLTAANIMVEGSIIGYESNVKSGGVGARYFGIGADTQYQLDQIAVNLRVVNVSTGEILSSVNTSKTILSYEVQAGVFRFIDYQRLLEGEIGYTSNEPVMLCLMSAIETGVIFLINDGIDRGLWDLQNKADRQNDILVKYRHMSVPPES

SEQ ID NO: 394 - protein, 262 aa, Citrobacter rodentium
CLTTPPKEAAKPTLMPRAQSYKDLTHLPVPTGKIFVSVYNIQDETGQFKPYPASNFSTAVPQSATAMLVTALKDSRWFIPLERQGLQNLLNERKIIRAAQENGTVAINNRIPLPSLTAANIMVEGSIIGYESNVKSGGAGARYFGIGADTQYQLDQIAVNLRVVNVSTGEILSSVNTSKTILSYEVQAGVFRFIDYQRLLEGEIGYTSNEPVMLCLMSAIETGVIFLINDGIDRGLWDLQNKADRQNDILVKYRQMSVPPES

SEQ ID NO: 395 - protein, 262 aa, Enterobacter asburiae
CLTAPPKEAAKPTLMPRAQSYRDLTHLPAPTGKIFVSVYNIQDETGQFKPYPASNFSTAVPQSATAMLVTALKDSHWFIPLERQGLQNLLNERKIIRAAQENGTVANNNRMPLQSLAAANVMIEGSIIGYESNVKSGGVGARYFGIGADTQYQLDQIAVNLRVVNVSTGEVLSSVNTSKTILSYEVQAGVFRFIDYQRLLEGEIGYTSNEPVMMCLMSAIETGVIFLINDGIDRGLWDLQNKADAQNPVLVKYRDMSVPPES

SEQ ID NO: 396 - DNA, 1830 bp, unidentified
  source 1..1830: mol_type = genomic DNA; note = Bacillus subtilis phage phi29
atgaaacacatgccgcgtaaaatgtatagctgcgcgtttgaaaccacgaccaaagtggaagattgtcgcgtttgggcctatggctacatgaacatcgaagatcattctgaatacaaaatcggtaacagtctggatgaatttatggcatgggtgctgaaagttcaggcggatctgtacttccacaacctgaaatttgatggcgcattcattatcaactggctggaacgtaatggctttaaatggagcgcggatggtctgccgaacacgtataataccattatctctcgtatgggccagtggtatatgattgatatctgcctgggctacaaaggtaaacgcaaaattcataccgtgatctatgatagcctgaaaaaactgccgtttccggtgaagaaaattgcgaaagatttcaaactgacggttctgaaaggcgatattgattatcacaaagaacgtccggttggttacaaaatcaccccggaagaatacgcatacatcaaaaacgatatccagatcatcgcagaagcgctgctgattcagtttaaacagggcctggatcgcatgaccgcgggcagtgatagcctgaaaggtttcaaagatatcatcacgaccaaaaaattcaaaaaagtgttcccgacgctgagcctgggtctggataaagaagttcgttatgcctaccgcggcggttttacctggctgaacgatcgtttcaaagaaaaagaaattggcgagggtatggtgtttgatgttaatagtctgtatccggcacagatgtacagccgcctgctgccgtatggcgaaccgatcgtgttcgagggtaaatatgtttgggatgaagattacccgctgcatattcagcacatccgttgtgaatttgaactgaaagaaggctatattccgaccattcagatcaaacgtagtcgcttctataagggtaacgaatacctgaaaagctctggcggtgaaatcgcggatctgtggctgagtaacgtggatctggaactgatgaaagaacactacgatctgtacaacgttgaatacatcagcggcctgaaatttaaagccacgaccggtctgttcaaagatttcatcgataaatggacctacatcaaaacgacctctgaaggcgcgattaaacagctggccaaactgatgctgaacagcctgtatggcaaattcgcctctaatccggatgtgaccggtaaagttccgtacctgaaagaaaatggcgcactgggttttcgcctgggcgaagaagaaacgaaagatccggtgtataccccgatgggtgttttcattacggcctgggcacgttacacgaccatcaccgcggcccaggcatgctatgatcgcattatctactgtgataccgattctattcatctgacgggcaccgaaatcccggatgtgattaaagatatcgttgatccgaaaaaactgggttattgggcccacgaaagtacgtttaaacgtgcaaaatacctgcgccagaaaacctacatccaggatatctacatgaaagaagtggatggcaaactggttgaaggttctccggatgattacaccgatatcaaattcagtgtgaaatgcgccggcatgacggataaaatcaaaaaagaagtgaccttcgaaaacttcaaagttggtttcagccgcaaaatgaaaccgaaaccggtgcaggttccgggcggtgtggttctggtggatgatacgtttaccattaaatctggcggtagtgcgtggagccatccgcagttcgaaaaaggcggtggctctggtggcggttctggcggtagtgcctggagccacccgcagtttgaaaaataataa

SEQ ID NO: 397 - protein, 608 aa, unidentified
  source 1..608: note = Bacillus subtilis phage phi29
MKHMPRKMYSCAFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIKSGGSAWSHPQFEKGGGSGGGSGGSAWSHPQFEK

SEQ ID NO: 398 - DNA, 1390 bp, Escherichia coli
  source 1..1390: mol_type = genomic DNA
atgatgaacgatggcaaacagcagagcaccttcctgtttcatgattatgaaaccttcggtacccatccggccctggatcgtccggcgcagtttgcggccattcgcaccgatagcgaattcaatgtgattggcgaaccggaagtgttttattgcaaaccggccgatgattatctgccgcagccgggtgcggtgctgattaccggtattaccccgcaggaagcgcgcgcgaaaggtgaaaacgaagcggcgtttgccgcgcgcattcatagcctgtttaccgtgccgaaaacctgcattctgggctataacaatgtgcgcttcgatgatgaagttacccgtaatatcttttatcgtaacttttatgatccgtatgcgtggagctggcagcatgataacagccgttgggatctgctggatgtgatgcgcgcgtgctatgcgctgcgcccggaaggcattaattggccggaaaacgatgatggcctgccgagctttcgtctggaacatctgaccaaagccaacggcattgaacatagcaatgcccatgatgcgatggccgatgtttatgcgaccattgcgatggcgaaactggttaaaacccgtcagccgcgcctgtttgattatctgtttacccaccgtaacaaacacaaactgatggcgctgattgatgttccgcagatgaaaccgctggtgcatgtgagcggcatgtttggcgcctggcgcggcaacaccagctgggtggccccgctggcctggcacccggaaaatcgtaacgccgtgattatggttgatctggccggtgatattagcccgctgctggaactggatagcgataccctgcgtgaacgcctgtataccgccaaaaccgatctgggcgataatgccgccgtgccggtgaaactggttcacattaacaaatgcccggtgctggcccaggcgaacaccctgcgcccggaagatgcggatcgtctgggtattaatcgccagcattgtctggataatctgaaaatcctgcgtgaaaacccgcaggtgcgtgaaaaagtggtggcgatcttcgcggaagcggaaccgttcaccccgagcgataacgtggatgcgcagctgtataacggcttctttagcgatgccgatcgcgcggcgatgaaaatcgttctggaaaccgaaccgcgcaatctgccggcgctggatattacctttgttgataaacgtattgaaaaactgctgtttaattatcgtgcgcgcaattttccgggtaccctggattatgccgaacagcagcgttggctggaacatcgtcgtcaggttttcaccccggaatttctgcagggttatgcggatgaactgcagatgctggttcagcagtatgccgatgataaagaaaaagtggcgctgc

SEQ ID NO: 399 - protein, 485 aa, Escherichia coli
MMNDGKQQSTFLFHDYETFGTHPALDRPAQFAAIRTDSEFNVIGEPEVFYCKPADDYLPQPGAVLITGITPQEARAKGENEAAFAARIHSLFTVPKTCILGYNNVRFDDEVTRNIFYRNFYDPYAWSWQHDNSRWDLLDVMRACYALRPEGINWPENDDGLPSFRLEHLTKANGIEHSNAHDAMADVYATIAMAKLVKTRQPRLFDYLFTHRNKHKLMALIDVPQMKPLVHVSGMFGAWRGNTSWVAPLAWHPENRNAVIMVDLAGDISPLLELDSDTLRERLYTAKTDLGDNAAVPVKLVHINKCPVLAQANTLRPEDADRLGINRQHCLDNLKILRENPQVREKVVAIFAEAEPFTPSDNVDAQLYNGFFSDADRAAMKIVLETEPRNLPALDITFVDKRIEKLLFNYRARNFPGTLDYAEQQRWLEHRRQVFTPEFLQGYADELQMLVQQYADDKEKVALLKALWQYAEEIVSGSGHHHHHH

SEQ ID NO: 400 - DNA, 804 bp, Escherichia coli
  source 1..804: mol_type = genomic DNA
atgaaatttgtctcttttaatatcaacggcctgcgcgccagacctcaccagcttgaagccatcgtcgaaaagcaccaaccggatgtgattggcctgcaggagacaaaagttcatgacgatatgtttccgctcgaagaggtggcgaagctcggctacaacgtgttttatcacgggcagaaaggccattatggcgtggcgctgctgaccaaagagacgccgattgccgtgcgtcgcggctttcccggtgacgacgaagaggcgcagcggcggattattatggcggaaatcccctcactgctgggtaatgtcaccgtgatcaacggttacttcccgcagggtgaaagccgcgaccatccgataaaattcccggcaaaagcgcagttttatcagaatctgcaaaactacctggaaaccgaactcaaacgtgataatccggtactgattatgggcgatatgaatatcagccctacagatctggatatcggcattggcgaagaaaaccgtaagcgctggctgcgtaccggtaaatgctctttcctgccggaagagcgcgaatggatggacaggctgatgagctgggggttggtcgataccttccgccatgcgaatccgcaaacagcagatcgtttctcatggtttgattaccgctcaaaaggttttgacgataaccgtggtctgcgcatcgacctgctgctcgccagccaaccgctggcagaatgttgcgtagaaaccggcatcgactatgaaatccgcagcatggaaaaaccgtccgatcacgcccccgtctgggcgaccttccgccgc

SEQ ID NO: 401 - protein, 268 aa, Escherichia coli
MKFVSFNINGLRARPHQLEAIVEKHQPDVIGLQETKVHDDMFPLEEVAKLGYNVFYHGQKGHYGVALLTKETPIAVRRGFPGDDEEAQRRIIMAEIPSLLGNVTVINGYFPQGESRDHPIKFPAKAQFYQNLQNYLETELKRDNPVLIMGDMNISPTDLDIGIGEENRKRWLRTGKCSFLPEEREWMDRLMSWGLVDTFRHANPQTADRFSWFDYRSKGFDDNRGLRIDLLLASQPLAECCVETGIDYEIRSMEKPSDHAPVWATFRR

SEQ ID NO: 402 - DNA, 1275 bp, Thermus thermophilus
  source 1..1275: mol_type = genomic DNA
atgtttcgtcgtaaagaagatctggatccgccgctggcactgctgccgctgaaaggcctgcgcgaagccgccgcactgctggaagaagcgctgcgtcaaggtaaacgcattcgtgttcacggcgactatgatgcggatggcctgaccggcaccgcgatcctggttcgtggtctggccgccctgggtgcggatgttcatccgtttatcccgcaccgcctggaagaaggctatggtgtcctgatggaacgcgtcccggaacatctggaagcctcggacctgtttctgaccgttgactgcggcattaccaaccatgcggaactgcgcgaactgctggaaaatggcgtggaagtcattgttaccgatcatcatacgccgggcaaaacgccgccgccgggtctggtcgtgcatccggcgctgacgccggatctgaaagaaaaaccgaccggcgcaggcgtggcgtttctgctgctgtgggcactgcatgaacgcctgggcctgccgccgccgctggaatacgcggacctggcagccgttggcaccattgccgacgttgccccgctgtggggttggaatcgtgcactggtgaaagaaggtctggcacgcatcccggcttcatcttgggtgggcctgcgtctgctggctgaagccgtgggctataccggcaaagcggtcgaagtcgctttccgcatcgcgccgcgcatcaatgcggcttcccgcctgggcgaagcggaaaaagccctgcgcctgctgctgacggatgatgcggcagaagctcaggcgctggtcggcgaactgcaccgtctgaacgcccgtcgtcagaccctggaagaagcgatgctgcgcaaactgctgccgcaggccgacccggaagcgaaagccatcgttctgctggacccggaaggccatccgggtgttatgggtattgtggcctctcgcatcctggaagcgaccctgcgcccggtctttctggtggcccagggcaaaggcaccgtgcgttcgctggctccgatttccgccgtcgaagcactgcgcagcgcggaagatctgctgctgcgttatggtggtcataaagaagcggcgggtttcgcaatggatgaagcgctgtttccggcgttcaaagcacgcgttgaagcgtatgccgcacgtttcccggatccggttcgtgaagtggcactgctggatctgctgccggaaccgggcctgctgccgcaggtgttccgtgaactggcactgctggaaccgtatggtgaaggtaacccggaaccgctgttcctg

SEQ ID NO: 403 - protein, 425 aa, Thermus thermophilus
MFRRKEDLDPPLALLPLKGLREAAALLEEALRQGKRIRVHGDYDADGLTGTAILVRGLAALGADVHPFIPHRLEEGYGVLMERVPEHLEASDLFLTVDCGITNHAELRELLENGVEVIVTDHHTPGKTPPPGLVVHPALTPDLKEKPTGAGVAFLLLWALHERLGLPPPLEYADLAAVGTIADVAPLWGWNRALVKEGLARIPASSWVGLRLLAEAVGYTGKAVEVAFRIAPRINAASRLGEAEKALRLLLTDDAAEAQALVGELHRLNARRQTLEEAMLRKLLPQADPEAKAIVLLDPEGHPGVMGIVASRILEATLRPVFLVAQGKGTVRSLAPISAVEALRSAEDLLLRYGGHKEAAGFAMDEALFPAFKARVEAYAARFPDPVREVALLDLLPEPGLLPQVFRELALLEPYGEGNPEPLFL

SEQ ID NO: 404 - DNA, 738 bp, unidentified
  source 1..738: mol_type = genomic DNA; note = Bacteriophage lambda
tccggaagcggctctggtagtggttctggcatgacaccggacattatcctgcagcgtaccgggatcgatgtgagagctgtcgaacagggggatgatgcgtggcacaaattacggctcggcgtcatcaccgcttcagaagttcacaacgtgatagcaaaaccccgctccggaaagaagtggcctgacatgaaaatgtcctacttccacaccctgcttgctgaggtttgcaccggtgtggctccggaagttaacgctaaagcactggcctggggaaaacagtacgagaacgacgccagaaccctgtttgaattcacttccggcgtgaatgttactgaatccccgatcatctatcgcgacgaaagtatgcgtaccgcctgctctcccgatggtttatgcagtgacggcaacggccttgaactgaaatgcccgtttacctcccgggatttcatgaagttccggctcggtggtttcgaggccataaagtcagcttacatggcccaggtgcagtacagcatgtgggtgacgcgaaaaaatgcctggtactttgccaactatgacccgcgtatgaagcgtgaaggcctgcattatgtcgtgattgagcgggatgaaaagtacatggcgagttttgacgagatcgtgccggagttcatcgaaaaaatggacgaggcactggctgaaattggttttgtatttggggagcaatggcgatctggctctggttccggcagcggttccgga

SEQ ID NO: 405 - protein, 226 aa, unidentified
  source 1..226: note = Bacteriophage lambda
MTPDIILQRTGIDVRAVEQGDDAWHKLRLGVITASEVHNVIAKPRSGKKWPDMKMSYFHTLLAEVCTGVAPEVNAKALAWGKQYENDARTLFEFTSGVNVTESPIIYRDESMRTACSPDGLCSDGNGLELKCPFTSRDFMKFRLGGFEAIKSAYMAQVQYSMWVTRKNAWYFANYDPRMKREGLHYVVIERDEKYMASFDEIVPEFIEKMDEALAEIGFVFGEQWR

SEQ ID NO: 406 - protein, 760 aa, Methanococcoides burtonii
MMIRELDIPRDIIGFYEDSGIKELYPPQAEAIEMGLLEKKNLLAAIPTASGKTLLAELAMIKAIREGGKALYIVPLRALASEKFERFKELAPFGIKVGISTGDLDSRADWLGVNDIIVATSEKTDSLLRNGTSWMDEITTVVVDEIHLLDSKNRGPTLEVTITKLMRLNPDVQVVALSATVGNAREMADWLGAALVLSEWRPTDLHEGVLFGDAINFPGSQKKIDRLEKDDAVNLVLDTIKAEGQCLVFESSRRNCAGFAKTASSKVAKILDNDIMIKLAGIAEEVESTGETDTAIVLANCIRKGVAFHHAGLNSNHRKLVENGFRQNLIKVISSTPTLAAGLNLPARRVIIRSYRRFDSNFGMQPIPVLEYKQMAGRAGRPHLDPYGESVLLAKTYDEFAQLMENYVEADAEDIWSKLGTENALRTHVLSTIVNGFASTRQELFDFFGATFFAYQQDKWMLEEVINDCLEFLIDKAMVSETEDIEDASKLFLRGTRLGSLVSMLYIDPLSGSKIVDGFKDIGKSTGGNMGSLEDDKGDDITVTDMTLLHLVCSTPDMRQLYLRNTDYTIVNEYIVAHSDEFHEIPDKLKETDYEWFMGEVKTAMLLEEWVTEVSAEDITRHFNVGEGDIHALADTSEWLMHAAAKLAELLGVEYSSHAYSLEKRIRYGSGLDLMELVGIRGVGRVRARKLYNAGFVSVAKLKGADISVLSKLVGPKVAYNILSGIGVRVNDKHFNSAPISSNTLDTLLDKNQKTFNDFQ

SEQ ID NO: 407 - protein, 707 aa, unidentified
  source 1..707: note = Cenarchaeum symbiosum
MRISELDIPRPAIEFLEGEGYKKLYPPQAAAAKAGLTDGKSVLVSAPTASGKTLIAAIAMISHLSRNRGKAVYLSPLRALAAEKFAEFGKIGGIPLGRPVRVGVSTGDFEKAGRSLGNNDILVLTNERMDSLIRRRPDWMDEVGLVIADEIHLIGDRSRGPTLEMVLTKLRGLRSSPQVVALSATISNADEIAGWLDCTLVHSTWRPVPLSEGVYQDGEVAMGDGSRHEVAATGGGPAVDLAAESVAEGGQSLIFADTRARSASLAAKASAVIPEAKGADAAKLAAAAKKIISSGGETKLAKTLAELVEKGAAFHHAGLNQDCRSVVEEEFRSGRIRLLASTPTLAAGVNLPARRVVISSVMRYNSSSGMSEPISILEYKQLCGRAGRPQYDKSGEAIVVGGVNADEIFDRYIGGEPEPIRSAMVDDRALRIHVLSLVTTSPGIKEDDVTEFFLGTLGGQQSGESTVKFSVAVALRFLQEEGMLGRRGGRLAATKMGRLVSRLYMDPMTAVTLRDAVGEASPGRMHTLGFLHLVSECSEFMPRFALRQKDHEVAEMMLEAGRGELLRPVYSYECGRGLLALHRWIGESPEAKLAEDLKFESGDVHRMVESSGWLLRCIWEISKHQERPDLLGELDVLRSRVAYGIKAELVPLVSIKGIGRVRSRRLFRGGIKGPGDLAAVPVERLSRVEGIGATLANNIKSQLRKGG

SEQ ID NO: 408 - protein, 720 aa, Thermococcus gammatolerans
MKVDELPVDERLKAVLKERGIEELYPPQAEALKSGALEGRNLVLAIPTASGKTLVSEIVMVNKLIQEGGKAVYLVPLKALAEEKYREFKEWEKLGLKVAATTGDYDSTDDWLGRYDIIVATAEKFDSLLRHGARWINDVKLVVADEVHLIGSYDRGATLEMILTHMLGRAQILALSATVGNAEELAEWLDASLVVSDWRPVQLRRGVFHLGTLIWEDGKVESYPENWYSLVVDAVKRGKGALVFVNTRRSAEKEALALSKLVSSHLTKPEKRALESLASQLEDNPTSEKLKRALRGGVAFHHAGLSRVERTLIEDAFREGLIKVITATPTLSAGVNLPSFRVIIRDTKRYAGFGWTDIPVLEIQQMMGRAGRPRYDKYGEAIIVARTDEPGKLMERYIRGKPEKLFSMLANEQAFRSQVLALITNFGIRSFPELVRFLERTFYAHQRKDLSSLEYKAKEVVYFLIENEFIDLDLEDRFIPLPFGKRTSQLYIDPLTAKKFKDAFPAIERNPNPFGIFQLIASTPDMATLTARRREMEDYLDLAYELEDKLYASIPYYEDSRFQGFLGQVKTAKVLLDWINEVPEARIYETYSIDPGDLYRLLELADWLMYSLIELYKLFEPKEEILNYLRDLHLRLRHGVREELLELVRLPNIGRKRARALYNAGFRSVEAIANAKPAELLAVEGIGAKILDGIYRHLGIEKRVTEEKPKRKGTLEDFLR

SEQ ID NO: 409 - protein, 799 aa, Methanospirillum hungatei
MEIASLPLPDSFIRACHAKGIRSLYPPQAECIEKGLLEGKNLLISIPTASGKTLLAEMAMWSRIAAGGKCLYIVPLRALASEKYDEFSKKGVIRVGIATGDLDRTDAYLGENDIIVATSEKTDSLLRNRTPWLSQITCIVLDEVHLIGSENRGATLEMVITKLRYTNPVMQIIGLSATIGNPAQLAEWLDATLITSTWRPVDLRQGVYYNGKIRFSDSERPIQGKTKHDDLNLCLDTIEEGGQCLVFVSSRRNAEGFAKKAAGALKAGSPDSKALAQELRRLRDRDEGNVLADCVERGAAFHHAGLIRQERTIIEEGFRNGYIEVIAATPTLAAGLNLPARRVIIRDYNRFASGLGMVPIPVGEYHQMAGRAGRPHLDPYGEAVLLAKDAPSVERLFETFIDAEAERVDSQCVDDASLCAHILSLIATGFAHDQEALSSFMERTFYFFQHPKTRSLPRLVADAIRFLTTAGMVEERENTLSATRLGSLVSRLYLNPCTARLILDSLKSCKTPTLIGLLHVICVSPDMQRLYLKAADTQLLRTFLFKHKDDLILPLPFEQEEEELWLSGLKTALVLTDWADEFSEGMIEERYGIGAGDLYNIVDSGKWLLHGTERLVSVEMPEMSQVVKTLSVRVHHGVKSELLPLVALRNIGRVRARTLYNAGYPDPEAVARAGLSTIARIIGEGIARQVIDEITGVKRSGIHSSDDDYQQKTPELLTDIPGIGKKMAEKLQNAGIITVSDLLTADEVLLSDVLGAARARKVLAFLSNSEKENSSSDKTEEIPDTQKIRGQSSWEDFGC

SEQ ID NO: 410 - protein, 1756 aa, Escherichia coli
MMSIAQVRSAGSAGNYYTDKDNYYVLGSMGERWAGKGAEQLGLQGSVDKDVFTRLLEGRLPDGADLSRMQDGSNKHRPGYDLTFSAPKSVSMMAMLGGDKRLIDAHNQAVDFAVRQVEALASTRVMTDGQSETVLTGNLVMALFNHDTSRDQEPQLHTHAVVANVTQHNGEWKTLSSDKVGKTGFIENVYANQIAFGRLYREKLKEQVEALGYETEVVGKHGMWEMPGVPVEAFSGRSQAIREAVGEDASLKSRDVAALDTRKSKQHVDPEIRMAEWMQTLKETGFDIRAYRDAADQRTEIRTQAPGPASQDGPDVQQAVTQAIAGLSERKVQFTYTDVLARTVGILPPENGVIERARAGIDEAISREQLIPLDREKGLFTSGIHVLDELSVRALSRDIMKQNRVTVHPEKSVPRTAGYSDAVSVLAQDRPSLAIVSGQGGAAGQRERVAELVMMAREQGREVQIIAADRRSQMNLKQDERLSGELITGRRQLLEGMAFTPGSTVIVDQGEKLSLKETLTLLDGAARHNVQVLITDSGQRTGTGSALMAMKDAGVNTYRWQGGEQRPATIISEPDRNVRYARLAGDFAASVKAGEESVAQVSGVREQAILTQAIRSELKTQGVLGHPEVTMTALSPVWLDSRSRYLRDMYRPGMVMEQWNPETRSHDRYVIDRVTAQSHSLTLRDAQGETQVVRISSLDSSWSLFRPEKMPVADGERLRVTGKIPGLRVSGGDRLQVASVSEDAMTVVVPGRAEPASLPVSDSPFTALKLENGWVETPGHSVSDSATVFASVTQMAMDNATLNGLARSGRDVRLYSSLDETRTAEKLARHPSFTVVSEQIKARAGETLLETAISLQKAGLHTPAQQAIHLALPVLESKNLAFSMVDLLTEAKSFAAEGTGFTELGGEINAQIKRGDLLYVDVAKGYGTGLLVSRASYEAEKSILRHILEGKEAVTPLMERVPGELMETLTSGQRAATRMILETSDRFTVVQGYAGVGKTTQFRAVMSAVNMLPASERPRVVGLGPTHRAVGEMRSAGVDAQTLASFLHDTQLQQRSGETPDFSNTLFLLDESSMVGNTEMARAYALIAAGGGRAVASGDTDQLQAIAPGQSFRLQQTRSAADVVIMKEIVRQTPELREAVYSLINRDVERALSGLESVKPSQVPRLEGAWAPEHSVTEFSHSQEAKLAEAQQKAMLKGEAFPDIPMTLYEAIVRDYTGRTPEAREQTLIVTHLNEDRRVLNSMIHDAREKAGELGKEQVMVPVLNTANIRDGELRRLSTWEKNPDALALVDNVYHRIAGISKDDGLITLQDAEGNTRLISPREAVAEGVTLYTPDKIRVGTGDRMRFTKSDRERGYVANSVWTVTAVSGDSVTLSDGQQTRVIRPGQERAEQHIDLAYAITAHGAQGASETFAIALEGTEGNRKLMAGFESAYVALSRMKQHVQVYTDNRQGWTDAINNAVQKGTAHDVLEPKPDREVMNAQRLFSTARELRDVAAGRAVLRQAGLAGGDSPARFIAPGRKYPQPYVALPAFDRNGKSAGIWLNPLTTDDGNGLRGFSGEGRVKGSGDAQFVALQGSRNGESLLADNMQDGVRIARDNPDSGVVVRIAGEGRPWNPGAITGGRVWGDIPDNSVQPGAGNGEPVTAEVLAQRQAEEAIRRETERRADEIVRKMAENKPDLPDGKTELAVRDIAGQERDRSAISERETALPESVLRESQREREAVREVARENLLQERLQQMERDMVRDLQKEKTLGGD

SEQ ID NO: 411 - protein, 726 aa, Methanococcoides burtonii
MSDKPAFMKYFTQSSCYPNQQEAMDRIHSALMQQQLVLFEGACGTGKTLSALVPALHVGKMLGKTVIIATNVHQQMVQFINEARDIKKVQDVKVAVIKGKTAMCPQEADYEECSVKRENTFELMETEREIYLKRQELNSARDSYKKSHDPAFVTLRDELSKEIDAVEEKARGLRDRACNDLYEVLRSDSEKFREWLYKEVRSPEEINDHAIKDGMCGYELVKRELKHADLLICNYHHVLNPDIFSTVLGWIEKEPQETIVIFDEAHNLESAARSHSSLSLTEHSIEKAITELEANLDLLADDNIHNLFNIFLEVISDTYNSRFKFGERERVRKNWYDIRISDPYERNDIVRGKFLRQAKGDFGEKDDIQILLSEASELGAKLDETYRDQYKKGLSSVMKRSHIRYVADFMSAYIELSHNLNYYPILNVRRDMNDEIYGRVELFTCIPKNVTEPLFNSLFSVILMSATLHPFEMVKKTLGITRDTCEMSYGTSFPEEKRLSIAVSIPPLFAKNRDDRHVTELLEQVLLDSIENSKGNVILFFQSAFEAKRYYSKIEPLVNVPVFLDEVGISSQDVREEFFSIGEENGKAVLLSYLWGTLSEGIDYRDGRGRTVIIIGVGYPALNDRMNAVESAYDHVFGYGAGWEFAIQVPTIRKIRQAMGRVVRSPTDYGARILLDGRFLTDSKKRFGKFSVFEVFPPAERSEFVDVDPEKVKYSLMNFFMDNDEQ

SEQ ID NO: 412 - protein, 439 aa, unidentified
  source 1..439: note = Bacteriophage T4
MTFDDLTEGQKNAFNIVMKAIKEKKHHVTINGPAGTGKTTLTKFIIEALISTGETGIILAAPTHAAKKILSKLSGKEASTIHSILKINPVTYEENVLFEQKEVPDLAKCRVLICDEVSMYDRKLFKILLSTIPPWCTIIGIGDNKQIRPVDPGENTAYISPFFTHKDFYQCELTEVKRSNAPIIDVATDVRNGKWIYDKVVDGHGVRGFTGDTALRDFMVNYFSIVKSLDDLFENRVMAFTNKSVDKLNSIIRKKIFETDKDFIVGEIIVMQEPLFKTYKIDGKPVSEIIFNNGQLVRIIEAEYTSTFVKARGVPGEYLIRHWDLTVETYGDDEYYREKIKIISSDEELYKFNLFLGKTAETYKNWNKGGKAPWSDFWDAKSQFSKVKALPASTFHKAQGMSVDRAFIYTPCIHYADVELAQQLLYVGVTRGRYDVFYV

SEQ ID NO: 413 - protein, 970 aa, unidentified
  source 1..970: note = Citromicrobium bathyomarinum
MLSVANVRSPSAAASYFASDNYYASADADRSGQWIGDGAKRLGLEGKVEARAFDALLRGELPDGSSVGNPGQAHRPGTDLTFSVPKSWSLLALVGKDERIIAAYREAVVEALHWAEKNAAETRVVEKGMVVTQATGNLAIGLFQHDTNRNQEPNLHFHAVIANVTQGKDGKWRTLKNDRLWQLNTTLNSIAMARFRVAVEKLGYEPGPVLKHGNFEARGISREQVMAFSTRRKEVLEARRGPGLDAGRIAALDTRASKEGIEDRATLSKQWSEAAQSIGLDLKPLVDRARTKALGQGMEATRIGSLVERGRAWLSRFAAHVRGDPADPLVPPSVLKQDRQTIAAAQAVASAVRHLSQREAAFERTALYKAALDFGLPTTIADVEKRTRALVRSGDLIAGKGEHKGWLASRDAVVTEQRILSEVAAGKGDSSPAITPQKAAASVQAAALTGQGFRLNEGQLAAARLILISKDRTIAVQGIAGAGKSSVLKPVAEVLRDEGHPVIGLAIQNTLVQMLERDTGIGSQTLARFLGGWNKLLDDPGNVALRAEAQASLKDHVLVLDEASMVSNEDKEKLVRLANLAGVHRLVLIGDRKQLGAVDAGKPFALLQRAGIARAEMATNLRARDPVVREAQAAAQAGDVRKALRHLKSHTVEARGDGAQVAAETWLALDKETRARTSIYASGRAIRSAVNAAVQQGLLASREIGPAKMKLEVLDRVNTTREELRHLPAYRAGRVLEVSRKQQALGLFIGEYRVIGQDRKGKLVEVEDKRGKRFRFDPARIRAGKGDDNLTLLEPRKLEIHEGDRIRWTRNDHRRGLFNADQARVVEIANGKVTFETSKGDLVELKKDDPMLKRIDLAYALNVHMAQGLTSDRGIAVMDSRERNLSNQKTFLVTVTRLRDHLTLVVDSADKLGAAVARNKGEKASAIEVTGSVKPTATKGSGVDQPKSVEANKAEKELTRSKSKTLDFGI

SEQ ID NO: 414 - protein, 262 aa, Yokenella regensburgei
CLTAPPKEAAKPTLMPRAQSYRDLTHLPLPSGKVFVSVYNIQDETGQFKPYPASNFSTAVPQSATAMLVTALKDSRWFVPLERQGLQNLLNERKIIRAAQENGTVADNNRIPLQSLTAANVMIEGSIIGYESNVKSGGVGARYFGIGADTQYQLDQIAVNLRVVNVSTGEVLSSVNTSKTILSYEVQAGVFRFVDYQRLLEGEIGYTSNEPVMLCLMSAIETGVIYLINDGIERGLWDLQQKADVDNPILARYRNMSAPPES

SEQ ID NO: 415 - protein, 262 aa, unidentified
  source 1..262: note = Cronobacter pulveris
CLTAPPKEAAKPTLMPRAQSYRDLTNLPDPKGKLFVSVYNIQDETGQFKPYPASNFSTAVPQSATSMLVTALKDSRWFIPLERQGLQNLLNERKIIRAAQENGTVAENNRMPLQSLVAANVMIEGSIIGYESNVKSGGVGARYFGIGGDTQYQLDQIAVNLRVVNVSTGEVLSSVNTSKTILSYEVQAGVFRFIDYQRLLEGEIGYTANEPVMLCLMSAIETGVIHLINDGINRGLWELKNKGDAKNTILAKYRSMAVPPES

SEQ ID NO: 416 - protein, 262 aa, Rahnella aquatilis
CLTAAPKEAARPTLLPRAPSYTDLTHLPSPQGRIFVSVYNIQDETGQFKPYPACNFSTAVPQSATAMLVSALKDSKWFIPLERQGLQNLLNERKIIRAAQENGSVAINNQRPLSSLVAANILIEGSIIGYESNVKSGGVGARYFGIGASTQYQLDQIAVNLRAVDVNTGEVLSSVNTSKTILSYEVQAGVFRFIDYQRLLEGELGYTTNEPVMLCLMSAIESGVIYLVNDGIERNLWQLQNPSEINSPILQRYKNNIVPAES

SEQ ID NO: 417 - protein, 259 aa, Kluyvera ascorbata
CITSPPKQAAKPTLLPRSQSYQDLTHLPEPQGRLFVSVYNISDETGQFKPYPASNFSTSVPQSATAMLVSALKDSNWFIPLERQGLQNLLNERKIIRAAQENGTVAVNNRTQLPSLVAANILIEGSIIGYESNVKSGGAGARYFGIGASTQYQLDQIAVNLRVVNVSTGEVLSSVNTSKTILSYEFQAGVFRYIDYQRLLEGEVGYTVNEPVMLCLMSAIETGVIYLVNDGISRNLWQLKNASDINSPVLEKYKSIIVP

SEQ ID NO: 418 - protein, 259 aa, Hafnia alvei
CLTAPPKQAAKPTLMPRAQSYQDLTHLPEPAGKLFVSVYNIQDETGQFKPYPASNFSTAVPQSATAMLVSALKDSGWFIPLERQGLQNLLNERKIIRAAQENGTAAVNNQHQLSSLVAANVLVEGSIIGYESNVKSGGAGARFFGIGASTQYQLDQIAVNLRVVDVNTGQVLSSVNTSKTILSYEVQAGVFRYIDYQRLLEGEIGYTTNEPVMLCVMSAIETGVIYLVNDGINRNLWTLKNPQDAKSSVLERYKSTIVP

SEQ ID NO: 419 - protein, 255 aa, unidentified
  source 1..255: note = Enterobacteriaceae bacterium strain FGI 57
CITTPPQEAAKPTLLPRDATYKDLVSLPQPRGKIYVAVYNIQDETGQFQPYPASNFSTSVPQSATAMLVSSLKDSRWFVPLERQGLNNLLNERKIIRAAQQNGTVGDNNASPLPSLYSANVIVEGSIIGYASNVKTGGFGARYFGIGGSTQYQLDQVAVNLRIVNVHTGEVLSSVNTSKTILSYEIQAGVFRFIDYQRLLEGEAGFTTNEPVMTCLMSAIEEGVIHLINDGINKKLWALSNAADINSEVLTRYRK

SEQ ID NO: 420 - protein, 258 aa, Plesiomonas shigelloides
ITEVPKEAAKPTLMPRASTYKDLVALPKPNGKIIVSVYSVQDETGQFKPLPASNFSTAVPQSGNAMLTSALKDSGWFVPLEREGLQNLLNERKIIRAAQENGTVAANNQQPLPSLLSANVVIEGAIIGYDSDIKTGGAGARYFGIGADGKYRVDQVAVNLRAVDVRTGEVLLSVNTSKTILSSELSAGVFRFIEYQRLLELEAGYTTNEPVMMCMMSALEAGVAHLIVEGIRQNLWSLQNPSDINNPIIQRYMKEDVP

SEQ ID NO: 421 - protein, 248 aa, unidentified
  source 1..248: note = Vibrio fischeri
PETSESPTLMQRGANYIDLISLPKPQGKIFVSVYDFRDQTGQYKPQPNSNFSTAVPQGGTALLTMALLDSEWFYPLERQGLQNLLTERKIIRAAQKKQESISNHGSTLPSLLSANVMIEGGIVAYDSNIKTGGAGARYLGIGGSGQYRADQVTVNIRAVDVRSGKILTSVTTSKTILSYEVSAGAFRFVDYKELLEVELGYTNNEPVNIALMSAIDSAVIHLIVKGVQQGLWRPANLDTRNNPIFKKY

SEQ ID NO: 422 - protein, 248 aa, Aliivibrio logei
PDASESPTLMQRGATYLDLISLPKPQGKIYVSVYDFRDQTGQYKPQPNSNFSTAVPQGGTALLTMALLDSEWFYPLERQGLQNLLTERKIIRAAQKKQESISNHGSTLPSLLSANVMIEGGIVAYDSNIKTGGAGARYLGIGGSGQYRADQVTVNIRAVDVRSGKILTSVTTSKTILSYELSAGAFRFVDYKELLEVELGYTNNEPVNIALMSAIDSAVIHLIVKGIEEGLWRPENQNGKENPIFRKY

SEQ ID NO: 423 - protein, 254 aa, Photobacterium sp.
  source 1..254: note = AK15
PETSKEPTLMARGTAYQDLVSLPLPKGKVYVSVYDFRDQTGQYKPQPNSNFSTAVPQGGAALLTTALLDSRWFMPLEREGLQNLLTERKIIRAAQKKDEIPTNHGVHLPSLASANIMVEGGIVAYDTNIQTGGAGARYLGVGASGQYRTDQVTVNIRAVDVRTGRILLSVTTSKTILSKELQTGVFKFVDYKDLLEAELGYTTNEPVNLAVMSAIDAAVVHVIVDGIKTGLWEPLRGEDLQHPIIQEYMNRSKP

SEQ ID NO: 424 - protein, 261 aa, Aeromonas veronii
CATHIGSPVADEKATLMPRSVSYKELISLPKPKGKIVAAVYDFRDQTGQYLPAPASNFSTAVTQGGVAMLSTALWDSQWFVPLEREGLQNLLTERKIVRAAQNKPNVPGNNANQLPSLVAANILIEGGIVAYDSNVRTGGAGAKYFGIGASGEYRVDQVTVNLRAVDIRSGRILNSVTTSKTVMSQQVQAGVFRFVEYKRLLEAEAGFSTNEPVQMCVMSAIESGVIRLIANGVRDNLWQLADQRDIDNPILQEYLQDNAP

SEQ ID NO: 425 - protein, 239 aa, Shewanella sp.
  source 1..239: note = ECSMB14101
ASSSLMPKGESYYDLINLPAPQGVMLAAVYDFRDQTGQYKPIPSSNFSTAVPQSGTAFLAQALNDSSWFIPVEREGLQNLLTERKIVRAGLKGDANKLPQLNSAQILMEGGIVAYDTNVRTGGAGARYLGIGAATQFRVDTVTVNLRAVDIRTGRLLSSVTTTKSILSKEITAGVFKFIDAQELLESELGYTSNEPVSLCVASAIESAVVHMIADGIWKGAWNLADQASGLRSPVLQKY

SEQ ID NO: 426 - protein, 233 aa, Pseudomonas putida
QDSETPTLTPRASTYYDLINMPRPKGRLMAVVYGFRDQTGQYKPTPASSFSTSVTQGAASMLMDALSASGWFVVLEREGLQNLLTERKIIRASQKKPDVAENIMGELPPLQAANLMLEGGIIAYDTNVRSGGEGARYLGIDISREYRVDQVTVNLRAVDVRTGQVLANVMTSKTIYSVGRSAGVFKFIEFKKLLEAEVGYTTNEPAQLCVLSAIESAVGHLLAQGIEQRLWQV

SEQ ID NO: 427 - protein, 234 aa, Shewanella violacea
  source 1..234: note = DSS12
MPKSDTYYDLIGLPHPQGSMLAAVYDFRDQTGQYKAIPSSNFSTAVPQSGTAFLAQALNDSSWFVPVEREGLQNLLTERKIVRAGLKGEANQLPQLSSAQILMEGGIVAYDTNIKTGGAGARYLGIGVNSKFRVDTVTVNLRAVDIRTGRLLSSVTTTKSILSKEVSAGVFKFIDAQDLLESELGYTSNEPVSLCVAQAIESAVVHMIADGIWKRAWNLADTASGLNNPVLQKY

SEQ ID NO: 428 - protein, 245 aa, Marinobacterium jannaschii
LTRRMSTYQDLIDMPAPRGKIVTAVYSFRDQSGQYKPAPSSSFSTAVTQGAAAMLVNVLNDSGWFIPLEREGLQNILTERKIIRAALKKDNVPVNNSAGLPSLLAANIMLEGGIVGYDSNIHTGGAGARYFGIGASEKYRVDEVTVNLRAIDIRTGRILHSVLTSKKILSREIRSDVYRFIEFKHLLEMEAGITTNDPAQLCVLSAIESAVAHLIVDGVIKKSWSLADPNELNSPVIQAYQQQRI

SEQ ID NO: 429 - protein, 234 aa, Chryseobacterium oranimense
  source 1..234: note = G311
PSDPERSTMGELTPSTAELRNLPLPNEKIVIGVYKFRDQTGQYKPSENGNNWSTAVPQGTTTILIKALEDSRWFIPIERENIANLLNERQIIRSTRQEYMKDADKNSQSLPPLLYAGILLEGGVISYDSNTMTGGFGARYFGIGASTQYRQDRITIYLRAVSTLNGEILKTVYTSKTILSTSVNGSFFRYIDTERLLEAEVGLTQNEPVQLAVTEAIEKAVRSLIIEGTRDKIW

SEQ ID NO: 430 - DNA, 12 bp, synthetic construct
  misc_feature 1..12: note = polynucleotide sequence used in Example 18
  source 1..12: mol_type = other DNA
tttttttttttt

SEQ ID NO: 431 - DNA, 27 bp, synthetic construct
  misc_feature 1..27: note = polynucleotide sequence used in Example 18
  source 1..27: mol_type = other DNA
ggttgtttctgttggtgctgatattgc

SEQ ID NO: 432 - DNA, 3635 bp, synthetic construct
  misc_feature 1..3635: note = polynucleotide sequence used in Example 18
  source 1..3635: mol_type = other DNA
gccatcagattgtgtttgttagtcgctgccatcagattgtgtttgttagtcgctttttttttttggaattttttttttggaattttttttttgcgctaacaacctcctgccgttttgcccgtgcatatcggtcacgaacaaatctgattactaaacacagtagcctggatttgttctatcagtaatcgaccttattcctaattaaatagagcaaatccccttattgggggtaagacatgaagatgccagaaaaacatgacctgttggccgccattctcgcggcaaaggaacaaggcatcggggcaatccttgcgtttgcaatggcgtaccttcgcggcagatataatggcggtgcgtttacaaaaacagtaatcgacgcaacgatgtgcgccattatcgcctagttcattcgtgaccttctcgacttcgccggactaagtagcaatctcgcttatataacgagcgtgtttatcggctacatcggtactgactcgattggttcgcttatcaaacgcttcgctgctaaaaaagccggagtagaagatggtagaaatcaataatcaacgtaaggcgttcctcgatatgctggcgtggtcggagggaactgataacggacgtcagaaaaccagaaatcatggttatgacgtcattgtaggcggagagctatttactgattactccgatcaccctcgcaaacttgtcacgctaaacccaaaactcaaatcaacaggcgccggacgctaccagcttctttcccgttggtgggatgcctaccgcaagcagcttggcctgaaagacttctctccgaaaagtcaggacgctgtggcattgcagcagattaaggagcgtggcgctttacctatgattgatcgtggtgatatccgtcaggcaatcgaccgttgcagcaatatctgggcttcactgccgggcgctggttatggtcagttcgagcataaggctgacagcctgattgcaaaattcaaagaagcgggcggaacggtcagagagattgatgtatgagcagagtcaccgcgattatctccgctctggttatctgcatcatcgtctgcctgtcatgggctgttaatcattaccgtgataacgccattacctacaaagcccagcgcgacaaaaatgccagagaactgaagctggcgaacgcggcaattactgacatgcagatgcgtcagcgtgatgttgctgcgctcgatgcaaaatacacgaaggagttagctgatgctaaagctgaaaatgatgctctgcgtgatgatgttgccgctggtcgtcgtcggttgcacatcaaagcagtctgtcagtcagtgcgtgaagccaccaccgcctccggcgtggataatgcagcctccccccgactggcagacaccgctgaacgggattatttcaccctcagagagaggctgatcactatgcaaaaacaactggaaggaacccagaagtatattaatgagcagtgcagatagagttgcccatatcgatgggcaactcatgcaattattgtgagcaatacacacgcgcttccagcggagtataaatgcctaaagtaataaaaccgagcaatccatttacgaatgtttgctgggtttctgttttaacaacattttctgcgccgccacaaattttggctgcatcgacagttttcttctgcccaattccagaaacgaagaaatgatgggtgatggtttcctttggtgctactgctgccggtttgttttgaacagtaaacgtctgttgagcacatcctgtaataagcagggccagcgcagtagcgagtagcatttttttcatggtgttattcccgatgctttttgaagttcgcagaatcgtatgtgtagaaaattaaacaaaccctaaacaatgagttgaaatttcatattgttaatatttattaatgtatgtcaggtgcgatgaatcgtcattgtattcccggattaactatgtccacagccctgacggggaacttctctgcgggagtgtccgggaataattaaaacgatgcacacagggtttagcgcgtacacgtattgcattatgccaacgccccggtgctgacacggaagaaaccggacgttatgatttagcgtggaaagatttgtgtagtgttctgaatgctctcagtaaatagtaatgaattatcaaaggtatagtaatatcttttatgttcatggatatttgtaacccatcggaaaactcctgctttagcaagattttccctgtattgctgaaatgtgatttctcttgatttcaacctatcataggacgtttctataagatgcgtgtttcttgagaatttaacatttacaacctttttaagtccttttattaacacggtgttatcgttttctaacacgatgtgaatattatctgtggctagatagtaaatataatgtgagacgttgtgacgttttagttcagaataaaacaattcacagtctaaatcttttcgcacttgatcgaatatttctttaaaaatggcaacctgagccattggtaaaaccttccatgtgatacgagggcgcgtagtttgcattatcgtttttatcgtttcaatctggtctgacctccttgtgttttgttgatgatttatgtcaaatattaggaatgttttcacttaatagtattggttgcgtaacaaagtgcggtcctgctggcattctggagggaaatacaaccgacagatgtatgtaaggccaacgtgctcaaatcttcatacagaaagatttgaagtaatattttaaccgctagatgaagagcaagcgcatggagcgacaaaatgaataaagaacaatctgctgatgatccctccgtggatctgattcgtgtaaaaaatatgcttaatagcaccatttctatgagttaccctgatgttgtaattgcatgtatagaacataaggtgtctctggaagcattcagagcaattgaggcagcgttggtgaagcacgataataatatgaaggattattccctggtggttgactgatcaccataactgctaatcattcaaactatttagtctgtgacagagccaacacgcagtctgtcactgtcaggaaagtggtaaaactgcaactcaattactgcaatgccctcgtaattaagtgaatttacaatatcgtcctgttcggagggaagaacgcgggatgttcattcttcatcacttttaattgatgtatatgctctcttttctgacgttagtctccgacggcaggcttcaatgacccaggctgagaaattcccggaccctttttgctcaagagcgatgttaatttgttcaatcatttggttaggaaagcggatgttgcgggttgttgttctgcgggttctgttcttcgttgacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgaaaaaaaaggtaccaaaaaaaacatcgtcgtgagtagtgaaccgtaagccgttctgtttatgtttcttggacactgattgacacggtttagtagaac

SEQ ID NO: 433 - DNA, 3636 bp, synthetic construct
  misc_feature 1..3636: note = polynucleotide sequence used in Example 18
  source 1..3636: mol_type = other DNA
ttttttttttttttttttttttttttttcaagaaacataaacagaacgtgcttacggttcactactcacgacgatgttttttttggtaccttttttttcaccggaaaggacccgtaaagtgataatgattatcatctacatatcacaacgtgcgtggaggccatcaaaccacgtcaaataatcaattatgacgcaggtatcgtattaattgatctgcatcaacttaacgtaaaaacaacttcagacaatacaaatcagcgacactgaatacggggcaacctcatgtcaacgaagaacagaacccgcagaacaacaacccgcaacatccgctttcctaaccaaatgattgaacaaattaacatcgctcttgagcaaaaagggtccgggaatttctcagcctgggtcattgaagcctgccgtcggagactaacgtcagaaaagagagcatatacatcaattaaaagtgatgaagaatgaacatcccgcgttcttccctccgaacaggacgatattgtaaattcacttaattacgagggcattgcagtaattgagttgcagttttaccactttcctgacagtgacagactgcgtgttggctctgtcacagactaaatagtttgaatgattagcagttatggtgatcagtcaaccaccagggaataatccttcatattattatcgtgcttcaccaacgctgcctcaattgctctgaatgcttccagagacaccttatgttctatacatgcaattacaacatcagggtaactcatagaaatggtgctattaagcatattttttacacgaatcagatccacggagggatcatcagcagattgttctttattcattttgtcgctccatgcgcttgctcttcatctagcggttaaaatattacttcaaatctttctgtatgaagatttgagcacgttggccttacatacatctgtcggttgtatttccctccagaatgccagcaggaccgcactttgttacgcaaccaatactattaagtgaaaacattcctaatatttgacataaatcatcaacaaaacacaaggaggtcagaccagattgaaacgataaaaacgataatgcaaactacgcgccctcgtatcacatggaaggttttaccaatggctcaggttgccatttttaaagaaatattcgatcaagtgcgaaaagatttagactgtgaattgttttattctgaactaaaacgtcacaacgtctcacattatatttactatctagccacagataatattcacatcgtgttagaaaacgataacaccgtgttaataaaaggacttaaaaaggttgtaaatgttaaattctcaagaaacacgcatcttatagaaacgtcctatgataggttgaaatcaagagaaatcacatttcagcaatacagggaaaatcttgctaaagcaggagttttccgatgggttacaaatatccatgaacataaaagatattactatacctttgataattcattactatttactgagagcattcagaacactacacaaatctttccacgctaaatcataacgtccggtttcttccgtgtcagcaccggggcgttggcataatgcaatacgtgtacgcgctaaaccctgtgtgcatcgttttaattattcccggacactcccgcagagaagttccccgtcagggctgtggacatagttaatccgggaatacaatgacgattcatcgcacctgacatacattaataaatattaacaatatgaaatttcaactcattgtttagggtttgtttaattttctacacatacgattctgcgaacttcaaaaagcatcgggaataacaccatgaaaaaaatgctactcgctactgcgctggccctgcttattacaggatgtgctcaacagacgtttactgttcaaaacaaaccggcagcagtagcaccaaaggaaaccatcacccatcatttcttcgtttctggaattgggcagaagaaaactgtcgatgcagccaaaatttgtggcggcgcagaaaatgttgttaaaacagaaacccagcaaacattcgtaaatggattgctcggttttattactttaggcatttatactccgctggaagcgcgtgtgtattgctcacaataattgcatgagttgcccatcgatatgggcaactctatctgcactgctcattaatatacttctgggttccttccagttgtttttgcatagtgatcagcctctctctgagggtgaaataatcccgttcagcggtgtctgccagtcggggggaggctgcattatccacgccggaggcggtggtggcttcacgcactgactgacagactgctttgatgtgcaaccgacgacgaccagcggcaacatcatcacgcagagcatcattttcagctttagcatcagctaactccttcgtgtattttgcatcgagcgcagcaacatcacgctgacgcatctgcatgtcagtaattgccgcgttcgccagcttcagttctctggcatttttgtcgcgctgggctttgtaggtaatggcgttatcacggtaatgattaacagcccatgacaggcagacgatgatgcagataaccagagcggagataatcgcggtgactctgctcatacatcaatctctctgaccgttccgcccgcttctttgaattttgcaatcaggctgtcagccttatgctcgaactgaccataaccagcgcccggcagtgaagcccagatattgctgcaacggtcgattgcctgacggatatcaccacgatcaatcataggtaaagcgccacgctccttaatctgctgcaatgccacagcgtcctgacttttcggagagaagtctttcaggccaagctgcttgcggtaggcatcccaccaacgggaaagaagctggtagcgtccggcgcctgttgatttgagttttgggtttagcgtgacaagtttgcgagggtgatcggagtaatcagtaaatagctctccgcctacaatgacgtcataaccatgatttctggttttctgacgtccgttatcagttccctccgaccacgccagcatatcgaggaacgccttacgttgattattgatttctaccatcttctactccggcttttttagcagcgaagcgtttgataagcgaaccaatcgagtcagtaccgatgtagccgataaacacgctcgttatataagcgagattgctacttagtccggcgaagtcgagaaggtcacgaatgaactaggcgataatggcgcacatcgttgcgtcgattactgtttttgtaaacgcaccgccattatatctgccgcgaaggtacgccattgcaaacgcaaggattgccccgatgccttgttcctttgccgcgagaatggcggccaacaggtcatgtttttctggcatcttcatgtcttacccccaataaggggatttgctctatttaattaggaataaggtcgattactgatagaacaaatccaggctactgtgtttagtaatcagatttgttcgtgaccgatatgcacgggcaaaacggcaggaggttgttagcgcaaaaaaaaaattccaaaaaaaaaattccaaaaaaaaaaagcgactaacaaacacaatctgatggcagcgactaacaaacacaatctgatggc

SEQ ID NO: 434 - DNA, 28 bp, synthetic construct
  misc_feature 1..28: note = polynucleotide sequence used in Example 18
  source 1..28: mol_type = other DNA
gcaatatcagcaccaacagaaacaacct

SEQ ID NO: 435 - protein, 10 aa, synthetic construct
  REGION 1..10: note = amino acid sequence of StepII(C)
SAWSHPQFEK

SEQ ID NO: 436 - protein, 15 aa, synthetic construct
  REGION 1..15: note = amino acid sequence of Pro
MQRLFLLVAVMLLSG

SEQ ID NO: 437 - DNA, 22 bp, synthetic construct
  misc_feature 1..22: note = Primer
  source 1..22: mol_type = other DNA
tctttaaccgccccgcctaaag

SEQ ID NO: 438 - DNA, 20 bp, synthetic construct
  misc_feature 1..20: note = Primer
  source 1..20: mol_type = other DNA
cattttttgccctcgttatc

SEQ ID NO: 439 - DNA, 67 bp, synthetic construct
  misc_feature 1..67: note = Primer
  source 1..67: mol_type = other DNA
aataactcaaccgatttttaagccccagcttcataaggaaaataatcgtgtaggctggagctgcttc

SEQ ID NO: 440 - DNA, 66 bp, synthetic construct
  misc_feature 1..66: note = Primer
  source 1..66: mol_type = other DNA
cgcttaaacagtaaaatgccggatgataattccggcttttttatctgcatatgaatatcctcctta

SEQ ID NO: 441 - DNA, 78 bp, synthetic construct
  misc_feature 1..78: note = Hairpin
  source 1..78: mol_type = other DNA
gcggggagcgtattagagttggatcggatgcagctggctactgacgtcatgacgtcagtagccagcatgcatccgatc

SEQ ID NO: 442 - protein, 239 aa, Pseudoalteromonas sp.
  source 1..239: note = (strain SM9913)
MGNLIPPSKQSAVTLEPTDVFSDLKSLPKPAGSIPVSVYSFRDQTGQYKPQTNVSSFSTAVTQGANSILVQALHESDWFTPVEREGLQNILTERKIIRAAQATNPNQGELPPLTTAKIILEGGIISYDTNVKTGGLGMEYFGIGASELYREDAISIYLRAVDVRTGQVLLSVATSKKVLSQEMRAGFFRYVSYKRLAEAEAGFSDNEPMSICVTQAIEKALTDLITKGIDKGLWAKQAT

SEQ ID NO: 443 - protein, 252 aa, Shewanella baltica
CSLIPKPDLNITPAEVNPLSEVMRGLQTQPGPKFPIPVAVYSFRDQTGQYKPQANVSSFSTAVTQGATSMLMQTLLDSKWFTPVEREGLQNLLTERKISNKQSGTKGDDVPVLSTARLLLEGGVISYETNTSTGGSGVEYYGIGASEMYREDQVTIYLRAVDVHTGKVMMSVSTSKRVLSQEMRAGLFRYTSLNRLAEAEIGFTTNEPVQFCVLQAIELAVAELIDKGIKQGYWSPTQVTQPVEKAQELSQS

SEQ ID NO: 444 - protein, 264 aa, unidentified
  source 1..264: note = Pseudomonas entomophila
CGLREPMPAEQDAETPTLTPRASTYYDLINMPRPRGRLMAVVYGFRDQTGQYKPTPASSFSTSVTQGAASMLMDALNASGWFVVLEREGLQNLLTERKIIRASQKKPDVAENIQGELPPLQAANLMLEGGIIAYDTNVRSGGEGARYLGIDISREYRVDQVTVNLRAVDVRTGQVLANVMTSKTIYSVGRSAGVFKFIEFKKLLEAEVGYTTNEPAQLCVLSAIEAAVGHLLAQGIERRLWQVAADGAGDKALLDKYLSQYQQP

SEQ ID NO: 445 - protein, 261 aa, Hafnia alvei
CAGMVATSENLEGAEATLTPRGATYQDLVSLPPPSGKIFVSVYDFRDQTGQYRPAPASTFSTAVTQGAAAMLTGSLSDSGWFIPLERVGLQNLLTERRIIRAEFERFGQPDTLPSLRAASVMLEGGIIAYESNIRTGGAGAEYFGIGASGQYQVDQVTVNLRAVEISTGEILANVTTTKTIYSKELRAGVYRFIDFSRLLEAEAGITANEPVQLAVMSAIESAVIHLIARGIENRLWNLAPGVSLQESILNDYLNAPIPML

SEQ ID NO: 446 - protein, 262 aa, Escherichia coli; identical to SEQ ID NO: 390

SEQ ID NO: 447 - protein, 263 aa, Vibrio orientalis
CSNSMTIPDADEAPTLMPRGATYQDLVQLPEPKGRILVSVYDFRDQTGQYKPQPNSNFSTAVPQGGTALLTASLLDSRWFIPLEREGLQNLLTERKIIRAAQKKGQAASNHGDDLSALSSANVVIEGGIVAYDSNIKTGGLGARYLGIGSSGKYRTDQVTVNLRAVNVRTGQILLSVTTSKTIFSHELTAGAFRFIDYKDLLEVEMGYTNNEPVNVAVMSAIDAAVIHLIVKGIGRGLWQPENKEDLQDETIQRYAKQTHQIL

SEQ ID NO: 448 - protein, 267 aa, Shewanella pealeana
CSTMSEIEQITPSSSLMPKSETYYDLIGLPAPQGSMVAAVYDFRDQTGQYKPIPSSNFSTAVPQSGTAFLAQALNDSSWFTPVEREGLQNLLTERKIVRAGLKGDAASLSQLNSAQILMEGGIVAYDTNIRTGGAGARYLGIGASGQFRVDSITVNLRAVDIRTGRLLSSVTTTKSVLSKELTAGVFKFIDAQELLESEIGYTSNEPVSLCVAQAIESAVVHMIADGIWKRAWNLADSQTGLENPILKKYWLEAHSVERVQARLEQG

SEQ ID NO: 449 - protein, 21 aa, synthetic construct
  REGION 1..21: note = TM1 hairpin (figure 43)
VKSGGVGARYFGIGADTQYQL

SEQ ID NO: 450 - protein, 27 aa, synthetic construct
  REGION 1..27: note = TM2 hairpin (figure 43)
LSYEVQAGVFRFIDYQRLLEGEVGYTS

SEQ ID NO: 451 - protein, 66 aa, synthetic construct
  REGION 1..66: note = Synthetic polypeptide
MGNLIPPSKQSAVTLEPTDVFSDLKSLPKPAGSIPVSVYSFRDQTGQYKPQTNVSSFSTAVTQGAN

SEQ ID NO: 452 - protein, 68 aa, synthetic construct
  REGION 1..68: note = Synthetic polypeptide
CSLIPKPDLNITPAEVNPLSEVMRGLQTQPGPKFPIPVAVYSFRDQTGQYKPQANVSSFSTAVTQGAT

SEQ ID NO: 453 - protein, 69 aa, synthetic construct
  REGION 1..69: note = Synthetic polypeptide
CGLREPMPAEQDAETPTLTPRASTYYDLINMPRPRGRLMAVVYGFRDQTGQYKPTPASSFSTSVTQGAA

SEQ ID NO: 454 - protein, 69 aa, synthetic construct
  REGION 1..69: note = Synthetic polypeptide
CAGMVATSENLEGAEATLTPRGATYQDLVSLPPPSGKIFVSVYDFRDQTGQYRPAPASTFSTAVTQGAA

SEQ ID NO: 455 - protein, 65 aa, synthetic construct
  REGION 1..65: note = Synthetic polypeptide
CLTAPPKQAAKPTLMPRAQSYQDLTHLPEPAGKLFVSVYNIQDETGQFKPYPASNFSTAVPQSAT

SEQ ID NO: 456 - protein, 65 aa, synthetic construct
  REGION 1..65: note = Synthetic polypeptide
CLTAPPKEAARPTLMPRAQSYKDLTHLPAPTGKIFVSVYNIQDETGQFKPYPASNFSTAVPQSAT

SEQ ID NO: 457 - protein, 67 aa, synthetic construct
  REGION 1..67: note = Synthetic polypeptide
CSNSMTIPDADEAPTLMPRGATYQDLVQLPEPKGRILVSVYDFRDQTGQYKPQPNSNFSTAVPQGGT

SEQ ID NO: 458 - protein, 65 aa, synthetic construct
  REGION 1..65: note = Synthetic polypeptide
CSTMSEIEQITPSSSLMPKSETYYDLIGLPAPGSMVAAVYDFRDQTQYKPIPSSNFSTAVPQSGT

SEQ ID NO: 459 - protein, 66 aa, synthetic construct
  REGION 1..66: note = Synthetic polypeptide
SILVQALHESDWFTPVEREGLQNILTERKIIRAAQATNPNQGELPPLTTAKIILEGGIISYDTNVK

SEQ ID NO: 460 - protein, 64 aa, synthetic construct
  REGION 1..64: note = Synthetic polypeptide
SMLMQTLLDSKWFTPVEREGLQNLLTERKISNKQSGTKGDDVPVLSTARLLLEGGVISYETNTS

SEQ ID NO: 461 - protein, 70 aa, synthetic construct
  REGION 1..70: note = Synthetic polypeptide
SMLMDALNASGWFVVLEREGLQNLLTERKIIRASQKKPDVAENIQGELPPLQAANLMLEGGIIAYDTNVR

SEQ ID NO: 462 - protein, 65 aa, synthetic construct
  REGION 1..65: note = Synthetic polypeptide
AMLTGSLSDSGWFIPLERVGLQNLLTERRIIRAEFERFGQPDTLPSLRASVMLEGGIIAYESNIR

SEQ ID NO: 463 - protein, 70 aa, synthetic construct
  REGION 1..70: note = Synthetic polypeptide
AMLVSALKDSGWFIPLERQGLQNLLNERKIIRAAQENGTVAVNNQRQLSSLVAANVLIEGSIIGYESNVK

SEQ ID NO: 464 - protein, 70 aa, synthetic construct
  REGION 1..70: note = Synthetic polypeptide
AMLVTALKDSRWFIPLERQGLQNLLNERKIIRAAQENGTVAINNRIPLQSLTAANIMVEGSIIGYESNVK

SEQ ID NO: 465 - protein, 70 aa, synthetic construct
  REGION 1..70: note = Synthetic polypeptide
ALLTASLLDSRWFIPLEREGLQNLLTERKIIRAAQKKGQAASNHGDDLSALSSANVVIEGGIVAYDSNIK

SEQ ID NO: 466 - protein, 64 aa, synthetic construct
  REGION 1..64: note = Synthetic polypeptide
AFLAQALNDSSWFTPVEREGLQNLLTERKIVRAGLKGDAASLSQLNSAQILMEGGIVAYDTNIR

SEQ ID NO: 467 - protein, 70 aa, synthetic construct
  REGION 1..70: note = Synthetic polypeptide
TGGLGMEYFGIGASELYREDAISIYLRAVDVRTGQVLLSVATSKKVLSQEMRAGFFRYVSYKRLAEAEAG

SEQ ID NO: 468 - protein, 70 aa, synthetic construct
  REGION 1..70: note = Synthetic polypeptide
TGGSGVEYYGIGASEMYREDQVTIYLRAVDVHTGKVMMSVSTSKRVLSQEMRAGLFRYTSLNRLAEAEIG

SEQ ID NO: 469 - protein, 69 aa, synthetic construct
  REGION 1..69: note = Synthetic polypeptide
SGGEGARYLGIDISREYRVDQVTVNLRAVDVRTGQVLANVMTSKTIYSVGRSAGVFKFIEFKKLLEAVG

SEQ ID NO: 470 - protein, 69 aa, synthetic construct
  REGION 1..69: note = Synthetic polypeptide
TGGAGAEYFGIGASGQYQVDQVTVNLRAVEISTGEILANVTTKTIYSKELRAGVYRFIDFSRLLEAEAG

SEQ ID NO: 471 - protein, 70 aa, synthetic construct
  REGION 1..70: note = Synthetic polypeptide
SGGAGARFFGIGASTQYQLDQIAVNLRVVDVNTGQVLSSVNTSKTILSYEVQAGVFRYIDYQRLLEGKIG

SEQ ID NO: 472 - protein, 70 aa, synthetic construct
  REGION 1..70: note = Synthetic polypeptide
SGGVGARYFGIGADTQYQLDQIAVNLRVVNVSTGEILSSVNTSKTILSYEVQAGVFRFIDYQRLLEGEVG

SEQ ID NO: 473 - protein, 70 aa, synthetic construct
  REGION 1..70: note = Synthetic polypeptide
TGGLGARYLGIGSSGKYRTDQVTVNLRAVNVRTGQILLSVTTSKTIFSHELTAGAFRFIDYKDLLEVEMG

SEQ ID NO: 474 - protein, 70 aa, synthetic construct
  REGION 1..70: note = Synthetic polypeptide
TGGAGARYLGIGASGQFRVDSITVNLRAVDIRTGRLLSSVTTTKSVLSKELTAGVFKFIDAQELLESEIG

SEQ ID NO: 475 - protein, 37 aa, synthetic construct
  REGION 1..37: note = Synthetic polypeptide
FSDNEPMSICVTQAIEKALTDLITKGIDKGLWAKQAT

SEQ ID NO: 476 - protein, 50 aa, synthetic construct
  REGION 1..50: note = Synthetic polypeptide
FTTNEPVQFCVLQAIELAVAELIDKGIKQGYWSPTQVTQPVEKAQELSQS

SEQ ID NO: 477 - protein, 55 aa, synthetic construct
  REGION 1..55: note = Synthetic polypeptide
YTTNEPAQLCVLSAIEAAVGHLLAQGIERRLWQVAADGAGDKALLDKYLSQYQQP

SEQ ID NO: 478 - protein, 56 aa, synthetic construct
  REGION 1..56: note = Synthetic polypeptide
ITANEPVQLAVMSAIESAVIHLIAEGIENRLWNLAPGVSLQESILNDYLNAPIPML

SEQ ID NO: 479 - protein, 57 aa, synthetic construct
  REGION 1..57: note = Synthetic polypeptide
YTTNEPVMLCVMSAIETGVIYLVNDGITRNLWSLKNAKDVTSPILERYKSTIVPIAS

SEQ ID NO: 480 - protein, 57 aa, synthetic construct
  REGION 1..57: note = Synthetic polypeptide
YTSNEPVMLCLMSAIETGVIFLINDGIDRGLWDLQNKAERQNDILVKYRHMSVPPES

SEQ ID NO: 481 - protein, 56 aa, synthetic construct
  REGION 1..56: note = Synthetic polypeptide
YTNNEPVNVAVMSAIDAAVIHLIVKGIGRGLWQPENKEDLQDETIQRYAKQTHQIL

SEQ ID NO: 482 - protein, 66 aa, synthetic construct
  REGION 1..66: note = Synthetic polypeptide
YTSNEPVSLCVAQAIESAVVHMIADGIWKRAWNLADSQTGLENPILKKYWLEAHSVERVQARLEQG

(19) United States
(12) Patent Application Publication
(10) Pub. No.: US 2026/0015392 A1
(43) Pub. Date: Jan. 15, 2026

(54) MUTANT CSGG PORES

(71) Applicants: Oxford Nanopore Technologies PLC, Oxford (GB); VIB VZW, Gent (BE); Vrije Universiteit Brussel, Brussels (BE)

(72) Inventors: Stefan Howorka, London (GB); Han Remaut, Roosbeek (BE); Lakmal Nishantha Jayasinghe, Oxford (GB); Elizabeth Jayne Wallace, Oxford (GB); James Anthony Clarke, Oxford (GB); Richard George Hambley, Oxford (GB); Jonathan Bankes Pugh, Oxford (GB)

(73) Assignees: Oxford Nanopore Technologies PLC, Oxford (BE); VIB VZW, Gent (BE); Vrije Universiteit Brussel, Brussels (BE)

(21) Appl. No.: 19/069,041

(22) Filed: Mar. 3, 2025

Related U.S. Application Data
(63) Continuation of application No. 17/194,821, filed on Mar. 8, 2021, now Pat. No. 12,275,761.
Application No. 17/194,821 is a continuation of application No. 16/522,591, filed on Jul. 25, 2019, now Pat. No. 11,034,734.
Application No. 16/522,591 is a continuation of application No. 15/507,947, filed on Mar. 1, 2017, now Pat. No. 10,400,014.

(30) Foreign Application Priority Data
Sep. 1, 2014 (GB) 1415455.3
Dec. 11, 2014 (GB) 1422079.2
Apr. 16, 2015 (GB) 1506489.2
Apr. 21, 2015 (GB) 1506754.9
May 14, 2015 (GB) 1508287.8
Jun. 25, 2015 (GB) 1511203.0
Aug. 27, 2015 (GB) 1515240.8

(51) Int. Cl. C07K 14/245; C12Q 1/68; C12Q 1/6869; C12Q 1/6874; G01N 27/447

(52) U.S. Cl. CPC C07K 14/245; C12Q 1/6869; C12Q 1/6874; G01N 27/447; C07K 2319/22; C12Q 1/68; C12Q 2565/631

(57) ABSTRACT

The invention relates to mutant forms of CsgG. The invention also related to analyte detection and characterisation using CsgG.

### FIELD OF THE INVENTION

The present invention relates to novel protein pores and their uses. In particular it relates to biological nanopores in nucleic acid sequencing applications, and molecular sensing.

The invention relates to mutant forms of CsgG. The invention also relates to analyte detection and characterisation using CsgG.

### BACKGROUND OF THE INVENTION

Protein pores are membrane spanning polypeptides and complexes that form a channel in the membrane through which ions and certain molecules may pass. The minimum diameter of the channel is typically in the nanometre (10⁻⁹ metre) range hence giving certain of these polypeptides the name “nanopores”.

Nanopores have great potential as biological sensors. When an electrical potential is applied across a membrane-bound nanopore, ions flow through the channel. This flow of ions can be measured as an electrical current. Suitable electrical measurement techniques using single channel recording equipment are described in, for example, WO 2000/28312 and D. Stoddart et al., Proc. Natl. Aced. Sci., 2010, 106, 7702-7. Multi-channel recording techniques are described, for example, in WO 2009/077734.

A molecule translating though the pore, or binding in or near the pore acts to obstruct and thereby reduce the ion flow through the channel. The degree of reduction in ion flow, as measured by the reduction in electrical current, is indicative of the size of the obstruction within, or in the vicinity of, the pore. The measured electrical current can therefore be used as a measure of the size or degree of obstruction to the channel. The changes in electrical current can be used to identify that a molecule, or part of a molecule, has bound at or near the pore (molecular sensing), or in certain systems, it can be used to determine the identity of a molecule that is present within the pore based on its size (nucleic acid sequencing).

The “Strand Sequencing” method is known for sequencing nucleic acids using biological nanopores. On passing a single polynucleotide strand through a nanopore, the bases on individual nucleotides are determined by the changes in measured electrical current as they pass transiently through the channel of the nanopore. This method offers significant time and cost savings over historic methods of nucleic acid sequencing.

Previously reported protein nanopores, such as the mutant MspA (Manrao et al., Nature Biotechnology, 2012, 30(4), 349-353) and alpha-hemolysin nanopores (Nat. Nanotechnol., 2009, 4(4), 265-70) have been used for nucleic acid sequencing using the “Strand Sequencing” approach. Similarly, for protein sensing other pores such as alpha-hemolysin (J Am Chem Soc., 2012, 134(5), 2781-7) and ClyA (Am. Chem. Soc. Nano. 2014, 8(12). 12826-35) (J. Am. Chem. Soc., 2013, 135(36), 13456-63) have also been adapted.

There remains a need for new nanopores that overcome the deficiencies of the prior art, not least in optimising the dimensions and characteristics of the pore for molecular sensing applications, and for example, nucleic acid sequencing applications.

Nanopore sensing is an approach to sensing that relies on the observation of individual binding or interaction events between analyte molecules and a receptor. Nanopore sensors can be created by placing a single pore of nanometer dimensions in an insulating membrane and measuring voltage-driven ionic transport through the pore in the presence of analyte molecules. The identity of an analyte is revealed through its distinctive current signature, notably the duration and extent of current block and the variance of current levels.

There is currently a need for rapid and cheap nucleic acid (e.g. DNA or RNA) sequencing technologies across a wide range of applications. Existing technologies are slow and expensive mainly because they rely on amplification techniques to produce large volumes of nucleic acid and require a high quantity of specialist fluorescent chemicals for signal detection. Nanopore sensing has the potential to provide rapid and cheap nucleic acid sequencing by reducing the quantity of nucleotide and reagents required.

Two of the essential components of sequencing nucleic acids using nanopore sensing are (1) the control of nucleic acid movement through the pore and (2) the discrimination of nucleotides as the nucleic acid polymer is moved through the pore. In the past, to achieve nucleotide discrimination the nucleic acid has been passed through a mutant of hemolysin. This has provided current signatures that have been shown to be sequence dependent. It has also been shown that a large number of nucleotides contribute to the observed current when a hemolysin pore is used, making a direct relationship between observed current and polynucleotide challenging.

While the current range for nucleotide discrimination has been improved through mutation of the hemolysin pore, a sequencing system would have higher performance if the current differences between nucleotides could be improved further. In addition, it has been observed that when the nucleic acids are moved through a pore, some current states show high variance. It has also been shown that some mutant hemolysin pores exhibit higher variance than others. While the variance of these states may contain sequence specific information, it is desirable to produce pores that have low variance to simplify the system. It is also desirable to reduce the number of nucleotides that contribute to the observed current.

### SUMMARY OF THE INVENTION

The inventors have identified the structure of the bacterial amyloid secretion channel CsgG. The CsgG channel is a trans-membrane oligomeric protein that forms a channel with a minimum diameter of approximately 0.9 nm. The structure of the CsgG nanopore renders it suitable for use in protein sensing applications, in particular in nucleic acid sequencing. Modified versions of the CsgG polypeptide may serve to further enhance the suitability of the channel for such particular applications.

The CsgG pore offers an advantage over existing protein pores such as ClyA or alpha-hemolysin in that the structure is favourable for DNA sequencing applications. The CsgG pore has a more favourable aspect ratio comprising a shorter trans-membrane channel than ClyA. The CsgG pore also has a wider channel opening compared to the alpha-hemolysin pore. This can facilitate the attachment of enzymes for certain applications, for example nucleic acid sequencing applications. In these embodiments, it can also minimize the length of the nucleic acid strand section positioned between the enzyme and the reading head (defined as the narrowest pore section) leading to an improved read-out signal. The narrow inner constriction of the channel of the CsgG pore also facilitates the translocation of single stranded DNA in embodiments of the invention involving nucleic acid sequencing. The constriction is composed of two annular rings formed by juxtaposition of tyrosine residues at position 51 (Tyr 51) in the adjacent protein monomers, and also the phenylalanine and asparagine residues at positions 56 and 55 respectively (Phe 56 and Asn 55). The dimensions of the constriction can be modified. ClyA has a much wider inner constriction which allows the passage of double stranded DNA which is currently not used for sequencing. The alpha-hemolysin pore has one 1.3 nm-wide inner constriction but also a 2 nm-wide beta barrel which features additional reading heads.

In a first aspect, the invention relates to a method for molecular sensing comprising:
a) providing a CsgG biological pore formed of at least one CsgG monomer within an insulating layer;
b) applying an electrical potential across the insulating layer thereby establishing flow of electrical current through the biological pore;
c) contacting the CsgG biological pore with a test substrate; and
d) measuring the electrical current flow through the biological pore.

Typically, the insulating layer is a membrane, such as a lipid bilayer. In an embodiment, the electrical current through the pore is carried by a flow of soluble ions from a first side of the insulating layer to the second side of the insulating layer.

In an embodiment of the invention, the molecular sensing is analyte detection. In a specific embodiment, the method for analyte detection comprises after step (d) the further step of determining the presence of the test substrate by a reduction in electrical current through the biological pore compared to the electrical current through the biological pore when the test substrate is absent.

In an alternative embodiment of the invention, the molecular sensing is nucleic acid sequencing. Typically, the type of nucleic acid sequenced by said method is DNA or RNA. In specific embodiments of the invention, the CsgG biological pore is adapted to accommodate additional accessory proteins. Typically, the additional accessory proteins are nucleic acid-processing enzymes selected from the group consisting of: DNA or RNA polymerases; isomerases; topoisomerases; gyrases; telomerases; exonucleases; and helicases.

In embodiments of the invention, the CsgG biological pore is a modified CsgG pore, wherein the modified CsgG pore has at least one modification to the monomeric wild-type E-coli CsgG polypeptide sequence in at least one of the CsgG monomers forming the CsgG pore. Typically, the same modification is made to all the CsgG monomers forming the CsgG pore. In specific embodiments of the invention, the modified CsgG monomer has a polypeptide sequence from positions 38 to 63 according to SEQ ID NOs 4 to 388.

In a second aspect, the invention relates to modified CsgG biological pore comprising at least one CsgG monomer, wherein the modified CsgG biological pore has no more than one channel constriction with a diameter in the range from 0.5 nm to 1.5 nm. Typically, the modification is between positions 38 to 63 of the CsgG monomeric polypeptide sequence. Suitably, the modification is at a position selected from: Tyr51; Asn55; and Phe 56. In specific embodiments, the modification is at position Tyr 51, or at both of positions Asn55 and Phe56.

In embodiments of the invention, the modification to the CsgG monomer is selected from the group consisting of substitution of the naturally occurring amino acid; deletion of the naturally occurring amino acid; and modification of the naturally-occurring amino acid side chain. Suitably, the modification reduces or removes the steric encumbrance of the unmodified amino acid. In specific embodiments, at least one CsgG monomer of the pore has a polypeptide sequence from positions 38 to 63 according to SEQ ID NOs 4 to 388.

In a third aspect, the invention relates to the isolated polypeptide encoding the at least one CsgG monomer of the modified CsgG biological pore of the second aspect of the invention.

In a fourth aspect, the Invention relates to isolated nucleic acids encoding the isolated polypeptides of the third aspect of the invention.

In a fifth aspect, the invention relates to a biosensor comprising:
a) An Insulating layer;
b) A CsgG biological pore within the insulating layer; and
c) Apparatus for measuring an electrical current through the biological pore.

In specific embodiments, the CsgG biological pore in the biosensor is a modified CsgG biological pore according to the second aspect of the invention.

In a sixth aspect, the invention relates to the use of a CsgG biological pore for biological sensing applications, wherein the biological sensing application is analyte detection or nucleic acid sequencing. In an embodiment of the sixth aspect of the invention, the nucleic acid sequencing is DNA sequencing or RNA sequencing.

The inventors have surprisingly demonstrated that CsgG and novel mutants thereof may be used to characterise analytes, such as polynucleotides. The invention concerns mutant CsgG monomers in which one or more modifications have been made to improve the ability of the monomer to interact with an analyte, such as a polynucleotide. The inventors have also surprisingly demonstrated that pores comprising the novel mutant monomers have an enhanced ability to interact with analytes, such as polynucleotides, and therefore display improved properties for estimating the characteristics of analytes, such as the sequence of polynucleotides. The mutant pores surprisingly display improved nucleotide discrimination. In particular, the mutant pores surprisingly display an increased current range, which makes it easier to discriminate between different nucleotides, and a reduced variance of states, which increases the signal-to-noise ratio. In addition, the number of nucleotides contributing to the current as the polynucleotide moves through the pore is decreased. This makes it easier to Identify a direct relationship between the observed current as the polynucleotide moves through the pore and the polynucleotide. In addition, the mutant pores may display an increased throughput, i.e. are more likely to interact with an analyte, such as a polynucleotide. This makes it easier to characterise analytes using the pores. The mutant pores may insert into a membrane more easily.

Accordingly, the invention provides a mutant CsgG monomer comprising a variant of the sequence shown in SEQ ID NO: 390, wherein the variant comprises a mutation at one or more of positions Y51, N55 and F56.

Accordingly, the invention provides a mutant CsgG monomer comprising a variant of the sequence shown in SEQ ID NO: 390, wherein the variant comprises one or more of the following: (i) one or more mutations at the following positions (i.e. mutations at one or more of the following positions) N40, D43, E44, S54, S57, Q62, R97, E101, E124, E131, R142, T150 and R192; (ii) mutations at Y51/N55, Y51/F56, N55/F56 or Y51/N55/F56; (iii) Q42R or Q42K; (iv) K49R; (v) N102R, N102F, N102Y or N102W; (vi) D149N, D149Q or D149R; (vii) E185N, E1850 or E185R; (viii) D195N, D1950 or D195R; (ix) E201N, E201Q or E201R; (x) E203N, E203Q or E203R; and (xi) deletion of one or more of the following positions F48, K49, P50, Y51, P52, A53, S54, N55, F56 and S57.

The invention also provides:
a construct comprising two or more covalently attached CsgG monomers, wherein at least one of the monomers is a mutant monomer of the invention;
a polynucleotide which encodes a mutant monomer of the invention or a construct of the invention;
a homo-oligomeric pore derived from CsgG comprising identical mutant monomers of the invention or identical constructs of the invention;
a hetero-oligomeric pore derived from CsgG comprising at least one mutant monomer of the invention or at least one construct of the invention;
a method for determining the presence, absence or one or more characteristics of a target analyte, comprising:
   a) contacting the target analyte with a CsgG pore or a mutant thereof such that the target analyte moves with respect to the pore; and
   b) taking one or more measurements as the analyte moves with respect to the pore and thereby determining the presence, absence or one or more characteristics of the analyte;
a method of forming a sensor for characterising a target polynucleotide, comprising forming a complex between a CsgG pore or a mutant thereof and a polynucleotide binding protein and thereby forming a sensor for characterising the target polynucleotide;
a sensor for characterising a target polynucleotide, comprising a complex between a CsgG pore or a mutant thereof and a polynucleotide binding protein;
use of a CsgG pore or a mutant thereof to determine the presence, absence or one or more characteristics of a target analyte;
a kit for characterising a target analyte comprising (a) a CsgG pore or a mutant thereof and (b) the components of a membrane;
an apparatus for characterising target analytes in a sample, comprising (a) a plurality of a CsgG pores or mutants thereof and (b) a plurality of membranes;
a method of characterising a target polynucleotide, comprising:
   a) contacting the polynucleotide with a CsgG pore or a mutant thereof, a polymerase and labelled nucleotides such that phosphate labelled species are sequentially added to the target polynucleotide by the polymerase, wherein the phosphate species contain a label specific for each nucleotide; and
   b) detecting the phosphate labelled species using the pore and thereby characterising the polynucleotide; and
a method of producing a mutant monomer of the invention or a construct of the invention, comprising expressing a polynucleotide of the invention in a suitable host cell and thereby producing a mutant monomer of the invention or a construct.

### DESCRIPTION OF THE FIGURES

FIG. 1 shows a side cross-sectional view of the structure of a CsgG nonamer in its channel conformation in ribbon and surface representation.

FIG. 2 shows a cross-section of CsgG channel constriction (i.e. the pore reading head in the context of nanopore sensing applications) and relevant diameter measurements.

FIG. 3 shows the structural motif that contributes to the pore constriction comprising three stacked concentric side-chain layers: Tyr 51, Asn 55 and Phe 56.

FIG. 4 shows the sequence homology in CsgG homologues, including the multiple sequence alignment of CsgG-like proteins (SEQ ID NO: 442 to SEQ ID NO: 448). The selected sequences were chosen from monophyletic clades across the phylogenetic tree of CsgG-like sequences (not shown), to give a representative view of sequence diversity. Secondary structure elements are shown as arrows or bars for β-strands and α-helices, respectively, and are based on the E. coli CsgG crystal structure. Importantly, the residues equivalent to E. coli Tyr 51, Asn55 and Phe 56 are highlighted by arrows. These residues form the pore's inner constriction, i.e. the pore reading head in the context of nanopore sensing applications.

FIG. 5 shows representative single-channel current recordings (a) and conductance histogram (b) of CsgG reconstituted in planar phospholipid bilayers and measured under an electrical field of +50 mV (n=33) or −50 mV (n=13).

FIG. 6 shows single-channel current recordings of PPB-reconstituted CsgG at +50 mV or −50 mV and supplemented with incremental concentrations of CsgE. Horizontal scale bars lie at 0 pA.

FIG. 7 shows a, Raw negative-stain EM image of C8E4/LDAO-solubilized CsgG. Arrows indicate the different particle populations as labelled in the size exclusion profile shown in g, being (I) aggregates of CsgG nonamers, (II) CsgG octadecamers and (III) CsgG nonamers. Scale bar, 20 nm. b, Representative class average for top and side views of the indicated oligomeric states. c, Rotational autocorrelation function graph of LDAO-solubilized CsgG in top view, showing nine-fold symmetry. d, Raw negative-stain EM image of CsgG_{C1S}. Arrows indicate the hexadecameric (IV) and octameric (V) particles observed by size-exclusion chromatography in g. e, Representative class average for side views of CsgG_{C1S} oligomers. No top views were observed for this construct. f, Table of elution volumes (EV) of CsgG_{C1S} and CsgG particles observed by size-exclusion chromatography shown in g, calculated molecular mass (MW_{calc}), expected molecular mass (MW_{CsgG}) corresponding CsgG oligomerization state (CsgGₙ) and the particles' symmetry as observed by negative-stain EM and X-ray crystallography. g, Size-exclusion chromatogram of CsgG_{C1S} (black) and C8E4/LDAO-solubilized CsgG (grey) run on Superdex 200 10/300 GL (GE Healthcare). h, i, Ribbon representation of crystallized oligomers in top and side view, showing the D₈ hexadecamers for CsgG_{C1S} (h) and D₉ octadecamers for membrane-extracted CsgG (i). One protomer is coloured in rainbow from N terminus (blue) to C terminus (red). The two C₈ octamers (CsgG_{C1S}) or C₉ nonamers (CsgG) that form the tail-to-tall dimers captured in the crystals are coloured blue and tan. r and θ give radius and interprotomer rotation, respectively.

FIG. 8 shows an electron density map at 2.8 Å for CsgG_{C1S} calculated using NCS-averaged and density-modified experimental SAD phases, and contoured at 1.5 g. The map shows the region of the channel construction (CL; a single protomer is labelled) and is overlaid on the final refined model. CsgG_{C1S} is a mutant CsgG where the N-terminal Cys of the mature CsgG sequence, i.e. Cys 1, is replaced by Ser, resulting in lack of lipid modification by the E. coli LOL pathway. This results in a soluble homooctameric oligomer that is present in a pre-pore conformation (see FIG. 42) contrary to the membrane-targeted homononameric pore formed by native, lipid-modified CsgG (FIG. 43).

FIG. 9 shows top (FIG. 9a) and side (FIG. 9b) views of the CsgG constriction modelled with a polyalanine chain threaded through the channel in an extended conformation, shown in a C-terminal to N-terminal direction. The modelled solvation of the polyalanine chain, position as in FIG. 9b, is shown in FIG. 9c with C-loops removed for clarity (shown solvent molecules are those within 10 Å of the full polyalanine chain).

FIG. 10: Illustrates CsgG from E. coli.

FIG. 11: Illustrates the dimensions of CsgG.

FIG. 12: Illustrates single G translocation at 10 Å/ns. There is a large barrier for entry of guanine into F56 ring in CsgG-Eco. *=G enters F56 ring. A=G stops interacting with 56 ring. B=G stops interacting with 55 ring. C=G stops interacting with 51 ring.

FIG. 13: Illustrates ssDNA translocation at 100 Å/ns. A larger force is required to pull the DNA through the constriction for CsgG-Eco.

FIG. 14: Illustrates ss DNA translocation at 10 Å/ns. CsgG-F56A-N55S and CsgG-F56A-N55S-Y51A mutants both have a lower barrier for ssDNA translocation.

FIGS. 15 to 17: Mutant pores showing increased range compared with wild-type (WT).

FIGS. 18 and 19: Mutant pores showing increased throughput compared with wild-type (WT).

FIGS. 20 and 21: Mutant pore showing increased insertion compared with wild-type (WT).

FIG. 22: shows the DNA construct X used in Example 18. The region labelled 1 corresponded to 30 SpC3 spacers. The region labelled 2 corresponded to SEQ ID NO: 415. The region labeled 3 corresponded to four iSp18 spacers. The region labelled 4 corresponded to SEQ ID NO: 416. The section labelled 5 corresponded to four 5-nitroindoles. The region labelled 6 corresponded to SEQ ID NO: 417. The region labelled 7 corresponded to SEQ ID NO: 418. The region labelled 8 corresponded to SEQ ID NO: 419 which had four iSp18 spacers (the region labelled 9) attached at the 3′ end of SEQ ID NO: 419. At the opposite end of the iSp18 spacers was a 3′ cholesterol tether (labelled 10). The region labelled 11 corresponded to four SpC3 spacers.

FIG. 23: shows an example chromatography trace of Strep trap (GE Healthcare) purification of CsgG protein (x-axis label=elution volume (mL), Y-axis label=Absorbance (mAu)). The sample was loaded In 25 mM Tris, 150 mM NaCl, 2 mM EDTA, 0.01% DDM and eluted with 10 mM desthiobiotin. The elution peak in which CsgG protein eluted is labelled as E1.

FIG. 24: shows an example of a typical SDS-PAGE visualisation of CsgG protein after the initial strep purification. A 4-20% TGX Gel (Bio Rad) was run at 300 V for 22 minutes in 1×TGS buffer. The gel was stained with Sypro Ruby stain. Lanes 1-3 show the main elution peak (labelled E1 in FIG. 23) which contained CsgG protein as indicated by the arrow. Lanes 4-6 corresponded to elution fractions of the tail of the main elution peak (labelled E1 in FIG. 23) which contained contaminants. M shows the molecular weight marker used which was a Novex Sharp Unstained (unit=kD).

FIG. 25: Shows an example of a size exclusion chromatogram (SEC) of CsgG protein (120 mL S200 GE healthcare, x-axis label=elution volume (mL), y-axis label=absorbance (mAu)). The SEC was carried out after strep purification and heating the protein sample. The running buffer for SEC was 25 mM Tris, 150 mM NaCl, 2 mM EDTA, 0.01% DDM, 0.1% SDS, pH 8.0 and the column was run at 1 mL/minute rate. The trace labelled X shows absorbance at 220 nm and the trace labelled Y shows absorbance at 280 nm. The peak labelled with a star was collected.

FIG. 26: shows an example of a typical SDS-PAGE visualisation of CsgG protein after SEC. A 4-20% TGX Gel (Bio Rad) was run at 300V for 22 minutes in 1×TGS buffer and the gel was stained with Sypro Ruby stain. Lane 1 shows CsgG protein sample after strep purification and heating but before SEC. Lanes 2-8 show fractions collected across the peak running approximately 48 mL-60 mL of FIG. 25 (mid peak=55 mL) and labelled with a star in FIG. 25. M shows the molecular weight marker used which was a Novex Sharp Unstained (unit=kD). The bar corresponding to the CsgG-Eco pore is indicated by an arrow.

FIGS. 27 to 33: Mutant pores showing increased range compared with wild-type (WT).

FIGS. 34 to 39: Mutant pores showing increased range compared with wild-type (WT).

FIG. 40 shows snap shots of the enzyme (T4 Dda—(E94C/C109A/C136A/A360C) (SEQ ID NO: 412 with mutations E94C/C109A/C136A/A360C and then (ΔM1)G1G2)) on top of the pore (CsgG-Eco-(Y51T/F56Q)-StrepII(C))9 (SEQ ID NO: 390 with mutations Y51T/F56Q where StepII(C) is SEQ ID NO: 435 and is attached at the C-terminus pore mutant No. 20)) taken at 0 and 20 ns during the simulations (Runs 1 to 3).

FIG. 41 shows snap shots of the enzyme (T4 Dda—(E94C/C109A/C136A/A360C) (SEQ ID NO: 412 with mutations E94C/C109A/C136A/A360C and then (ΔM1)G1G2)) on top of the pore (CsgG-Eco-(Y51T/F56Q)-StrepII(C))9 (SEQ ID NO: 390 with mutations Y51T(F56Q where StepII(C) is SEQ ID NO: 435 and is attached at the C-terminus pore mutant No. 20)) taken at 30 and 40 ns during the simulations (Runs 1 to 3).

FIG. 42 shows X-ray structure of CsgG_{C1S} in pre-pore conformation. a, Ribbon diagram of the CsgG_{C1S} monomer coloured as a blue to red rainbow from N terminus to C terminus. Secondary structure elements are labelled according to the ABD-like fold, with the additional N-terminal and C-terminal a-helices and the extended loop connecting β1 and α1 labelled αN, αC and C-loop (CL), respectively. b, Side view of the CsgG_{C1S}C8 octamer with subunits differentiated by colour and one subunit oriented and coloured as in a.

FIG. 43 shows the structure of CsgG in its channel conformation, a, Amide I region (1,700-1,600 cm⁻¹) of ATR-FTIR spectra of CsgGC1S (blue) and membrane extracted CsgG (red). b, TM1 and TM2 sequence (SEQ ID NO: 449 and SEQ ID NO: 450) (bilayer-facing residues in blue) and Congo red binding of E. coli BW25141ΔcsgG complemented with wild-type csgG (WT), empty vector or csgG lacking the underlined fragments of TM1 or TM2. Data are representative of three biological replicates. c, Overlay of CsgG monomer in pre-pore (light blue; TM1 pink, TM2 purple) and channel conformation (tan; TM1 green, TM2 orange). CL, C-loop. d, e, Side view (d) and cross-sectional view (e) of CsgG nonamers in ribbon and surface representation; helix 2, the core domain and TM hairpins are shown in blue, light blue and tan, respectively. A single protomer is coloured as in FIG. 42a. Magenta spheres show the position of Leu 2. OM, outer membrane.

FIG. 44 shows the CsgG channel constriction. a, Cross-section of CsgG channel constriction and its solvent-excluded diameters. b, The constriction is composed of three stacked concentric side-chain layers: Tyr 51, Asn 55 and Phe 56, preceded by Phe 48 from the periplasmic side. c, CsgG channel topology. d, Congo red binding of E. coli BW25141ΔcsgG complemented with csgG (WT), empty vector or csgG carrying indicated constrictions mutants. Data are representative of six biological replicates. e, f, Representative single channel current recordings (e) and conductance histogram (f) of CsgG reconstituted in planar phospholipid bilayers and measured under an electrical field of +50 mV (n=33) or −50 mV (n=13).

FIG. 45 shows a model of CsgG transport mechanism. a, NativePAGE of CsgE (E), CsgG (G) and CsgG supplemented with excess CsgE (E+G), showing the formation of a CsgG-CsgE complex (E−G*). Data are representative of seven experiments, encompassing four protein batches. b, SDS-PAGE of CsgE (E), CsgG (G) and the E−G* complex recovered from native PAGE. Data are representative of two repetitions. M, molecular mass markers. c, Selected class averages of CsgG-CsgE particles. From left to right: top and side view visualized by cryo-EM, and comparison of negatively stained side views with CsgG nonamers. d, Cryo-EM averages of top and tilted side-viewed CsgE particles. Rotational autocorrelation shows nine-fold symmetry. e, Three-dimensional reconstruction of CsgG-CsgE (24A° resolution, 1.221 single particles) shows a nonameric particle comprising CsgG (blue) and an additional density assigned as a CsgE nonamer (orange). f, Single-channel current recordings of PPB reconstituted CsgG at +50 mV or −50 mV and supplemented with incremental concentrations of CsgE Horizontal scale bars lie at 0 pA. g, Tentative model for CsgG-mediated protein secretion. CsgG and CsgE are proposed to form a secretion complex that entraps CsgA (discussed in FIG. 54), generating an entropic potential over the channel. After capture of CsgA in the channel constriction, a DS-rectified Brownian diffusion facilitates the progressive translocation of the polypeptide across the outer membrane.

FIG. 46 shows the Curti biosynthesis pathway in E. coli. The major curli subunit CsgA (light green) is secreted from the cell as a soluble monomeric protein. The minor curli subunit CsgB (dark green) is associated with the outer membrane (OM) and acts as a nucleator for the conversion of CsgA from a soluble protein to amyloid deposit. CsgG (orange) assembles into an oligomeric curli-specific translocation channel in the outer membrane. CsgE (purple) and CsgF (light blue) form soluble accessory proteins required for productive CsgA and CsgB transport and deposition. CsgC forms a putative oxidoreductase of unknown function. All curli proteins have putative Sec signal sequences for transport across the cytoplasmic (inner) membrane (IM).

FIG. 47 shows the in-solution oligomerization states of CsgG and CsgG_{C1S} analysed by size-exclusion chromatography and negative-stain electron microscopy. a, Raw negative-stain EM image of C8E4/LDAO solubilized CsgG. Arrows indicate the different particle populations as labeled in the size exclusion profile shown in g, being (1) aggregates of CsgG nonamers, (II) CsgG octadecamers and (III) CsgG nonamers. Scale bar, 20 nm. b, Representative class average for top and side views of the indicated oligomeric states. c, Rotational autocorrelation function graph of LDAO solubilized CsgG in top view, showing nine-fold symmetry. d, Raw negative stain EM image of CsgG_{C1S}. Arrows indicate the hexadecameric (IV) and octameric (V) particles observed by size-exclusion chromatography in g. e, Representative class average for side views of CsgG_{C1S} oligomers. No top views were observed for this construct. f, Table of elution volumes (EV) of CsgG_{C1S} and CsgG particles observed by size-exclusion chromatography shown in g, calculated molecular mass (MWcalc), expected molecular mass (MWCsgG) corresponding CsgG oligomerization state (CsgGₙ) and the particles' symmetry as observed by negative-stain EM and X-ray crystallography. g, Size-exclusion chromatogram of CsgG_{C1S} (black) and C8E4/LDAO-solubilized CsgG (grey) run on Superdex 200 101300 GL (GE Healthcare). h, i, Ribbon representation of crystallized oligomers in top and side view, showing the D8 hexadecamers for CsgG_{C1S} (h) and D9 octadecamers for membrane-extracted CsgG (i). One protomer is coloured in rainbow from N terminus (blue) to C terminus (red). The two C8 octamers (CsgG_{C1S}) or C9 nonamers (CsgG) that form the tail-to-tail dimers captured in the crystals are coloured blue and tan. r and h give radius and interprotomer rotation, respectively.

FIG. 48 shows a comparison of CsgG with structural homologues and interprotomer contacts in CsgG. a, b, Ribbon diagram for the CsgG_{C1S} monomer (for example CsgG in pre-pore conformation) (a) and the nucleotide-binding-domain-like domain of ToIB (b) (PDB 2hqs), both coloured in rainbow from N terminus (blue) to C terminus (red). Common secondary structure elements are labelled equivalently. c, CsgG_{C1S} (grey) in superimposition with, from left to right, Xanthomonas campestris rare lipoprotein B (PDB 2r76, coloured pink), Shewanella oneidensis hypothetical lipoprotein DUF330 (PDB 2iqi, coloured pink) and Escherichia coli ToIB (PDB 2hqs, coloured pink and yellow for the N-terminal and b-propeller domains, respectively). CsgG-specific structural elements are labelled and coloured as in the upper left panel. d, e, Ribbon diagram of two adjacent protomers as found in the CsgG structure, viewed along the plane of the bilayer, either from outside (c) or inside (d) the oligomer. One protomer is shown in rainbow (dark blue to red) from N terminus to C terminus; a second protomer is shown in light blue (core domain), blue (helix 2) and tan (TM domain). Four main oligomerization interfaces are apparent: b6-b39 main-chain interactions inside the b-barrel, the constriction loop (CL), side-chain packing of helix 1 (α1) against b1-b3-b4-b5, and helix-helix packing of helix 2 (α2). The 18-residue N-terminal loop connecting the lipid anchor (a magenta sphere shows the Ca position of Leu 2) and N-terminal helix (αN) is also seen to wrap over the adjacent two protomers. The projected position of the lipid anchor is expected to lie against the TM1 and TM2 hairpins of the +2 protomer (not shown for clarity).

FIG. 49 shows Cys accessibility assays for selected surface residues in the CsgG oligomers. a-c, Ribbon representation of CsgG nonamers shown In periplasmic (a), side (b) and extracellular (c) views. One protomer is coloured in rainbow from N terminus (blue) to C terminus (red). Cysteine substitutions are labelled and the equivalent locations of the S atoms are shown as spheres, coloured according to accessibility to MAL-PEG (5.000 Da) labelling in E. coli outer membranes. d, Western blot of MAL-PEG reacted samples analysed on SDS-PAGE, showing 5 kDa increase on MALPEG binding of the introduced cysteine. Accessible (11 and 111), moderately accessible (1) and inaccessible (2) sites are coloured green, orange and red, respectively, in a-e. For Arg 97 and Arg 110 a second species at 44 kDa is present, corresponding to a fraction of protein In which both the introduced and native cysteine became labelled. Data are representative of four independent experiments from biological replicates. e, Side view of the dimerization interface in the D9 octadecamer as present in the X-ray structure. Introduced cysteines in the dimerization interface or inside the lumen of the D9 particle are labelled. In membrane-bound CsgG, these residues are accessible to MAL-PEG, demonstrating that the D9 particles are an artifact of concentrated solutions of membrane-extracted CsgG and that the C9 complex forms the physiologically relevant species. Residues in the C-terminal helix (aC; Lys 242, Asp 248 and His 255) are found to be inaccessible to poorly accessible, indicating that aC may form additional contacts with the E. coli cell envelope, possibly the peptidoglycan layer.

FIG. 50 shows molecular dynamics simulation of CsgG constriction with model polyalanine chain. a, b, Top (a) and side (b) views of the CsgG constriction modelled with a polyalanine chain threaded through the channel In an extended conformation, here shown In a C-terminal to N-terminal direction. Substrate passage through the CsgG transporter is itself not sequence specific^{ref16,23}. For clarity, a polyalanine chain was used for modelling the putative interactions of a passing polypeptide chain. The modelled area is composed of nine concentric CsgG C-loops, each comprising residues 47-58. Side chains lining the constriction are shown in stick representation, with Phe 51 coloured slate blue, Asn 55 (amide-clamp) cyan, and Phe 48 and Phe 56 (Φ-clamp) in light and dark orange, respectively. N, O and H atoms (only hydroxyl or side-chain amide H atoms are shown) are coloured blue, red and white, respectively. The polyalanine chain is coloured green, blue, red and white for C, N, O and H atoms, respectively. Solvent molecules (water) within 10 Å of the polyalanine residues inside the constriction (residues labelled 11 to 15) are shown as red dots. c, Modelled solvation of the polyalanine chain, position as in b and with C-loops removed for clarity (shown solvent molecules are those within 10 Å of the full polyalanine chain). At the height of the amide-clamp and Φ-clamp, the solvation of the polyalanine chain is reduced to a single water shell that bridges the peptide backbone and amide-clamp side chains. Most side chains in the Tyr 51 ring have rotated towards the solvent in comparison with their inward, centre-pointing position observed in the CsgG (and the CsgG_{C1S}) X-ray structure. The model is the result of a 40 ns all-atom explicit solvent molecular dynamics simulation with GROMACS^{ref53} using the AMBER99SB-ILDN54 force field and with the Ca atoms of the residues at the extremity of the C-loop (Gln 47 and Thr 58) positionally restricted.

FIG. 51 shows sequence conservation in CsgG homologues. a, Surface representation of the CsgG nonamer coloured according to sequence similarity (coloured yellow to blue from low to high conservation score) and viewed from the periplasm (far left), the side (middle left), the extracellular milieu (middle right) or as a cross-sectional side view (far right). The figures show that the regions of highest sequence conservation map to the entry of the periplasmic vestibule, the vestibular side of the constriction loop and the luminal surface of the TM domain. b, Multiple sequence alignment of CsgGlike lipoproteins. The selected sequences were chosen from monophyletic clades across the phylogenetic three of CsgG-like sequences (not shown), to give a representative view of sequence diversity. Secondary structure elements are shown as arrows or bars for β-strands and α-helices, respectively, and are based on the E. coli CsgG crystal structure. c, d, CsgG protomer in secondary structure representation (c) and a cross-sectional side view (d) of the CsgG nonamer in surface representation, both coloured grey and with three continuous blocks of high sequence conservation coloured red (HCR1), blue (HCR2) and yellow (HCR3). HCR1 and HCR2 shape the vestibular side of the constriction loop; HCR3 corresponds to helix 2, lying at the entry of the periplasmic vestibule. Inside the constriction, Phe 56 is 100% conserved, whereas Asn 55 can be conservatively replaced by Ser or Thr, for example by a small polar side chain that can act as hydrogen-bond donor/acceptor. The concentric side-chain ring at the exit of the constriction (Tyr 51) is not conserved. The presence of the Phe-ring at the entrance of the constriction is topologically similar to the Phe 427-ring (referred to as the 0-clamp) in the anthrax protective antigen PA63, in which it was shown to catalyse polypeptide capture and passage^{ref20}. MST of toxB superfamily proteins reveals a conserved motif D(D/Q)(F)(S/N)S at the height of the Phe-ring. This is similar to the S(Q/N/T)(F)ST motif seen in curli-like transporters. Although an atomic resolution structure of PA63 in pore conformation is not yet available, available structures suggest the Phe-ring may similarly be followed by a conserved hydrogen-bond donor/acceptor (Ser/Asn 428) as a subsequent concentric ring in the translocation channel (note that the orientation of the element is inverted in both transporters).

FIG. 52 shows single-channel current analysis of CsgG and CsgG:CsgE pores. a, Under negative field potential, CsgG pores show two conductance states. The upper left and right panels show a representative single-channel current trace of, respectively, the normal (measured at +50, 0 and −50 mV) and the low-conductance forms (measured at 0, +50 and −50 mV). No conversions between both states were observed during the total observation time (n=22), indicating that the conductance states have long lifetimes (second to minute timescale). The lower left panel shows a current histogram for the normal and low-conductance forms of CsgG pores acquired at +50 and −50 mV (n=33). I-V curves for CsgG pores with regular and low conductance are shown in the lower right panel. The data represent averages and standard deviations from at least four independent recordings. The nature or physiological existence of the low-conductance form is unknown. b, Electrophysiology of CsgG channels titrated with the accessory factor CsgE. The plots display the fraction of open, intermediate and closed channels as a function of CsgE concentration. Open and closed states of CsgG are illustrated in FIG. 45f. Increasing the concentration of CsgE to more than 10 nM leads to the closure of CsgG pores. The effect occurs at +50 mV (left) and −50 mV (right), ruling out the possibility that the pore blockade is caused by electrophoresis of CsgE (calculated pl 4.7) Into the CsgG pore. An infrequent (5%) Intermediate state has roughly half the conductance of the open channel. It may represent CsgE-induced incomplete closures of the CsgG channel; alternatively, it could represent the temporary formation of a CsgG dimer caused by the binding of residual CsgG monomer from the electrolyte solution to the membrane-embedded pore. The fraction for the three states was obtained from all-point histogram analysis of single-channel current traces. The histograms yielded peak areas for up to three states, and the fraction for a given state was obtained by dividing the corresponding peak area by the sum of all other states in the recording. Under negative field potential, two open conductance states are discerned, similar to the observations for CsgG (see a). Because both open channel variations were blocked by higher CsgE concentrations, the ‘open’ traces in b combine both conductance forms. The data in the plot represent averages and standard deviations from three independent recordings. c, The crystal structure, size-exclusion chromatography and EM show that detergent extracted CsgG pores form non-native tail-to-tail stacked dimers (for example, two nonamers as D9 particle; FIG. 47) at higher protein concentration. These dimers can also be observed in single-channel recordings. The upper panel shows the single channel current trace of a stacked CsgG pore at +50, 0 and −50 mV (left to right). The lower left panel shows a current histogram of dimeric CsgG pores recorded at +50 and −50 mV. The experimental conductances of +16.2±1.8 and −16.0±3.0 pA (n=15) at +50 and −50 mV, respectively, are near the theoretically calculated value of 23 pA. The lower right panel shows an I-V curve for the stacked CsgG pores. The data represent averages and standard deviations from six independent recordings. d, The ability of CsgE to bind and block stacked CsgG pores was tested by electrophysiology. Shown are single-channel current traces of stacked CsgG pore in the presence of 10 or 100 nM CsgE at +50 mV (upper) and −50 mV (lower). The current traces indicate that otherwise saturating concentrations of CsgE do not lead to pore closure for stacked CsgG dimers. These observations are in good agreement with the mapping of the CsgG-CsgE contact zone to helix 2 and the mouth of the CsgG periplasmic cavity as discerned by EM and site-directed mutagenesis (FIG. 45 and FIG. 52).

FIG. 53 shows CsgE oligomer and CsgG-CsgE complex. a, Size exclusion chromatography of CsgE (Superose 6, 16/600; running buffer 20 mM Tris-HCl pH8, 100 mM NaCl, 2.5% glycerol) shows an equilibrium of two oligomeric states, 1 and 2, with an apparent molecular mass ratio of 9.16:1. Negative-stain EM inspection of peak 1 shows discrete CsgE particles (five representative class averages are shown in the inset, ordered by increasing tilt angles) compatible in size with nine CsgE copies. b, Selected class average of CsgE oligomer observed in top view by cryo-EM and its rotational autocorrelation show the presence of C9 symmetry. c, FSC analysis of CsgG-CsgE cryo-EMmodel Three-dimensional reconstruction achieved a resolution of 24 Å as determined by FSC at a threshold of 0.5 correlation using 125 classes corresponding to 1,221 particles. d, Overlay of CsgG-CsgE cryo-EM density and the CsgG nonamer observed in the X-ray structure. The overlays are shown viewed from the side as semi-transparent density (left) or as across-sectional view. e, Congo red binding of E. coli BW25141ΔcsgG complemented with wild-type csgG (WT), empty vector (ΔcsgG) or csgGhelix 2 mutants (single amino acid replacements labelled in single-letter code). Data are representative of four biological replicates. f, Effect of bile salt toxicity on E. coli LSR12 complemented with csgG (WT) or on csgG carrying different helix 2 mutations, complemented with (1) or without (2) csgE. Tenfold serial dilution starting from 107 bacteria were spotted on McConkey agar plates. Expression of the CsgG pore in the outer membrane leads to an increased bile salt sensitivity that can be blocked by co-expression of CsgE (n=6, three biological replicates, with two repetitions each). g, Cross-sectional view of CsgG X-ray structure in molecular surface representation. CsgG mutants without an effect on Congo red binding or toxicity are shown in blue; mutants that interfere with CsgE-mediated rescue of bile salt sensitivity are indicated in red.

FIG. 54 shows assembly and substrate recruitment of the CsgG secretion complex. The curli transporter CsgG and the soluble secretion cofactor CsgE form a secretion complex with 9:9 stoichiometry that encloses a 24,000 Å³ chamber that is proposed to entrap the CsgA substrate and facilitate its entropy-driven diffusion across the outer membrane (OM; see the text and FIG. 45). On theoretical grounds, three putative pathways (a-c) for substrate recruitment and assembly of the secretion complex can be envisaged. a, A ‘catch-and-cap’ mechanism entails the binding of CsgA to the apo CsgG translocation channel (1), leading to a conformational change in the latter that exposes a high-affinity binding platform for CsgE binding (2). CsgE binding leads to capping of the substrate cage. On secretion of CsgA, CsgG would fall back into Its low-affinity conformation, leading to CsgE dissociation and liberation of the secretion channel for a new secretion cycle. b, In a ‘dock and-trap’ mechanism, periplasmic CsgA is first captured by CsgE (1), causing the latter to adopt a high-affinity complex that docks onto the CsgG translocation pore (2), enclosing CsgA in the secretion complex. CsgA binding could be directly to CsgE oligomers or to CsgE monomers, the latter leading to subsequent oligomerization and CsgGbinding. Secretion of CsgA leads CsgE to fall back into its low-affinity conformation and to dissociate from the secretion channel. c, CsgG and CsgE form a constitutive complex, in which CsgE conformational dynamics cycle between open and closed forms In the course of CsgA recruitment and secretion. Currently published or available data do not allow us to discriminate between these the putative recruitment modes or derivatives thereof, or to put forward one of them.

FIG. 55 shows data collection statistics and electron density maps of CsgG_{C1S} and CsgG. a, Data collection statistics for CsgG_{C1S} and CsgG X-ray structures. b, Electron density map at 2.8 Å for CsgG_{C1S} calculated using NCS-averaged and density-modified experimental SAD phases, and contoured at 1.5a. The map shows the region of the channel construction (CL; a single protomer is labeled) and is overlaid on the final refined model. c, Electron density map (resolutions 3.6, 3.7 and 3.8 Å A along reciprocal vectors a*, b* and c*, respectively) in the CsgG TM domain region, calculated from NCS averaged and density-modified molecular replacement phases (TM loops were absent from the Input model); B-factor sharpened by −20 Å² and contoured at 1.0σ. The figure shows the TM1 (Lys 135-Leu 154) and TM2 (Leu 182-Asn 209) region of a single CsgG protomer, overlaid on the final refined model.

FIG. 56 shows a single channel current trace (left) and zoomed region thereof (right) of a CsgG WT protein interacting with a DNA hairpin carrying a single-stranded DNA overhang. The trace shows the current which alters in response to the potential measured at +50 mV or −50 mV intervals (indicated by arrows). The downward current blockades in the last +50 mV segment represent the simultaneous lodging of the hairpin duplex inside the pore lumen and threading of the single-stranded hairpin end into inner pore construction leading to an almost complete current blockade. Reversal of the electrical field to −50 mV results in the electrophoretic unblocking of the pore. A new +50 mV episode results again in DNA hairpin lodging/threading and pore blockage. On the +50 mV segments, unfolding of the hairpin structure can lead to the termination of the current blockade indicated by the reversal of the current blockade. The hairpin with the sequence was 3′ GCGGGGA GCGTATT AGAGTTG GATCGGATGCA GCTGGCTACTGACGTCA TGACGTCAGTAGCCAGCATGCATCCGATC-5′ was added to the cis side of the chamber at a final concentration of 10 nM.

FIG. 57 shows the purification and channel properties of CsgG-ΔPYPA, a mutant CsgG pore where the PYPA sequence (residues 50-53) at the constriction residue Y51 is mutated to GG.

### DESCRIPTION OF THE SEQUENCE LISTING

SEQ ID NO: 1 shows amino acid sequence of wild-type E. coli CsgG including signal sequence (Uniprot accession number P0AEA2).

SEQ ID NO: 2 shows polynucleotide sequence of wild-type E. coli CsgG including signal sequence (Gene ID: 12932538).

SEQ ID NO: 3 shows the amino acid sequence of the wild-type E. coli CsgG from positions 53 to 77 of SEQ ID NO:2. This corresponds to the amino acid sequence from positions 38 to 63 of the mature wild-type E. coli CsgG monomer (i.e. lacking the signal sequence).

SEQ ID NOs 4 to 388 show the amino acid sequence from positions 38 to 63 of the modified monomers of CsgG lacking the signal sequence.

SEQ ID NO: 389 shows the codon optimised polynucleotide sequence encoding the wild-type CsgG monomer from Escherchia coli Str. K-12 substr. MC4100. This monomer lacks the signal sequence.

SEQ ID NO: 390 shows the amino acid sequence of the mature form of the wild-type CsgG monomer from Escherchia coli Str. K-12 substr. MC4100. This monomer lacks the signal sequence. The abbreviation used for this CsgG=CsgG−Eco.

SEQ ID NO: 391 shows the amino acid sequence of YP_001453594.1: 1-248 of hypothetical protein CKO_02032 [Citrobacter koseri ATCC BAA-895], which is 99% identical to SEQ ID NO: 390.

SEQ ID NO: 392 shows the amino acid sequence of WP_001787128.1: 16-238 of curli production assembly/transport component CsgG, partial [Salmonella enterica], which is 98% to SEQ ID NO: 390.

SEQ ID NO: 393 shows the amino acid sequence of KEY44978.1|: 16-277 of curli production assembly/transport protein CsgG [Citrobacter amalonaticus], which is 98% identical to SEQ ID NO: 390.

SEQ ID NO: 394 shows the amino acid sequence of YP_003364699.1: 16-277 of curli production assembly/transport component [Citrobacter rodentium ICC168], which is 97% identical to SEQ ID NO: 390.

SEQ ID NO: 395 shows the amino acid sequence of YP_004828099.1: 16-277 of curli production assembly/transport component CsgG [Enterobacter asburise LF7a], which is 94% identical to SEQ ID NO: 390.

SEQ ID NO: 396 shows the polynucleotide sequence encoding the Phi29 DNA polymerase.

SEQ ID NO: 397 shows the amino acid sequence of the Phi29 DNA polymerase.

SEQ ID NO: 398 shows the codon optimised polynucleotide sequence derived from the sbcB gene from E. coli. It encodes the exonuclease I enzyme (EcoExo I) from E. coli SEQ ID NO: 399 shows the amino acid sequence of exonuclease I enzyme (EcoExo I) from E. coli.

SEQ ID NO: 400 shows the codon optimised polynucleotide sequence derived from the xthA gene from E. coli. It encodes the exonuclease III enzyme from E. coli.

SEQ ID NO: 401 shows the amino acid sequence of the exonuclease III enzyme from E. coli. This enzyme performs distributive digestion of 5′ monophosphate nucleosides from one strand of double stranded DNA (dsDNA) in a 3′-5′ direction. Enzyme initiation on a strand requires a 5′ overhang of approximately 4 nucleotides.

SEQ ID NO: 402 shows the codon optimised polynucleotide sequence derived from the reJ gene from T. thermophilus. It encodes the RecJ enzyme from T. thermophilus (TthRecJ-cd).

SEQ ID NO: 403 shows the amino acid sequence of the RecJ enzyme from T. thermophilus (TthRecJ-cd). This enzyme performs processive digestion of 5′ monophosphate nucleosides from ssDNA in a 5′-3′ direction. Enzyme initiation on a strand requires at least 4 nucleotides.

SEQ ID NO: 404 shows the codon optimised polynucleotide sequence derived from the bacteriophage lambda exo (redX) gene. It encodes the bacteriophage lambda exonuclease.

SEQ ID NO: 405 shows the amino acid sequence of the bacteriophage lambda exonuclease. The sequence is one of three identical subunits that assemble into a trimer. The enzyme performs highly processive digestion of nucleotides from one strand of dsDNA, in a 5′-3′direction (http://www.neb.com/nebecomm/products/productM0262.asp). Enzyme Initiation on a strand preferentiality requires a 5′ overhang of approximately 4 nucleotides with a 5′ phosphate.

SEQ ID NO: 406 shows the amino acid sequence of Hel308 Mbu.

SEQ ID NO: 407 shows the amino acid sequence of Hel308 Csy.

SEQ ID NO: 408 shows the amino acid sequence of Hel308 Tga.

SEQ ID NO: 409 shows the amino acid sequence of Hel308 Mhu.

SEQ ID NO: 410 shows the amino acid sequence of Tral Eco.

SEQ ID NO: 411 shows the amino acid sequence of XPD Mbu.

SEQ ID NO: 412 shows the amino acid sequence of Dda 1993.

SEQ ID NO: 413 shows the amino acid sequence of Trwc Cba.

SEQ ID NO: 414 shows the amino acid sequence of WP_006819418.1: 19-280 of transporter [Yokenella regensburgei], which is 91% identical to SEQ ID NO: 390.

SEQ ID NO: 415 shows the amino acid sequence of WP_024556654.1: 16-277 of curli production assembly/transport protein CsgG [Cronobacter pulveris], which is 89% identical to SEQ ID NO: 390.

SEQ ID NO: 416 shows the amino acid sequence of YP_005400916.1: 16-277 of curli production assembly/transport protein CsgG [Rahnella aquatilis HX2], which is 84% identical to SEQ ID NO: 390.

SEQ ID NO: 417 shows the amino acid sequence of KFC99297.1: 20-278 of CsgG family curli production assembly/transport component [Kluyvera ascorbata ATCC 33433], which is 82% identical to SEQ ID NO: 390.

SEQ ID NO: 418 shows the amino acid sequence of KFC86716.1|: 16-274 of CsgG family curli production assembly/transport component [Hafnia alvei ATCC 13337], which is 81% identical to SEQ ID NO: 390.

SEQ ID NO: 419 shows the amino acid sequence of YP_007340845.1|: 16-270 of uncharacterised protein involved in formation of curli polymers [Enterobacteriaceae bacterium strain FGI 57], which is 76% identical to SEQ ID NO: 390.

SEQ ID NO: 420 shows the amino acid sequence of WP_010861740.1: 17-274 of curli production assembly/transport protein CsgG [Plesiomonas shigelloides], which is 70% identical to SEQ ID NO: 390.

SEQ ID NO: 421 shows the amino acid sequence of YP_205788.1: 23-270 of curli production assembly/transport outer membrane lipoprotein component CsgG [Vibrio fischeri ES114], which is 60% identical to SEQ ID NO: 390

SEQ ID NO: 422 shows the amino acid sequence of WP_017023479.1: 23-270 of curli production assembly protein CsgG [Aliivibrio logei], which is 59% identical to SEQ ID NO: 390.

SEQ ID NO: 423 shows the amino acid sequence of WP_007470398.1: 22-275 of Curli production assembly/transport component CsgG [Photobacterium sp. AK15], which is 57% identical to SEQ ID NO: 390.

SEQ ID NO: 424 shows the amino acid sequence of WP_021231638.1: 17-277 of curli production assembly protein CsgG [Aeromonas veronii], which is 56% identical to SEQ ID NO: 390. SEQ ID NO: 425 shows the amino acid sequence of WP_033538267.1: 27-265 of curli production assembly/transport protein CsgG [Shewanella sp. ECSMB14101], which is 56% identical to SEQ ID NO: 390.

SEQ ID NO: 426 shows the amino acid sequence of WP_003247972.1: 30-262 of curli production assembly protein CsgG [Pseudomonas putida], which is 54% identical to SEQ ID NO: 390.

SEQ ID NO: 427 shows the amino acid sequence of YP_003557438.1: 1-234 of curli production assembly/transport component CsgG [Shewanella violacea DSS12], which is 53% identical to SEQ ID NO: 390.

SEQ ID NO: 428 shows the amino acid sequence of WP_027859066.1: 36-280 of curli production assembly/transport protein CsgG [Marinobacterium jannaschii], which is 53% identical to SEQ ID NO: 390.

SEQ ID NO: 429 shows the amino acid sequence of CEJ70222.1: 29-262 of Curli production assembly/transport component CsgG [Chryseobacterium oranimense G311], which is 50% identical to SEQ ID NO: 390.

SEQ ID NO: 430 shows a polynucleotide sequence used in Example 18.

SEQ ID NO: 431 shows a polynucleotide sequence used in Example 18.

SEQ ID NO: 432 shows a polynucleotide sequence used in Example 18.

SEQ ID NO: 433 shows a polynucleotide sequence used in Example 18.

SEQ ID NO: 434 shows a polynucleotide sequence used in Example 18. Attached to the 3′ end of SEQ ID NO: 434 is six iSp18 spacers which are attached at the opposite end to two thymines and a 3′ cholesterol TEG.

SEQ ID NO: 435 shows the amino acid sequence of StepII(C).

SEQ ID NO: 436 shows the amino acid sequence of Pro.

SEQ ID NO: 437 to 440 show primers from example 1.

SEQ ID NO: 441 shows the Hairpin from example 21.

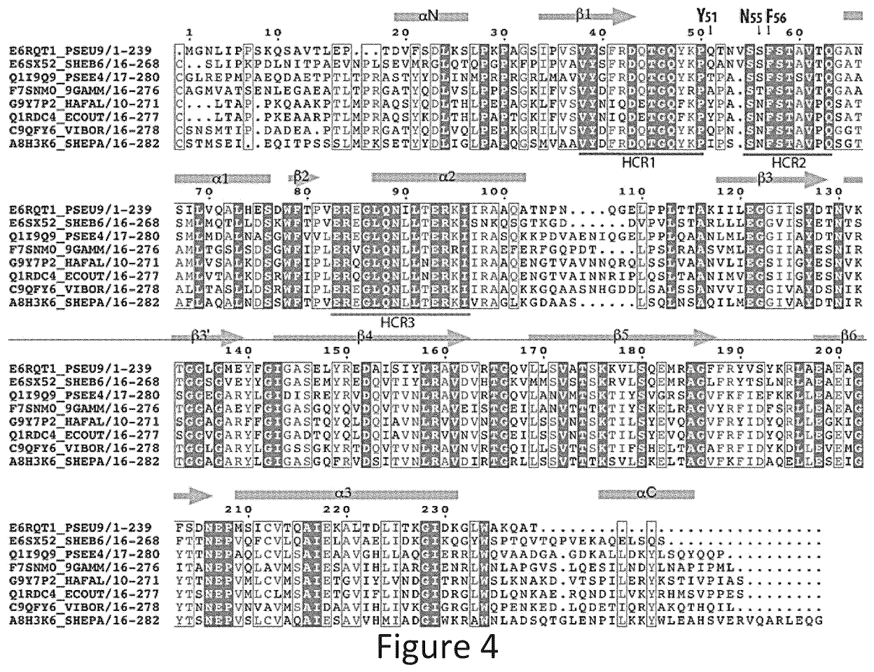

SEQ ID NO: 442 to 448 show the sequences from FIG. 4.

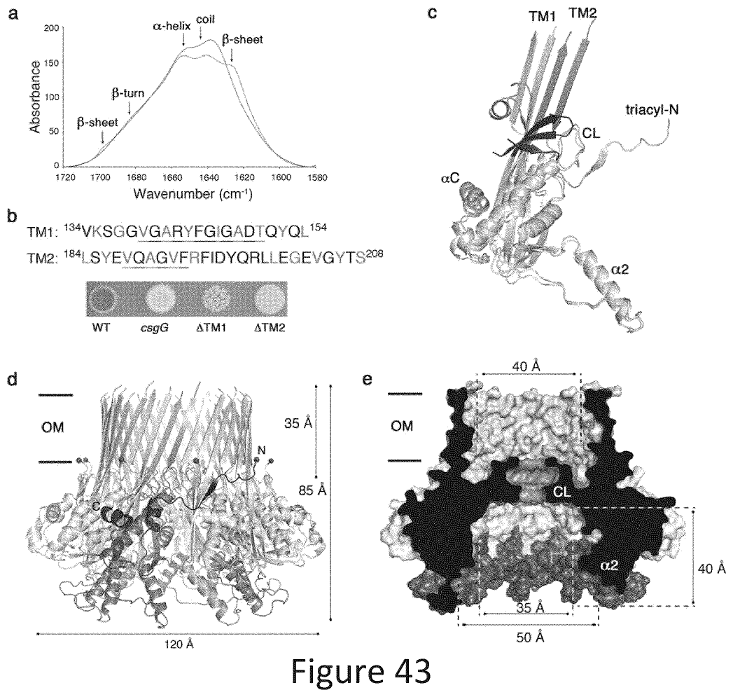

SEQ ID NO: 449 and 450 show the sequences from FIG. 43.

### DETAILED DESCRIPTION OF THE INVENTION

Unless otherwise indicated, the practice of the present invention employs conventional techniques of chemistry, molecular biology, microbiology, recombinant DNA technology, and chemical methods, which are within the capabilities of a person of ordinary skill in the art. Such techniques are also explained in the literature, for example. M. R. Green, J. Sambrook, 2012, Molecular Cloning: A Laboratory Manual, Fourth Edition, Books 1-3, Cold Spring Harbor Laboratory Press, Cold Spring Harbor. NY; Ausubel, F. M. et al. (1995 and periodic supplements: Current Protocols in Molecular Biology, ch. 9, 13, and 16, John Wiley & Sons, New York, N. Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridisation: Principles and Practice, Oxford University Press: M. J. Gait (Editor), 1984, Oligonucleotide Synthesis: A Practical Approach, IRL Press; and D. M. J. Lilley and J. E. Dahlberg, 1992, Methods of Enzymology: DNA Structure Part A: Synthesis and Physical Analysis of DNA Methods in Enzymology, Academic Press. Each of these general texts is herein incorporated by reference.

Prior to setting forth the invention, a number of definitions are provided that will assist in the understanding of the invention. All references cited herein are incorporated by reference in their entirety. Unless otherwise defined, all technical and scientific terms used herein have the same meaning as commonly understood by one of ordinary skill in the art to which this invention belongs.

As used herein, the term “comprising” means any of the recited elements are necessarily included and other elements may optionally be included as well. “Consisting essentially of” means any recited elements are necessarily included, elements that would materially affect the basic and novel characteristics of the listed elements are excluded, and other elements may optionally be included. “Consisting of” means that all elements other than those listed are excluded. Embodiments defined by each of these terms are within the scope of this invention.

The term “nucleic acid” as used herein, is a single or double stranded covalently-linked sequence of nucleotides in which the 3′ and 5′ ends on each nucleotide are joined by phosphodiester bonds. The polynucleotide may be made up of deoxyribonucleotide bases or ribonucleotide bases. Nucleic acids may include DNA and RNA, and may be manufactured synthetically in vitro or isolated from natural sources. Nucleic acids may further include modified DNA or RNA, for example DNA or RNA that has been methylated, or RNA that has been subject to post-translational modification, for example 5-capping with 7-methylguanosine, 3′-processing such as cleavage and polyadenylation, and splicing. Nucleic acids may also include synthetic nucleic acids (XNA), such as hexitol nucleic acid (HNA), cyclohexene nucleic acid (CeNA), threose nucleic acid (TNA), glycerol nucleic acid (GNA), locked nucleic acid (LNA) and peptide nucleic acid (PNA). Sizes of nucleic acids, also referred to herein as “polynucleotides” are typically expressed as the number of base pairs (bp) for double stranded polynucleotides, or in the case of single stranded polynucleotides as the number of nucleotides (nt). One thousand bp or nt equal a kilobase (kb). Polynucleotides of less than around 40 nucleotides in length are typically called “oligonucleotides” and may comprise primers for use in manipulation of DNA such as via polymerase chain reaction (PCR).

The term “amino acid” in the context of the present invention is used in its broadest sense and is meant to include naturally occurring L a-amino acids or residues. The commonly used one and three letter abbreviations for naturally occurring amino acids are used herein: A=Ala; C=Cys; D=Asp; E=Glu; F=Phe; G=Gly; H=His; I=Ile; K=Lys; L=Leu; M=Met; N=Asn; P=Pro; Q=Gln; R=Arg; S=Ser; T=Thr; V=Val; W=Trp; and Y=Tyr (Lehninger, A. L., (1975) Biochemistry, 2d ed., pp. 71-92, Worth Publishers, New York). The general term “amino acid” further includes D-amino acids, retro-inverso amino acids as well as chemically modified amino acids such as amino acid analogues, naturally occurring amino acids that are not usually incorporated Into proteins such as norleucine, and chemically synthesised compounds having properties known in the art to be characteristic of an amino acid, such as β-amino acids. For example, analogues or mimetics of phenylalanine or proline, which allow the same conformational restriction of the peptide compounds as do natural Phe or Pro, are included within the definition of amino acid. Such analogues and mimetics are referred to herein as “functional equivalents” of the respective amino acid. Other examples of amino acids are listed by Roberts and Vellaccio, The Peptides: Analysis, Synthesis, Biology, Gross and Meiehofer, eds., Vol. 5 p. 341, Academic Press, Inc., N.Y. 1983, which is incorporated herein by reference.

A “polypeptide” is a polymer of amino acid residues joined by peptide bonds, whether produced naturally or in vitro by synthetic means. Polypeptides of less than around 12 amino acid residues in length are typically referred to as “peptides” and those between about 12 and about 30 amino acid residues in length may be referred to as “oligopeptides”. The term “polypeptide” as used herein denotes the product of a naturally occurring polypeptide, precursor form or proprotein. Polypeptides can also undergo maturation or post-translational modification processes that may include, but are not limited to: glycosylation, proteolytic cleavage, lipidization, signal peptide cleavage, propeptide cleavage, phosphorylation, and such like. The term “protein” is used herein to refer to a macromolecule comprising one or more polypeptide chains.

A “biological pore” is a trans-membrane protein structure defining a channel or hole that allows the translocation of molecules and ions from one side of the membrane to the other. The translocation of ionic species through the pore may be driven by an electrical potential difference applied to either side of the pore. A “nanopore” is a biological pore in which the minimum diameter of the channel through which molecules or ions pass is in the order of nanometres (10⁻⁹ metres).

For all aspects and embodiments of the present invention, a polynucleotide can comprise a polynucleotide that has at least 50%, 60%, 70%, 80%, 90%, 95% or 99% complete sequence identity to wild-type E. coli CsgG as shown in SEQ ID NO: 2. Likewise, the polypeptide can comprise a polypeptide that has at least 50%, 60%, 70%, 80%, 90%, 95% or 99% complete sequence identity to wild-type E. coli CsgG as shown in SEQ ID NO: 1. A polypeptide can comprise a polypeptide that contains the PFAM domain PF03783, which is characteristic for CsgG-like proteins. A list of presently known CsgG homologues and CsgG architectures can be found at http://pfam.xfam.org//family/PF03783. Sequence identity can thus also be to a fragment or portion of the full length polynucleotide or polypeptide. Hence, a sequence may have only 50% overall sequence Identity with a sequence of the invention but a particular region, domain or subunit could share 80%, 90%, or as much as 99% sequence identity with sequences of the invention. According to the present invention, homology to the nucleic acid sequence of SEQ ID NO: 2 is not limited simply to sequence identity. Many nucleic acid sequences can demonstrate biologically significant homology to each other despite having apparently low sequence identity. In the present invention homologous nucleic acid sequences are considered to be those that will hybridise to each other under conditions of low stringency (M. R. Green, J. Sambrook, 2012, Molecular Cloning: A Laboratory Manual, Fourth Edition, Books 1-3, Cold Spring Harbor Laboratory Press, Cold Spring Harbor, NY).

The term “vector” is used to denote a DNA molecule that is either linear or circular, into which another nucleic acid (typically DNA) sequence fragment of appropriate size can be Integrated. Such DNA fragment(s) can include additional segments that provide for transcription of a gene encoded by the DNA sequence fragment. The additional segments can include and are not limited to: promoters, transcription terminators, enhancers, internal ribosome entry sites, untranslated regions, polyadenylation signals, selectable markers, origins of replication and such like. A variety of suitable promoters for prokaryotic (e.g., the [beta]-lactamase and lactose promoter systems, alkaline phosphatase, the tryptophan (trp) promoter system, lac, tac, T3, T7 promoters for E. coli) and eukaryotic (e.g., simian virus early or late promoter, Rous sarcoma virus long terminal repeat promoter, cytomegalovirus promoter, adenovirus late promoter, EG-1a promoter) hosts are available. Expression vectors are often derived from plasmids, cosmids, viral vectors and yeast artificial chromosomes; vectors are often recombinant molecules containing DNA sequences from several sources. Specific embodiments of the present invention provide for an expression vector that encodes a wild type or modified CsgG polypeptide as described herein. The term “operably linked”, when applied to DNA sequences, for example in an nucleic acid vector such as mentioned above, indicates that the sequences are arranged so that they function cooperatively in order to achieve their intended purposes, i.e. a promoter sequence allows for initiation of transcription that proceeds through an associated coding sequence as far as a termination sequence.

The trans-membrane protein structure of a biological pore may be monomeric or oligomeric in nature. Typically, the pore comprises a plurality of polypeptide subunits arranged around a central axis thereby forming a protein-lined channel that extends substantially perpendicular to the membrane in which the nanopore resides. The number of polypeptide subunits is not limited. Typically, the number of subunits is from 5 to up to 30, suitably the number of subunits is from 6 to 10. Alternatively, the number of subunits is not defined as in the case of perfringolysin or related large membrane pores. The portions of the protein subunits within the nanopore that form protein-lined channel typically comprise secondary structural motifs that may include one or more trans-membrane β-barrel, and/or α-helix sections.

It is to be understood that different applications of the disclosed products and methods may be tailored to the specific needs in the art. It is also to be understood that the terminology used herein is for the purpose of describing particular embodiments of the invention only, and is not intended to be limiting.

In addition as used in this specification and the appended claims, the singular forms “a”, “an”, and “the” include plural referents unless the content clearly dictates otherwise. Thus, for example, reference to “a polynucleotide” includes two or more polynucleotides, reference to “a polynucleotide binding protein” includes two or more such proteins, reference to “a helicase” includes two or more helicases, reference to “a monomer” refers to two or more monomers, reference to “a pore” includes two or more pores and the like.

All publications, patents and patent applications cited herein, whether supra or infra, are hereby incorporated by reference in their entirety.

The present invention relates in part to the bacterial amyloid secretion channel (CsgG), its method of manufacture and its use in nucleic acid sequencing applications, and molecular sensing.

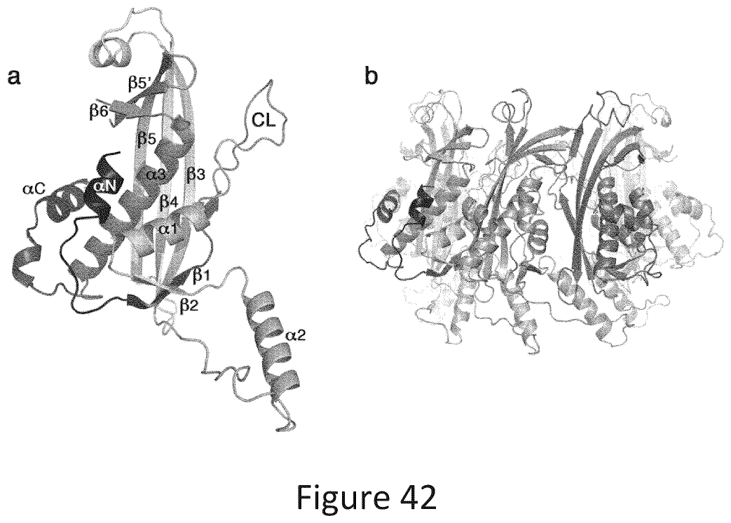

CsgG is a membrane lipoprotein present in the outer membrane of E. coli (Uniprot accession no. P0AEA2; Gene ID: 12932538). In the outer lipid membrane, CsgG forms a nanopore comprising an oligomeric complex of nine CsgG monomer subunits. By virtue of the type II (lipoprotein) signal sequence, the CsgG preprotein is translocated across the SEC translocon and subsequently becomes triacylated at the N-terminal Cys residue of the mature CsgG (i.e. CsgG with cleaved type II signal sequence). Triacylated, or “lipidated” CsgG is transported to the outer membrane of a Gram-negative host, where it inserts into the bilayer as a nonameric pore A non-lipidated form of CsgG, e g CsgG_{C1S}, exists in the periplasm as a soluble protein In a pre-pore conformation (FIG. 42).

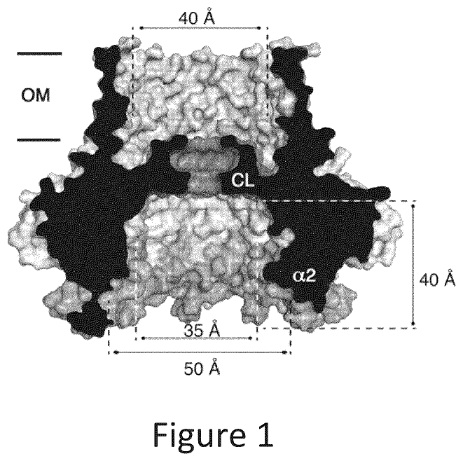

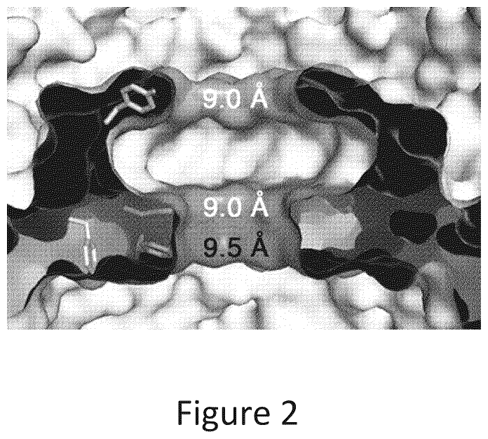

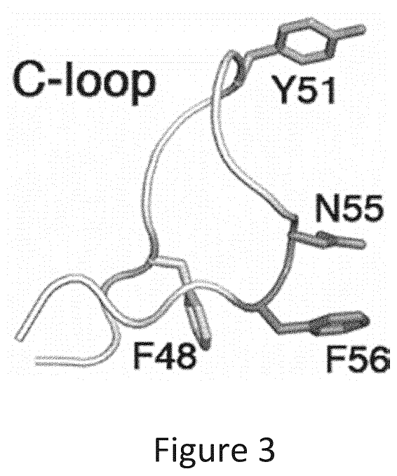

The X-ray structure of the wild type CsgG nanopore (Goyal et al., Nature, 2014, 516(7530), 250-3) shows that it has a width of 120 Å and a height of 85 Å (hereinafter, the term “width” of the nanopore will relate to its dimension parallel with the membrane surface, and the term “height” of the nanopore will relate to its dimension perpendicular to the membrane). The CsgG pore complex traverses the membrane through a 36-stranded β-barrel to provide a 40 Å inner diameter channel (FIG. 1). The assembled monomers of the CsgG channel each possess a conserved 12-residue loop (C-loop, “CL”; FIG. 2), which co-operate so as to form a constriction in the channel to a diameter of approximately 9.0 Å (FIGS. 1 and 2). The constriction in the wild type CsgG nanopore is composed of three stacked concentric rings formed by the side chains of amino acid residues Tyr51, Asn55 and Phe56 of each of the CsgG monomers present in the CsgG oligomer (FIG. 3). This numbering of residues is based upon the mature protein which lacks the native 15 amino acid signal sequence at the N-terminal. The mature protein therefore corresponds to residues 16 to 277 of SEQ ID NO:1. Tyr51 is at position 66 in SEQ ID NO:1, Asn is at position 70 in SEQ ID NO:1 and Phe56 is at position 71 in SEQ ID NO:1.

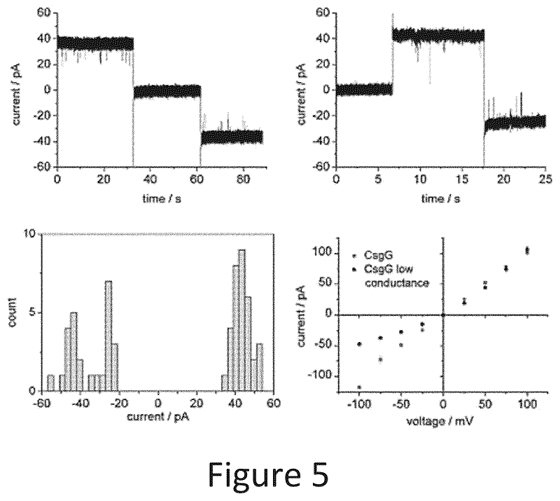

The constriction acts to limit the passage of ions and other molecules through the CsgG channel. Single-channel current recordings of CsgG reconstituted in planar phospholipid bilayers led to a steady current of 43.1±4.5 pA (n=33) or −45.1±4.0 pA (n=13) using standard electrolyte conditions and a potential of +50 mV or −50 mV, respectively (FIG. 5).

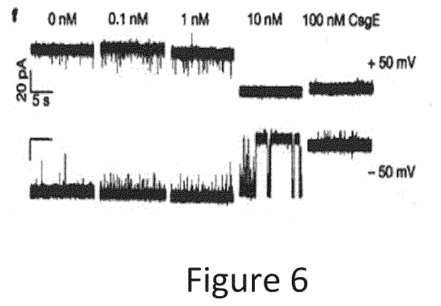

Current flow through the CsgG channel can be effectively blocked by the addition of stoichiometric quantities of the periplasmic factor CsgE (Uniprot accession no. POAE95; Examples 10 to 12). Without wishing to be bound by theory, current evidence points to a mechanism whereby CsgE forms a complex with the CsgG pore acting to cap one end of the channel. The significant reduction in the flow of ions through the CsgG channel may be measured using standard single-channel recording techniques (Examples 12 and 13, FIG. 6). The inventors have found that measured parameters for the current flow (maximum current, and ability to monitor current variation) render the nanopore suitable for use in nucleic acid sequencing and molecular sensing applications according to one embodiment of the invention.

Accordingly, the present invention relates in part to methods and uses of the CsgG nanopore protein complex in nucleic acid sequencing based on variations of electrical measurements of the current flowing through a nanopore.

Nucleic acids are particularly suitable for nanopore sequencing. The naturally-occurring nucleic acid bases In DNA and RNA may be distinguished by their physical size. As a nucleic acid molecule, or individual base, passes through the channel of a nanopore, the size differential between the bases causes a directly correlated reduction in the ion flow through the channel. The variation in ion flow may be recorded. Suitable electrical measurement techniques for recording ion flow variations are described in, for example. WO 2000/28312 and D. Stoddart et al., Proc. Natl. Acad. Sci., 2010, 106, pp 7702-7 (single channel recording equipment); and, for example, in WO 2009/077734 (multi-channel recording techniques). Through suitable calibration, the characteristic reduction in ion flow can be used to identify the particular nucleotide and associated base traversing the channel in real-time.

The size of the narrowest constriction in a transmembrane channel is typically a key factor in determining suitability of a nanopore for nucleic acid sequencing applications. If the constriction is too small, the molecule to be sequenced will not be able to pass through. However, to achieve a maximal effect on ion flow through the channel, at its narrowest point (i.e, at a constriction) the channel should not be too large. Ideally, any constriction should be as close as possible in diameter to the size of the base passing through. For sequencing of nucleic acids and nucleic acid bases, suitable constriction diameters are in the nanometre range (10⁻⁹ metre range). Suitably, the diameter should be in the region of 0.5 to 1.5 nm, typically, the diameter is in the region of 0.7 to 1.2 nm. The constriction in wild type E. coli CsgG has a diameter of approximately 9 Å (0.9 nm). The inventors have deduced that the size and configuration of the constriction in the CsgG channel is suitable for nucleic acid sequencing.

For applications related to nucleic acid sequencing, the CsgG nanopore may be used in wild-type form or may be further modified, such as by directed mutagenesis of particular amino acid residues, to further enhance the desired properties of the nanopore In use. For example, In embodiments of the present invention mutations are contemplated to alter the number, size, shape, placement or orientation of the constriction within the channel. Modified mutant CsgG nanopore complex may be prepared by known genetic engineering techniques that result in the insertion, substitution and/or deletion of specific targeted amino acid residues in the polypeptide sequence. In the case of the oligomeric CsgG nanopore, the mutations may be made in each monomeric polypeptide subunit, or any one of the monomers, or all of the monomers. Suitably, in one embodiment of the invention the mutations described are made to all monomeric polypeptides within the oligomeric protein structure.

According to an embodiment of the invention, a modified mutant CsgG nanopore is provided where the number of channel constrictions within the pore is reduced.

The wild type E. coli CsgG pore includes two channel constrictions (see FIG. 1). These are formed by (i) amino acid residues Phe56 and Asn55, and (ii) amino acid residue Tyr 51, as part of a wider structure comprising additional amino acids from position 54 and to 53, as well as the C-loop motif (FIGS. 2 and 3).

In typical nanopore nucleic acid sequencing, the open-channel ion flow is reduced as the individual nucleotides of the nucleic sequence of Interest sequentially pass through the channel of the nanopore due to the partial blockage of the channel by the nucleotide. It is this reduction in ion flow that is measured using the suitable recording techniques described above. The reduction in ion flow may be calibrated to the reduction in measured ion flow for known nucleotides through the channel resulting in a means for determining which nucleotide is passing through the channel, and therefore, when done sequentially, a way of determining the nucleotide sequence of the nucleic acid passing through the nanopore. For the accurate determination of individual nucleotides, it has typically required for the reduction in ion flow through the channel to be directly correlated to the size of the Individual nucleotide passing through the single constriction (or “reading head”). It will be appreciated that sequencing may be performed upon an intact nucleic acid polymer that is ‘threaded’ through the pore via the action of an associated polymerase, for example. Alternatively, sequences may be determined by passage of nucleotide triphosphate bases that have been sequentially removed from a target nucleic acid in proximity to the pore (see for example WO 2014/187924).

When two or more constrictions are present and spaced apart each constriction may interact or “read” separate nucleotides within the nucleic acid strand at the same time. In this situation, the reduction in ion flow through the channel will be the result of the combined restriction in flow of all the constrictions containing nucleotides. Hence, in some instances a double constriction may lead to a composite current signal. In certain circumstances, the current read-out for one constriction, or “reading head”, may not be able to be determined individually when two such reading heads are present.

The wild-type pore structure of CsgG may be re-engineered via recombinant genetic techniques to widen, alter, or remove one of the two constrictions to leave a single constriction within the channel, thus, defining a single reading head. The constriction motif in the CsgG oligomeric pore is located at amino acid residues at position 38 to 63 in the wild type monomeric E. coli CsgG polypeptide. The wild-type amino acid sequence of this region is provided as SEQ ID NO: 3. In considering this region, mutations at any of the amino acid residue positions 50 to 53, 54 to 56 and 58 to 59 are contemplated as within the remit of the present invention. Based on sequence similarity with CsgG homologues (FIG. 4), amino acid residue positions 38 to 49, 53, 57, and 61 to 63 are considered to be highly conserved and therefore may be less suitable for substitution or other modification. Due to the key positioning of the sidechains of Tyr51, Asn55, and Phe56 within the channel of the wild-type CsgG structure, mutation at these positions may be advantageous in order to modify or alter the characteristics of the reading head.

Mutations at a given position of the monomeric CsgG protein may result in the substitution of the wild-type amino acid at that position with any other natural or unnatural amino acid. In one embodiment of the invention, it is desirable to widen or remove a constriction; suitably the amino acid sidechain in the modified CsgG protein will be selected so as to be less sterically encumbering than the amino acid sidechain in the wild type structure which it replaces. The replacement amino acid residue at a given position may have similar electrostatic properties, or it may have different electrostatic properties Suitably, the replacement amino acid sidechain will possess a similar electrostatic charge to the amino acid sidechain in the wild type structure which it replaces in order to minimise disruption to secondary structure or the properties of the channel.

The selection of replacement amino acid may be based on a BLOSUM62 matrix which provides a standard methodology for calculating the likelihood of an amino acid being substituted for another based on a large multiple sequence alignment. Examples of BLOSUM62 matrices are freely available to the skilled person on the Internet; see for example the website of the National Center for Biotechnology Information (http://www.ncbi.nlm.nih.gov/Class/Structure/aalaa_explorer.cgi).

For Tyr51 in the wild-type CsgG structure, which acts to form a first constriction in the CsgG channel, substitution with any amino acid is provided. In particular, in certain embodiments of the invention Tyr51 may be substituted with alanine, glycine, valine, leucine, isoleucine, asparagine, glutamine, and phenylalanine (SEQ ID: 39-318). In embodiments of the invention, substitution of Tyr51 with alanine or glycine is particularly suitable (SEQ ID: 39-108). In embodiments, residues 50 to 53 (PYPA in the wild-type sequence) may be replaced with glycine-glycine (GG) (SEQ ID: 354-388).

For Asn55, which contributes to the second constriction in the CsgG channel, substitution with any amino acid is provided. In particular, in certain embodiments of the invention Asn55 may be substituted with alanine, glycine, valine, serine or threonine (SEQ ID: 9-33, 44-68, 79-103 114-138, 149-173, 184-208, 219-243, 254-278, 289-313 and 324-348).

For Phe56 which forms part of the second constriction in the CsgG channel, substitution with any amino acid is provided. In particular, in certain embodiments of the invention Phe56 may be substituted with alanine, glycine, valine, leucine, isoleucine, asparagine, and glutamine (SEQ ID: 5-13, 15-18, 20-23, 25-28, 30-33, 40-43, 45-48, 50-53, 55-58, 60-63, 65-68, 75-78, 80-83, 85-88, 90-93, 95-98, 100-103, 110-113, 115-118, 120-123, 125-128, 130-133, 135-138, 145-148, 150-153, 155-158, 160-163, 165-168, 170-173, 180-183, 185-188, 190-193, 195-198, 200-203, 205-208, 215-218, 220-223, 225-228, 230-233, 235-238, 240-243, 250-253, 255-258, 260-263, 265-268, 270-273, 275-578, 285-288, 290-293, 295-298, 300-303, 305-308, 310-313, 320-323, 325-328, 330-333, 335-338, 340-343 and 345-348). In embodiments of the invention, substitution of Phe56 with alanine and glycine is particularly suitable (SEQ ID: 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 6, 11, 16, 21, 26, 31, 36, 41, 46, 51, 56, 61, 66, 71, 76, 81, 86, 91, 96, 101, 106, 111, 116, 121, 126, 131, 136, 141, 146, 151, 156, 161, 166, 171, 176, 181, 186, 191, 196, 201, 206, 211, 216, 221, 226, 231, 238, 241, 246, 251, 256, 261, 266, 271, 276, 281, 286, 291, 296, 301, 306, 311, 316, 321, 326, 331, 336, 341 346, 351, 356, 361, 366, 371, 376, 381 and 386).

In a given mutant CsgG protein, substitution of Tyr51 may be performed at the same time as either one of positions 55 and 56 (SEQ ID: 44, 49, 54, 59, 64, 79, 84, 89, 94, 99, 114, 119, 124, 129, 134, 149, 154, 159, 164, 169, 184, 189, 194, 199, 204, 219, 224, 229, 234, 239, 254, 259, 264, 269, 274, 289, 294, 299, 304, 309, 359, 364, 369, 374, 379, 40, 41, 42, 75, 76 77, 110, 111, 112, 145, 146, 147, 180, 181, 182, 215, 216, 217, 250, 251, 252, 285, 286 and 287), wherein at least one constriction of suitable dimensions within the channel is maintained. Alternatively, substitution of Tyr51 is mutually exclusive to substitution at both of positions Asn55 and Phe56 (SEQ ID: 39, 74, 109, 144, 179, 214, 249, 284, 354, 10, 11, 12, 15, 16, 17, 20, 21, 22, 25, 26, 27, 30, 31 and 32

Alternatively, one or more of the Tyr51, Asn55 or Phe56 in the wild type CsgG protein may be deleted (SEQ ID: 319-353, 34-38, 69-73, 104-108, 139-143, 174-178, 209-213, 244-248, 279-283, 314-318, 384-388, 8, 13, 18, 23, 28, 33, 43, 48, 53, 58, 63, 68, 78, 83, 88, 93, 98, 103, 113, 118, 123, 128, 133, 138, 148, 153, 158, 163, 168, 173, 183, 188, 193, 198, 203, 208, 218, 223, 228, 233, 238, 243, 253, 258, 263, 268, 273, 278, 288, 293, 298, 303, 308 and 313). To maintain at least one constriction in the channel, in a given embodiment, deletion of amino acid residue Tyr51 is mutually exclusive to deletion of both amino acid residues Asn55 and Phe56 (SEQ ID: 319-322, 324-327, 329-332, 334-337, 339-342, 344-347, 38, 73, 108, 143, 178, 213, 248, 283 and 318). Certain neighbouring amino acid residues at positions 53 and 54 and 48 and 49 may also be deleted.

It is to be understood that the present invention provides embodiments where the above modifications may be made in isolation, or in any combination.

Removal of either the constriction at Tyr51 or the constriction at Asn55/Tyr56 results in a single constriction within the CsgG channel. Without wishing to be bound by theory, it is postulated that the constriction at Asn55/Tyr56 would have higher conformational stability than the constriction at Tyr51 which may be desirable. However, the Asn55/Tyr56 constriction could be too high (as measured along the central pore axis) in comparison to the nucleotides. This may lead to poor resolution of individual base pairs in translocating DNA strands.

The opposite is likely true for the Tyr51 constriction. After the removal of the Asn55/Tyr56 constriction, the remaining ring of Tyr51 residues in the oligomer may be conformationally less stable than in the native structure. However, the Tyr51 constriction is shorter (when measured along the central pore axis) and likely more capable of providing a constriction within the channel that may distinguish between individual bases.

In either embodiment, the presence of a single narrow constriction (when measured along the central pore axis) is likely to reduce the complexity of the electrical current readings when the pore is utilised in nucleic acid sequencing applications. Modulations in the observed electrical current occurring during nucleic acid translocation through the pore will, hence, solely reflect the passage of separate nucleotides through a single constriction, or “reading head”.

The effective removal of one constriction may also increase the open-channel current of the pore variant. An increased open-channel current would be advantageous as a higher background conductance leads to better resolved current blockade levels for the different nucleic acid base pair signals. In this way, modifications to the reading head can improve the suitability of the biological pore for both nucleic acid sequencing and other molecular sensing applications.

As an alternative embodiment, or in addition to the sequence modifications described above, it is also provided that the Asn55/Phe56 constriction may be further adapted to tune its height (as measured along the central pore axis). Such further adaptation of the Asn55/Phe56 constriction may or may not be accompanied by mutations of the Tyr51 or other positions within the CsgG channel. Suitably, further adaptation of the Asn55/Phe56 constriction is contemplated as part of mutations that widen or remove the constriction formed by the Tyr51 residue.

In the wild-type form, the Asn55/Phe56 channel constriction is composed of two amino acid rings positioned vertically adjacent to each other. The constriction, as a result, has a length of more than 1 nm. A 1 nm long constriction may not allow the resolution of the electrical signals generated from the ion flow to the separate bases in translocating nucleic acid strands. Typically, the constriction(s) of known nanopores used for nucleic acid sequencing typically has a length less than 1 nm. For example, the MspA nanopore which is used for DNA sequencing has a constriction height of 0.6 nm (as measured along the central pore axis; Manrao et al., Nature Biotechnology, 2012, 30(4), 349-353).

To reduce the height of the Asn55/Phe56 constriction (as measured along the central pore axis), either of the two residues may be substituted or deleted leading to a widening of the top or bottom of the constriction.

For Asn55, which forms part of the second constriction in the CsgG channel, substitution with any amino acid is contemplated. In particular, substitution with alanine, glycine, valine, serine or threonine (SEQ ID: 9-33, 44-68, 79-103 114-138, 149-173, 184-208, 219-243, 254-278, 289-313 and 324-348).

For Phe 56 which forms part of the second constriction in the CsgG channel, substitution with any amino acid is contemplated. In particular, substitution with alanine, glycine, valine, leucine, isoleucine, asparagine, and glutamine (SEQ ID: 5-13, 15-18, 20-23, 25-28, 30-33, 40-43, 45-48, 50-53, 55-58, 60-63, 65-68, 75-78, 80-83, 85-88, 90-93, 95-98, 100-103, 110-113, 115-118, 120-123, 125-128, 130-133, 135-138, 145-148, 150-153, 155-158, 160-163, 165-168, 170-173, 180-183, 185-188, 190-193, 195-198, 200-203, 205-208, 215-218, 220-223, 225-228, 230-233, 235-238, 240-243, 250-253, 255-258, 260-263, 265-268, 270-273, 275-578, 285-288, 290-293, 295-298, 300-303, 305-308, 310-313, 320-323, 325-328, 330-333, 335-338, 340-343 and 345-348). In embodiments of the invention, substitution of Phe56 with alanine and glycine is particularly suitable (SEQ ID: 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 6, 11, 16, 21, 26, 31, 36, 41, 46, 51, 56, 61, 66, 71, 76, 81, 86, 91, 96, 101, 106, 111, 116, 121, 126, 131, 136, 141, 146, 151, 156, 161, 166, 171, 176, 181, 186, 191, 196, 201, 206, 211, 216, 221, 226, 231, 236, 241, 246, 251, 256, 261, 266, 271, 276, 281, 286, 291, 296, 301, 306, 311, 316, 321, 326, 331, 336, 341 346, 351, 356, 361, 366, 371, 376, 381 and 386).

Modifications to tune the minimum diameter of the constriction of the wild-type CsgG pore are also contemplated. The minimum diameter of both constrictions in the CsgG pore is approximately 0.9 nm (9 Å), which is less than the diameter of 1.2 nm for the constriction in the known MspA nanopore that shows utility for DNA sequencing (Manrao et al., Nature Biotechnology, 2012, 30(4), 349-353). Any of the above mutations above that provide the remaining constriction in the modified CsgG pore with a minimum diameter of 0.5 to 1.5 nm would be suitable.

Any of the modifications listed above may beneficially alter the hydrophilicity and charge distribution of the amino acids at the constriction to improve the passage and non-covalent interaction with the translocating nucleic acid strand in order to improve the current read-out. Any of the mutations listed above may also beneficially alter the hydrophilicity and charge distribution close to the channel constriction in order to optimise the flow of electrolyte ions through the constriction and achieve a better discrimination among the passing nucleotides of the translocating nucleic acid strand.

Further modifications of the CsgG protein are contemplated that may result in changing the surface charge distribution within the channel lumen. In one embodiment of the invention, these modifications may be made to avoid undesired electrostatic adsorption of the translocating nucleic acid to the channel wall. Since a nucleic acid is negatively charged and the CsgG channel lumen contains some positive charges (FIG. 1), it is postulated that electrostatic interaction may interfere with the threading or translocation during nucleic acid sequencing. Suitably positively charged amino acid residues such as lysine, histidine and arginine may be substituted with neutral or negatively charged side chains in order to further Improve the efficiency of nucleic acid translocation through the pore, and thus the clarity of electrical current readout.

Modification of the channel lumen of wild-type E. coli CsgG or mutant CsgG to facilitate the translocation or threading of a nucleic acid strand (or individual nucleotides) into the pore constriction is also provided by an embodiment of the present invention. The membrane-spanning section of CsgG with the inner constriction resembles a barrel with a lid featuring a central hole. The threading could be facilitated by adding additional loops into the pore lumen which is closest to the barrel and lid.

A specific embodiment of the invention provides that the CsgG pore may be comprised of one or more monomers, dimers or oligomers that are covalently attached. By way of non-limiting example, monomers may be genetically fused in any configuration, such as by their terminal amino acids. In this instance, the amino terminus of one monomer may be fused to the carboxy terminus of another monomer.

According to an embodiment of the invention, it is also provided that the CsgG pore may be adapted to accommodate additional accessory proteins that may have beneficial properties on the passage of molecules through the pore. The adaptations to the pore may facilitate anchoring of nucleic acid-processing enzymes. Nucleic acid-processing enzymes may include DNA or RNA polymerases; isomerases; topoisomerases; gyrases; telomerases; and helicases. Associated of one or more of these enzymes with the nanopore can have benefits in terms of enhanced threading of the nucleic acid into the pore, and in controlling the speed at which a nucleic acid strand translates through the pore (Manrao et at, Nature Biotechnology, 2012, 30(4), 349-353). Controlling the translocation speed of the nucleic acid strand through the pore has the advantage of providing an Improved response in terms of the electrical current measurement of the ion flow that is more suitable for reading and more uniform.

In embodiments of the invention, it is envisaged that modifications in the extra-membranous regions of the nanopore may help facilitate the docking of a suitable nucleic acid-processing enzyme, such as a DNA-polymerase, inside or adjacent to the lumen of the channel via the provision of one or more binding/anchoring sites. Suitable anchoring sites may comprise electrostatic patches for electrostatic binding of the enzyme; one or more cysteine residues to allow for covalent coupling; and/or an altered inner width of the transmembrane channel section to provide a steric anchor.

Further adaptations of the CsgG wild type pore for use in nucleic acid sequencing that are provided by the present invention in specific embodiments that are set out in more detail below.

In embodiments of the invention, the extra-membranous region of the CsgG pore (bottom portion as shown in FIG. 1) may be truncated or removed to facilitate the exit of the nucleic strand on the other side of the channel lumen. Truncation or removal of the extra-membranous region can also improve the current resolution of the electrical signal from the ion flow in the channel. The latter benefit is brought about by lowering the resistance caused to ion flow by the extra-membranous region. In the present CsgG pore, the transmembrane channel, the inner constriction, and cap region represent three areas of resistance in series. Removing or lowering the contribution of one of these will increase the open-channel current and hence improve the electrical current resolution. Such alteration can include the deletion of α-helix 2 (α2), the C-terminal a-helix (αC) (FIG. 1), and/or a combination thereof.

In further embodiments of the invention, the membrane facing amino acids on the outer face of the wild-type E. coli CsgG pore may be modified to facilitate the insertion of the pore into the membrane. In certain embodiments, it is provided that single amino-acid substitutions may replace wild-type residues with suitable more hydrophobic analogues. For example, one or more of the residues Ser136, Gly138, Gly140, Ala148, Ala 188 or Gly202 could be changed to Ala, Val, Leu, or lie. In addition, aromatic residues such as tyrosine or tryptophan can substitute appropriate wild-type amino-acid so that they are positioned at the interface the hydrophobic membrane and the hydrophilic solvent. For example, one or more of the residues Leu154 or Leu182 could be substituted by Tyr, Phe or Trp.

In embodiments of the invention, the thermal stability of the protein pore may be increased. This results in an advantageous increase in the shelf-life of the nanopore in sequencing devices. In embodiments, an Increase in thermal stability of the protein pore is attained by the modification of beta-turn sequences or improving electrostatic interactions at the protein surface. In one embodiment, the β-hairpin in the trans-membrane regions could be stabilized by a covalent disulfide formation across two adjacent β-strands within or between adjacent β-hairpins. Examples of such cross-strand cysteine pairs could be: Val139-Asp203; Gly139-Gly205; Lys135-Thr207; Glu201-Ala141; Gly147-Gly189; Asp149-Gln187; Gln151-Glu185; Thr207-Glu185; Gly205-Gln187; Asp203-Gly189; Ala153-Lys135; Gly137-Gln151; Val139-Asp149 or Ala141-Gly147.

In further embodiments of the invention, the codon usage of the polypeptide sequence may be modified to allow expression of the CsgG protein at high level and with a low error rate according to the methods described in Biotechnol. J., 2011, 6(6), 650-659. The modification may also target any secondary structures of the mRNA.

The present invention also provides for the alteration of the protein sequence to improve its protease stability. This may be achieved by removal of flexible loop regions, for example the deletion of α-helix 2 (α2), the C-terminal a-helix (αC) (FIG. 1), and/or a combination thereof.

In embodiments of the invention, the CsgG polypeptide sequence/expression system is altered to avoid the possible aggregation of the protein.

The present invention also provides for the alteration of the polypeptide sequence to improve the folding efficiency of the protein. Suitable techniques are provided In Biotechnol. J., 2011, 6(6), 650-659.

One embodiment of the invention further provides for the replacement of or addition of a bioaffinity tag to facilitate the purification of the CsgG protein. The published structure of the CsgG pore contains a StrepII tag (Goyal et al., Nature, 2014, 516(7530), 250-3). Embodiments of the invention comprise other bioaffinity tags, such as Histidine-tag to facilitate the purification via metal chelate affinity chromatography. In alternative embodiments of the invention, the tag may include a FLAG-tag or an epitope tag, such as a Myc- or HA-tag. In a further embodiment of the invention, the nanopore may be modified by biotinylation with biotin or an analogue thereof (e.g. desthiobiotin), thereby facilitating purification via interaction with streptavidin.

In embodiments of the invention, negative charges at the protein terminus may be added to increase the net charge of the polypeptide and facilitate the migration of the protein in polyacrylamide gel electrophoresis. This may lead to the improved separation of heteroligomers of the CsgG in the case where these species are of interest (see Howorka et al. Proc. Nat. Acad. Sci., 2001, 98(23), 12996-13001). A heterooligomer can be useful to introduce a single-cysteine residue per pore which may be advantageous in facilitating the attachment of a suitable nucleic acid-processing enzyme, such as a DNA-polymerase as described above.

The present invention relates in part to the use of the wild type or modified CsgG nanopore in molecular sensing applications based on variations of electrical measurements of current flowing through a nanopore.

The binding of a molecule in the channel of the CsgG pore, or in the vicinity of either opening of the channel will have an effect on the open-channel ion flow through the pore. In a similar manner to the nucleic acid sequencing application described above, variation in the open-channel Ion flow can be measured using suitable measurement techniques by the change in electrical current (for example, WO 2000/28312 and D. Stoddart et al., Proc. Natl. Acad. Sci., 2010, 106, 7702-7 or WO 2009/077734). The degree of reduction in ion flow, as measured by the reduction in electrical current, is related to the size of the obstruction within, or in the vicinity of, the pore. Binding of a molecule of interest, also referred to as an analyte, in or near the pore therefore provides a detectable and measurable event, thereby forming the basis of a biological sensor. Suitable molecules for nanopore sensing include nucleic acids; proteins; peptides; and small molecules such as pharmaceuticals, toxins or cytokines.

Detecting the presence of biological molecules finds application in personalised drug development, medicine, diagnostics, life science research, environmental monitoring and in the security and/or the defence industry.

In embodiments of the invention, the wild type or modified E. coli CsgG nanopore, or homologue thereof, disclosed herein may serve as a molecular sensor. Procedures for analyte detection are described in Howorka et al. Nature Biotechnology (2012) Jun. 7; 30(6):506-7. The analyte molecule that is to be detected may bind to either face of the channel, or within the lumen of the channel itself. The position of binding may be determined by the size of the molecule to be sensed. The wild-type CsgG pore may act as sensor, or embodiments of the invention, the CsgG pore is modified via recombinant or chemical methods to increase the strength of binding, the position of binding, or the specificity of binding of the molecule to be sensed. Typical modifications include addition of a specific binding moiety complimentary to the structure of the molecule to be sensed. Where the analyte molecule comprises a nucleic acid, this binding moiety may comprise a cyclodextrin or an oligonucleotide; for small molecules this may be a known complimentary binding region, for example the antigen binding portion of an antibody or of a non-antibody molecule, including a single chain variable fragment (scFv) region or an antigen recognition domain from a T-cell receptor (TCR); or for proteins, it may be a known ligand of the target protein. In this way the wild type or modified E. coli CsgG nanopore, or homologue thereof, may be rendered capable of acting as a molecular sensor for detecting presence in a sample of suitable antigens (including epitopes) that may include cell surface antigens, including receptors, markers of solid tumours or haematologic cancer cells (e.g. lymphoma or leukaemia), viral antigens, bacterial antigens, protozoal antigens, allergens, allergy related molecules, albumin (e.g. human, rodent, or bovine), fluorescent molecules (including fluorescein), blood group antigens, small molecules, drugs, enzymes, catalytic sites of enzymes or enzyme substrates, and transition state analogues of enzyme substrates.

Modifications may be achieved using known genetic engineering and recombinant DNA techniques. The positioning of any adaptation would be dependent on the nature of the molecule to be sensed, for example, the size, three-dimensional structure, and its biochemical nature. The choice of adapted structure may make use of computational structural design. A series of bespoke CsgG nanopores is envisaged each adapted specifically to the sensing application to which it is destined. Determination and optimization of protein-protein interactions or protein-small molecule interactions can be investigated using technologies such as a BIAcore® which detects molecular Interactions using surface plasmon resonance (BIAcore. Inc., Piscataway, NJ; see also www.biacore.com).

In an embodiment, the CsgG pore can be that of the water soluble, octameric form where the N-terminal Cys residue is replaced by an alternative amino acid in order to prevent the lipidation of the protein N-terminus. In an alternative embodiment, the protein can be expressed in the cytoplasm by removal of the N-terminal leader sequence in order to avoid processing by the bacterial lipidation pathway.

The method of manufacture of the CsgG monomeric soluble protein, octameric soluble protein and the oligomeric lipidated CsgG pore is described in Goyal et al. (Nature, 2014; 516(7530): 250-3), which is incorporated herein by reference, and in Examples 1 and 2.

Mutant CsgG monomers

The present invention provides mutant CsgG monomers. The mutant CsgG monomers may be used to form the pores of the invention. A mutant CsgG monomer is a monomer whose sequence varies from that of a wild-type CsgG monomer and which retains the ability to form a pore. Methods for confirming the ability of mutant monomers to form pores are well-known in the art and are discussed in more detail below.

The mutant monomers have improved polynucleotide reading properties i.e. display improved polynucleotide capture and nucleotide discrimination. In particular, pores constructed from the mutant monomers capture nucleotides and polynucleotides more easily than the wild type. In addition, pores constructed from the mutant monomers display an increased current range, which makes it easier to discriminate between different nucleotides, and a reduced variance of states, which increases the signal-to-noise ratio. In addition, the number of nucleotides contributing to the current as the polynucleotide moves through pores constructed from the mutants is decreased. This makes it easier to identify a direct relationship between the observed current as the polynucleotide moves through the pore and the polynucleotide sequence. In addition, pores constructed from the mutant monomers may display an increased throughput, i.e. are more likely to interact with an analyte, such as a polynucleotide. This makes it easier to characterise analytes using the pores. Pores constructed from the mutant monomers may insert Into a membrane more easily.

A mutant monomer of the invention comprises a variant of the sequence shown in SEQ ID NO: 390. SEQ ID NO: 390 is the wild-type CsgG monomer from Escherichia coli Str. K-12 substr. MC4100. A variant of SEQ ID NO: 390 is a polypeptide that has an amino acid sequence which varies from that of SEQ ID NO: 390 and which retains its ability to form a pore. The ability of a variant to form a pore can be assayed using any method known in the art. For instance, the variant may be inserted into an amphiphilic layer along with other appropriate subunits and its ability to oligomerise to form a pore may be determined. Methods are known in the art for Inserting subunits Into membranes, such as amphiphilic layers. For example, subunits may be suspended in a purified form in a solution containing a triblock copolymer membrane such that it diffuses to the membrane and is inserted by binding to the membrane and assembling into a functional state.

In all of the discussion herein, the standard one letter codes for amino acids are used. These are as follows: alanine (A), arginine (R), asparagine (N), aspartic acid (D), cysteine (C), glutamic acid (E), glutamine (Q), glycine (G), histidine (H), isoleucine (I), leucine (L), lysine (K), methionine (M), phenylalanine (F), proline (P), serine (S), threonine (T), tryptophan (W), tyrosine (Y) and valine (V). Standard substitution notation is also used, i.e. Q42R means that Q at position 42 is replaced with R.

In one embodiment, the mutant monomers of the invention comprise a variant of SEQ ID NO: 390 comprising one or more of the following (i) one or more mutations at the following positions (i.e. mutations at one or more of the following positions) N40, D43, E44, S54, S57. Q62, R97, E101, E124, E131, R142, T150 and R192, such as one or more mutations at the following positions (i.e. mutations at one or more of the following positions) N40, D43, E44, S54, S57, Q62, E101, E131 and T150 or N40, D43, E44, E101 and E131; (ii) mutations at Y51/N55, Y51/F56, N55/F56 or Y51/N55/F56; (iii) Q42R or Q42K; (iv) K49R; (v) N102R, N102F, N102Y or N102W; (vi) D149N, D149Q or D149R; (vii) E185N, E185Q or E185R; (viii) D195N, D195Q or D195R; (ix) E201N, E201Q or E201R; (x) E203N, E203Q or E203R; and (xi) deletion of one or more of the following positions F48, K49. P50, Y51, P52, A53, S54, N55, F56 and S57. The variant may comprise any combination of (i) to (xi). In particular, the variant may comprise {i} {ii} {iii} {iv} {v} {vi} {vii} {viii} {ix} {x} {xi} {i,ii} {i,iii} {i,iv} {i,v} {i,vi} {i,vii} {i,viii} {i,ix} {i,x} {i,xi} {ii,iii} {ii,iv} {ii,v} {ii,vi} {ii,vii} {ii,viii} {ii,ix} {ii,x} {ii,xi} {iii,iv} {iii,v} {iii,vi} {iii,vii} {iii,viii} {iii,ix} {iii,x} {iii,xi} {iv,v} {iv,vi} {iv,vii} {iv,viii} {iv,ix} {iv,x} {iv,xi} {v,vi} {v,vii} {v,vii} {v,ix} {v,x} {v,xi} {vi,vii} {vi,viii} {vi,ix} {vi,x} {vi,xi} {vii,viii} {vii,ix} {vii,x} {vii,x} {viii,ix} {viii,x} {viii,xi} {ix,x} {ix,xi} {x,xi} {i,ii,iii} {i,ii,iv} {i,ii,v} {i,ii,vi} {i,ii,vii} {i,ii,viii}{i,ii,ix} {i,ii,x} {i,ii,xi} {i,iii,iv} {i,iii,v} {i,ii,vi} {i,iii,vii} {i,iii,viii} {i,iii,ix} {i,iii,x} {i,iii,xi} {i,iv,v} {i,iv,vi} {i,iv,vii}{i,iv,viii} {i,iv,ix} {i,iv,x} {i,iv,xi} {i,v,vi} {i,v,vii} {i,v,viii} {i,v,ix} {i,v,x} {i,v,xi} {i,vi,vii} {i,vi,viii} {i,vi,ix} {i,vi,x} {i,vi,xi} {i,vii,viii} {i,vii,ix} {i,vii,x} {i,vii,xi} {i,viii,ix} {i,viii,x} {i,viii,xi} {i,ix,x} {i,ix,xi} {i,x,xi} {ii,iii,iv} {ii, iii,v} {ii, iii,vi} {ii,iii,vii} {ii,iii,viii} {ii,iii,ix} {ii,iii,x} {ii,iii,xi} {ii,iv,v} {ii,iv,vi} {ii,iv,vii} {ii,iv,viii} {ii,iv,ix} {ii,iv,x} {ii,iv,xi} {ii,v,vi} {ii,v,vii} {ii,v,viii} {ii,v,ix} {ii,v,x} {ii,v,xi} {ii,vi,vii} {ii,vi,viii} {ii,vi,ix} {ii,vi,x} {ii,vi,xi} {ii,vii,viii} {ii,vii,ix} {ii,vii,x} {ii,vii,xi} {ii,viii,ix} {ii,viii,x} {ii,viii,xi} {ii,ix,x} {ii,ix,xi} {ii,x,xi} {iii,iv,v} {iii,iv,vi} {iii,iv,vii} {iii,iv,viii} {iii,iv,ix} {iii,iv,x} {iii,iv,xi} {iii,v,vi} {iii,v,vii} {iii,v,viii} {iii,v,ix} {iii,v,x} {iii,v,xi} {iii,vi,vii} {iii,vi,viii} {iii,vi,ix} {iii,vi,x} {iii,vi,xi} {iii,vii,viii} {iii,vii,ix} {iii,vii,x} {iii,vii,xi} {iii,viii,ix} {iii,viii,x} {i,ii,iii,iv} {i,ii,iii,v} {i,ii,iii,vi} {i,ii,iii,vii} {i,ii,iii,viii} {i,ii,iii,ix} {i,ii,iii,x} {i,ii,iii,xi} {i,ii,iv,v} {i,ii,iv,vi} {i,ii,iv,vii} {i,ii,iv,viii} {i,ii,iv,ix} {i,ii,iv,x} {i,ii,iv,xi} {i,ii,v,vi} {i,ii,v,vii} {i,ii,v,viii} {i,ii,v,ix} {i,ii,v,x} {i,ii,v,xi} {i,ii,vi,vii} {i,ii,vi,viii} {i,ii,vi,ix} {i,ii,vi,x} {i,ii,vi,xi} {i,ii,vii,viii} {i,ii,vii,ix} {i,ii,vii,x} {i,ii,vii,xi} {i,ii,viii,ix} {i,ii,viii,x} {i,ii,viii,xi} {i,ii,ix,x} {i,ii,ix,xi} {i,ii,x,xi} {i,iii,iv,v} {i,iii,iv,vi} {i,iii,iv, vii} {i,iii,iv,viii} {i,iii,iv,ix} {i,iii,iv,x} {i,iii,iv,xi} {i,iii,v,vi} {i,iii,v,vii} {i,ii,v,viii} {i,ii,v,ix} {i,iii,v,x} {i,iii,v,xi} {i,iii, vi,vii} {i,iii,vi,viii} {i,ii,vi,ix} {i,iii,vi,x} {i,iii,vi,xi} {i,iii,vii,viii} {i,iii,vii,ix} {i,iii,vii,x} {i,iii,vii,xi} {i,ii,viii,ix} {i,iii,viii,x} {i,iii,viii,xi} {i,iii,ix,x} {i,iii,ix,xi} {i,iii,x,xi} {i,iv,v,vi} {i,iv,v,vii} {i,iv,v,viii} {i,iv,v,ix} {i,iv,v,x} {i,iv,v,xi} {i,iv,vi,vii} {i,iv,vi,viii} {i,iv,vi,ix} {i,iv,vi,x} {i,iv,vi,xi} {i,iv,vii,viii} {i,iv,vii,ix} {i,iv,vii,x} {i,iv,vii,xi} {i,iv,viii,ix} {i,iv,viii,x} {i,iv,viii,xi} {i,iv,ix,x} {i,iv,ix,xi} {i,iv,x,xi} {i,v,vi,vii} {i,v,vi,viii} {i,v,vi,ix} {i,v,vi,x} {i,v,vi,xi} {i,v,vii,viii} {i,v,vii,ix} {i,v,vii,x} {i,v,vii,xi} {i,v,viii,ix} {i,v,viii,x} {i,v,viii,xi} {i,v,ix,x} {i,v,ix,xi} {i,v,x,xi} {i,vi,vii,viii} {i,vi,vii,ix} {i,vi,vii,x} {i,vi,vii,xi} {i,vi,viii,ix} {i,vi,viii,x} {i,vi,viii,xi} {i,vi,ix,x} {i,vi,ix,xi} {i,vi,x,xi} {i,vii, viii,ix} {i,vii,viii,x} {i,vii,viii,xi} {i,vii,ix,x} {i,vii,ix,xi} {i,vii,x,xi} {i,viii,ix,x} {i,viii,ix,xi} {i,viii,x,xi} {i,ix,x,xi} {ii,iii,iv,v} {ii,iii,iv,vi} {ii,iii,iv,vii} {ii,iii,iv,viii} {i,iii,iv,ix} {ii,iii,iv,x} {ii,iii,iv,xi} {ii,iii,v,vi} {ii,iii,v,vii} {ii,iii,v,viii} {ii,iii,v,ix} {ii,iii,v,x} {ii,iii,v,xi} {ii,iii,vi,vii} {ii,iii,vi,viii} {ii,iii,vi,ix} {ii,iii,vi,x} {ii,iii,vi,xi} {ii,iii,vii,viii} {ii,iii,vii,ix} {ii,iii,vii,x} {ii,iii,vii,xi} {ii,iii,viii,ix} {ii,iii,viii,x} {i,ii,viii,xi} {ii,iii,ix,x} {ii,iii,ix,xi} {ii,iii,x,xi} {ii,iv,v,vi} {ii,iv,v,vil} {ii,iv,v,viii} {ii,iv,v,ix} {ii,iv,v,x} {ii,iv,v,xi} {ii,iv,vi,vii} {ii,iv,vi,viii} {ii,iv,vi,ix} {ii,iv,vi,x} {ii,iv,vi,xi} {ii,iv,vii,viii} {ii,iv,vii,ix} {ii,iv,vii,x} {ii,iv,vii,xi} {ii,iv,viii,ix} {ii,iv,viii,x} {ii,iv,viii,xi} {ii,iv,ix,x} {ii,iv,ix,xi} {ii,iv,x,xi} {ii,v,vi,vii} {ii,v,vi,viii} {ii,v,vi,ix} {ii,v,vi,x} {ii,v,vi,xi} {ii,v,vii,viii} {ii,v,vii,ix} {ii,v,vii,x} {ii,v,vii,xi} {ii,v,viii,ix} {ii,v,viii,x} {ii,v,viii,xi} {ii,v,ix,x} {ii,v,ix,xi} {ii,v,x,xi} {ii,vi,vii,viii} {ii,vi,vii,ix} {ii,vi,vii,x} {ii,vi,vii,xi} {ii,vi,viii,ix} {ii,vi,viii,x} {ii,vi,viii,xi} {ii,vi,ix,x} {ii,vi,ix,xi} {ii,vi,x,xi} {ii,vii,viii,ix} {ii,vii,viii,x} {ii,vii,viii,xi} {ii,vii,ix,x} {ii,vii,ix,xi} {ii,vii,x,xi} {ii,viii,ix,x} {ii,viii,ix,xi} {ii,viii,x,xi} {ii,ix,x,xi} {iii,iv,v,vi} {iii,iv,v,vii} {iii,iv,v,viii} {iii,iv,v,ix} {iii,iv,v,x} {iii,iv,v,xi} {iii,iv,vi,vii} {iii,iv,vi,viii} {iii,iv,vi,ix} {iii,iv,vi,x} {iii,iv,vi,xi} {iii,iv,vii,viii} {iii,iv,vii,ix} {iii,iv,vii,x} {iii,iv,vii,xi} {iii,iv,viii,ix} {iii,iv,viii,x} {iii,iv,viii,xi} {iii,iv,ix,x} {iii,iv,ix,xi} {iii,iv,x,xi} {iii,v,vi,vii} {iii,v,vi,viii} {iii,v,vi,ix} {iii,v,vi,x} {iii,v,vi,xi} {iii,v,vii,viii} {ii,v,vii,ix} {iii,v,vii,x} {iii,v,vii,xi} {iii,v,viii,ix} {iii,v,viii,x} {iii,v,viii,xi} {iii,v,ix,x} {iii,v,ix,xi} {iii,v,x,xi} {iii,vi,vii,viii} {iii,vi,vii,ix} {iii,vi,vii,x} {iii,vi,vii xi} {iii,vi,viii,ix} {iii,vi,viii,x} {iii,vi,viii,xi} {iii,vi,ix,x} {iii,vi,ix,xi} {iii,vi,x,xi} {iii,vii,viii,ix} {iii,vii,viii,x} {iii,vii,viii,xi} {iii,vii,ix,x} {iii,vii,ix,xi} {iii,vii,x,xi} {iii,viii,ix,x} {iii,viii,ix,xi} {iii,viii,x,xi} {iii,ix,x,xi} {iv,v,vi,vii} {iv,v,vi,viii} {iv,v,vi,ix} {iv,v,vi,x} {iv,v,vi,xi} {iv,v,vii,viii} {iv,v,vii,ix} {iv,v,vii,x} {iv,v,vii,xi} {iv,v,viii,ix} {iv,v,viii,x} {iv,v,viii,xi} {iv,v,ix,x} {iv,v,ix,xi} {iv,v,x,xi} {iv,vi,vii,viii} {iv,vi,vii,ix} {iv,vi,vii,x} {iv,vi,vii,xi} {iv,vi,viii,ix} {iv,vi,viii,x} {iv,vi,viii,xi} {iv,vi,ix,x} {iv,vi,ix,xi} {iv,vi,x,xi} {iv,vii,viii,ix} {iv,vii,viii,x} {iv,vii,viii,xi} {iv,vii,ix,x} {iv,vii,ix,xi} {iv,vii,x,xi} {iv,viii,ix,x} {iv,viii,ix,xi} {iv,viii,x,xi} {iv,ix,x,xi} {v,vi,vii,viii} {v,vi,vii,ix} {v,vi,vii,x} {v,vi,vii,xi} {v,vi,viii,ix} {v,vi,viii,x} {v,vi,viii,xi} {v,vi,ix,x} {v,vi,ix,xi} {v,vi,x,xi} {v,vii,viii,ix} {v,vi,vii,x} {v,vi,vii,xi} {v,vii,ix,x} {v,vi,ix,xi} {v,vii,x,xi} {v,viix,x} {v,vix,xi} {v,viii,xxi} {v,ix,x,xi} {vi,vii,viii,ix} {vi,vii,viii,x} {vi,vii,viii,xi} {vi,vii,ix,x} {vi,vii,ix,xi} {vi,vii,x,xi} {vi,viii,ix,x} {vi,viii,ix,xi} {vi,viii,x,xi} {vi,ix,x,xi} {vii,viii,ix,x} {vii,viii,ix,xi} {vii,viii,x,xi} {vii,ix,x,xi} {viii,ix,x,xi} {i,ii,iii,iv,v} {i,ii,iii,iv,vi} {i,ii,iii,iv,vii} {i,ii,iii,iv,viii} {i,ii,iii,iv,ix} {i,ii,iii,iv,x} {i,ii,iii,iv,xi} {i,ii,iii,v,vi} {i,ii,iii,v,vii} {i,ii,iii,v,viii} {i,ii,iii,v,ix} {i,ii,iii,v,x} {i,ii,iii,v,xi} {i,ii,iii,vi,vii} {i,ii,iii,vi,viii} {i,ii,iii,vi,ix} {i,ii,iii,vi,x} {i,ii,iii,vi,xi} {i,ii,iii,vii,viii} {i,ii,iii,vii,ix} {i,ii,iii,vii,x} {i,ii,iii,vii,xi} {i,ii,iii,viii,ix} {i,ii,iii,viii,x} {i,ii,iii,viii,xi} {i,ii,iii,ix,x} {i,ii,iii,ix,xi} {i,ii,iii,x,xi} {i,ii,iv,v,vi} {i,ii,iv,v,vii} {i,ii,iv,v,viii} {i,ii,iv,v,ix} {i,ii,iv,v,x} {i,ii,iv,v,xi} {i,ii,iv,vi,vii} {i,ii,iv,vi,viii} {i,ii,iv,vi,ix} {i,ii,iv,vi,x} {i,ii,iv,vi,xi} {i,ii,iv,vii,viii} {i,ii,iv,vii,ix} {i,ii,iv,vii,x} {i,ii,iv,vii,xi} {i,ii,iv,viii,ix} {i,ii,iv,viii,x} {i,ii,iv,viii,xi} {i,ii,iv,ix,x} {i,ii,iv,ix,xi} {i,ii,iv,x,xi} {i,ii,v,vi,vii} {i,ii,v,vi,viii} {i,ii,v,vi,ix} {i,ii,v,vi,x} {i,ii,v,vi,xi} {i,ii,v,vii,viii} {i,ii,v,vii,ix} {i,ii,v,vii,x} {i,ii,v,vii,xi} {i,ii,v,viii,ix} {i,ii,v,viii,x} {i,ii,v,viii,xi} {i,ii,v,ix,x} {i,ii,v,ix,xi} {i,ii,v,x,xi} {i,ii,vi,vii,viii} {i,ii,vi,vii,ix} {i,ii,vi,vii,x} {i,ii,vi,vii,xi} {i,ii,vi,viii,ix} {i,ii,vi,viii,x} {i,ii,vi,viii,xi} {i,ii,vi,ix,x} {i,ii,vi,ix,xi} {i,ii,vi,x,xi} {i,ii,vii,viii,ix} {i,ii,vii,viii,x} {i,ii,vii,viii,xi} {i,ii,vii,ix,x} {i,ii,vii,ix,xi} {i,ii,vii,x,xi} {i,ii,viii,ix,x} {i,ii,viii,ix,xi} {i,ii,viii,x,xi} {i,ii,ix,x,xi} {i,iii,iv,v,vi} {i,iii,iv,v,vii} {i,iii,iv,v,viii} {i,iii,iv,v,ix} {i,iii,iv,v,x} {i,iii,iv,v,xi} {i,iii,iv,vi,vii} {i,iii,iv,vi,viii} {i,iii,iv,vi,ix} {i,iii,iv,vi,x} {i,iii,iv,vi,xi} {i,iii,iv,vii,viii} {i,iii,iv,vii,ix} {i,iii,iv,vii,x} {i,iii,iv,vii,xi} {i,iii,iv,viii,ix} {i,iii,iv,viii,x} {i,iii,iv,viii,xi} {i,iii,iv,ix,x} {i,iii,iv,ix,xi} {i,iii,iv,x,xi} {i,iii,v,vi,vii} {i,iii,v,vi,viii} {i,iii,v,vi,ix} {i,iii,v,vi,x} {i,iii,v,vi,xi} {i,ii,v,vii,viii} {i,iii,v,vii,ix} {i,iii,v,vii,x} {i,iii,v,vii,xi} {i,iii,v,viii,ix} {i,iii,v,viii,x} {i,iii,v,viii,xi} {i,iii,v,ix,x} {i,iii,v,ix,xi} {i,iii,v,x,xi} {i,iii,vi,vii,viii} {i,iii,vi,vii,ix} {i,iii,vi,vii,x} {i,iii,vi,vii,xi} {i,iii,vi,viii,ix} {i,iii,vi,viii,x} {i,iii,vi,viii,xi} {i,iii,vi,ix,x} {i,iii,vi,ix,xi} {i,iii,vi,x,xi} {i,iii,vii,viii,ix} {i,iii,vii,viii,x} {i,iii,vii,viii,xi} {i,iii,vii,ix,x} {i,iii,vii,ix,xi} {i,iii,vii,x,xi} {i,iii,viii,ix,x} {i,iii,viii,ix,xi} {i,iii,viii,x,xi} {i,iii,ix,x,xi} {i,iv,v,vi,vii} {i,iv,v,vi,viii} {i,iv,v,vi,ix} {i,iv,v,vi,x} {i,iv,v,vi,xi} {i,iv,v,vii,viii} {i,iv,v,vii,ix} {i,iv,v,vii,x} {i,iv,v,vii,xi} {i,iv,v,viii,ix} {i,iv,v,viii,x} {i,iv,v,viii,xi} {i,iv,v,ix,x} {i,iv,v,ix,xi} {i,iv,v,x,xi} {i,iv,vi,vii,viii} {i,iv,vi,vii,ix} {i,iv,vi,vii,x} {i,iv,vi,vii,xi} {i,iv,vi,viii,ix} {i,iv,vi,viii,x} {i,iv,vi,viii,xi} {i,iv,vi,ix,x} {i,iv,vi,ix,xi} {i,iv,vi,x,xi} {i,iv,vii,viii,ix} {i,iv,vii,viii,x} {i,iv,vii,viii,xi} {i,iv,vii,ix,x} {i,iv,vii,ix,xi} {i,iv,vii,x,xi} {i,iv,viii,ix,x} {i,iv,viii,ix,xi} {i,iv,viii,x,xi} {i,iv,ix,x,xi} {i,v,vi,vii,viii} {i,v,vi,vii,ix} {i,v,vi,vii,x} {i,v,vi,vii,xi} {i,v,vi,viii,ix} {i,v,vi,viii,x} {i,v,vi,viii,xi} {i,v,vi,ix,x} {i,v,vi,ix,xi} {i,v,vi,x,xi} {i,v,vii,viii,ix} {i,v,vii,viii,x} {i,v,vii,viii,xi} {i,v,vii,ix,x} {i,v,vii,ix,xi} {i,v,vii,x,xi} {i,v,viii,ix,x} {i,v,viii,ix,xi} {i,v,viii,x,xi} {i,v,ix,x,xi} {i,vi,vii,viii,ix} {i,vi,vii,viii,x} {i,vi,vii,viii,xi} {i,vi,vii,ix,x} {i,vi,vii,ix,xi} {i,vi,vii,x,xi} {i,vi,viii,ix,x} {i,vi,viii,ix,xi} {i,vi,viii,x,xi} {i,vi,ix,x,xi} {i,vii,viii,ix,x} {i,vii,viii,ix,xi} {i,vii,viii,x,xi} {i,vii,ix,x,xi} {i,viii,ix,x,xi} {ii,iii,iv,v,vi} {ii,iii,iv,v,vii} {ii,iii,iv,v,viii} {ii,iii,iv,v,ix} {ii,iii,iv,v,x} {ii,iii,iv,v,xi} {ii,iii,iv,vi,vii} {ii,iii,iv,vi,viii} {ii,iii,iv,vi,ix} {ii,iii,iv,vi,x} {ii,iii,iv,vi,xi} {ii,iii,iv,vii,viii} {il,iii,iv,vii,ix} {ii,iii,iv,vii,x} {ii,iii,iv,vii,xi} {ii,iii,iv,viii,ix} {ii,iii,iv,viii,x} {ii,iii,iv,viii,xi} {ii,iii,iv,ix,x} {ii,iii,iv,ix,xi} {ii,iii,iv,x,xi} {ii,iii,v,vi,vii} {ii,iii,v,vi,viii} {ii,iii,v,vi,ix} {ii,iii,v,vi,x} {ii,iii,v,vi,xi} {ii,iii,v,vii,viii} {ii,iii,v,vii,ix} {ii,iii,v,vii,x} {ii,iii,v,vii,xi} {ii,iii,v,viii,ix} {ii,iii,v,viii,x} {ii,iii,v,viii,xi} {ii,iii,v,ix,x} {ii,iii,v,ix,xi} {ii,iii,v,x,xi} {ii,iii,vi,vii,viii} {ii,iii,vi,vii,ix} {ii,iii,vi,vii,x} {i,iii,vi,vii,xi} {ii,iii,vi,viii,ix} {ii,iii,vi,viii,x} {ii,iii,vi,viii,xi} {ii,iii,vi,ix,x} {ii,iii,vi,ix,xi} {ii,iii,vi,x,xi} {ii,iii,vii,viii,ix} {ii,iii,vii,viii,x} {ii,iii,vii,viii,xi} {ii,iii,vii,ix,x} {ii,iii,vii,ix,xi} {ii,iii,vii,x,xi} {ii,iii,viii,ix,x} {ii,iii,viii,ix,xi} {ii,iii,viii,x,xi} {i,iii,ix,x,xi} {ii,iv,v,vi,vii} {ii,iv,v,vi,viii} {ii,iv,v,vi,ix} {ii,iv,v,vi,x} {i,iv,v,vi,xi} {il,iv,v,vii,viii} {ii,iv,v,vii,ix} {ii,iv,v,vii,x} {ii,iv,v,vii,xi} {ii,iv,v,viii,ix} {ii,iv,v,viii,x} {ii,iv,v,viii,xi} {ii,iv,v,ix,x} {ii,iv,v,ix,xi} {i,iv,v,x,xi} {ii,iv,vi,vii,viii} {ii,iv,vi,vii,ix} {ii,iv,vi,vii,x} {ii,iv,vi,vii,xi} {ii,iv,vi,viii,ix} {ii,iv,vi,viii,x} {ii,iv,vi,viii,xi} {ii,iv,vi,ix,x} {ii,iv,vi,ix,xi} {ii,iv,vi,x,xi} {ii,iv,vii,viii,ix} {ii,iv,vii,viii,x} {ii,iv,vii,viii,xi} {ii,iv,vii,ix,x} {ii,iv,vii,ix,xi} {ii,iv,vii,x,xi} {ii,iv,viii,ix,x} {ii,iv,viii,ix,xi} {ii,iv,viii,x,xi} {ii,iv,ix,x,xi} {ii,v,vi,vii,viii} {ii,v,vi,vii,ix} {ii,v,vi,vii,x} {ii,v,vi,vii,xi} {ii,v,vi,viii,ix} {ii,v,vi,viii,x} {ii,v,vi,viii,xi} {ii,v,vi,ix,x} {ii,v,vi,ix,xi} {ii,v,vi,x,xi} {ii,v,vii,viii,ix} {ii,v,vii,viii,x} {ii,v,vii,viii,xi} {ii,v,vii,ix,x} {ii,v,vii,ix,xi} {ii,v,vii,x,xi} {ii,v,viii,ix,x} {ii,v,viii,ix,xi} {ii,v,viii,x,xi} {ii,v,ix,x,xi} {ii,vi,vii,viii,ix} {ii,vi,vii,viii,x} {ii,vi,vii,viii,xi} {ii,vi,vii,ix,x} {ii,vi,vii,ix,xi} {ii,vi,vii,x,xi} {ii,vi,viii,ix,x} {ii,vi,viii,ix,xi} {ii,vi,viii,x,xi} {ii,vi,ix,x,xi} {ii,vii,viii,ix,x} {ii,vii,viii,ix,xi} {ii,vii,viii,x,xi} {ii,vii,ix,x,xi} {ii,viii,ix,x,xi} {iii,iv,v,vi,vii} {iii,iv,v,vi,viii} {iii,iv,v,vi,ix} {iii,iv,v,vi,x} {iii,iv,v,vi,xi} {iii,iv,v,vii,viii} {iii,iv,v,vii,ix} {iii,iv,v,vii,x} {iii,iv,v,vii,xi} {iii,iv,v,viii,ix} {iii,iv,v,viii,x} {iii,iv,v,viii,xi} {iii,iv,v,ix,x} {iii,iv,v,ix,xi} {iii,iv,v,x,xi} {iii,iv,vi,vii,viii} {iii,iv,vi,vii,ix} {iii,iv,vi,vii,x} {iii,iv,vi,vii,xi} {iii,iv,vi,viii,ix} {iii,iv,vi,viii,x} {iii,iv,vi,viii,xi} {iii,iv,vi,ix,x} {iii,iv,vi,ix,xi} {iii,iv,vi,x,xi} {iii,iv,vii,viii,ix} {iii,iv,vii,viii,x} {iii,iv,vii,viii,xi} {iii,iv,vii,ix,x} {iii,iv,vii,ix,xi} {iii,iv,vii,x,xi} {iii,iv,viii,ix,x} {iii,iv,viii,ix,xi} {iii,iv,viii,x,xi} {iii,iv,ix,x,xi} {iii,v,vi,vii,viii} {iii,v,vi,vii,ix} {iii,v,vi,vii,x} {iii,v,vi,vii,xi} {iii,v,vi,viii,ix} {ii,v,vi,viii,x} {iii,v,vi, viii, xi} {iii,v,vi,ix,x} {iii,v,vi,ix,xi} {iii,v,vi,x,xi} {iii,v,vii,viii,ix} {iii,v,vii,viii,x} {iii,v,vii,viii,xi} {iii,v,vii,ix,x} {iii,v,vii,ix,xi} {iii,v,vii,x,xi} {iii,v,viii,ix,x} {iii,v,viii,ix,xi} {iii,v,viii,x,xi} {iii,v,ix,x,xi} {iii,vi,vii,viii,ix} {iii,vi,vii,viii,x} {iii,vi,vii,viii,xi} {iii,vi,vii,ix,x} {iii,vi,vii,ix,xi} {iii,vi,vii,x,xi} {iii,vi,viii,ix,x} {iii,vi,viii,ix,xi} {iii,vi, viii,x, xi} {iii,vi,ix,x,xi} {iii,vii,viii,ix,x} {iii,vii,viii,ix,xi} {iii,vii,viii,x,xi} {iii,vii,ix,x,xi} {iii,viii,ix,x,xi} {iv,v,vi,vii,viii} {iv,v,vi,vii,ix} {iv,v,vi,vii,x} {iv,v,vi,vii,xi} {iv,v,vi,viii,ix} {iv,v,vi,viii,x} {iv,v,vi,viii,xi} {iv,v,vi,ix,x} {iv,v,vi,ix,xi} {iv,v,vi,x,xi} {iv,v,vii,viii,ix} {iv,v,vii,viii,x} {iv,v,vii,viii,xi} {iv,v,vii,ix,x} {iv,v,vii,ix,xi} {iv,v,vii,x,xi} {iv,v,viii,ix,x} {iv,v,viii,ix,xi} {iv,v,viii,x,xi} {iv,v,ix,x,xi} {iv,vi,vii,viii,ix} {iv,vi,vii,viii,x} {iv,vi,vii,viii,xi} {iv,vi,vii,ix,x} {iv,vi,vii,ix,xi} {iv,vi,vii,x,xi} {iv,vi,viii,ix,x} {iv,vi,viii,ix,xi} {iv,vi,viii,x,xi} {iv,vi,ix,x,xi} {iv,vii,viii,ix,x} {iv,vii,viii,ix,xi} {iv,vii,viii,x,xi} {iv,vii,ix,x,xi} {iv,viii,ix,x,xi} {v,vi,vii,viii,ix} {v,vi,vii,viii,x} {v,vi,vii,viii,xi} {v,vi,vii,ix,x} {v,vi,vii,ix,xi} {v,vi,vii,x,xi} {v,vi,viii,ix,x} {v,vi,viii,ix,xi} {v,vi,viii,x,xi} {v,vi,ix,x,xi} {v,vii,viii,ix,x} {v,vii,viii,ix,xi} {v,vii,viii,x,xi} {v,vii,ix,x,xi} {v,viii,ix,x,xi} {vi,vii,viii,ix,x} {vi,vii,viii,ix,xi} {vi,vii,viii,x,xi} {vi,vii,ix,x,xi} {vi,viii,ix,x,xi} {vii,viii,ix,x,xi} {i,ii,iii,iv,v,vi} {i,ii,iii,iv,v,vii} {i,ii,iii,iv,v,viii} {i,ii,iii,iv,v,ix} {i,ii,iii,iv,v,x} {i,ii,iii,iv,v,xi} {i,ii,iii,iv,vi,vii} {i,ii,iii,iv,vi,viii} {i,ii,iii,iv,vi,ix} {i,ii,iii,iv,vi,x} {i,ii,iii,iv,vi,xi} {i,ii,iii,iv,vii,viii} {i,ii,iii,iv,vii,ix} {i,ii,iii,iv,vii,x} {i,ii,iii,iv,vii,xi} {i,ii,iii,iv,viii,ix} {i,ii,iii,iv,viii,x} {i,ii,iii,iv,viii,xi} {i,ii,iii,iv,ix,x} {i,ii,iii,iv,ix,xi} {i,ii,iii,iv,x,xi} {i,ii,iii,v,vi,vii} {i,ii,iii,v,vi,viii} {i,ii,iii,v,vi,ix} {i,ii,iii,v,vi,x} {i,ii,iii,v,vi,xi} {i,ii,iii,v,vii,viii} {i,ii,iii,v,vii,ix} {i,ii,iii,v,vii,x} {i,ii,iii,v,vii,xi} {i,ii,iii,v,viii,ix} {i,ii,iii,v,viii,x} {i,ii,iii,v,viii,xi} {i,ii,iii,v,ix,x} {i,ii,iii,v,ix,xi} {i,ii,iii,v,x,xi} {i,ii,iii,vi,vii,viii} {i,ii,iii,vi,vii,ix} {i,ii,iii,vi,vii,x} {i,ii,iii,vi,vii,xi} {i,ii,iii,vi,viii,ix} {i,ii,iii,vi,viii,x} {i,ii,iii,vi,viii,xi} {i,ii,iii,vi,ix,x} {i,ii,iii,vi,ix,xi} {i,ii,iii,vi,x,xi} {i,ii,iii,vii,viii,ix} {i,ii,iii,viii,x,xi} {i,ii,iii,ix,x,xi} {i,ii,iv,v,vi,vii} {i,ii,iv,v,vi,viii} {i,ii,iv,v,vi,ix} {i,ii,iv,v,vi,x} {i,ii,iv,v,vi,xi} {i,ii,iv,v,vii,viii} {i,ii,iv,v,vii,ix} {i,ii,iv,v,vii,x} {i,ii,iv,v,vii,xi} {i,ii,iv,v,viii,ix} {i,ii,iv,v,viii,x} {i,ii,iv,v,viii,xi} {i,ii,iv,v,ix,x} {i,ii,iv,v,ix,xi} {i,ii,iv,v,x,xi} {i,ii,iv,vi,vii,viii} {i,ii,iv,vi,vii,ix} {i,ii,iv,vi,vii,x} {i,ii,iv,vi,vii,xi} {i,ii,iv,vi,viii,ix} {i,ii,iv,vi,viii,x} {i,ii,iv,vi,viii,xi} {i,ii,iv,vi,ix,x} {i,ii,iv,vi,ix,xi} {i,il,iv,vi,x,xi} {i,ii,iv,vii,viii,ix} {i,ii,iv,vii,viii,x} {i,ii,iv,vii,viii,xi} {i,ii,iv,vii,ix,x} {i,ii,iv,vii,ix,xi} {i,ii,iv,vii,x,xi} {i,ii,iv,viii,ix,x} {i,ii,iv,viii,ix,xi} {i,ii,iv,viii,x,xi} {i,ii,iv,ix,x,xi} {i,ii,v,vi,vii,viii} {i,ii,v,vi,vii,ix} {i,ii,v,vi,vii,x} {i,ii,v,vi,vii,xi} {i,ii,v,vi,viii,ix} {i,ii,v,vi,viii,x} {i,ii,v,vi,viii,xi} {i,ii,v,vi,ix,x} {i,ii,v,vi,ix,xi} {i,ii,v,vi,x,xi} {i,ii,v,vii,viii,ix} {i,ii,v,vii,viii,x} {i,ii,v,vii,viii,xi} {i,ii,v,vii,ix,x} {i,ii,v,vii,ix,xi} {i,ii,v,vii,x,xi} {i,ii,v,viii,ix,x} {i,ii,v,viii,ix,xi} {i,ii,v,viii,x,xi} {i,ii,v,ix,x,xi} {i,ii,vi,vii,viii,ix} {i,ii,vi,vii,viii,x} {i,ii,vi,vii,viii,xi} {i,ii,vi,vii,ix,x} {i,ii,vi,vii,ix,xi} {i,ii,vi,vii,x,xi} {i,ii,vi,viii,ix,x} {i,ii,vi,viii,ix,xi} {i,ii,vi,viii,x,xi} {i,ii,vi,ix,x,xi} {i,ii,vii,viii,ix,x} {i,ii,vii,viii,ix,xi} {i,ii,vii,viii,x,xi} {i,ii,vii,ix,x,xi} {i,ii,viii,ix,x,xi} {i,iii,iv,v,vi,vii} {i,iii,iv,v,vi,viii} {i,iii,iv,v,vi,ix} {i,iii,iv,v,vi,x} {i,iii,iv,v,vi,xi} {i,iii,iv,v,vii,viii} {i,iii,iv,v,vii,ix} {i,iii,iv,v,vii,x} {i,iii,iv,v,vii,xi} {i,iii,iv,v,viii,ix} {i,iii,iv,v,viii,x} {i,iii,iv,v,viii,xi} {i,iii,iv,v,ix,x} {i,iii,iv,v,ix,xi} {i,iii,iv,v,x,xi} {i,iii,iv,vi,vii,viii} {i,iii,iv,vi,vii,ix} {i,iii,iv,vi,vii,x} {i,iii,iv,vi,vii,xi} {i,iii,iv,vi,viii,ix} {i,iii,iv,vi,viii,x} {i,iii,iv,vi,viii,xi} {i,iii,iv,vi,ix,x} {i,iii,iv,vi,ix,xi} {i,iii,iv,vi,x,xi} {i,iii,iv,vii,viii,ix} {i,iii,iv,vii,viii,x} {i,iii,iv,vii,viii,xi} {i,iii,iv,vii,ix,x} {i,iii,iv,vii,ix,xi} {i,iii,iv,vii,x,xi} {i,iii,iv,viii,ix,x} {i,iii,iv,viii,ix,xi} {i,iii,iv,viii,x,xi} {i,iii,iv,ix,x,xi} {i,iii,v,vi,vii,viii} {i,iii,v,vi,vii,ix} {i,iii,v,vi,vii,x} {i,iii,v,vi,vii,xi} {i,iii,v,vi,xiii,ixi} {i,iii,v,vi,viii,x} {i,iii,v,vi,viii,xi} {i,iii,v,vi,ix,x} {i,iii,v,vi,ix,xi} {i,i,v,vi,x,xi} {i,ii,v,vii,viii,ix} {i,iii,v,vii,vi,x} {i,iii,v,vii,viii,xi} {i,iii,v,vii,ix,x} {i,ii,v,vii,ix,xi} {i,iii,v,vii,x,xi} {i,iii,v,viii,ix,x} {i,ii,v,viii,ix,xi} {i,iii,v,viii,x,xi} {i,iii,v,ix,x,xi} {i,iii,vi,vii,viii,ix} {i,iii,vi,vii,viii,x} {i,iii,vi,vii,viii,xi} {i,iii,vi,vii,ix,x} {i,iii,vi,vii,ix,xi} {i,iii,vi,vii,x,xi} {i,iii,vi,viii,ix,x} {i,iii,vi,viii, ix,xi} {i,iii,vi,viii,x,xi} {i,iii,vi,ix,x,xi} {i,iii,vii,viii,ix,x} {i,iii,vii,viii,ix,xi} {i,iii,vii,viii,x,xi} {i,iii,vii,ix,x,xi} {i,iii,viii,ix,x,xi} {i,iv,v,vi,vii,viii} {i,iv,v,vi,vii,ix} {i,iv,v,vi,vii,x} {i,iv,v,vi,vii,xi} {i,iv,v,vi,viii,ix} {i,iv,v,vi,viii,x} {i,iv,v,vi,viii,xi} {l,iv,v,vi,ix,x} {i,iv,v,vi,ix,xi} {i,iv,v,vi,x,xi} {i,iv,v,vii,viii,ix} {i,iv,v,vii,viii,x} {i,iv,v,vii,viii,xi} {i,iv,v,vii,ix,x} {i,iv,v,vii,ix,xi} {Liv,v,vii,x,xi} {i,iv,v,viii,ix,x} {i,iv,v,viii,ix,xi} {i,iv,v,viii,x,xi} {i,iv,v,ix,x,xi} {i,iv,vi,vii,viii,ix} {i,iv,vi,vii,viii,x} {i,iv,vi,vii,viii,xi} {i,iv,vi,vii,ix,x} {i,iv,vi,vii,ix,xi} {i,iv,vi,vii,x,xi} {i,iv,vi,viii,ix,x} {i,iv,vi,viii,ix,xi} {i,iv,vi,viii,x,xi} {i,iv,vi,ix,x,xi} {i,iv,vii,viii,ix,x} {i,iv,vii,viii,ix,xi} {i,iv,vii,viii,x,xi} {i,iv,vii,ix,x,xi} {i,iv,viii,ix,x,xi} {i,v,vi,vii,viii,ix} {i,v,vi,vii,viii,x} {i,v,vi,vii,viii,xi} {i,v,vi,vii,ix,x} {i,v,vi,vii,ix,xi} {i,v,vi,vii,x,xi} {i,v,vi,viii,ix,x} {i,v,vi,viii,ix,xi} {i,v,vi,viii,x,xi} {i,v,vi,ix,x,xi} {i,v,vii,viii,ix,x} {i,v,vii,viii,ix,xi} {i,v,vii,viii,x,xi} {i,v,vii,ix,x,xi} {i,v,viii,ix,x,xi} {i,vi,vii,viii,ix,x} {i,vi,vii,viii,ix,xi} {i,vi,vii,viii,x,xi} {i,vi,vii,ix,x,xi} {i,vi,viii,ix,x,xi} {i,vii,viii,ix,x,xi} {ii,iii,iv,v,vi,vii} {ii,iii,iv,v,vi,viii} {ii,iii,iv,v,vi,ix} {ii,iii,iv,v,vi,x} {ii,iii,iv,v,vi,xi} {ii,iii,iv,v,vii,viii} {ii,iii,iv,v,vii,ix} {ii,iii,iv,v,vii,x} {ii,iii,iv,v,vii,xi} {ii,iii,iv,v,viii,ix} {ii,iii,iv,v,viii,x} {ii,iii,iv,v,viii,xi} {ii,iii,iv,v,ix,x} {ii,iii,iv,v,ix,xi} {ii,iii,iv,v,x,xi} {ii,iii,iv,vi,vii,viii} {ii,iii,iv,vi,vii,ix} {ii,iii,iv,vi,vii,x} {ii,iii,iv,vi,vii,xi} {ii,iii,iv,vi,viii,ix} {ii,iii,iv,vi,viii,x} {ii,iii,iv,vi,viii,xi} {ii,iii,iv,vi,ix,x} {ii,iii,iv,vi,ix,xi} {ii,iii,iv,vi,x,xi} {ii,iii,iv,vii,viii,ix} {ii,iii,iv,vii,viii,x} {ii,iii,iv,vii,viii,xi} {ii,iii,iv,vii,ix,x} {ii,iii,iv,vii,ix,xi} {ii,iii,iv,vii,x,xi} {ii,iii,iv,viii,ix,x} {ii,iii,iv,viii,ix,xi} {ii,iii,iv,viii,x,xi} {ii,iii,iv,ix,x,xi} {ii,iii,v,vi,vii,viii} {ii,iii,v,vi,vii,ix} {ii,iii,v,vi,vii,x} {ii,iii,v,vi,vii,xi} {ii,iii,v,vi,viii,ix} {ii,iii,v,vi,viii,x} {ii,iii,v,vi,viii,xi} {ii,iii,v,vi,ix,x} {ii,iii,v,vi,ix,xi} {ii,iii,v,vi,x,xi} {ii,iii,v,vii,viii,ix} {ii,iii,v,vii,viii,x} {ii,iii,v,vii,viii,xi} {ii,iii,v,vii,ix,x} {ii,iii,v,vii,ix,xi} {ii,iii,v,vii,x,xi} {ii,iii,v,viii,ix,x} {ii,iii,v,viii,ix,xi} {ii,iii,v,viii,x,xi} {ii,iii,v,ix,x,xi} {ii,iii,vi,vii,viii,ix} {ii,iii,vi,vii, viii,x} {ii,iii,vi,vii,viii,xi} {ii,iii,vi,vii,ix,x} {ii,iii,vi,vii, ix,xi} {ii,iii,vi,vii,x,xi} {ii,iii,vi,viii,ix,x} {ii,iii,vi,viii,ix,xi} {ii,iii,vi,viii,x,xi} {ii,iii,vi,ix,x,xi} {ii,iii,vii,viii,ix,x} {ii,iii,vii,viii,ix,xi} {ii,iii,vii,viii,x,xi} {ii,iii,vii,ix,x,xi} {ii,iii,viii,ix,x,xi} {ii,iv,v,vi,vii,viii} {ii,iv,v,vi,vii,ix} {ii,iv,v,vi,vii,x} {ii,iv,v,vi,vii,xi} {ii,iv,v,vi,viii,ix} {ii,iv,v,vi,viii,x} {ii,iv,v,vi,viii,xi} {ii,iv,v,vi,ix,x} {ii,iv,v,vi,ix,xi} {ii,iv,v,vi,x,xi} {ii,iv,v,vii,viii,ix} {ii,iv,v,vii,viii,x} {ii,iv,v,vii,viii,xi} {ii,iv,v,vii,ix,x} {ii,iv,v,vii,ix,xi} {ii,iv,v,vii,x,xi} {ii,iv,v,viii,ix,x} {ii,iv,v,viii,ix,xi} {ii,iv,v,viii,x,xi} {ii,iv,v,ix,x,xi} {ii,iv,vi,vii,viii,ix} {ii,iv,vi,vii,viii,x} {ii,iv,vi,vii,viii,xi} {ii,iv,vi,vii,ix,x} {ii,iv,vi,vii,ix,xi} {ii,iv,vi,vii,x,xi} {ii,iv,vi,viii,ix,x} {ii,iv,vi,viii,ix,xi} {ii,iv,vi,viii,x,xi} {ii,iv,vi,ix,x,xi} {ii,iv,vii,viii,ix,x} {ii,iv,vii,viii,ix,xi} {ii,iv,vii,viii,x,xi} {ii,iv,vii,ix,x,xi} {ii,iv,viii,ix,x,xi} {ii,v,vi,vii,viii,ix} {ii,v,vi,vii,viii,x} {ii,v,vi,vii,viii,xi} {ii,v,vi,vii,ix,x} {i,v,vi,vii,ix,xi} {ii,v,vi,vii,x,xi} {ii,v,vi,viii,ix,x} {ii,v,vi,viii,ix,xi} {ii,v,vi,viii,x,xi} {ii,v,vi,ix,x,xi} {ii,v,vii,viii,ix,x} {ii,v,vii,viii,ix,xi} {ii,v,vii,viii,x,xi} {ii,v,vii,ix,x,xi} {ii,v,viii,ix,x,xi} {ii,vi,vii,viii,ix,x} {ii,vi,vii,viii,ix,xi} {ii,vi,vii,viii,x,xi} {ii,vi,vii,ix,x,xi} {ii,vi,viii,ix,x,xi} {ii vii,viii,ix,x,xi} {iii,iv,v,vi,vii,viii} {iii,iv,v,vi,vii,ix} {iii,iv,v,vi,vii,x} {iii,iv,v,vi,vii,xi} {iii,iv,v,vi,viii,ix} {iii,iv,v,vi,viii,x} {iii,iv,v,vi,viii,xi} {iii,iv,v,vi,ix,x} {iii,iv,v,vi,ix,xi} {iii,iv,v,vi,x,xi} {iii,iv,v,vii,viii,ix} {iii,iv,v,vii,viii,x} {iii,iv,v,vii,viii,xi} {iii,iv,v,vii,ix,x} {iii,iv,v,vii,ix,xi} {iii,iv,v,vii,x,xi} {iii,iv,v,viii,ix,x} {iii,iv,v,viii,ix,xi} {iii,iv,v,viii,x,xi} {iii,iv,v,ix,x,xi} {iii,iv,vi,vii,viii,ix} {iii,iv,vi,vii,viii,x} {iii,iv,vi,vii,viii,xi} {iii,iv,vi,vii,ix,x} {iii,iv,vi,vii,ix,xi} {iii,iv,vi,vii,x,xi} {iii,iv,vi,viii,ix,x} {iii,iv,vi,viii,ix,xi} {iii,iv,vi,viii,x,xi} {iii,iv,vi,ix,x,xi} {iii,iv,vii,viii,ix,x} {iii,iv,vii,viii,ix,xi} {iii,iv,vii,viii,x,xi} {iii,iv,vii,ix,x,xi} {iii,iv,viii,ix,x,xi} {iii,v,vi,vii,viii,ix} {iii,v,vi,vii,viii,x} {iii,v,vi,vii,viii,xi} {iii,v,vi,vii,ix,x} {iii,v,vi,vii,ix,xi} {iii,v,vi,vii,x,xi} {iii,v,vi,viii,ix,x} {iii,v,vi,viii,ix,xi} {iii,v,vi,viii,x,xi} {iii,v,vi,ix,x,xi} {iii,v,vii,viii,ix,x} {iii,v,vii,viii,ix,xi} {lii,v,vii,viii,x,xi} {iii,v,vii,ix,x,xi} {iii,v,viii,ix,x,xi} {iii,vi,vii,viii,ix,x} {iii,vi,vii,viii,ix,xi} {iii,vi,vii,viii,x,xi} {iii,vi,vii,ix,x,xi} {iii,vi,viii,ix,x,xi} {iii,vii,viii,ix,x,xi} {iv,v,vi,vii,viii,ix} {iv,v,vi,vii,viii,x} {iv,v,vi,vii,viii,xi} {iv,v,vi,vii,ix,x} {iv,v,vi,vii,ix,xi} {iv,v,vi,vii,x,xi} {iv,v,vi,viii,ix,x} {iv,v,vi,viii,ix,xi} {iv,v,vi,viii,x,xi} {iv,v,vi,ix,x,xi} {iv,v,vii,viii,ix,x} {iv,v,vii,ix,x,xi} {iv,v,viii,ix,x,xi} {iv,v,vii,ix,x,xi} {iv,v,viii,ix,x,xi} {iv,vi,vii,viii,ix,x} {iv,vi,vii,viii,ix,xi} {iv,vi,vii,viii,x,xi} {iv,vi,vii,ix,x,xi} {iv,vi,viii,ix,x,xi} {iv,vii,viii,ix,x,xi} {v,vi,vii,viii,ix,x} {v,vi,vii,viii,ix,xi} {v,vi,vii,viii,x,xi} {v,vi,vii,ix,x,xi} {v,vi,viii,ix,x,xi} {v,vii,viii,ix,x,xi} {v,vii,viii,ix,x,xi} {i,ii,iii,iv,v,vi,vii} {i,ii,iii,iv,v,vi,viii} {i,ii,iii,iv,v,vi,xi} {i,ii,iii,iv,v,vi,x} {i,ii,iii,iv,v,vi,xi} {i,ii,iii,iv,v,vii,viii} {i,ii,iii,iv,v,vii,ix} {i,ii,iii,iv,v,vii,x} {i,ii,iii,iv,v,vii,xi} {i,ii,iii,iv,v,viii,ix} {i,ii,iii,iv,v,viii,x} {i,ii,iii,iv,v,viii,xi} {i,ii,iii,iv,v,ix,x} {i,ii,iii,iv,v,ix,xi} {i,ii,iii,iv,v,x,xi} {i,ii,iii,iv,vi,viii,xi} {i,ii,iii,iv,vi,ix,x} {i,ii,iii,iv,vi,ix,xi} {i,ii,iii,iv,vi,x,xi} {i,ii,iii,iv,vii,viii,ix} {i,ii,iii,iv,vi,viii,x} {i,ii,iii,iv,vii,viii,xi} {i,ii,iii,iv,vii,ix,x} {i,ii,iii,iv,vii,ix,xi} {i,ii,iii,iv,vii,x,xi} {i,ii,iii,iv,viii,ix,x} {i,ii,iii,iv,viii,ix,xi} {i,ii,iii,iv,vii,viii,xi} {i,ii,iii,iv,vii,ix,x} {i,ii,iii,iv,vii,ix,xi} {i,ii,iii,iv,vii,x,xi} {i,ii,iii,iv,viii,ix,x} {i,ii,iii,iv,viii,ix,xi} {i,ii,iii,iv,viii,x,xi} {i,ii,iii,iv,ix,x,xi} {i,ii,iii,v,vi,vii,viii} {i,ii,iii,v,vi,vii,xi} {i,ii,iii,v,vi,vii,x} {i,ii,iii,v,vi,vii,xi} {i,ii,iii,v,vi,viii,x} {i,ii,iii,v,vi,viii,x} {i,ii,iii,v,vi,viii,xi} {i,ii,iii,v,vi,ix,x} {i,ii,iii,v,vi,ix,xi} {i,ii,iii,v,vi,x,xi} {i,ii,iii,v,vii,viii,ix} {i,ii,iii,v,vii,viii,x} {i,ii,iii,v,vii,viii,xi} {i,ii,iii,v,vii,ix,x} {i,ii,iii,v,vii,ix,xi} {i,ii,iii,v,vii,x,xi} {i,ii,iii,v,viii,ix,x} {i,ii,iii,v,viii,ix,xi} {i,ii,iii,vi,viii,x,xi} {i,ii,iii,v,ix,x,xi} {i,ii,iii,vi,vii,viii,ix} {i,ii,iii,vi,vii,viii,x} {i,ii,iii,vi,vii,viii,xi} {i,ii,iii,vi,vii,ix,x} {i,ii,iii,vi,vii,ix,xi} {i,ii,iii,vi,vii,x,xi} {i,ii,iii,vi,viii,ix,x} {i,ii,iii,vi,viii,ix,xi} {i,ii,iii,vi,viii,x,xi} {i,ii,iii,vi,ix,x,xi} {i,ii,iii,vii,viii,ix,x} {i,ii,iii,vii,viii,ix,xi} {i,ii,iii,vii,viii,x,xi} {i,ii,iii,vii,ix,x,xi} {i,ii,iii,viii,ix,x,xi} {i,ii,iv,v,vi,vii,viii} {i,ii,iv,v,vi,vii,ix} {i,ii,iv,v,vi,vii,x} {i,ii,iv,v,vi,vii,xi} {i,ii,iv,v,vi,viii,ix} {i,ii,iv,v,vi,viii,x} {i,ii,iv,v,vi,viii,xi} {i,ii,iv,v,vi,ix,x} {i,ii,iv,v,vi,ix,xi} {i,ii,iv,v,vi,x,xi} {i,ii,iv,v,vii,viii,ix} {i,ii,iv,v,vii,viii,x} {i,ii,iv,v,vii,viii,xi} {i,ii,iv,v,vii,ix,x} {i,ii,iv,v,vii,ix,xi} {i,ii,iv,v,vii,x,xi} {i,ii,iv,v,viii,ix,x} {i,ii,iv,v,viii,ix,xi} {i,ii,iv,v,viii,x,xi} {i,ii,iv,v,ix,x,xi} {i,ii,iv,vi,vii,viii,ix} {i,ii,iv,vi,vii,viii,x} {i,ii,iv,vi,vii,viii,xi} {i,ii,iv,vi,vii,ix,x} {i,ii,iv,vi, vii,ix,xi} {i,ii,iv,vi,vii,x,xi} {i,ii,iv,vi,viii,ix,x} {i,ii,iv,vi,viii,ix,xi} {i,ii,iv,vi,viii,x,xi} {i,ii,iv,vi,ix,x,xi} {i,ii,iv,vii,viii,ix,x} {i,ii,iv,vii,viii,ix,xi} {i,ii,iv,vii,viii,x,xi} {i,ii,iv,vii,ix,x,xi} {i,ii,iv,viii,ix,x,xi} {i,ii,v,vi,vii,viii,ix} {i,ii,v,vi,vii,viii,x} {i,ii,v,vi,vii,viii,xi} {i,ii,v,vi,vii,ix,x} {i,ii,v,vi,vii,ix,xi} {i,ii,v,vi,vii,x,xi} {i,ii,v,vi,viii,ix,x} {i,ii,v,vi,viii,ix,xi} {i,ii,v,vi,viii,x,xi} {i,ii,v,vi,ix,x,xi} {i,ii,v,vii,viii,ix,x} {i,ii,v,vii,viii,ix,xi} {i,ii,v,vii,viii,x,xi} {i,ii,v,vii,ix,x,xi} {i,ii,v,viii,ix,x,xi} {i,ii,vi,vii,viii,ix,x} {i,ii,vi,vii,viii,ix,xi} {i,ii,vi,vii,viii,x,xi} {i,ii,vi,vii,ix,x,xi} {i,ii,vi,viii,ix,x,xi} {i,ii,vii,viii,ix,x,xi} {i,iii,iv,v,vi,vii,viii} {i,iii,iv,v,vi,vii,ix} {i,iii,iv,v,vi,vii,x} {i,iii,iv,v,vi,vii,xi} {i,iii,iv,v,vi,viii,ix} {i,iii,iv,v,vi,viii,x} {i,iii,iv,v,vi,viii,xi} {i,iii,iv,v,vi,ix,x} {i,iii,iv,v,vi,ix,xi} {i,iii,iv,v,vi,x,xi} {i,iii,iv,v,vii,viii,ix} {i,iii,iv,v,vii,viii,x} {i,iii,iv,v,vii,viii,xi} {i,iii,iv,v,vii,ix,x} {i,iii,iv,v,vii,ix,xi} {i,iii,iv,v,vii,x,xi} {i,iii,iv,v,viii,ix,x} {i,iii,iv,v,viii,ix,xi} {i,iii,iv,v,viii,x,xi} {i,iii,iv,v,ix,x,xi} {i,iii,iv,vi,vii,viii,ix} {i,iii,iv,vi,vii,viii,x} {i,iii,iv,vi,vii,viii,xi} {i,iii,iv,vi,vii,ix,x} {i,iii,iv,vi,vii,ix,xi} {i,iii,iv,vi,vii,x,xi} {i,iii,iv,vi,viii,ix,x} {i,iii,iv,vi,viii,ix,xi} {i,iii,iv,vi,viii,x,xi} {i,iii,iv,vi,ix,x,xi} {i,iii,iv,vii,viii,ix,x} {i,iii,iv,vii,viii,ix,xi} {i,iii,iv,vii,viii,x,xi} {i,iii,iv,vii,ix,x,xi} {i,iii,iv,viii,ix,x,xi} {i,iii,v,vi,vii,viii,ix} {i,iii,v,vi,vii,viii,x} {i,iii,v,vi,vii,viii,xi} {i,iii,v,vi,vii,ix,x} {i,iii,v,vi,vii,ix,xi} {i,iii,v,vi,vii,x,xi} {i,iii,v,vi,viii,ix,x} {i,iii,v,vi,viii,ix,xi} {i,iii,v,vi,viii,x,xi} {i,iii,v,vi,ix,x,xi} {i,iii,v,vii,viii,ix,x} {i,iii,v,vii,viii,ix,xi} {i,iii,v,vii,viii,x,xi} {i,iii,v,vii,ix,x,xi} {i,iii,v,viii,ix,x,xi} {i,iii,vi,vii,viii,ix,x} {i,iii,vi,vii,viii,ix,xi} {i,iii,vi,vii,viii,x,xi} {i,iii,vi,vii,ix,x,xi} {i,iii,vi,viii,ix,x,xi} {i,iii,vii,viii,ix,x,xi} {i,iv,v,vi,vii,viii,ix} {i,iv,v,vi,vii,viii,x} {i,iv,v,vi,vii,viii,xi} {i,iv,v,vi,vii,ix,x} {i,iv,v,vi,vii,ix,xi} {i,iv,v,vi,vii,x,xi} {i,iv,v,vi,viii,ix,x} {i,iv,v,vi,viii,ix,xi} {i,iv,v,vi,viii,x,xi} {i,iv,v,vi,ix,x,xi} {i,iv,v,vii,viii,ix,x} {i,iv,v,vii,viii,ix,xi} {i,iv,v,vii,viii,x,xi} {i,iv,v,vii,ix,x,xi} {i,iv,v,viii,ix,x,xi} {i,iv,vi,vii,viii,ix,x} {i,iv,vi,vii,viii,ix,xi} {i,iv,vi,vii,viii,x,xi} {i,iv,vi,vii,ix,x,xi} {i,iv,vi,viii,ix,x,xi} {i,iv,vii,viii,ix,x,xi} {i,v,vi,vii,viii,ix,x} {i,v,vi,vii,viii,ix,xi} {i,v,vi,vii,viii,x,xi} {i,v,vi,vii,ix,x,xi} {i,v,vi,viii,ix,x,xi} {i,v,vii,viii,ix,x,xi} {i,vi,vii,viii,ix,x,xi} {ii,iii,iv,v,vi,vii,viii} {ii,iii,iv,v,vi,vii,ix} {ii,iii,iv,v,vi,vii,x} {ii,iii,iv,v,vi,vii,xi} {ii,iii,iv,v,vi,viii,ix} {ii,iii,iv,v,vi,viii,x} {ii,iii,iv,v,vi,viii,xi} {ii,iii,iv,v,vi,ix,x} {ii,iii,iv,v,vi,ix,xi} {ii,iii,iv,v,vi,x,xi} {ii,iii,iv,v,vii,viii,ix} {ii,iii,iv,v,vii,viii,x} {ii,iii,iv,v,vii,viii,xi} {ii,iii,iv,v,vii,ix,x} {ii,iii,iv,v,vii,ix,xi} {i,iii,iv,v,vii,x,xi} {ii,iii,iv,v,viii,ix,x} {ii,iii,iv,v,viii,ix,xi} {ii,iii,iv,v,viii,x,xi} {ii,iii,iv,v,ix,x,xi} {ii,iii,iv,vi,vii,viii,ix} {ii,iii,iv,vi,vii,viii,x} {ii,iii,iv,vi,vii,viii,xi} {ii,iii,iv,vi,vii,ix,x} {ii,iii,iv,vi,vii,ix,xi} {ii,iii,iv,vi,vii,x,xi} {ii,iii,iv,vi,viii, ix,x} {ii,iii,iv,vi,viii,ix,xi} {ii,iii,iv,vi,viii,x,xi} {ii,iii,iv,vi,ix,x,xi} {ii,iii,iv,vii,viii,ix,x} {ii,iii,iv,vii,viii,ix,xi} {ii,iii,iv,vii,viii,x,xi} {ii,iii,iv,vii,ix,x,xi} {ii,iii,iv,viii,ix,x,xi} {ii,iii,v,vi,vii,viii,ix} {ii,iii,v,vi,vii,viii,x} {ii,iii,v,vi,vii,viii,xi} {ii,iii,v,vi,vii,ix,x} {ii,iii,v,vi,vii,ix,xi} {ii,iii,v,vi,vii,x,xi} {ii,iii,v,vi,viii,ix,x} {ii,iii,v,vi,viii,ix,xi} {ii,iii,v,vi,viii,x,xi} {ii,iii,v,vi,ix,x,xi} {ii,iii,v,vii,viii,ix,x} {ii,iii,v,vii,viii,ix,xi} {ii,iii,v,vii,viii,x,xi} {ii,iii,v,vii,ix,x,xi} {ii,iii,v,viii,ix,x,xi} {ii,iii,vi,vii,viii,ix,x} {ii,iii,vi,vii,viii,ix,xi} {ii,iii,vi,vii,viii,x,xi} {ii,iii,vi,vii,ix,x,xi} {ii,iii,vi,viii,ix,x,xi} {ii,iii,vii,viii,ix,x,xi} {ii,iv,v,vi,vii,viii,ix} {ii,iv,v,vi,vii,viii,x} {ii,iv,v,vi,vii,viii,xi} {ii,iv,v,vi,vii,ix,x} {ii,iv,v,vi,vii,ix,xi} {ii,iv,v,vi,vii,x,xi} {ii,iv,v,vi,viii,ix,x} {ii,iv,v,vi,viii,ix,xi} {ii,iv,v,vi,viii,x,xi} {ii,iv,v,vi,ix,x,xi} {ii,iv,v,vii,viii,ix,x} {ii,iv,v,vii,viii,ix,xi} {ii,iv,v,vii,viii,x,xi} {ii,iv,v,vii,ix,x,xi} {ii,iv,v,viii,ix,x,xi} {ii,iv,vi,vii,viii,ix,x} {ii,iv,vi,vii,viii,ix,xi} {ii,iv,vi,vii,viii,x,xi} {ii,iv,vi,vii,ix,x,xi} {ii,iv,vi,viii,ix,x,xi} {ii,iv,vii,viii,ix,x,xi} {ii,v,vi,vii,viii,ix,x} {ii,v,vi,vii,viii,ix,xi} {ii,v,vi,vii,viii,x,xi} {ii,v,vi,vii,ix,x,xi} {ii,v,vi,viii,ix,x,xi} {ii,v,vii,viii,ix,x,xi} {ii,vi,vii,viii,ix,x,xi} {iii,iv,v,vi,vii,viii,ix} {iii,iv,v,vi,vii,viii,x} {iii,iv,v,vi,vii,viii,xi} {iii,iv,v,vi,vii,ix,x} {iii,iv,v,vi,vii,ix,xi} {iii,iv,v,vi,vii,x,xi} {iii,iv,v,vi,viii,ix,x} {iii,iv,v,vi,viii,ix,xi} {iii,iv,v,vi,viii,x,xi} {iii,iv,v,vi,ix,x,xi} {iii,iv,v,vii,viii,ix,x} {iii,iv,v,vii,viii,ix,xi} {iii,iv,v,vii,viii,x,xi} {iii,iv,v,vii,ix,x,xi} {iii,iv,v,viii,ix,x,xi} {iii,iv,vi,vii,viii,ix,x} {iii,iv,vi,vii,viii,ix,xi} {iii,iv,vi,vii,viii,x,xi} {iii,iv,vi,vii,ix,x,xi} {iii,iv,vi,viii,ix,x,xi} {iii,iv,vii,viii,ix,x,xi} {iii,v,vi,vii,viii,ix,x} {iii,v,vi,vii,viii,ix,xi} {ii,v,vi,vii,viii,x,xi} {iii,v,vi,vii,ix,x,xi} {iii,v,vi,viii,ix,x,xi} {iii,v,vii,viii,ix,x,xi} {iii,vi,vii,viii,ix,x,xi} {iv,v,vi,vii,viii,ix,x} {iv,v,vi,vii,viii,ix,xi} {iv,v,vi,vii,viii,x,xi} {iv,v,vi,vii,ix,x,xi} {iv,v,vi,viii,ix,x,xi} {iv,v,vii,viii,ix,x,xi} {iv,vi,vii,viii,ix,x,xi} {v,vi,vii,viii,ix,x,xi} {i,ii,iii,iv,v,vi,vii,viii} {i,ii,iii,iv,v,vi,vii,ix} {i,ii,iii,iv,v,vi,vii,x} {i,ii,iii,iv,v,vi,vii,xi} {i,ii,iii,iv,v,vi,viii,ix} {i,ii,iii,iv,v,vi,viii,x} {i,ii,iii,iv,v,vi,viii,xi} {i,ii,iii,iv,v,vi,x,xi} {i,ii,iii,iv,v,vi,ix,xi} {i,ii,iii,iv,v,vi,x,xi} {i,ii,iii,iv,v,vii,x,xi} {i,ii,iii,iv,v,viii,ix,x} {i,ii,iii,iv,v,viii,ix,xi} {i,ii,iii,iv,v,vii,ix,x} {i,ii,iii,iv,v,vii,ix,xi} {i,ii,iii,iv,v,vii,x,xi} {i,ii,iii,iv,v,viii,ix,x} {i,ii,iii,iv,v,viii,ix,xi} {i,ii,iii,iv,v,viii,x,xi} {i,ii,iii,iv,v,ix,x,xi} {i,ii,iii,iv,vi,vii,viii,ix} {i,ii,iii,iv,vi,vii,viii,x} {i,ii,iii,iv,vi,vii,viii,xi} {i,ii,iii,iv,vi,vii,ix,x} {i,ii,iii,iv,vi,vii,ix,xi} {i,ii,iii,iv,vi,vii,x,xi} {i,ii,iii,iv,vi,viii,ix,x} {i,ii,iii,iv,vi,viii,ix,xi} {i,ii,iii,iv,vi,viii,x,xi} {i,ii,iii,iv,vi,ix,x,xi} {i,ii,iii,iv,vii,viii,ix,x} {i,ii,iii,iv,vii,viii,ix,xi} {i,ii,iii,iv,vii,viii,x,xi} {i,ii,iii,iv,vii,ix,x,xi} {i,ii,iii,vi,viii,ix,x,xi} {i,ii,iii,v,vi,vii,viii,ix} {i,ii,iii,v,vi,vii,viii,x} {i,ii,iii,v,vi,viiixi} {i,ii,iii,v,vi,vii,ix,x} {i,ii,iii,v,vi,vii,ix,xi} {i,ii,iii,v,vi,vii,x,xi} {i,ii,iii,v,viii,ix,x} {i,ii,iii,v,vi,viii,ix,xi} {i,ii,iii,v,vi,viii,x,xi} {i,ii,iii,v,vi,ix,x,xi} {i,ii,iii,v,vii,viii,ix,x} {i,ii,iii,v,vii,viii,ix,x} {i,ii,iii,v,vi,viii,ix,xi} {i,ii,iii,v,vii,ix,x,xi} {i,ii,iii,v,vii,ix,x,xi} {i,ii,iii,vi,vii,viii,ix,x} {i,ii,iii,vi,vii,viii,ix,xi} {i,ii,iii,vi,vii,viii,x,xi} {i,ii,iii,vi,vii,ix,x,xi} {i,ii,iii,vi,viii,ix,x,xi} {i,ii,iv,vi,viii,ix,x,xi} {i,ii,iv,v,vi,vii,viii,ix} {i,ii,iv,v,vi,vii,viii,x} {i,ii,iv,v,vi,vii,viii,xi} {i,ii,iv,v,vi,vii,ix,x} {i,ii,iv,v,vi,vii,ix,xi} {i,ii,iv,v,vi,vii,x,xi} {i,ii,iv,v,vi,viii,ix,x} {i,ii,iv,v,vi,viii,ix,xi} {i,ii,iv,v,vi,vii,x,xi} {i,ii,iv,v,vi,ix,x,xi} {i,ii,iv,v,vii,viii,ix,x} {i,ii,iv,v,vii,viii,ix,xi} {i,ii,iv,v,vii,viii,x,xi} {i,ii,iv,v,vii,ix,x,xi} {i,ii,iv,v,viii,ix,x,xi} {i,ii,iv,vi,vii,viii,ix,x} {i,ii,iv,vi,vii,viii,ix,xi} {i,ii,iv,vi,vii,viii,x,xi} {i,ii,iv,vi,vii,ix,x,xi} {i,ii,iv,vi, viii,ix,x,xi} {i,ii,iv,vii,viii,ix,x,xi} {i,ii,v,vi,vii,viii,ix,x} {i,ii,v,vi,vii,viii,ix,xi} {i,ii,v,vi,vii,viii,x,xi} {i,ii,v,vi,vii,ix,x,xi} {i,ii,v,vi,viii,ix,x,xi} {i,ii,v,vii,viii,ix,x,xi} {i,ii,vi,vii,viii,ix,x,xi} {i,iii,iv,v,vi,vii,viii,ix} {i,iii,iv,v,vi,vii,viii,x} {i,iii,iv,v,vi,vii,viii,xi} {i,iii,iv,v,vi,vii,ix,x} {i,iii,iv,v,vi,vii,ix,xi} {i, iii,iv,v,vi,vii,x,xi} {i,iii,iv,v,vi,viii,ix,x} {i,iii,iv,v,vi,viii,ix,xi} {i,iii,iv,v,vi,viii,x,xi} {i,iii,iv,v,vi,ix,x,xi} {i,iii,iv,v,vii,viii,ix,xi} {i,iii,iv,v,vii,viii,x,xi} {i,iii,iv,v,vii,ix,x,xi} {i,iii,iv,vi,viii,ix,x,xi} {i,iii,iv,vi,vii,viii,ix,x} {i,iii,iv,vi,vii,viii,ix,xi} {i,iii,iv,vi,vii,viii,x,xi} {i,iii,iv,vi,vii,ix,x,xi} {i,iii,iv,vi,viii,ix,x,xi} {i,iii,iv,vii,viii,ix,x,xi} {i,iii,v,vi,vii,viii,ix,x} {i,iii,vi,vii,viii,ix,xi} {i,iii,v,vi,vii,viii,x,xi} {i,iv,v,vi,vii,viii,ix,xi} {i,iv,v,vi,vi,vii,x,xi} {i,iv,v,vi,vii,ix,x,xi} {i,iv,v,vi,viii,ix,x,xi} {i,iv,v,vii,viW,ix,x,xi} {i,iv,v,vi,vii,viii,ix,xi} {i,iv,v,vi,vi,vii,x,xi} {i,iv,v,vi,vii,ix,x,xi} {i,iv,v,vi,viii,ix,x,xi} {i,iv,v,vii,viW,ix,x,xi} {i,iv,vi,vii,viii,ix,x,xi} {i,v,vi,vii,viii,ix,x,xi} {ii,iii,iv,v,vi,vii,viii,ix} {ii,iii,iv,v,vi,vii,viii,x} {ii,iii,iv,v,vi,vii,viii,xi} {ii,iii,iv,v,vi,vii,ix,x} {ii,iii,iv,v,vi,vii,ix,xi} {ii,iii,iv,v,vi,vii,x,xi} {ii,ji,iv,v,vi,viii,ix,x} {i,Hi,iv,v,vi,viii,ix,xi}{ii,iii,iv,v,vi,vii,x,xi} {i,iii,iv,v,vi,ix,x,xi} {ii,iii,iv,v,vi,viii,ix,x} {ii,iii,iv,v,vii,viii,ix,xi} {iii,iv,v,vii,viii,x,xi} {ii,iii,iv,v,vii,ix,x,xi} {ii,iii,iv,v,viii,ix,x,xi} {ii,iii,iv,vi,vii,viii,ix,x} {ii,iii,iv,vi,vii,viii,ix,xi} {ii,iii,iv,vi,vii,viii,x,xi} {ii,iii,iv,vi,vii,ix,x,xi} {ii,iii,iv,vi,viii,ix,x,xi} {ii,iii,iv,vii,viii,ix,x,xi} {ii,iii,v,vi,vii,viii,ix,x} {ii,iii,v,vi,vii,viii,ix,xi} {ii,iii,v,vi,vii,viii,x,xi} {ii,iii,v,vl,vii,ix,x,xi} {ii,iii,v,vi,vii,ix,x,xi} {ii,iii,v,vii,viii,ix,x,xi} {ii,iii,vi,vii,viii,ix,x,xi} {ii,iv,v,vi,vii,viii,ix,x} {ii,iv,v,vi,vii,viii,ix,xi} {ii,iv,v,vi,vii,viii,x,xi} {ii,iv,vi,vii,ix,x,xi} {ii,iv,v,vi,viii,ix,x,xi} {ii,iv,v,vii,viii,ix,x,xi} {ii,iv,vi,vii,viii,ix,x,xi} {ii,v,vi,vii,viii,ix,x,xi} {iii,iv,v,vi,vii,viii,ix,x} {iii,iv,v,vi,vii,viii,ix,xi} {iii,iv,v,vi,vii,viii,x,xi} {iii,iv,v,vi,vii,ix,x,xi} {iii,iv,v,vi,viii,ix,x,xi} {iii,iv,v,vii,viii,ix,x,xi} {iii,iv,vi,vii,viii,ix,x,xi} {iii,v,vi,vii,viii,ix,x,xi} {iv,v,vi,vii,vii,ix,x,xi} {i,ii,iii,iv,v,vi,vi,viii,ix} {i,ii,iii,iv,v,vi,vii,viii,x} {i,ii,iii,iv,v,vi,vii,viii,xi} {i,ii,iii,iv,v,vi,vii,ix,x} {i,ii,ii,iv,v,vi,vii,ix,xi} {i,ii,ii,iv,v,vi,vi,x,xi} {i,ii,iii,iv,v,vi,viii,ix,x} {i,ii,iii,iv,v,vi,viii,ix,xi} {i,ii,iii,iv,v,vi,viii,x,xi} {i,ii,iii,iv,v,vi,ix,x,xi} {i,ii,iii,iv,v,vi,viii,ix,x} {i,ii,iii,iv,v,vii,viii,ix,xi} {i,ii,iii,iv,v,vii,viii,x,xi} {i,ii,iii,iv,v,vii,ix,x,xi} {i,ii,ii,iv,v,vii,ix,x,xi} {i,ii,iii,iv,vi,vii,viii,ix,x} {i,i,ii,iv,vi,vi,viii,ix,xi} {i,ii,iii,iv,vi,vii,viii,x,xi} {i,ii,iii,iv,vi,vii,ix,x,xi} {i,ii,iii,iv,vi,viii,ix,x,xi} {i,ii,iii,iv,vii,viii,ix,x,xi} {i,ii,ii,v,vi,vii,viii,ix,x} {i,ii,iii,v,vi,vii,viii,ix,xi} {i,ii,iii,v,vi,vii,viii,x,xi} {i,ii,iii,v,vi,vii,ix,x,xi} {i,ii,iii,v,vi,viii,ix,x,xi} {ii,iii,v,vii,vii,ix,x,xi} {i,ii,iii,vi,vii,vii,ix,x,xi} {i,ii,iv,v,vi,vii,viii,ix,x} {i,ii,iv,v,vi,vii,viii,ix,xi} {i,ii,iv,v,vi,vii,viii,x,xi} {i,ii,iv,v,vi,vii,ix,x,xi} {i,ii,iv,v,vi,viii,ix,x,xi} {i,iii,v,vi,vii,viii,ix,x,xi} {i,ii,iv,vi,vii,viii,ix,x,xi} {i,ii,v,vi,vii,viii,ix,x,xi} {i,iii,iv,v,vi,vii,viii,ix,x} {i,iii,iv,v,vi,vii,viii,ix,xi} {i,iii,iv,v,vi,vii,viii,x,xi} {i,iii,iv,v,vi,vi,ix,x,xi} {i,iii,iv,v,vi,viii,ix,x,xi} {i,iii,iv,v,vii,viii,ix,x,xi} {i,ii,iv,vi,vii,viii,ix,x,xi} {i,iii,v,vi,vii,viii,ix,x,xi} {i,iv,v,vi,vii,viii,ix,x,xi} {ii,iii,iv,v,vi,vii,viii,ix,x} {ii,iii,iv,v,vi,vii,viii,ix,xi} {ii,iii,iv,v,vi,vii,viii,x,xi} {ii,iii,iv,v,vi,vii,ix,x,xi} {ii,iii,iv,v,vi,viii,ix,x,xi} {ii,iii,iv,v,vii,viii,ix,x,xi} {ii,iii,iv,vi,vii,viii,ix,x,xi} {ii,iii,v,vi,vii,viii,ix,x,xi} {ii,iv,v,vi,vii,viii,ix,x,xi} {iii,iv,v,vi,vii,viii,ix,x,xi} {i,ii,iii,iv,v,vi,vii,viii,ix,x} {i,ii,iii,iv,v,vi,vii,viii,ix,xi} {i,ii,iii,iv,v,vi,vii,viii,x,xi} {i,ii,ii,iv,v,vi,vii,ix,x,xi} {i,ii,iii,iv,v,vi,viii,ix,x,xi} {i,ii,iii,iv,v,vii,viii,ix,x,xi} {i,ii,iii,iv,vi,vii,viii,ix,x,xi} {i,ii,iii,v,vi,vii,viii,ix,x,xi} {i,ii,iv,v,vi,vii,viii,ix,x,xi} {i,iii,iv,v,vi,vii,viii,ix,x,xi} {ii,iii,iv,v,vi,vii,viii,ix,x,xi} or {i,ii,iii,v,vi,vii,viii,ix,x,xi}.

If the variant comprises any one of (i) and (iii) to (xi), it may further comprise a mutation at one or more of Y51, N55 and F56, such as at Y51, N55, F56, Y51/N55, Y51/F56, N55/F56 or Y51/N55/F56.

In (i), the variant may comprises mutations at any number and combination of N40, D43, E44, S54, S57, Q62, R97, E101, E124, E131, R142, T150 and R192. In (i), the variant preferably comprises one or more mutations at the following positions (i.e. mutations at one or more of the following positions) N40, D43, E44, S54, S57, Q62, E101, E131 and T150. In (i), the variant preferably comprises one or more mutations at the following positions (i.e. mutations at one or more of the following positions) N40, D43, E44, E101 and E131. In (i), the variant preferably comprises a mutation at S54 and/or S57.

In (i), the variant more preferably comprises a mutation at (a) S54 and/or S57 and (b) one or more of Y51, N55 and F56, such as at Y51, N55, F56, Y51/N55, Y51/F56, N55/F56 or Y51/N55/F56. If S54 and/or S57 are deleted in (xi), it/they cannot be mutated in (i) and vice versa. In (i), the variant preferably comprises a mutation at T150, such as T150I. Alternatively the variant preferably comprises a mutation at (a) T150 and (b) one or more of Y51, N55 and F56, such as at Y51, N55, F56, Y51/N55, Y51/F56, N55/F56 or Y51/N55/F56. In (i), the variant preferably comprises a mutation at Q62, such as Q62R or Q62K. Alternatively the variant preferably comprises a mutation at (a) Q62 and (b) one or more of Y51, N55 and F56, such as at Y51, N55, F56, Y51/N55, Y51/F56, N55/F56 or Y51/N55/F56.

The variant may comprise a mutation at D43, E44, Q62 or any combination thereof, such as D43, E44, Q62, D43/E44, D43/Q62, E44/Q62 or D431E44/Q62. Alternatively the variant preferably comprises a mutation at (a) D43, E44, Q62, D43/E44, D43/Q62, E44/Q62 or D43/E44/Q62 and (b) one or more of Y51, N55 and F56, such as at Y51, N55, F56, Y51/N55, Y51/F56, N55/F56 or Y51/N55/F56.

In (ii) and elsewhere in this application, the / symbol means “and” such that Y51/N55 is Y51 and N55. In (ii), the variant preferably comprises mutations at Y51/N55. It has been proposed that the constriction in CsgG is composed of three stacked concentric rings formed by the side chains of residues Y51, N55 and F56 (Goyal et al, 2014, Nature, 516, 250-253). Mutation of these residues in (ii) may therefore decrease the number of nucleotides contributing to the current as the polynucleotide moves through the pore and thereby make it easier to identify a direct relationship between the observed current (as the polynucleotide moves through the pore) and the polynucleotide. Y56 may be mutated in any of the ways discussed below with reference to variants and pores useful in the method of the invention.

In (v), the variant may comprise N102R, N102F, N102Y or N102W. The variant preferably comprises (a) N102R, N102F, N102Y or N102W and (b) a mutation at one or more of Y51, N55 and F56, such as at Y51, N55, F56, Y51/N55, Y51/F56, N55/F56 or Y51/N55/F56.

In (xi), any number and combination of K49, P50, Y51, P52, A53, S54, N55, F56 and S57 may be deleted. Preferably one or more of K49, P50, Y51, P52, A53, S54, N55 and S57 may be deleted. If any of Y51, N55 and F56 are deleted in (xi), it/they cannot be mutated in (ii) and vice versa.

In (i), the variant preferably comprises one of more of the following substitutions N40R, N40K, D43N, D43Q, D43R, D43K, E44N, E44Q, E44R, E44K, S54P, S57P, Q62R, Q62K, R97N, R97G, R97L, E101N, E101Q, E101R, E101K, E101F, E101Y, E101W, E124N, E124Q, E124R, E124K, E124F, E124Y, E124W, E131D, R142E, R142N, T150I, R192E and R192N, such as one or more of N40R, N40K, D43N, D430, D43R, D43K, E44N, E440, E44R, E44K, S54P, S57P, Q62R, Q62K, E101N, E101Q, E101R, E101K, E101F, E101Y, E101W, E131D and T150I, or one or more of N40R, N40K, D43N, D43Q, D43R, D43K, E44N, E44Q, E44R, E44K, E101N, E101Q, E101R, E101K, E101F, E101Y, E101W and E131D. The variant may comprise any number and combination of these substitutions. In (i), the variant preferably comprises S54P and/or S57P. In (I), the variant preferably comprises (a) S54P and/or S57P and (b) a mutation at one or more of Y51, N55 and F56, such as at Y51, N55, F56, Y51/N55, Y51/F56, N55/F56 or Y51/N55/F56. The mutations at one or more of Y51, N55 and F56 may be any of those discussed below. In (i), the variant preferably comprises F56A/S57P or S54P/F56A.

The variant preferably comprises T150I. Alternatively the variant preferably comprises a mutation at (a) T150I and (b) one or more of Y51, N55 and F56, such as at Y51, N55, F56, Y51/N55, Y51/F56, N55/F56 or Y51/N55/F56.

In (i), the variant preferably comprises Q62R or Q62K. Alternatively the variant preferably comprises (a) Q62R or Q62K and (b) a mutation at one or more of Y51, N55 and F56, such as at Y51, N55, F56, Y51/N55, Y51/F56, N55/F56 or Y51/N55/F56. The variant may comprise D43N, E44N, Q62R or Q62K or any combination thereof, such as D43N, E44N, Q62R, Q62K, D43N/E44N, D43N/Q62R, D43N/Q62K, E44N/Q62R, E44N/Q62K, D43N/E44N/Q62R or D43N/E44N/Q62K. Alternatively the variant preferably comprises (a) D43N, E44N, Q62R, Q62K, D43N/E44N, D43N/Q62R, D43N/Q62K, E44N/Q62R, E44N/Q62K, D43N/E44N/Q62R or D43N/E44N/Q62K and (b) a mutation at one or more of Y51, N55 and F56, such as at Y51, N55, F56, Y51/N55, Y51/F56, N55/F56 or Y51/N55/F56.

In (i), the variant preferably comprises D43N.

In (i), the variant preferably comprises E101R, E101S, E101F or E101N.

In (i), the variant preferably comprises E124N, E124Q, E124R, E124K, E124F, E124Y, E124W or E124D, such as E124N.

In (i), the variant preferably comprises R142E and R142N.

In (i), the variant preferably comprises R97N, R97G or R97L.

In (i), the variant preferably comprises R192E and R192N.

In (ii), the variant preferably comprises F56N/N55Q, F56N/N55R, F56N/N55K, F56N/N55S, F56N/N55G, F56N/N55A, F56N/N55T, F56Q/N55Q, F56Q/N55R, F56Q/N55K, F56Q/N55S, F560/N55G, F56Q/N55A, F560/N55T, F56R/N55Q, F56R/N55R, F56R/N55K, F56R/N55S, F56R/N55G, F56R/N55A, F56R/N55T, F56S/N55Q, F56S/N55R, F56S/N55K, F56S/N55S, F56S/N55G, F56S/N55A, F56S/N55T, F56G/N550, F56G/N55R, F56G/N55K, F56G/N55S, F56G/N55G, F56G/N55A, F56G/N55T, F56A/N550, F56A/N55R, F56A/N55K, F56A/N55S, F56A/N55G, F56A/N55A, F56A/N55T, F56K/N55Q, F56K/N55R, F56K/N55K, F56K/N55S, F56K/N55G, F56K/N55A, F56K/N55T, F56N/Y51L, F56N/Y51V, F56N/Y51A, F56N/Y51N, F56N/Y51Q, F56N/Y51S, F56N/Y51G, F56Q/N51L, F56Q/Y51V, F56Q/Y51A, F56Q/Y51N, F56Q/Y51Q, F56Q/Y51S, F56Q/Y51G, F56R/Y51L, F56R/Y51V, F56R/Y51A, F56R/Y51N, F56R/Y51Q, F56R/Y51S, F56R/Y51G, F56S/Y51L, F56S/Y51V, F56S/Y51A, F56S/Y51N, F56S/Y51Q, F56S/Y51S, F56S/Y51G, F56G/Y51L, F56G/Y51V, F56G/Y51A, F56G/Y51N, F56G/Y51Q, F56G/Y51S, F56G/Y51G, F56A/Y51L, F56A/Y51V, F56A/Y51A, F56A/Y51N, F56A/Y51Q, F56A/Y51S, F56A/Y51G, F56K/Y51L, F56K/Y51V, F56K/Y51A, F56K/Y51N, F56K/Y51Q, F56K/Y51S, F56K/Y51G, N55Q/Y51L, N550/Y51V, N55Q/Y51A, N55Q/Y51N, N55Q/Y51Q, N55Q/Y51S, N55Q/Y51G, N55R/Y51L, N55R/Y51V, N55R/Y51A, N55R/Y51N, N55R/Y51Q, N55R/Y51S, N55R/Y51G, N55K/Y51L, N55K/Y51V, N55K/Y51A, N55K/Y51N, N55K/Y51Q, N55K/Y51S, N55K/N51G, N55S/Y51L, N55S/Y51V, N55S/Y51A, N55S/Y51N, N55S/Y51Q, N55S/Y51S, N55S/Y51G, N55G/Y51L, N55G/Y51V, N55G/Y51A, N55G/Y51N, N55G/Y51Q, N55G/Y51S, N55G/Y51G, N55A/Y51L, N55A/Y51V, N55A/Y51A, N55A/Y51N, N55A/Y51Q, N55A/Y51S, N55A/Y51G, N55T/Y51L, N55T/Y51V, N55T/Y51A, N55T/Y51N, N55T/Y51Q, N55T/Y51S, N55T/Y51G, F56N/N55Q/Y51L, F56N/N55Q/Y51V, F56N/N55Q/Y51A, F56N/N55Q/Y51N, F56N/N55Q/Y51Q, F56N/N55Q/Y51S, F56N/N55Q/Y51G, F56N/N55R/Y51L, F56N/N55R/Y51V, F56N/N55R/Y51A, F56N/N55R/Y51N, F56N/N55R/Y51Q, F56N/N55R/Y51S, F56N/N55R/Y51G, F56N/N55K/Y51L, F56N/N55K/Y51V, F56N/N55K/Y51A, F56N/N55K/Y51N, F56N/N55K/Y51Q, F56N/N55K/Y51S, F56N/N55K/Y51G, F56N/N55S/Y51L, F56N/N55S/Y51V, F56N/N55S/Y51A, F56N/N55S/Y51N, F56N/N55S/Y51Q, F56N/N55S/Y51S, F56N/N55S/Y51G, F56N/N55G/Y51L, F56N/N55G/Y51V, F56N/N55G/Y51A, F56N/N55G/Y51N, F56N/N55G/Y51Q, F56N/N55G/Y51S, F56N/N55G/Y51G, F56N/N55A/Y51L, F56N/N55A/Y51V, F56N/N55A/Y51A, F56N/N55A/Y51N, F56N/N55A/Y51Q, F56N/N55A/Y51S, F56N/N55A/Y51G, F56N/N55T/Y51L, F56N/N55T/Y51V, F56N/N55T/Y51A, F56N/N55T/Y51N, F56N/N55T/Y51Q, F56N/N55T/Y51S, F56N/N55T/Y51G, F56Q/N55Q/Y51L, F56Q/N55Q/Y51V, F56Q/N55Q/Y51A, F56Q/N55Q/Y51N, F56Q/N55Q/Y51Q, F56Q/N55Q/Y51S, F56Q/N55Q/Y51G, F560/N55R/Y51L, F56Q/N55R/Y51V, F56Q/N55R/Y51A, F560N55R/Y51N, F56Q/N55R/Y51Q, F560/N55R/Y51S, F56Q/N55R/Y51G, F560/N55K/Y51L, F58Q/N55K/Y51V, F56Q/N55K/Y51A, F56Q/N55K/Y51N, F56Q/N55K/Y51Q, F560/N55K/Y51S, F56Q/N55K/Y51G, F56Q/N55S/Y51L, F56Q/N55S/Y51V, F56Q/N55S/Y51A, F56Q/N55S/Y51N, F56Q/N55S/Y51Q, F56Q/N55S/Y51S, F560/N55S/Y51G, F56Q/N55G/Y51L, F56Q/N55G/Y51V, F56Q/N55G/Y51A, F56Q/N55G/Y51N, F56Q/N55G/Y51Q, F56Q/N55G/Y51S, F56Q/N55G/Y51G, F560/N55A/Y51L, F56Q/N55A/Y51V, F56Q/N55A/Y51A, F56Q/N55A/Y51N, F56Q/N55A/Y51Q, F56Q/N55A/Y51S, F56Q/N55A/Y51G, F56Q/N55T/Y51L, F56Q/N55T/Y51V, F56Q/N55T/Y51A, F56Q/N55T/Y51N, F56Q/N55T/Y51Q, F56Q/N55T/Y51S, F56Q/N55T/Y51G, F56R/N55Q/Y51L, F56R/N55Q/Y51V, F56R/N55Q/Y51A, F56R/N55Q/Y51N, F56R/N55Q/Y51Q, F56R/N55Q/Y51S, F56R/N55Q/Y51G, F56R/N55R/Y51L, F56R/N55R/Y51V, F56R/N55R/Y51A, F56R/N55R/Y51N, F56R/N55R/Y51Q, F56R/N55R/Y51S, F56R/N55R/Y51G, F56R/N55K/Y51L, F56R/N55K/Y51V, F56R/N55K/Y51A, F56R/N55K/Y51N, F56R/N55K/Y51Q, F56R/N55K/Y51S, F56R/N55K/Y51G, F56R/N55S/Y51L, F56R/N55S/Y51V, F56R/N55S/Y51A, F56R/N55S/Y51N, F56R/N55S/Y51Q, F56R/N55S/Y51S, F56R/N55S/Y51G, F56R/N55G/Y51L, F56R/N55G/Y51V, F56R/N55G/Y51A, F56R/N55G/Y51N, F56R/N55G/Y51Q, F56R/N55G/Y51S, F56R/N55G/Y51G, F56R/N55A/Y51L, F56R/N55A/Y51V, F56R/N55A/Y51A, F56R/N55A/Y51N, F56R/N55A/Y51Q, F56R/N55A/Y51S, F56R/N55A/Y51G, F56R/N55T/Y51L, F56R/N55T/Y51V, F56R/N55T/Y51A, F56R/N55T/Y51N, F56R/N55T/Y51Q, F56R/N55T/Y51S, F56R/N55T/Y51G, F56S/N55Q/Y51L, F56S/N55Q/Y51V, F56S/N55Q/Y51A, F56S/N55Q/Y51N, F56S/N55Q/Y51Q, F56S/N55Q/Y51S, F56S/N55Q/Y51G, F56S/N55R/Y51L, F56S/N55R/Y51V, F56S/N55R/Y51A, F56S/N55R/Y51N, F56S/N55R/Y51Q, F56S/N55R/Y51S, F56S/N55R/Y51G, F56S/N55K/Y51L, F56S/N55K/Y51V, F56S/N55K/Y51A, F56S/N55K/Y51N, F56S/N55K/Y51Q, F56S/N55K/Y51S, F56S/N55K/Y51G, F56S/N55S/Y51L, F56S/N55S/Y51V, F56S/N55S/Y51A, F56S/N55S/Y51N, F56S/N55S/Y51Q, F56S/N55S/Y51S, F56S/N55S/Y51G, F56S/N55G/Y51L, F56S/N55G/Y51V, F56S/N55G/Y51A, F56S/N55G/Y51N, F56S/N55G/Y51Q, F56S/N55G/Y51S, F56S/N55G/Y51G, F56S/N55A/Y51L, F56S/N55A/Y51V, F56S/N55A/Y51A, F56S/N55A/Y51N, F56S/N55A/Y51Q, F56S/N55A/Y51S, F56S/N55A/Y51G, F56S/N55T/Y51L, F56S/N55T/Y51V, F56S/N55T/Y51A, F56S/N55T/Y51N, F56S/N55T/Y51Q, F56S/N55T/Y51S, F56S/N55T/Y51G, F56G/N55Q/Y51L, F56G/N55Q/Y51V, F56G/N55Q/Y51A, F56G/N55Q/Y51N, F56G/N55Q/Y51Q, F56G/N55Q/Y51S, F56G/N55Q/Y51G, F56G/N55R/Y51L, F56G/N55R/Y51V, F56G/N55R/Y51A, F56G/N55R/Y51N, F56G/N55R/Y51Q, F56G/N55R/Y51S, F56G/N55R/Y51G, F56G/N55K/Y51L, F56G/N55K/Y51V, F56G/N55K/Y51A, F56G/N55K/Y51N, F56G/N55K/Y51Q, F56G/N55K/Y51S, F56G/N55K/Y51G, F56G/N55S/Y51L, F56G/N55S/Y51V, F56G/N55S/Y51A, F56G/N55S/Y51N, F56G/N55S/Y51Q, F56G/N55S/Y51S, F56G/N55S/Y51G, F56G/N55G/Y51L, F56G/N55G/Y51V, F56G/N55G/Y51A, F56G/N55G/Y51N, F56G/N55G/Y51Q, F56G/N55G/Y51S, F56G/N55G/Y51G, F56G/N55A/Y51L, F56G/N55A/Y51V, F56G/N55A/Y51A, F56G/N55A/Y51N, F56G/N55A/Y51Q, F56G/N55A/Y51S, F56G/N55A/Y51G, F56G/N55T/Y51L, F56G/N55T/Y51V, F56G/N55T/Y51A, F56G/N55T/Y51N, F56G/N55T/Y51Q, F56G/N55T/Y51S, F56G/N55T/Y51G, F56A/N55Q/Y51L, F56A/N55Q/Y51V, F56A/N55Q/Y51A, F56N/N55Q/Y51N, F56A/N55Q/Y51Q, F56A/N55Q/Y51S, F56A/N55Q/Y51G, F56A/N55R/Y51L, F56A/N55R/Y51V, F56N/N55R/Y51A, F56A/N55R/Y51N, F56A/N55R/Y51Q, F56A/N55R/Y51S, F56A/N55R/Y51G, F56A/N55K/Y51L, F56N/N55K/Y51V, F56A/N55K/Y51A, F56A/N55K/Y51N, F56A/N55K/Y51Q, F56A/N55K/Y51S, F56N/N55K/Y51G, F56A/N55S/Y51L, F56A/N55S/Y51V, F56N55S/Y51A, F56A/N55S/Y51N, F56A/N55S/Y51Q, F56A/N55S/Y51S, F56A/N55S/Y51G, F56A/N55G/Y51L, F56A/N55G/Y51V, F56A/N55G/Y51A, F56N/N55G/Y51N, F56A/N55G/Y51Q, F56N/N55G/Y51S, F56A/N55G/Y51G, F56A/N55A/Y51L, F56A/N55A/Y51V, F56A/N55A/Y51A, F56A/N55A/Y51N, F56N/N55A/Y51Q, F56A/N55A/Y51S, F56A/N55N/Y51G, F56N/N55T/Y51L, F56N/N55T/Y51V, F56N/N55T/Y51A, F56A/N55T/Y51N, F56A/N55T/Y51Q, F56A/N55T/Y51S, F56A/N55T/Y51G, F56K/N55Q/Y51L, F56K/N55Q/Y51V, F56K/N55Q/Y51A, F56K/N55Q/Y51N, F56K/N55Q/Y51Q, F56K/N55Q/Y51S, F56K/N55Q/Y51G, F56K/N55R/Y51L, F56K/N55R/Y51V, F56K/N55R/Y51A, F56K/N55R/Y51N, F56K/N55R/Y51Q, F56K/N55R/Y51S, F56K/N55R/Y51G, F56K/N55K/Y51L, F56K/N55K/Y51V, F56K/N55K/Y51A, F56K/N55K/Y51N, F56K/N55K/Y51Q, F56K/N55K/Y51S, F56K/N55K/Y51G, F56K/N55S/Y51L, F56K/N55S/Y51V, F56K/N55S/Y51A, F56K/N55S/Y51N, F56K/N55S/Y51Q, F56K/N55S/Y51S, F56K/N55S/Y51G, F56K/N55G/Y51L, F56K/N55G/Y51V, F56K/N55G/Y51A, F56K/N55G/Y51N, F56K/N55G/Y51Q, F56K/N55G/Y51S, F56K/N55G/Y51G, F56K/N55A/Y51L, F56K/N55A/Y51V, F56K/N55A/Y51A, F56K/N55A/Y51N, F56K/N55N/Y51Q, F56K/N55A/Y51S, F56K/N55A/Y51G, F56K/N55T/Y51L, F56K/N55T/Y51V, F56K/N55T/Y51A, F56K/N55T/Y51N, F56K/N55T/Y51Q, F56K/N55T/Y51S, F56K/N55T/Y51G, F56E/N55R, F56E/N55K, F56D/N55R, F56D/N55K, F56R/N55E, F56R/N55D, F56K/N55E or F56K/N55D.

In (ii), the variant preferably comprises Y51R/F56Q, Y51N/F56N, Y51M/F58Q, Y51LF56Q, Y51I/F56Q, Y51V/F56Q, Y51A/F56Q, Y51P/F56Q, Y51G/F56Q, Y51C/F560, Y51Q/F56Q, Y51N/F56Q, Y51S/F56Q, Y51E/F560, Y51D/F56Q, Y51K/F560 or Y51H/F56Q.

In (ii), the variant preferably comprises Y51T/F560, Y51Q/F560 or Y51A/F56Q.

In (ii), the variant preferably comprises Y51T/F56F, Y51T/F56M, Y51T/F56L, Y51T/F56I, Y51T/F56V, Y51T/F56A, Y51T/F56P, Y51T/F56G, Y51T/F56C, Y51T/F56Q, Y51T/F56N, Y51T/F56T, Y51T/F56S, Y51T/F56E, Y51T/F56D, Y51T/F56K, Y51T/F56H or Y51T/F56R.

In (ii), the variant preferably comprises Y51T/N55Q, Y51T/N55S or Y51T/N55A.

In (ii), the variant preferably comprises Y51A/F56F, Y51A/F56L, Y51A/F56I, Y51A/F56V, Y51N/F56A, Y51A/F56P, Y51A/F56G, Y51A/F56C, Y51A/F56Q, Y51A/F56N, Y51N/F56T, Y51A/F56S, Y51A/F56E, Y51A/F56D, Y51A/F56K, Y51A/F56H or Y51A/F56R.

In (ii), the variant preferably comprises Y51C/F56A, Y51E/F56A, Y51D/F56A, Y51K/F56A, Y51H/F56A, Y51Q/F56A, Y51N/F56A, Y51S/F56A, Y51P/F56A or Y51V/F56A.

In (xi), the variant preferably comprises deletion of Y51/P52, Y51/P52/A53, P50 to P52, P50 to A53, K49 to Y51, K49 to A53 and replacement with a single proline (P), K49 to S54 and replacement with a single P, Y51 to A53, Y51 to S54, N55/F56, N55 to S57, N55/F56 and replacement with a single P, N55/F56 and replacement with a single glycine (G), N55/F56 and replacement with a single alanine (A), N55/F56 and replacement with a single P and Y51N, N55/F56 and replacement with a single P and Y51Q, N55/F56 and replacement with a single P and Y51S, N55/F56 and replacement with a single G and Y51N, N55/F56 and replacement with a single G and Y51Q, N55/F56 and replacement with a single G and Y51S, N55/F56 and replacement with a single A and Y51N, N55/F56 and replacement with a single A/Y510 or N55/F56 and replacement with a single A and Y51S.

The variant more preferably comprises D195N/E203N, D195Q/E203N, D195N/E203Q, D195Q/E203Q, E201N/E203N, E201Q/E203N, E201N/E203Q, E201Q/E203Q, E185N/E203Q, E185Q/E203Q, E185N/E203N, E185Q/E203N, D195N/E201N/E203N, D195Q/E201N/E203N, D195N/E201Q/E203N, D195N/E201N/E203Q, D195Q/E201Q/E203N, D195Q/E201N/E203Q, D195N/E201Q/E203Q, D195Q/E201Q/E203Q, D149N/E201N, D149Q/E201N, D149N/E201Q, D149Q/E201Q, D149N/E201N/D195N, D149Q/E201N/D195N, D149N/E201Q/D195N, D149N/E201N/D195Q, D149Q/E201Q/D195N, D149Q/E201N/D195Q, D149N/E201Q/D195Q, D149Q/E201Q/D195Q, D149N/E203N, D149Q/E203N, D149N/E203Q, D149Q/E203Q, D149N/E185N/E201N, D149Q/E185N/E201N, D149N/E185Q/E201N, D149N/E185N/E201Q, D149Q/E185Q/E201N, D149Q/E185N/E201Q, D149N/E185Q/E201Q, D149Q/E185Q/E201Q, D149N/E185N/E203N, D149Q/E185N/E203N, D149N/E185Q/E203N, D149N/E185N/E203Q, D149Q/E185Q/E203N, D149Q/E185N/E203Q, D149N/E185Q/E203Q, D149Q/E185Q/E203Q, D149N/E185N/E201N/E203N, D149Q/E185N/E201N/E203N, D149N/E185Q/E201N/E203N, D149N/E185N/E201Q/E203N, D149N/E185N/E201N/E203Q, D149Q/E185Q/E201N/E203N, D149Q/E185N/E201Q/E203N, D149Q/E185N/E201N/E203Q, D149N/E185Q/E201Q/E203N, D149N/E185Q/E201N/E203Q, D149N/E185N/E201Q/E203Q, D149Q/E185Q/E201Q/E203Q, D149Q/E185Q/E201N/E203Q, D149Q/E185N/E201Q/E203Q, D149N/E185Q/E201Q/E203Q, D149Q/E185Q/E201Q/E203N, D149N/E185N/D195N/E201N/E203N, D149Q/E185N/D195N/E201N/E203N, D149N/E185Q/D195N/E201N/E203N, D149N/E185N/D195Q/E201N/E203N, D149N/E185N/D195N/E201Q/E203N, D149N/E185N/D195N/E201N/E203Q, D149Q/E185Q/D195N/E201N/E203N, D149Q/E185N/D195Q/E201N/E203N, D149Q/E185N/D195N/E201Q/E203N, D149Q/E185N/D195N/E201N/E203Q, D149N/E185Q/D195Q/E201N/E203N, D149N/E185Q/D195N/E201Q/E203N, D149N/E185Q/D195N/E201N/E203Q, D149N/E185N/D195Q/E201Q/E203N, D149N/E185N/D195Q/E201N/E203Q, D149N/E185N/D195N/E201Q/E203Q, D149Q/E185Q/D195Q/E201N/E203N, D149Q/E185Q/D195N/E201Q/E203N, D149Q/E185Q/D195N/E201N/E203Q, D149Q/E185N/D195Q/E201Q/E203N, D149Q/E185N/D195Q/E201N/E203Q, D149Q/E185N/D195N/E201Q/E203Q, D149N/E185Q/D195Q/E201Q/E203N, D149N/E185Q/D195Q/E201N/E203Q, D149N/E185Q/D195N/E201Q/E203Q, D149N/E185N/D195Q/E201Q/E203Q, D149Q/E185Q/D195Q/E201Q/E203N, D149Q/E185Q/D195Q/E201N/E203Q, D149Q/E185Q/D195N/E201Q/E203Q, D149Q/E185N/D195Q/E201Q/E203Q, D149N/E185Q/D195Q/E201Q/E203Q, D149Q/E185Q/D1950/E201Q/E203Q, D149N/E185R/E201N/E203N, D149Q/E185R/E201N/E203N, D149N/E185R/E201Q/E203N, D149N/E185R/E201N/E203Q, D149Q/E185R/E201Q/E203N, D149Q/E185R/E201N/E203Q, D149N/E185R/E201Q/E203Q, D149Q/E185R/E201Q1E203Q, D149R/E185N/E201N/E203N, D149R/E185Q/E201N/E203N. D149R/E185N/E201Q/E203N, D149R/E185N/E201N/E203Q, D149R/E185Q/E201Q/E203N, D149R/E185Q/E201N/E203Q, D149R/E185N/E201Q/E203Q, D149R/E185Q/E201Q/E203Q, D149R/E185N/D195N/E201N/E203N, D149R/E185Q/D195N/E201N/E203N, D149R/E185N/D195Q/E201N/E203N, D149R/E185N/D195N/E201Q/E203N, D149R/E185Q/D195N/E201N/E203Q, D149R/E185Q/D195Q/E201N/E203N, D149R/E185Q/D195N/E201Q/E203N, D149R/E185Q/D195N/E201N/E203Q, D149R/E185N/D195Q/E201Q/E203N, D149R/E185N/D195Q/E201N/E203Q, D149R/E185N/D195N/E201Q/E203Q, D149R/E185Q/D195Q/E201Q/E203N, D149R/E185Q/D195Q/E201N/E203Q, D149R/E185Q/D195N/E201Q/E203Q, D149R/E185N/D195Q/E201Q/E203Q, D149R/E185Q/D195Q/E201Q/E203Q, D149N/E185R/D195N/E201N/E203N, D149Q/E185R/D195N/E201N/E203N, D149N/E185R/D195Q/E201N/E203N, D149N/E185R/D195N/E201Q/E203N, D149N/E185R/D195N/E201N/E203Q, D149Q/E185R/D195Q/E201N/E203N, D149Q/E185R/D195N/E201Q/E203N, D1490/E185R/D195N/E201N/E203Q, D149N/E185R/D195Q/E201Q/E203N, D149N/E185R/D195Q/E201N/E203Q, D149N/E185R/D195N/E201Q/E203Q, D149Q/E185R/D195Q/E201Q/E203N, D149Q/E185R/D195Q/E201N/E203Q, D149Q/E185R/D195N/E201Q/E203Q, D149N/E185R/D195Q/E201Q/E203Q, D149Q/E185R/D195Q/E201Q/E203Q, D149N/E185R/D195N/E201R/E203N, D149Q/E185R/D195N/E201R/E203N, D149N/E185R/D195Q/E201R/E203N, D149N/E185R/D195N/E201R/E203Q, D149Q/E185R/D195Q/E201R/E203N, D149Q/E185R/D195N/E201R/E203Q, D149N/E185R/D1950/E201R/E203Q, D149Q/E185R/D195Q/E201R/E203Q, E131D/K49R, E101N/N102F, E101N/N102Y, E101N/N102W, E101F/N102F, E101F/N102Y, E101F/N102W, E101Y/N102F, E101Y/N102Y, E101Y/N102W, E101W/N102F, E101W/N102Y, E101W/N102W, E101N/N102R, E101F/N102R, E101Y/N102R or E101W/N102F.

Preferred variants of the Invention which form pores In which fewer nucleotides contribute to the current as the polynucleotide moves through the pore comprise Y51A/F56A, Y51A/F56N, Y51I/F56A, Y51L/F56A, Y51T/F56A, Y51I/F56N, Y51L/F56N or Y51T/F56N or more preferably Y51l/F56A, Y51L/F56A or Y51T/F56A. As discussed above, this makes it easier to identify a direct relationship between the observed current (as the polynucleotide moves through the pore) and the polynucleotide.

Preferred variants which form pores displaying an increased range comprise mutations at the following positions:
Y51, F56, D149, E185, E201 and E203;
N55 and F56;
Y51 and F56;
Y51, N55 and F56; or
F56 and N102.

Preferred variants which form pores displaying an increased range comprise:
Y51N, F56A, D149N, E185R, E201N and E203N;
N55S and F56Q;
Y51A and F56A;
Y51A and F56N;
Y51I and F56A;
Y51L and F56A;
Y51T and F56A;
Y51I and F56N;
Y51L and F56N;
Y51T and F56N;
Y51T and F560;
Y51A, N55S and F56A;
Y51A, N55S and F56N;
Y51T, N55S and F56Q; or
F56Q and N102R.

Preferred variants which form pores in which fewer nucleotides contribute to the current as the polynucleotide moves through the pore comprise mutations at the following positions:
N55 and F56, such as N55X and F56Q, wherein X is any amino acid; or
Y51 and F56, such as Y51X and F560, wherein X is any amino acid.

Preferred variants which form pores displaying an increased throughput comprise mutations at the following positions:
D149, E185 and E203;
D149, E185, E201 and E203; or
D149, E185, D195, E201 and E203.

Preferred variants which form pores displaying an increased throughput comprise:
D149N, E185N and E203N;
D149N, E185N, E201N and E203N;
D149N, E185R, D195N, E201N and E203N; or
D149N, E185R, D195N, E201R and E203N.

Preferred variants which form pores in which capture of the polynucleotide is increased comprise the following mutations:
D43N/Y51T/F56Q;
E44N/Y51T/F56Q;
D43N/E44N/Y51T/F56Q;
Y51T/F56Q/Q62R;
D43N/Y51T/F56Q/Q62R;
E44N/Y51T/F56Q/Q62R; or
D43N/E44N/Y51T/F56Q/Q62R.

Preferred variants comprise the following mutations:
D149R/E185R/E201R/E203R or Y51T/F56Q/D149R/E185R/E201R/E203R;
D149N/E185N/E201N/E203N or Y51T/F56Q/D149N/E185N/E201N/E203N;
E201R/E203R or Y51T/F56Q/E201R/E203R
E201N/E203R or Y51T/F56Q/E201N/E203R;
E203R or Y51T/F56Q/E203R;
E203N or Y51T/F56Q/E203N;
E201R or Y51T/F56Q/E201R;
E201N or Y51T/F56Q/E201N;
E185R or Y51T/F56Q/E185R;
E185N or Y51T/F56Q/E185N;
D149R or Y51T/F56Q/D149R;
D149N or Y51T/F56Q/D149N;
R142E or Y51T/F56Q/R142E;
R142N or Y51T/F56Q/R142N;
R192E or Y51T/F56Q/R192E; or
R192N or Y51T/F56Q/R192N.

Preferred variants comprise the following mutations:
Y51A/F56Q/E101N/N102R;
Y51A/F56Q/R97N/N102G;
Y51A/F56Q/R97N/N102R;
Y51A/F56Q/R97N;
Y51A/F56Q/R97G;
Y51A/F56Q/R97L;
Y51A/F56Q/N102R;
Y51A/F56Q/N102F;
Y51A/F56Q/N102G;
Y51A/F56Q/E101R;
Y51A/F56Q/E101F;
Y51A/F56Q/E101N; or
Y51A/F56Q/E101G

The invention also provides a mutant CsgG monomer comprising a variant of the sequence shown in SEQ ID NO: 390, wherein the variant comprises a mutation at T150. A preferred variant which forms a pore displaying an increased insertion comprises T150I. A mutation at T150, such as T150I, may be combined with any of the mutations or combinations of mutations discussed above.

The invention also provides a mutant CsgG monomer comprising a variant of the sequence shown in SEQ ID NO: 390 comprising the combination of mutations present in a variant disclosed in the Examples.

Methods for introducing or substituting naturally-occurring amino acids are well known in the art. For instance, methionine (M) may be substituted with arginine (R) by replacing the codon for methionine (ATG) with a codon for arginine (CGT) at the relevant position in a polynucleotide encoding the mutant monomer. The polynucleotide can then be expressed as discussed below.

Methods for introducing or substituting non-naturally-occurring amino acids are also well known in the art. For instance, non-naturally-occurring amino acids may be introduced by including synthetic aminoacyl-tRNAs in the IVTT system used to express the mutant monomer. Alternatively, they may be introduced by expressing the mutant monomer in E. coli that are auxotrophic for specific amino acids in the presence of synthetic (i.e. non-naturally-occurring) analogues of those specific amino acids. They may also be produced by naked ligation if the mutant monomer is produced using partial peptide synthesis.

### Other Monomers of the Invention

In another embodiment, the invention provides a mutant CsgG monomer comprising a variant of the sequence shown in SEQ ID NO: 390, wherein the variant comprises a mutation at one or more of positions Y51, N55 and F56. The variant may comprise a mutation at Y51; N55; F56; Y51/N55; Y51/F56; N55/F56; or Y51/N55/F56. The variant preferably comprises a mutation at Y51, N55 or F56.

The variant may comprise any of the specific mutations at one or more of positions Y51, N55 and F56 discussed above and in any combination. One or more Y51, N55 and F56 may be substituted with any amino acid, Y51 may be substituted with F, M, L, I, V, A, P, G, C, Q, N, T, S, E, D, K, H or R, such as A, S, T, N or Q, N55 may be substituted with F, M, L, I, V, A, P, G, C, Q, T, S, E, D, K, H or R, such as A, S, T or Q, F56 may be substituted with M, L, I, V, A, P, G, C, Q, N, T, S, E, D, K, H or R, such as A, S, T. N or Q.

The variant may further comprise one or more of the following: (i) one or more mutations at the following positions (i.e. mutations at one or more of the following positions) (i) N40, D43, E44, S54, S57, Q62, R97, E101, E124, E131, R142, T150 and R192; (iii) Q42R or Q42K; (iv) K49R; (v) N102R, N102F, N102Y or N102W; (vi) D149N, D1490 or D149R; (vii) E185N, E185Q or E185R; (viii) D195N, D1950 or D195R; (ix) E201N, E201Q or E201R; (x) E203N, E203Q or E203R; and (xi) deletion of one or more of the following positions F48, K49, P50, Y51, P52, A53, S54, N55, F56 and S57. The variant may comprise any of the combinations of (i) and (iii) to (xi) discussed above. The variant may comprise any of the embodiments discussed above for (i) and (iii) to (xi).

### Variants

In addition to the specific mutations discussed above, the variant may include other mutations. Over the entire length of the amino acid sequence of SEQ ID NO: 390, a variant will preferably be at least 50% homologous to that sequence based on amino acid identity. More preferably, the variant may be at least 55%, at least 60%, at least 65%, at least 70%, at least 75%, at least 80%, at least 85%, at least 90% and more preferably at least 95%, 97% or 99% homologous based on amino acid identity to the amino acid sequence of SEQ ID NO: 390 over the entire sequence. There may be at least 80%, for example at least 85%, 90% or 95%, amino acid Identity over a stretch of 100 or more, for example 125, 150, 175 or 200 or more, contiguous amino acids (“hard homology”).

Standard methods in the art may be used to determine homology. For example the UWGCG Package provides the BESTFIT program which can be used to calculate homology, for example used on its default settings (Devereux et al (1984) Nucleic Acids Research 12, p 387-395). The PILEUP and BLAST algorithms can be used to calculate homology or line up sequences (such as identifying equivalent residues or corresponding sequences (typically on their default settings)), for example as described in Altschul S. F. (1993) J Mol Evol 36:290-300; Altschul, S. F et al (1990) J Mol Biol 215:403-10. Software for performing BLAST analyses is publicly available through the National Center for Biotechnology Information (http://www.ncbi.nlm.nih.gov/).

SEQ ID NO: 390 is the wild-type CsgG monomer from Escherichia coli Str. K-12 substr. MC4100. The variant of SEQ ID NO: 390 may comprise any of the substitutions present in another CsgG homologue. Preferred CsgG homologues are shown in SEQ ID NOs: 391 to 395 and 414 to 429.

The variant may comprise combinations of one or more of the substitutions present in SEQ ID NOs: 391 to 395 and 414 to 429 compared with SEQ ID NO: 390.

Amino acid substitutions may be made to the amino acid sequence of SEQ ID NO: 390 in addition to those discussed above, for example up to 1, 2, 3, 4, 5, 10, 20 or 30 substitutions.

Conservative substitutions replace amino acids with other amino acids of similar chemical structure, similar chemical properties or similar side-chain volume. The amino acids introduced may have similar polarity, hydrophilicity, hydrophobicity, basicity, acidity, neutrality or charge to the amino acids they replace. Alternatively, the conservative substitution may introduce another amino acid that is aromatic or aliphatic in the place of a pre-existing aromatic or aliphatic amino acid. Conservative amino acid changes are well-known in the art and may be selected in accordance with the properties of the 20 main amino acids as defined in Table 1 below. Where amino acids have similar polarity, this can also be determined by reference to the hydropathy scale for amino acid side chains in Table 2.

TABLE 1
Chemical properties of amino acids

| | | | |
|---|---|---|---|
| Ala | aliphatic, hydrophobic, neutral | Met | hydrophobic, neutral |
| Cys | polar, hydrophobic, neutral | Asn | polar, hydrophilic, neutral |
| Asp | polar, hydrophilic, charged (−) | Pro | hydrophobic, neutral |
| Glu | polar, hydrophilic, charged (−) | Gln | polar, hydrophilic, neutral |
| Phe | aromatic, hydrophobic, neutral | Arg | polar, hydrophilic, charged (+) |
| Gly | aliphatic, neutral | Ser | polar, hydrophilic, neutral |
| His | aromatic, polar, hydrophilic, charged (+) | Thr | polar, hydrophilic, neutral |
| Ile | aliphatic, hydrophobic, neutral | Val | aliphatic, hydrophobic, neutral |
| Lys | polar, hydrophilic, charged(+) | Trp | aromatic, hydrophobic, neutral |
| Leu | aliphatic, hydrophobic, neutral | Tyr | aromatic, polar, hydrophobic |

TABLE 2
Hydropathy scale

| Side Chain | Hydropathy |
|---|---|
| Ile | 4.5 |
| Val | 4.2 |
| Leu | 3.8 |
| Phe | 2.8 |
| Cys | 2.5 |
| Met | 1.9 |
| Ala | 1.8 |
| Gly | −0.4 |
| Thr | −0.7 |
| Ser | −0.8 |
| Trp | −0.9 |
| Tyr | −1.3 |
| Pro | −1.6 |
| His | −3.2 |
| Glu | −3.5 |
| Gln | −3.5 |
| Asp | −3.5 |
| Asn | −3.5 |
| Lys | −3.9 |
| Arg | −4.5 |

One or more amino acid residues of the amino acid sequence of SEQ ID NO: 390 may additionally be deleted from the polypeptides described above. Up to 1, 2, 3, 4, 5, 10, 20 or 30 or more residues may be deleted.

Variants may include fragments of SEQ ID NO: 390. Such fragments retain pore forming activity. Fragments may be at least 50, at least 100, at least 150, at least 200 or at least 250 amino acids in length. Such fragments may be used to produce the pores. A fragment preferably comprises the membrane spanning domain of SEQ ID NO: 390, namely K135-Q153 and S183-S208.

One or more amino acids may be alternatively or additionally added to the polypeptides described above. An extension may be provided at the amino terminal or carboxy terminal of the amino acid sequence of SEQ ID NO: 390 or polypeptide variant or fragment thereof. The extension may be quite short, for example from 1 to 10 amino acids in length. Alternatively, the extension may be longer, for example up to 50 or 100 amino acids. A carrier protein may be fused to an amino acid sequence according to the invention. Other fusion proteins are discussed in more detail below.

As discussed above, a variant is a polypeptide that has an amino acid sequence which varies from that of SEQ ID NO: 390 and which retains its ability to form a pore. A variant typically contains the regions of SEQ ID NO: 390 that are responsible for pore formation. The pore forming ability of CsgG, which contains a β-barrel, is provided by β-sheets in each subunit. A variant of SEQ ID NO: 390 typically comprises the regions in SEQ ID NO: 390 that form β-sheets, namely K135-0153 and S183-S208. One or more modifications can be made to the regions of SEQ ID NO: 390 that form β-sheets as long as the resulting variant retains its ability to form a pore. A variant of SEQ ID NO: 390 preferably includes one or more modifications, such as substitutions, additions or deletions, within its u-helices and/or loop regions.

The monomers derived from CsgG may be modified to assist their identification or purification, for example by the addition of a streptavidin tag or by the addition of a signal sequence to promote their secretion from a cell where the monomer does not naturally contain such a sequence. Other suitable tags are discussed in more detail below. The monomer may be labelled with a revealing label. The revealing label may be any suitable label which allows the monomer to be detected. Suitable labels are described below.

The monomer derived from CsgG may also be produced using D-amino acids. For instance, the monomer derived from CsgG may comprise a mixture of L-amino acids and D-amino acids. This is conventional in the art for producing such proteins or peptides.

The monomer derived from CsgG contains one or more specific modifications to facilitate nucleotide discrimination. The monomer derived from CsgG may also contain other non-specific modifications as long as they do not interfere with pore formation. A number of non-specific side chain modifications are known in the art and may be made to the side chains of the monomer derived from CsgG. Such modifications include, for example, reductive alkylation of amino acids by reaction with an aldehyde followed by reduction with NaBH₄, amidination with methylacetimidate or acylation with acetic anhydride.

The monomer derived from CsgG can be produced using standard methods known in the art.

The monomer derived from CsgG may be made synthetically or by recombinant means. For example, the monomer may be synthesised by in vitro translation and transcription (IVTT). Suitable methods for producing pores and monomers are discussed in International Application Nos. PCT/GB09/001690 (published as WO 2010/004273), PCT/GB09/001679 (published as WO 2010/004265) or PCT/GB10/000133 (published as WO 2010/086603). Methods for inserting pores into membranes are discussed.

In some embodiments, the mutant monomer is chemically modified. The mutant monomer can be chemically modified in any way and at any site. The mutant monomer is preferably chemically modified by attachment of a molecule to one or more cysteines (cysteine linkage), attachment of a molecule to one or more lysines, attachment of a molecule to one or more non-natural amino acids, enzyme modification of an epitope or modification of a terminus. Suitable methods for carrying out such modifications are well-known in the art. The mutant monomer may be chemically modified by the attachment of any molecule. For instance, the mutant monomer may be chemically modified by attachment of a dye or a fluorophore

In some embodiments, the mutant monomer is chemically modified with a molecular adaptor that facilitates the interaction between a pore comprising the monomer and a target nucleotide or target polynucleotide sequence. The presence of the adaptor improves the host-guest chemistry of the pore and the nucleotide or polynucleotide sequence and thereby improves the sequencing ability of pores formed from the mutant monomer. The principles of host-guest chemistry are well-known in the art. The adaptor has an effect on the physical or chemical properties of the pore that improves its interaction with the nucleotide or polynucleotide sequence. The adaptor may alter the charge of the barrel or channel of the pore or specifically interact with or bind to the nucleotide or polynucleotide sequence thereby facilitating its interaction with the pore.

The molecular adaptor is preferably a cyclic molecule, a cyclodextrin, a species that is capable of hybridization, a DNA binder or interchelator, a peptide or peptide analogue, a synthetic polymer, an aromatic planar molecule, a small positively-charged molecule or a small molecule capable of hydrogen-bonding.

The adaptor may be cyclic. A cyclic adaptor preferably has the same symmetry as the pore. The adaptor preferably has eight-fold or nine-fold symmetry since CsgG typically has eight or nine subunits around a central axis. This is discussed in more detail below.

The adaptor typically interacts with the nucleotide or polynucleotide sequence via host-guest chemistry. The adaptor is typically capable of interacting with the nucleotide or polynucleotide sequence The adaptor comprises one or more chemical groups that are capable of Interacting with the nucleotide or polynucleotide sequence. The one or more chemical groups preferably interact with the nucleotide or polynucleotide sequence by non-covalent interactions, such as hydrophobic interactions, hydrogen bonding, Van der Waal's forces, r-cation interactions and/or electrostatic forces. The one or more chemical groups that are capable of interacting with the nucleotide or polynucleotide sequence are preferably positively charged. The one or more chemical groups that are capable of interacting with the nucleotide or polynucleotide sequence more preferably comprise amino groups. The amino groups can be attached to primary, secondary or tertiary carbon atoms. The adaptor even more preferably comprises a ring of amino groups, such as a ring of 6, 7 or 8 amino groups. The adaptor most preferably comprises a ring of eight amino groups. A ring of protonated amino groups may interact with negatively charged phosphate groups In the nucleotide or polynucleotide sequence.

The correct positioning of the adaptor within the pore can be facilitated by host-guest chemistry between the adaptor and the pore comprising the mutant monomer. The adaptor preferably comprises one or more chemical groups that are capable of interacting with one or more amino acids in the pore. The adaptor more preferably comprises one or more chemical groups that are capable of Interacting with one or more amino acids in the pore via non-covalent interactions, such as hydrophobic interactions, hydrogen bonding. Van der Waal's forces. π-cation interactions and/or electrostatic forces.

The chemical groups that are capable of interacting with one or more amino acids in the pore are typically hydroxyls or amines. The hydroxyl groups can be attached to primary, secondary or tertiary carbon atoms. The hydroxyl groups may form hydrogen bonds with uncharged amino acids in the pore. Any adaptor that facilitates the Interaction between the pore and the nucleotide or polynucleotide sequence can be used.

Suitable adaptors include, but are not limited to, cyclodextrins, cyclic peptides and cucurbiturils. The adaptor is preferably a cyclodextrin or a derivative thereof. The cyclodextrin or derivative thereof may be any of those disclosed in Eliseev, A. V., and Schneider, H-J. (1994) J. Am. Chem. Soc. 116, 6081-6088. The adaptor is more preferably heptakis-6-amino-β-cyclodextrin (am₇-βCD), 6-monodeoxy-6-monoamino-β-cyclodextrin (am₁-βCD) or heptakis-(6-deoxy-6-guanidino)-cyclodextrin (gu₇-βCD). The guanidino group in gu₇-βCD has a much higher pKa than the primary amines in am₇-βCD and so it is more positively charged. This gu₇-βCD adaptor may be used to increase the dwell time of the nucleotide in the pore, to increase the accuracy of the residual current measured, as well as to increase the base detection rate at high temperatures or low data acquisition rates.

If a succinimidyl 3-(2-pyridyldithio)propionate (SPDP) crosslinker is used as discussed in more detail below, the adaptor is preferably heptakis(6-deoxy-6-amino)-6-N-mono(2-pyridyl)dithiopropanoyl-β-cyclodextrin (am₆amPDP₁-βCD).

More suitable adaptors include γ-cyclodextrins, which comprise 9 sugar units (and therefore have nine-fold symmetry). The γ-cyclodextrin may contain a linker molecule or may be modified to comprise all or more of the modified sugar units used in the β-cyclodextrin examples discussed above.

The molecular adaptor is preferably covalently attached to the mutant monomer. The adaptor can be covalently attached to the pore using any method known in the art. The adaptor is typically attached via chemical linkage. If the molecular adaptor is attached via cysteine linkage, the one or more cysteines have preferably been Introduced to the mutant, for Instance In the barrel, by substitution. The mutant monomer may be chemically modified by attachment of a molecular adaptor to one or more cysteines in the mutant monomer. The one or more cysteines may be naturally-occurring, i.e. at positions 1 and/or 215 in SEQ ID NO: 390. Alternatively, the mutant monomer may be chemically modified by attachment of a molecule to one or more cysteines introduced at other positions. The cysteine at position 215 may be removed, for instance by substitution, to ensure that the molecular adaptor does not attach to that position rather than the cysteine at position 1 or a cysteine introduced at another position.

The reactivity of cysteine residues may be enhanced by modification of the adjacent residues. For instance, the basic groups of flanking arginine, histidine or lysine residues will change the pKa of the cysteines thiol group to that of the more reactive S′ group. The reactivity of cysteine residues may be protected by thiol protective groups such as dTNB. These may be reacted with one or more cysteine residues of the mutant monomer before a linker is attached.

The molecule may be attached directly to the mutant monomer. The molecule is preferably attached to the mutant monomer using a linker, such as a chemical crosslinker or a peptide linker.

Suitable chemical crosslinkers are well-known in the art. Preferred crosslinkers include 2,5-dioxopyrrolidin-1-yl 3-(pyridin-2-yldisulfanyl)propanoate, 2,5-dioxopyrrolidin-1-yl 4-(pyridin-2-yldisulfanyl)butanoate and 2,5-dioxopyrrolidin-1-yl 8-(pyridin-2-yldisulfanyl)octananoate. The most preferred crosslinker is succinimidyl 3-(2-pyridyldithio)propionate (SPDP). Typically, the molecule is covalently attached to the bifunctional crosslinker before the molecule/crosslinker complex is covalently attached to the mutant monomer but it is also possible to covalently attach the bifunctional crosslinker to the monomer before the bifunctional crosslinker/monomer complex is attached to the molecule.

The linker is preferably resistant to dithiothreitol (DTT). Suitable linkers Include, but are not limited to, iodoacetamide-based and Maleimide-based linkers.

In other embodiment, the monomer may be attached to a polynucleotide binding protein. This forms a modular sequencing system that may be used in the methods of sequencing of the invention. Polynucleotide binding proteins are discussed below.

The polynucleotide binding protein is preferably covalently attached to the mutant monomer. The protein can be covalently attached to the monomer using any method known in the art. The monomer and protein may be chemically fused or genetically fused. The monomer and protein are genetically fused if the whole construct is expressed from a single polynucleotide sequence. Genetic fusion of a monomer to a polynucleotide binding protein is discussed in International Application No. PCT/GB09/001679 (published as WO 2010/004265).

If the polynucleotide binding protein is attached via cysteine linkage, the one or more cysteines have preferably been introduced to the mutant by substitution. The one or more cysteines are preferably introduced into loop regions which have low conservation amongst homologues indicating that mutations or insertions may be tolerated. They are therefore suitable for attaching a polynucleotide binding protein. In such embodiments, the naturally-occurring cysteine at position 251 may be removed. The reactivity of cysteine residues may be enhanced by modification as described above.

The polynucleotide binding protein may be attached directly to the mutant monomer or via one or more linkers. The molecule may be attached to the mutant monomer using the hybridization linkers described in International Application No. PCT/GB10/000132 (published as WO 2010/086602). Alternatively, peptide linkers may be used. Peptide linkers are amino acid sequences. The length, flexibility and hydrophilicity of the peptide linker are typically designed such that it does not to disturb the functions of the monomer and molecule. Preferred flexible peptide linkers are stretches of 2 to 20, such as 4, 6, 8, 10 or 16, serine and/or glycine amino acids. More preferred flexible linkers include (SG)₁, (SG)₂, (SG)₃, (SG)₄, (SG)₅ and (SG)₈ wherein S is serine and G is glycine. Preferred rigid linkers are stretches of 2 to 30, such as 4, 6, 8, 16 or 24, proline amino acids. More preferred rigid inkers include (P)₁₂ wherein P is proline.

The mutant monomer may be chemically modified with a molecular adaptor and a polynucleotide binding protein

The molecule (with which the monomer is chemically modified) may be attached directly to the monomer or attached via a linker as disclosed in International Application Nos. PCT/GB09/001690 (published as WO 2010/004273). PCT/GB09/001679 (published as WO 20101004265) or PCT/GB10/000133 (published as WO 2010/086603).

Any of the proteins described herein, such as the mutant monomers and pores of the invention, may be modified to assist their identification or purification, for example by the addition of histidine residues (a his tag), aspartic acid residues (an asp tag), a streptavidin tag, a flag tag, a SUMO tag, a GST tag or a MBP tag, or by the addition of a signal sequence to promote their secretion from a cell where the polypeptide does not naturally contain such a sequence. An alternative to introducing a genetic tag is to chemically react a tag onto a native or engineered position on the protein. An example of this would be to react a gel-shift reagent to a cysteine engineered on the outside of the protein. This has been demonstrated as a method for separating hemolysin hetero-oligomers (Chem Biol. 1997 July; 4(7):497-505).

Any of the proteins described herein, such as the mutant monomers and pores of the invention, may be labelled with a revealing label. The revealing label may be any suitable label which allows the protein to be detected. Suitable labels include, but are not limited to, fluorescent molecules, radioisotopes, e.g. ¹²⁵I, ³⁵S, enzymes, antibodies, antigens, polynucleotides and ligands such as biotin.

Any of the proteins described herein, such as the monomers or pores of the Invention, may be made synthetically or by recombinant means. For example, the protein may be synthesised by in vitro translation and transcription (IVTT). The amino acid sequence of the protein may be modified to include non-naturally occurring amino acids or to increase the stability of the protein When a protein is produced by synthetic means, such amino acids may be introduced during production. The protein may also be altered following either synthetic or recombinant production.

Proteins may also be produced using D-amino acids. For instance, the protein may comprise a mixture of L-amino acids and D-amino acids. This is conventional in the art for producing such proteins or peptides.

The protein may also contain other non-specific modifications as long as they do not interfere with the function of the protein. A number of non-specific side chain modifications are known in the art and may be made to the side chains of the protein(s). Such modifications include, for example, reductive alkylation of amino acids by reaction with an aldehyde followed by reduction with NaBH₄, amidination with methylacetimidate or acylation with acetic anhydride.

Any of the proteins described herein, including the monomers and pores of the invention, can be produced using standard methods known in the art. Polynucleotide sequences encoding a protein may be derived and replicated using standard methods in the art. Polynucleotide sequences encoding a protein may be expressed in a bacterial host cell using standard techniques in the art. The protein may be produced in a cell by in situ expression of the polypeptide from a recombinant expression vector. The expression vector optionally carries an inducible promoter to control the expression of the polypeptide. These methods are described in Sambrook, J. and Russell, D. (2001). Molecular Cloning: A Laboratory Manual, 3rd Edition. Cold Spring Harbor Laboratory Press, Cold Spring Harbor, NY.

Proteins may be produced in large scale following purification by any protein liquid chromatography system from protein producing organisms or after recombinant expression. Typical protein liquid chromatography systems include FPLC, AKTA systems, the Bio-Cad system, the Bio-Rad BioLogic system and the Gilson HPLC system.

### Constructs

The invention also provides a construct comprising two or more covalently attached CsgG monomers, wherein at least one of the monomers is a mutant monomer of the invention. The construct of the invention retains its ability to form a pore. This may be determined as discussed above. One or more constructs of the invention may be used to form pores for characterising, such as sequencing, polynucleotides. The construct may comprise at least 2, at least 3, at least 4, at least 5, at least 6, at least 7, at least 8, at least 9 or at least 10 monomers. The construct preferably comprises two monomers. The two or more monomers may be the same or different.

At least one monomer in the construct is a mutant monomer of the invention. 2 or more, 3 or more, 4 or more, 5 or more, 6 or more, 7 or more, 8 or more, 9 or more or 10 or more monomers in the construct may be mutant monomers of the invention. All of the monomers in the construct are preferably mutant monomers of the invention. The mutant monomers may be the same or different. In a preferred embodiment, the construct comprises two mutant monomers of the invention.

The mutant monomers of the invention in the construct are preferably approximately the same length or are the same length. The barrels of the mutant monomers of the invention in the construct are preferably approximately the same length or are the same length. Length may be measured in number of amino acids and/or units of length.

The construct may comprise one or more monomers which are not mutant monomers of the invention. CsgG mutant monomers which are non mutant monomers of the invention include monomers comprising SEQ ID NO: 390, 391, 392, 393, 394, 395, 414, 415, 416, 417, 418, 419, 420, 421, 422, 423, 424, 425, 426, 427, 428 or 429 or a comparative variant of SEQ ID NO: 390, 391, 392, 393, 394, 395, 414, 415, 416, 417, 418, 419, 420, 421, 422, 423, 424, 425, 426, 427, 428 or 429 in which none of the amino acids/positions discussed above have been mutated. At least one monomer in the construct may comprise SEQ ID NO: 390, 391, 392, 393, 394, 395, 414, 415, 416, 417, 418, 419, 420, 421, 422, 423, 424, 425, 426, 427, 428 or 429 or a comparative variant of the sequence shown in SEQ ID NO: 390, 391, 392, 393, 394, 395, 414, 415, 416, 417, 418, 419, 420, 421, 422, 423, 424, 425, 426, 427, 428 or 429. A comparative variant of SEQ ID NO: 390, 391, 392, 393, 394, 395, 414, 415, 416, 417, 418, 419, 420, 421, 422, 423, 424, 425, 426, 427, 428 or 429 is at least 50% homologous to 390, 391, 392, 393, 394, 395, 414, 415, 416, 417, 418, 419, 420, 421, 422, 423, 424, 425, 426, 427, 428 or 429, 40 or 41 over its entire sequence based on amino acid identity. More preferably, the comparative variant may be at least 55%, at least 60%, at least 65%, at least 70%, at least 75%, at least 80%, at least 85%, at least 90% and more preferably at least 95%, 97% or 99% homologous based on amino acid identity to the amino acid sequence of SEQ ID NO: 390, 391, 392, 393, 394, 395, 414, 415, 416, 417, 418, 419, 420, 421, 422, 423, 424, 425, 426, 427, 428 or 429 over the entire sequence.

The monomers in the construct are preferably genetically fused. Monomers are genetically fused if the whole construct is expressed from a single polynucleotide sequence. The coding sequences of the monomers may be combined in any way to form a single polynucleotide sequence encoding the construct.

The monomers may be genetically fused in any configuration. The monomers may be fused via their terminal amino acids. For instance, the amino terminus of the one monomer may be fused to the carboxy terminus of another monomer. The second and subsequent monomers in the construct (in the amino to carboxy direction) may comprise a methionine at their amino terminal ends (each of which is fused to the carboxy terminus of the previous monomer). For instance, if M is a monomer (without an amino terminal methionine) and mM is a monomer with an amino terminal methionine, the construct may comprise the sequence M-mM, M-mM-mM or M-mM-mM-mM. The presences of these methionines typically results from the expression of the start codons (i.e. ATGs) at the 5′ end of the polynucleotides encoding the second or subsequent monomers within the polynucleotide encoding entire construct. The first monomer in the construct (in the amino to carboxy direction) may also comprise a methionine (e.g. mM-mM, mM-mM-mM or mM-mM-mM-mM).

The two or more monomers may be genetically fused directly together. The monomers are preferably genetically fused using a linker. The linker may be designed to constrain the mobility of the monomers. Preferred linkers are amino acid sequences (i.e. peptide linkers). Any of the peptide linkers discussed above may be used.

In another preferred embodiment, the monomers are chemically fused. Two monomers are chemically fused if the two parts are chemically attached, for instance via a chemical crosslinker. Any of the chemical crosslinkers discussed above may be used. The linker may be attached to one or more cysteine residues introduced into a mutant monomer of the invention. Alternatively, the linker may be attached to a terminus of one of the monomers in the construct.

If a construct contains different monomers, crosslinkage of monomers to themselves may be prevented by keeping the concentration of linker in a vast excess of the monomers. Alternatively, a “lock and key” arrangement may be used in which two linkers are used. Only one end of each linker may react together to form a longer linker and the other ends of the linker each react with a different monomers. Such linkers are described in International Application No. PCT/GB10/000132 (published as WO 2010/086602).

### Polynucleotides

The present invention also provides polynucleotide sequences which encode a mutant monomer of the invention. The mutant monomer may be any of those discussed above. The polynucleotide sequence preferably comprises a sequence at least 50%, 60%, 70%, 80%, 90% or 95% homologous based on nucleotide identity to the sequence of SEQ ID NO: 389 over the entire sequence. There may be at least 80%, for example at least 85%, 90% or 95% nucleotide identity over a stretch of 300 or more, for example 375, 450, 525 or 600 or more, contiguous nucleotides (“hard homology”). Homology may be calculated as described above. The polynucleotide sequence may comprise a sequence that differs from SEQ ID NO: 389 on the basis of the degeneracy of the genetic code.

The present invention also provides polynucleotide sequences which encode any of the genetically fused constructs of the Invention. The polynucleotide preferably comprises two or more variants of the sequence shown in SEQ ID NO: 389. The polynucleotide sequence preferably comprises two or more sequences having at least 50%, 60%, 70%, 80%, 90% or 95% homology to SEQ ID NO: 389 based on nucleotide identity over the entire sequence. There may be at least 80%, for example at least 85%, 90% or 95% nucleotide identity over a stretch of 600 or more, for example 750, 900, 1050 or 1200 or more, contiguous nucleotides (“hard homology”). Homology may be calculated as described above.

Polynucleotide sequences may be derived and replicated using standard methods in the art. Chromosomal DNA encoding wild-type CsgG may be extracted from a pore producing organism, such as Escherichia coli. The gene encoding the pore subunit may be amplified using PCR involving specific primers. The amplified sequence may then undergo site-directed mutagenesis. Suitable methods of site-directed mutagenesis are known In the art and include, for example, combine chain reaction. Polynucleotides encoding a construct of the invention can be made using well-known techniques, such as those described in Sambrook, J. and Russell, D. (2001). Molecular Cloning: A Laboratory Manual, 3rd Edition. Cold Spring Harbor Laboratory Press, Cold Spring Harbor, NY.

The resulting polynucleotide sequence may then be incorporated into a recombinant replicable vector such as a cloning vector. The vector may be used to replicate the polynucleotide in a compatible host cell. Thus polynucleotide sequences may be made by introducing a polynucleotide into a replicable vector, Introducing the vector into a compatible host cell, and growing the host cell under conditions which bring about replication of the vector. The vector may be recovered from the host cell. Suitable host cells for cloning of polynucleotides are known in the art and described in more detail below.

The polynucleotide sequence may be cloned Into suitable expression vector. In an expression vector, the polynucleotide sequence is typically operably linked to a control sequence which is capable of providing for the expression of the coding sequence by the host cell. Such expression vectors can be used to express a pore subunit.

The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequences. Multiple copies of the same or different polynucleotide sequences may be introduced into the vector.

The expression vector may then be introduced into a suitable host cell. Thus, a mutant monomer or construct of the invention can be produced by inserting a polynucleotide sequence into an expression vector, introducing the vector into a compatible bacterial host cell, and growing the host cell under conditions which bring about expression of the polynucleotide sequence. The recombinantly-expressed monomer or construct may self-assemble into a pore in the host cell membrane. Alternatively, the recombinant pore produced in this manner may be removed from the host cell and inserted into another membrane. When producing pores comprising at least two different monomers or constructs, the different monomers or constructs may be expressed separately in different host cells as described above, removed from the host cells and assembled into a pore in a separate membrane, such as a rabbit cell membrane or a synthetic membrane.

The vectors may be for example, plasmid, virus or phage vectors provided with an origin of replication, optionally a promoter for the expression of the said polynucleotide sequence and optionally a regulator of the promoter. The vectors may contain one or more selectable marker genes, for example a tetracycline resistance gene. Promoters and other expression regulation signals may be selected to be compatible with the host cell for which the expression vector is designed. A T7, trc, lac, are or λ_{L} promoter is typically used.

The host cell typically expresses the monomer or construct at a high level. Host cells transformed with a polynucleotide sequence will be chosen to be compatible with the expression vector used to transform the cell. The host cell is typically bacterial and preferably Escherichia coli. Any cel with a λ DE3 lysogen, for example C41 (DE3), BL21 (DE3), JM109 (DE3), B834 (DE3), TUNER, Origami and Origami B, can express a vector comprising the T7 promoter. In addition to the conditions listed above any of the methods cited in Cao et al, 2014, PNAS, Structure of the nonameric bacterial amyloid secretion channel, doi—1411942111 and Goyal et al, 2014, Nature, 516, 250-253 structural and mechanistic insights into the bacterial amyloid secretion channel CsgG may be used to express the CsgG proteins.

The invention also comprises a method of producing a mutant monomer of the invention or a construct of the invention. The method comprises expressing a polynucleotide of the invention in a suitable host cell. The polynucleotide is preferably part of a vector and is preferably operably linked to a promoter.

### Pores

The invention also provides various pores. The pores of the invention are ideal for characterising, such as sequencing, polynucleotide sequences because they can discriminate between different nucleotides with a high degree of sensitivity. The pores can surprisingly distinguish between the four nucleotides in DNA and RNA. The pores of the invention can even distinguish between methylated and unmethylated nucleotides. The base resolution of pores of the invention is surprisingly high. The pores show almost complete separation of all four DNA nucleotides. The pores further discriminate between deoxycytidine monophosphate (dCMP) and methyl-dCMP based on the dwell time in the pore and the current flowing through the pore.

The pores of the Invention can also discriminate between different nucleotides under a range of conditions. In particular, the pores will discriminate between nucleotides under conditions that are favourable to the characterising, such as sequencing, of nucleic acids. The extent to which the pores of the invention can discriminate between different nucleotides can be controlled by altering the applied potential, the salt concentration, the buffer, the temperature and the presence of additives, such as urea, betaine and DTT. This allows the function of the pores to be fine-tuned, particularly when sequencing. This is discussed in more detail below. The pores of the invention may also be used to identify polynucleotide polymers from the interaction with one or more monomers rather than on a nucleotide by nucleotide basis.

A pore of the invention may be isolated, substantially isolated, purified or substantially purified. A pore of the invention is isolated or purified if it is completely free of any other components, such as lipids or other pores. A pore is substantially isolated if it is mixed with carriers or diluents which will not interfere with its intended use. For instance, a pore is substantially isolated or substantially purified if it is present in a form that comprises less than 10%, less than 5%, less than 2% or less than 1% of other components, such as triblock copolymers, lipids or other pores. Alternatively, a pore of the invention may be present in a membrane. Suitable membranes are discussed below.

A pore of the invention may be present as an individual or single pore. Alternatively, a pore of the invention may be present in a homologous or heterologous population of two or more pores.

### Homo-Oligomeric Pores

The invention also provides a homo-oligomeric pore derived from CsgG comprising identical mutant monomers of the invention. The homo-oligomeric pore may comprise any of the mutants of the invention. The homo-oligomeric pore of the invention is ideal for characterising, such as sequencing, polynucleotides. The homo-oligomeric pore of the invention may have any of the advantages discussed above.

The homo-oligomeric pore may contain any number of mutant monomers. The pore typically comprises at least 7, at least 8, at least 9 or at least 10 identical mutant monomers, such as 7, 8, 9 or 10 mutant monomers. The pore preferably comprises eight or nine identical mutant monomers. One or more, such as 2, 3, 4, 5, 6, 7, 8, 9 or 10, of the mutant monomers is preferably chemically modified as discussed above.

Methods for making pores are discussed in more detail below.

### Hetero-Oligomeric Pores

The invention also provides a hetero-oligomeric pore derived from CsgG comprising at least one mutant monomer of the invention. The hetero-oligomeric pore of the invention is ideal for characterising, such as sequencing, polynucleotides. Hetero-oligomeric pores can be made using methods known in the art (e.g. Protein Sci. 2002 July; 11(7):1813-24).

The hetero-oligomeric pore contains sufficient monomers to form the pore. The monomers may be of any type. The pore typically comprises at least 7, at least 8, at least 9 or at least 10 monomers, such as 7, 8, 9 or 10 monomers The pore preferably comprises eight or nine monomers.

In a preferred embodiment, all of the monomers (such as 10, 9, 8 or 7 of the monomers) are mutant monomers of the invention and at least one of them differs from the others. In a more preferred embodiment, the pore comprises eight or nine mutant monomers of the invention and at least one of them differs from the others. They may all differ from one another.

The mutant monomers of the invention in the pore are preferably approximately the same length or are the same length. The barrels of the mutant monomers of the invention in the pore are preferably approximately the same length or are the same length. Length may be measured in number of amino acids and/or units of length.

In another preferred embodiment, at least one of the mutant monomers is not a mutant monomer of the invention. In this embodiment, the remaining monomers are preferably mutant monomers of the invention. Hence, the pore may comprise 9, 8, 7, 6, 5, 4, 3, 2 or 1 mutant monomers of the invention. Any number of the monomers In the pore may not be a mutant monomer of the invention. The pore preferably comprises seven or eight mutant monomers of the invention and a monomer which is not a monomer of the invention. The mutant monomers of the invention may be the same or different.

The mutant monomers of the invention in the construct are preferably approximately the same length or are the same length. The barrels of the mutant monomers of the invention in the construct are preferably approximately the same length or are the same length. Length may be measured in number of amino acids and/or units of length.

The pore may comprise one or more monomers which are not mutant monomers of the invention. CsgG monomers which are not mutant monomers of the invention include monomers comprising SEQ ID NO: 390, 391, 392, 393, 394, 395, 414, 415, 416, 417, 418, 419, 420, 421, 422, 423, 424, 425, 426, 427, 428 or 429 or a comparative variant of SEQ ID NO: 390, 391, 392, 393, 394, 395, 414, 415, 416, 417, 418, 419, 420, 421, 422, 423, 424, 425, 426, 427, 428 or 429 in which none of the amino acids/positions discussed above in relation to the invention have been mutated/substituted. A comparative variant of SEQ ID NO: 390, 391, 392, 393, 394, 395, 414, 415, 416, 417, 418, 419, 420, 421, 422, 423, 424, 425, 426, 427, 428 or 429 is typically at least 50% homologous to SEQ ID NO: 390, 391, 392, 393, 394, 395, 414, 415, 416, 417, 418, 419, 420, 421, 422, 423, 424, 425, 426, 427, 428 or 429 over its entire sequence based on amino acid identity. More preferably, the comparative variant may be at least 55%, at least 60%, at least 65%, at least 70%, at least 75%, at least 80%, at least 85%, at least 90% and more preferably at least 95%, 97% or 99% homologous based on amino acid identity to the amino acid sequence of SEQ ID NO: 390, 391, 392, 393, 394, 395, 414, 415, 416, 417, 418, 419, 420, 421, 422, 423, 424, 425, 426, 427, 428 or 429 over the entire sequence.

In all the embodiments discussed above, one or more, such as 2, 3, 4, 5, 6, 7, 8, 9 or 10, of the mutant monomers is preferably chemically modified as discussed above.

Methods for making pores are discussed in more detail below.

### Construct-Containing Pores

The invention also provides a pore comprising at least one construct of the invention. A construct of the invention comprises two or more covalently attached monomers derived from CsgG wherein at least one of the monomers is a mutant monomer of the invention. In other words, a construct must contain more than one monomer. The pore contains sufficient constructs and, if necessary, monomers to form the pore. For instance, an octameric pore may comprise (a) four constructs each comprising two constructs, (b) two constructs each comprising four monomers or (b) one construct comprising two monomers and six monomers that do not form part of a construct. For instance, an nonameric pore may comprise (a) four constructs each comprising two constructs and one monomer that does not form part of a construct. (b) two constructs each comprising four monomers and a monomer that does not form part of a construct or (b) one construct comprising two monomers and seven monomers that do not form part of a construct. Other combinations of constructs and monomers can be envisaged by the skilled person.

At least two of the monomers in the pore are in the form of a construct of the invention. The construct, and hence the pore, comprises at least one mutant monomer of the invention. The pore typically comprises at least 7, at least 8, at least 9 or at least 10 monomers, such as 7, 8, 9 or 10 monomers, in total (at least two of which must be in a construct). The pore preferably comprises eight or nine monomers (at least two of which must be in a construct).

The construct containing pore may be a homo-oligomer (i.e. include identical constructs) or be a hetero-oligomer (i.e. where at least one construct differs from the others).

A pore typically contains (a) one construct comprising two monomers and (b) 5, 6, 7 or 8 monomers. The construct may be any of those discussed above. The monomers may be any of those discussed above, including mutant monomers of the invention, monomers comprising SEQ ID NO: 390, 391, 392, 393, 394, 395, 414, 415, 416, 417, 418, 419, 420, 421, 422, 423, 424, 425, 426, 427, 428 or 429 and mutant monomers comprising a comparative variant of SEQ ID NO: 390, 391, 392, 393, 394, 395, 414, 415, 416, 417, 418, 419, 420, 421, 422, 423, 424, 425, 426, 427, 428 or 429 as discussed above.

Another typical pore comprises more than one construct of the invention, such as two, three or four constructs of the invention. If necessary, such pores further comprise sufficient additional monomers or constructs to form the pore. The additional monomer(s) may be any of those discussed above, including mutant monomers of the invention, monomers comprising SEQ ID NO: 390, 391, 392, 393, 394, 395, 414, 415, 416, 417, 418, 419, 420, 421, 422, 423, 424, 425, 426, 427, 428 or 429 and mutant monomers comprising a comparative variant of SEQ ID NO: 390, 391, 392, 393, 394, 395, 414, 415, 416, 417, 418, 419, 420, 421, 422, 423, 424, 425, 426, 427, 428 or 429 as discussed above. The additional construct(s) may be any of those discussed above or may be a construct comprising two or more covalently attached CsgG monomers each comprising a monomer comprising SEQ ID NO: 390, 391, 392, 393, 394, 395, 414, 415, 416, 417, 418, 419, 420, 421, 422, 423, 424, 425, 426, 427, 428 or 429 or a comparative variant of SEQ ID NO: 390, 391, 392, 393, 394, 395, 414, 415, 416, 417, 418, 419, 420, 421, 422, 423, 424, 425, 426, 427, 428 or 429 as discussed above.

A further pore of the invention comprises only constructs comprising 2 monomers, for example a pore may comprise 4, 5, 6, 7 or 8 constructs comprising 2 monomers. At least one construct is a construct of the invention, i.e. at least one monomer in the at least one construct, and preferably each monomer in the at least one construct, is a mutant monomer of the invention. All of the constructs comprising 2 monomers may be constructs of the invention.

A specific pore according to the invention comprises four constructs of the invention each comprising two monomers, wherein at least one monomer in each construct, and preferably each monomer in each construct, is a mutant monomer of the invention. The constructs may oligomerise into a pore with a structure such that only one monomer of each construct contributes to the channel of the pore. Typically the other monomers of the construct will be on the outside of the channel of the pore. For example, pores of the invention may comprise 7, 8, 9 or 10 constructs comprising 2 monomers where the channel comprises 7, 8, 9 or 10 monomers.

Mutations can be introduced into the construct as described above. The mutations may be alternating, i.e. the mutations are different for each monomer within a two monomer construct and the constructs are assembled as a homo-oligomer resulting in alternating modifications. In other words, monomers comprising MutA and MutB are fused and assembled to form an A-B:A-BA-B:A-B pore. Alternatively, the mutations may be neighbouring, i.e. identical mutations are introduced into two monomers in a construct and this is then oligomerised with different mutant monomers or constructs. In other words, monomers comprising MutA are fused follow by oligomerisation with MutB-containing monomers to form A-A:B:B:B:B:B:B.

One or more of the monomers of the invention in a construct-containing pore may be chemically-modified as discussed above.

### Analyte Characterisation

The invention provides a method of determining the presence, absence or one or more characteristics of a target analyte. The method involves contacting the target analyte with a CsgG pore or a mutant thereof, such as a pore of the invention, such that the target analyte moves with respect to, such as through, the pore and taking one or more measurements as the analyte moves with respect to the pore and thereby determining the presence, absence or one or more characteristics of the analyte. The target analyte may also be called the template analyte or the analyte of interest.

The method comprises contacting the target analyte with a CsgG pore or a mutant thereof, such as a pore of the invention, such that the target analyte moves through the pore. The pore typically comprises at least 7, at least 8, at least 9 or at least 10 monomers, such as 7, 8, 9 or 10 monomers. The pore preferably comprises eight or nine identical monomers One or more, such as 2, 3, 4, 5, 6, 7, 8, 9 or 10, of the monomers is preferably chemically modified as discussed above.

The CsgG pore may be derived from any organism. The CsgG pore may comprise monomers comprising the sequence shown in SEQ ID NO: 390, 391, 392, 393, 394, 395, 414, 415, 416, 417, 418, 419, 420, 421, 422, 423, 424, 425, 426, 427, 428 or 429. The CsgG pore may comprise at least 7, at least 8, at least 9 or at least 10 monomers, such as 7, 8, 9 or 10 monomers, each comprising the sequence shown in SEQ ID NO: 390, 391, 392, 393, 394, 395, 414, 415, 416, 417, 418, 419, 420, 421, 422, 423, 424, 425, 426, 427, 428 or 429 (i.e. the pore is a homo-oligomer comprising identical monomers from the same organism). The CsgG may comprise any combination of monomers each comprising a sequence shown in SEQ ID NO: 390, 391, 392, 393, 394, 395, 414, 415, 416, 417, 418, 419, 420, 421, 422, 423, 424, 425, 426, 427, 428 or 429. For instance, the pore may comprise 7 monomers comprising the sequence shown In SEQ ID NO: 390 and two monomers comprising the sequence shown in SEQ ID NO: 391.

The CsgG mutant may comprise any number of mutant monomers, such as at least 7, at least 8, at least 9 or at least 10 monomers, such as 7, 8, 9 or 10 monomers. The mutant monomers may comprise a comparative variant of the sequence shown in SEQ ID NO: 390, 391, 392, 393, 394, 395, 414, 415, 416, 417, 418, 419, 420, 421, 422, 423, 424, 425, 426, 427, 428 or 429. Comparative variants are discussed above. Comparative variants must be capable of forming a pore and may have any of the % homologies discussed above with reference to the pores of the Invention.

The CsgG mutant preferably comprises nine monomers and at least one of the monomers is a variant of the sequence shown in SEQ ID NO: 390 comprising (a) mutations at one or more of the following positions N40, Q42, D43, E44, K49, Y51, S54, N55, F56, S57, Q62, E101, N102, E124, E131, R142, D149, T150, E185, R192, D195, E201 and E203, such as mutations at one or more of the following positions N40, Q42, D43, E44, K49, Y51, S54, N55, F56, S57, Q62, E101, N102, E131, D149, T150, E185, D195, E201 and E203 or one or mutations at one or more of the following positions N40, Q42, D43, E44, K49, Y51, N55, F56, E101, N102, E131, D149, T150, E185, D195, E201 and E203; and/or (b) deletion of one or more of the following positions F48, K49, P50, Y51, P52, A53, S54, N55, F56 and 857. The variant may comprise (a); (b); or (a) and (b). In (a), any number and combination of positions N40, Q42, D43, E44, K49, Y51, S54, N55, F56, S57, Q62, E101, N102, R124, E131, R142, D149, E185, R192, D195, E201 and E203 may be mutated. As discussed above, mutating one or more of Y51, N55 and F56 may decrease the number of nucleotides contributing to the current as the polynucleotide moves through the pore and thereby make it easier to identify a direct relationship between the observed current as the polynucleotide moves through the pore and the polynucleotide.

In (b), any number and combination of F48, K49, P50, Y51, P52, A53, S54, N55, F56 and S57 may be deleted. The variant may comprise any of the specific mutations/substitutions or combinations thereof discussed above with reference to the mutant monomers of the invention.

Preferred variants for use in the method of the invention comprise one or more of the following substitutions (a) F56N, F56Q, F56R, F56S, F56G, F56A or F56K or F56A, F56P, F56R, F56H, F56S, F56Q, F56I, F56L, F56T or F56G; (b) N55Q, N55R, N55K, N55S, N55G, N55A or N55T; (c) Y51L, Y51V, Y51A, Y51N, Y51Q, Y51S or Y51G; (d) T150I; (e) S54P; and (f) S57P. The variant may comprise any number and combination of (a) to (f).

Preferred variants for use in the method of the invention comprise Q62R or Q62K.

Preferred variants for use in the method of the invention comprise mutations at D43, E44, Q62 or any combination thereof, such as D43, E44, Q62, D43/E44, D43/Q62, E44/Q62 or D431E44/Q62.

The variant may comprise a mutation at the following positions:
Y51, F56, D149, E185, E201 and E203, such as Y51N, F56A, D149N, E185R, E201N and E203N;
N55, such as N55A or N55S;
Y51, such as Y51N or Y51T;
S54, such as S54P;
S57, such as S57P;
F56, such as F56N, F56Q, F56R, F56S, F56G, F56A or F56K or F56A, F56P, F56R, F56H, F56S, F560, F56I, F56I, F56T or F56G;
Y51 and F56, such as Y51A and F56A, Y51A and F56N, Y51I and F56A, Y51L and F56A, Y51T and F56A, Y51T and F560, Y51I and F56N, Y51L and F56N or Y51T and F56N, preferably, Y51I and F56A, Y51L and F56A or Y51T and F56A, more preferably Y51T and F56Q or more preferably Y51X and F560, wherein X is any amino acid;
N55 and F56, such as N55X and F56Q, wherein X is any amino acid;
Y51, N55 and F56, such as Y51A, N55S and F56A, Y51A, N55S and F56N or Y51T, N55S and F56Q;
S54 and F56, such as S54P and F56A or S54P and F56N;
F56 and S57, such as F56A and S57P or F56N and S57P;
D149, E185 and E203, such as D149N, E185N and E203N;
D149, E185, E201 and E203, such as D149N, E185N, E201N and E203N;
D149, E185, D195, E201 and E203, such as D149N, E185R, D195N, E201N and E203N or
D149N, E185R, D195N, E201R and E203N; F56 and N102, such as F560 and N102R;
(a) Q62, such as Q62R or Q62K, and (b) one or more of Y51, N55 and F56, such as at Y51, N55, F56, Y51/N55, Y51/F56, N551F56 or Y51/N55/F56, such as Y51T/F56Q/Q62R;
(i) D43, E44, Q62 or any combination thereof, such as D43, E44, Q62, D43/E44, D43/Q62, E44/Q62 or D43/E44/Q62 and (ii) one or more of Y51, N55 and F56, such as at Y51, N55, F56, Y51/N55, Y51/F56, N55/F56 or Y51/N55/F56, such as D43N/Y51T/F56Q, E44N/Y51T/F56Q, D43N/E44N/Y51T/F56Q, D43N/Y51T/F56Q/Q62R, E44N/Y51T/F56Q/Q62R or D43N/E44N/Y51T/F56Q/Q62R; or
T150, such as T150I.

Preferred pores for use in the method of the invention comprise at least 7, at least 8, at least 9 or at least 10 monomers, such as 7, 8, 9 or 10 monomers, each of which comprises a variant of SEQ ID NO: 390 which comprises one or more of the following substitutions (a) F56N, F560, F56R, F56S, F56G, F56A or F56K or F56A, F56P, F56R, F56H, F56S, F560, F56I, F56L, F56T or F56G; (b) N55Q, N55R, N55K, N55S, N55G, N55A or N55T; (c) Y51L, Y51V, Y51A, Y51N, Y51Q, Y51S or Y51G; (d) T150I; (e) S54P; and (f) S57P. The variants may comprise any number and combination of (a) to (f). The monomers are preferably identical in these preferred pores.

Preferred pores for use in the method of the Invention comprise at least 7, at least 8, at least 9 or at least 10 monomers, such as 7, 8, 9 or 10 monomers, each of which comprises a variant of SEQ ID NO: 390 which comprises:
Y51N, F56A, D149N, E185R, E201N and E203N;
N55A;
N55S;
Y51N;
S54P;
S57P;
F56N, F560, F56R, F56S, F56G, F56A or F56K;
F58A, F56P, F56R, F56H, F56S, F56Q, F56I, F56L, F56T or F56G;
Y51A and F56A;
Y51A and F56N;
Y51I and F56A;
Y51L and F56A;
Y51T and F56A;
Y51T and F560;
Y51I and F56N;
Y51L and F56N;
Y51T and F56N;
N55S and F56Q;
Y51A, N55S and F58A;
Y51A, N55S and F56N;
Y51T, N55S and F56Q;
S54P and F56A;
S54P and F56N;
F56A and S57P;
F56N and S57P;
D149N, E185N and E203N;
D149N, E185N, E201N and E203N;
D149N, E185R, D195N, E201N and E203N;
D149N, E185R, D195N, E201R and E203N;
T150I;
F56Q and N102R;
F56 and N102, such as F560 and N102R;
Y51T/F56Q/Q62R;
D43N/Y51T/F56Q;
E44N/Y51T/F56Q;
D43N/E44N/Y51T/F56Q
D43N/Y51T/F56Q/Q62R;
E44N/Y51T/F56Q/Q62R; or
D43N/E44N/Y51T/F56Q/Q62R.

The CsgG mutant for use in the method of the invention preferably comprises nine monomers and at least one of the monomers is a variant of the sequence shown in SEQ ID NO: 390 comprising a mutation at one or more of positions Y51, N55 and F56. Preferred pores for use in the method of the invention comprise at least 7, at least 8, at least 9 or at least 10 monomers, such as 7, 8, 9 or 10 monomers, each of which comprises a variant of SEQ ID NO: 390 comprising a mutation at one or more of positions Y51, N55 and F56. The monomers are preferably Identical In these preferred pores. The variant may comprise a mutation at Y51; N55; F56; Y51/N55; Y51/F56; N55/F56; or Y51/N55/F56. The variant may comprise any of the specific mutations at one or more of positions Y51, N55 and F56 discussed above and in any combination. One or more Y51, N55 and F56 may be substituted with any amino acid, Y51 may be substituted with F, M, L, I, V, A, P, G, C, Q, N, T, S, E, D, K, H or R, such as A, S, T, N or Q, N55 may be substituted with F, M, L, I, V, A, P, G, C, Q, T, S, E, D, K, H or R, such as A, S, T or Q, F56 may be substituted with M, L, I, V, A, P, G, C, Q, N, T, S, E, D, K, H or R, such as A, S, T, N or Q. The variant may further comprise one or more of the following: (1) one or more mutations at the following positions (i.e. mutations at one or more of the following positions) (i) N40, D43, E44, S54, S57, Q62, R97, E101, E124, E131, R142, T150 and R192; (iii) Q42R or Q42K; (iv) K49R; (v) N102R, N102F, N102Y or N102W; (vi) D149N, D149Q or D149R; (vii) E185N, E185Q or E185R; (viii) D195N, D195Q or D195R; (Ix) E201N, E201Q or E201R; (x) E203N, E203Q or E203R; and (xi) deletion of one or more of the following positions F48, K49, P50, Y51, P52, A53, S54, N55, F56 and S57. The variant may comprise any of the combinations of (i) and (iii) to (xi) discussed above. The variant may comprise any of the embodiments discussed above for (i) and (iii) to (xi).

(1) Preferred variants for use in the method of the invention comprise or (2) preferred pores for use in the method of the invention comprise at least 7, at least 8, at least 9 or at least 10 monomers, such as 7, 8, 9 or 10 monomers, each of which comprises a variant of SEQ ID NO: 390 which comprise: Y51R/F56Q, Y51N/F56N, Y51M/F56Q, Y51L/F56Q, Y51I/F56Q, Y51V/F56Q, Y51A/F56Q, Y51P/F56Q, Y51G/F56Q, Y51C/F56Q, Y51Q/F56Q, Y51N/F56Q, Y51S/F56Q, Y51E/F56Q, Y51D/F56Q, Y51K/F56Q or Y51H/F56Q.

(1) Preferred variants for use in the method of the invention comprise or (2) preferred pores for use in the method of the invention comprise at least 7, at least 8, at least 9 or at least 10 monomers, such as 7, 8, 9 or 10 monomers, each of which comprises a variant of SEQ ID NO: 390 which comprise: Y51T/F560, Y51Q/F560 or Y51A/F56Q.

(1) Preferred variants for use in the method of the invention comprise or (2) preferred pores for use in the method of the invention comprise at least 7, at least 8, at least 9 or at least 10 monomers, such as 7, 8, 9 or 10 monomers, each of which comprises a variant of SEQ ID NO: 390 which comprise: Y51T/F56F, Y51T/F56M, Y51T/F56L, Y51T/F56I, Y51T/F56V, Y51T/F56A, Y51T/F56P, Y51T/F56G, Y51T/F56C, Y51T/F560, Y51T/F56N, Y51T/F56T, Y51T/F56S, Y51T/F56E, Y51T/F56D, Y51T/F56K, Y51T/F56H or Y51T/F56R.

(1) Preferred variants for use in the method of the invention comprise or (2) preferred pores for use in the method of the invention comprise at least 7, at least 8, at least 9 or at least 10 monomers, such as 7, 8, 9 or 10 monomers, each of which comprises a variant of SEQ ID NO: 390 which comprise: Y51T/N55Q, Y51T/N55S or Y51T/N55A.

(1) Preferred variants for use in the method of the invention comprise or (2) preferred pores for use in the method of the invention comprise at least 7, at least 8, at least 9 or at least 10 monomers, such as 7, 8, 9 or 10 monomers, each of which comprises a variant of SEQ ID NO: 390 which comprise: Y51A/F56F, Y51A/F56L, Y51A/F56I, Y51A/F56V, Y51A/F56A, Y51A/F56P, Y51A/F56G, Y51A/F56C, Y51A/F56Q, Y51A/F56N, Y51A/F56T, Y51A/F56S, Y51A/F56E, Y51A/F56D, Y51A/F56K, Y51A/F56H or Y51A/F56R.

(1) Preferred variants for use in the method of the invention comprise or (2) preferred pores for use in the method of the invention comprise at least 7, at least 8, at least 9 or at least 10 monomers, such as 7, 8, 9 or 10 monomers, each of which comprises a variant of SEQ ID NO: 390 which comprise: Y51C/F56A, Y51E/F56A, Y51D/F56A, Y51K/F56A, Y51H/F56A, Y51Q/F56A, Y51N/F56A, Y51S/F56A, Y51P/F56A or Y51V/F56A.

(1) Preferred variants for use in the method of the invention comprise or (2) preferred pores for use in the method of the Invention comprise at least 7, at least 8, at least 9 or at least 10 monomers, such as 7, 8, 9 or 10 monomers, each of which comprises a variant of SEQ ID NO: 390 which comprise:
D149R/E185R1E201R/E203R or Y51T/F56Q/D149R/E185R/E201R/E203R;
D149N/E185N/E201N/E203N or Y51T/F56Q/D149N/E185N/E201N/E203N.
E201R/E203R or Y51T/F56Q/E201R/E203R
E201N/E203R or Y51T/F56Q/E201N/E203R;
E203R or Y51T/F56Q/E203R;
E203N or Y51T/F56Q/E203N;
E201R or Y51T/F56Q/E201R;
E201N or Y51T/F56Q/E201N;
E185R or Y51T/F56Q/E185R;
E185N or Y51T/F56Q/E185N;
D149R or Y51T/F56Q/D149R;
D149N or Y51T/F56Q/D149N;
R142E or Y51T/F56Q/R142E;
R142N or Y51T/F56Q/R142N;
R192E or Y51T/F56Q/R192E; or
R192N or Y51T/F56Q/R192N.

(1) Preferred variants for use in the method of the invention comprise or (2) preferred pores for use in the method of the invention comprise at least 7, at least 8, at least 9 or at least 10 monomers, such as 7, 8, 9 or 10 monomers, each of which comprises a variant of SEQ ID NO: 390 which comprise:
Y51A/F56Q/E101N/N102R;
Y51A/F56Q/R97N/N102G;
Y51A/F56Q/R97N/N102R;
Y51N/F56Q/R97N;
Y51A/F56Q/R97G;
Y51N/F56Q/R97L;
Y51A/F56Q/N102R;
Y51N/F56Q/N102F;
Y51A/F56Q/N102G;
Y51N/F56Q/E101R;
Y51A/F56Q/E101F;
Y51A/F56Q/E101N; or
Y51A/F56Q/E101G.

The monomers are preferably identical in these preferred pores.

Preferred pores for use in the method of the invention comprise at least 7, at least 8, at least 9 or at least 10 monomers, such as 7, 8, 9 or 10 monomers, each of which comprises a variant of SEQ ID NO: 390 which comprises F56A, F56P, F56R, F56H, F56S, F56Q, F56I, F56L, F56T or F56G. The monomers are preferably identical in these preferred pores.

The CsgG mutant may comprise any of the variants of SEQ ID NO: 390 disclosed in the Examples or may be any of the pores disclosed In the Examples.

The CsgG mutant is most preferably a pore of the invention.

Steps (a) and (b) are preferably carried out with a potential applied across the pore. As discussed in more detail below, the applied potential typically results in the formation of a complex between the pore and a polynucleotide binding protein. The applied potential may be a voltage potential. Alternatively, the applied potential may be a chemical potential. An example of this is using a salt gradient across an amphiphilic layer. A salt gradient is disclosed in Holden et al., J Am Chem Soc. 2007 Jul. 11:129(27):8650-5.

The method is for determining the presence, absence or one or more characteristics of a target analyte. The method may be for determining the presence, absence or one or more characteristics of at least one analyte. The method may concern determining the presence, absence or one or more characteristics of two or more analytes. The method may comprise determining the presence, absence or one or more characteristics of any number of analytes, such as 2, 5, 10, 15, 20, 30, 40, 50, 100 or more analytes. Any number of characteristics of the one or more analytes may be determined, such as 1, 2, 3, 4, 5, 10 or more characteristics.

The target analyte is preferably a metal Ion, an Inorganic salt, a polymer, an amino acid, a peptide, a polypeptide, a protein, a nucleotide, an oligonucleotide, a polynucleotide, a dye, a bleach, a pharmaceutical, a diagnostic agent, a recreational drug, an explosive or an environmental pollutant. The method may concern determining the presence, absence or one or more characteristics of two or more analytes of the same type, such as two or more proteins, two or more nucleotides or two or more pharmaceuticals. Alternatively, the method may concern determining the presence, absence or one or more characteristics of two or more analytes of different types, such as one or more proteins, one or more nucleotides and one or more pharmaceuticals.

The target analyte can be secreted from cells. Alternatively, the target analyte can be an analyte that is present inside cells such that the analyte must be extracted from the cells before the invention can be carried out.

The analyte is preferably an amino acid, a peptide, a polypeptides and/or a protein. The amino acid, peptide, polypeptide or protein can be naturally-occurring or non-naturally-occurring. The polypeptide or protein can include within them synthetic or modified amino acids. A number of different types of modification to amino acids are known in the art. Suitable amino acids and modifications thereof are above. For the purposes of the invention, it is to be understood that the target analyte can be modified by any method available in the art.

The protein can be an enzyme, an antibody, a hormone, a growth factor or a growth regulatory protein, such as a cytokine. The cytokine may be selected from Interleukins, preferably IFN-1, IL-1, IL-2, IL-4, IL-5, IL-6, IL-10, IL-12 and IL-13, interferons, preferably IL-γ, and other cytokines such as TNF-α. The protein may be a bacterial protein, a fungal protein, a virus protein or a parasite-derived protein.

The target analyte is preferably a nucleotide, an oligonucleotide or a polynucleotide. Nucleotides and polynucleotides are discussed below. Oligonucleotides are short nucleotide polymers which typically have 50 or fewer nucleotides, such 40 or fewer, 30 or fewer, 20 or fewer, 10 or fewer or 5 or fewer nucleotides. The oligonucleotides may comprise any of the nucleotides discussed below, including the abasic and modified nucleotides.

The target analyte, such as a target polynucleotide, may be present in any of the suitable samples discussed below.

The pore is typically present in a membrane as discussed below. The target analyte may be coupled or delivered to the membrane using of the methods discussed below.

Any of the measurements discussed below can be used to determine the presence, absence or one or more characteristics of the target analyte. The method preferably comprises contacting the target analyte with the pore such that the analyte moves with respect to, such as moves through, the pore and measuring the current passing through the pore as the analyte moves with respect to the pore and thereby determining the presence, absence or one or more characteristics of the analyte.

The target analyte is present if the current flows through the pore in a manner specific for the analyte (i.e. if a distinctive current associated with the analyte is detected flowing through the pore). The analyte is absent if the current does not flow through the pore in a manner specific for the nucleotide. Control experiments can be carried out in the presence of the analyte to determine the way in which if affects the current flowing through the pore.

The invention can be used to differentiate analytes of similar structure on the basis of the different effects they have on the current passing through a pore. Individual analytes can be identified at the single molecule level from their current amplitude when they interact with the pore. The invention can also be used to determine whether or not a particular analyte is present in a sample. The invention can also be used to measure the concentration of a particular analyte in a sample. Analyte characterisation using pores other than CsgG is known in the art.

### Polynucleotide Characterisation

The invention provides a method of characterising a target polynucleotide, such as sequencing a polynucleotide. There are two main strategies for characterising or sequencing polynucleotides using nanopores, namely strand characterisation/sequencing and exonuclease characterisation/sequencing. The method of the invention may concern either method.

In strand sequencing, the DNA is translocated through the nanopore either with or against an applied potential. Exonucleases that act progressively or processively on double stranded DNA can be used on the cis side of the pore to feed the remaining single strand through under an applied potential or the trans side under a reverse potential. Likewise, a helicase that unwinds the double stranded DNA can also be used in a similar manner. A polymerase may also be used. There are also possibilities for sequencing applications that require strand translocation against an applied potential, but the DNA must be first “caught” by the enzyme under a reverse or no potential. With the potential then switched back following binding the strand will pass cis to trans through the pore and be held in an extended conformation by the current flow. The single strand DNA exonucleases or single strand DNA dependent polymerases can act as molecular motors to pull the recently translocated single strand back through the pore in a controlled stepwise manner, trans to cis, against the applied potential.

In one embodiment, the method of characterising a target polynucleotide involves contacting the target sequence with a pore and a helicase enzyme. Any helicase may be used in the method. Suitable helicases are discussed below. Helicases may work in two modes with respect to the pore. First, the method is preferably carried out using a helicase such that it controls movement of the target sequence through the pore with the field resulting from the applied voltage. In this mode the 5′ end of the DNA is first captured in the pore, and the enzyme controls movement of the DNA into the pore such that the target sequence is passed through the pore with the field until it finally translocates through to the trans side of the bilayer. Alternatively, the method is preferably carried out such that a helicase enzyme controls movement of the target sequence through the pore against the field resulting from the applied voltage. In this mode the 3′ end of the DNA is first captured in the pore, and the enzyme controls movement of the DNA through the pore such that the target sequence is pulled out of the pore against the applied field until finally ejected back to the cis side of the bilayer.

In exonuclease sequencing, an exonuclease releases individual nucleotides from one end of the target polynucleotide and these individual nucleotides are identified as discussed below. In another embodiment, the method of characterising a target polynucleotide involves contacting the target sequence with a pore and an exonuclease enzyme. Any of the exonuclease enzymes discussed below may be used in the method. The enzyme may be covalently attached to the pore as discussed below.

Exonucleases are enzymes that typically latch onto one end of a polynucleotide and digest the sequence one nucleotide at a time from that end. The exonuclease can digest the polynucleotide in the 5′ to 3′ direction or 3′ to 5′ direction. The end of the polynucleotide to which the exonuclease binds is typically determined through the choice of enzyme used and/or using methods known in the art. Hydroxyl groups or cap structures at either end of the polynucleotide may typically be used to prevent or facilitate the binding of the exonuclease to a particular end of the polynucleotide.

The method involves contacting the polynucleotide with the exonuclease so that the nucleotides are digested from the end of the polynucleotide at a rate that allows characterisation or identification of a proportion of nucleotides as discussed above. Methods for doing this are well known in the art. For example, Edman degradation is used to successively digest single amino acids from the end of polypeptide such that they may be identified using High Performance Liquid Chromatography (HPLC). A homologous method may be used in the present invention.

The rate at which the exonuclease functions is typically slower than the optimal rate of a wild-type exonuclease. A suitable rate of activity of the exonuclease in the method of the invention involves digestion of from 0.5 to 1000 nucleotides per second, from 0.6 to 500 nucleotides per second, 0.7 to 200 nucleotides per second, from 0.8 to 100 nucleotides per second, from 0.9 to 50 nucleotides per second or 1 to 20 or 10 nucleotides per second. The rate is preferably 1, 10, 100, 500 or 1000 nucleotides per second. A suitable rate of exonuclease activity can be achieved in various ways. For example, variant exonucleases with a reduced optimal rate of activity may be used in accordance with the invention.

In the strand characterisation embodiment, the method comprises contacting the polynucleotide with a CsgG pore or mutant thereof, such as a pore of the invention, such that the polynucleotide moves with respect to, such as through, the pore and taking one or more measurements as the polynucleotide moves with respect to the pore, wherein the measurements are Indicative of one or more characteristics of the polynucleotide, and thereby characterising the target polynucleotide.

In the exonucleotide characterisation embodiment, the method comprises contacting the polynucleotide with a CsgG pore or mutant thereof, such as a pore of the invention, and an exonucleoase such that the exonuclease digests individual nucleotides from one end of the target polynucleotide and the individual nucleotides move with respect to, such as through, the pore and taking one or more measurements as the individual nucleotides move with respect to the pore, wherein the measurements are indicative of one or more characteristics of the individual nucleotides, and thereby characterising the target polynucleotide.

An individual nucleotide is a single nucleotide. An individual nucleotide is one which is not bound to another nucleotide or polynucleotide by a nucleotide bond. A nucleotide bond involves one of the phosphate groups of a nucleotide being bound to the sugar group of another nucleotide. An individual nucleotide is typically one which is not bound by a nucleotide bond to another polynucleotide of at least 5, at least 10, at least 20, at least 50, at least 100, at least 200, at least 500, at least 1000 or at least 5000 nucleotides. For example, the individual nucleotide has been digested from a target polynucleotide sequence, such as a DNA or RNA strand. The nucleotide can be any of those discussed below.

The individual nucleotides may interact with the pore in any manner and at any site. The nucleotides preferably reversibly bind to the pore via or in conjunction with an adaptor as discussed above. The nucleotides most preferably reversibly bind to the pore via or in conjunction with the adaptor as they pass through the pore across the membrane. The nucleotides can also reversibly bind to the barrel or channel of the pore via or in conjunction with the adaptor as they pass through the pore across the membrane.

During the interaction between the individual nucleotide and the pore, the nucleotide typically affects the current flowing through the pore in a manner specific for that nucleotide. For example, a particular nucleotide will reduce the current flowing through the pore for a particular mean time period and to a particular extent. In other words, the current flowing through the pore is distinctive for a particular nucleotide. Control experiments may be carried out to determine the effect a particular nucleotide has on the current flowing through the pore. Results from carrying out the method of the invention on a test sample can then be compared with those derived from such a control experiment in order to identify a particular nucleotide in the sample or determine whether a particular nucleotide is present in the sample. The frequency at which the current flowing through the pore is affected in a manner indicative of a particular nucleotide can be used to determine the concentration of that nucleotide in the sample. The ratio of different nucleotides within a sample can also be calculated. For instance, the ratio of dCMP to methyl-dCMP can be calculated.

The method involves measuring one or more characteristics of the target polynucleotide. The target polynucleotide may also be called the template polynucleotide or the polynucleotide of interest.

This embodiment also uses a CsgG pore or mutant thereof, such as a pore of the invention. Any of the pores and embodiments discussed above with reference to the target analyte may be used.

### Polynucleotide

A polynucleotide, such as a nucleic acid, is a macromolecule comprising two or more nucleotides. The polynucleotide or nucleic acid may comprise any combination of any nucleotides. The nucleotides can be naturally occurring or artificial. One or more nucleotides In the polynucleotide can be oxidized or methylated. One or more nucleotides in the polynucleotide may be damaged. For instance, the polynucleotide may comprise a pyrimidine dimer. Such dimers are typically associated with damage by ultraviolet light and are the primary cause of skin melanomas. One or more nucleotides in the polynucleotide may be modified, for instance with a label or a tag. Suitable labels are described below. The polynucleotide may comprise one or more spacers.

A nucleotide typically contains a nucleobase, a sugar and at least one phosphate group. The nucleobase and sugar form a nucleoside.

The nucleobase is typically heterocyclic. Nucleobases include, but are not limited to, purines and pyrimidines and more specifically adenine (A), guanine (G), thymine (T), uracil (U) and cytosine (C).

The sugar is typically a pentose sugar. Nucleotide sugars include, but are not limited to, ribose and deoxyribose. The sugar is preferably a deoxyribose.

The polynucleotide preferably comprises the following nucleosides: deoxyadenosine (dA), deoxyuridine (dU) and/or thymidine (dT), deoxyguanosine (dG) and deoxycytidine (dC).

The nucleotide is typically a ribonucleotide or deoxyribonucleotide. The nucleotide typically contains a monophosphate, diphosphate or triphosphate. The nucleotide may comprise more than three phosphates, such as 4 or 5 phosphates. Phosphates may be attached on the 5′ or 3′ side of a nucleotide. Nucleotides include, but are not limited to, adenosine monophosphate (AMP), guanosine monophosphate (GMP), thymidine monophosphate (TMP), uridine monophosphate (UMP), 5-methylcytidine monophosphate, 5-hydroxymethylcytidine monophosphate, cytidine monophosphate (CMP), cyclic adenosine monophosphate (cAMP), cyclic guanosine monophosphate (cGMP), deoxyadenosine monophosphate (dAMP), deoxyguanosine monophosphate (dGMP), deoxythymidine monophosphate (dTMP), deoxyuridine monophosphate (dUMP), deoxycytidine monophosphate (dCMP) and deoxymethylcytidine monophosphate. The nucleotides are preferably selected from AMP. TMP, GMP, CMP, UMP, dAMP, dTMP, dGMP, dCMP and dUMP.

A nucleotide may be abasic (i.e. lack a nucleobase). A nucleotide may also lack a nucleobase and a sugar (i.e. is a C3 spacer).

The nucleotides in the polynucleotide may be attached to each other in any manner. The nucleotides are typically attached by their sugar and phosphate groups as in nucleic acids. The nucleotides may be connected via their nucleobases as in pyrimidine dimers.

The polynucleotide may be single stranded or double stranded. At least a portion of the polynucleotide is preferably double stranded.

The polynucleotide can be a nucleic acid, such as deoxyribonucleic acid (DNA) or ribonucleic acid (RNA). The polynucleotide can comprise one strand of RNA hybridised to one strand of DNA. The polynucleotide may be any synthetic nucleic acid known in the art, such as peptide nucleic acid (PNA), glycerol nucleic acid (GNA), threose nucleic acid (TNA), locked nucleic acid (LNA) or other synthetic polymers with nucleotide side chains. The PNA backbone is composed of repeating N-(2-aminoethyl)-glycine units linked by peptide bonds. The GNA backbone is composed of repeating glycol units linked by phosphodiester bonds. The TNA backbone is composed of repeating threose sugars linked together by phosphodiester bonds. LNA is formed from ribonucleotides as discussed above having an extra bridge connecting the 2′ oxygen and 4′ carbon In the ribose moiety. Bridged nucleic acids (BNAs) are modified RNA nucleotides. They may also be called constrained or inaccessible RNA. BNA monomers can contain a five-membered, six-membered or even a seven-membered bridged structure with a “fixed” C3′-endo sugar puckering. The bridge is synthetically incorporated at the 2′,4′-position of the ribose to produce a 2′,4′-BNA monomer.

The polynucleotide is most preferably ribonucleic nucleic acid (RNA) or deoxyribonucleic acid (DNA).

The polynucleotide can be any length. For example, the polynucleotide can be at least 10, at least 50, at least 100, at least 150, at least 200, at least 250, at least 300, at least 400 or at least 500 nucleotides or nucleotide pairs in length. The polynucleotide can be 1000 or more nucleotides or nucleotide pairs, 5000 or more nucleotides or nucleotide pairs in length or 100000 or more nucleotides or nucleotide pairs in length.

Any number of polynucleotides can be investigated. For instance, the method of the invention may concern characterising 2, 3, 4, 5, 6, 7, 8, 9, 10, 20, 30, 50, 100 or more polynucleotides. If two or more polynucleotides are characterised, they may be different polynucleotides or two instances of the same polynucleotide.

The polynucleotide can be naturally occurring or artificial. For instance, the method may be used to verify the sequence of a manufactured oligonucleotide. The method is typically carried out in vitro.

### Sample

The polynucleotide is typically present in any suitable sample. The invention is typically carried out on a sample that is known to contain or suspected to contain the polynucleotide. Alternatively, the invention may be carried out on a sample to confirm the identity of a polynucleotide whose presence in the sample is known or expected.

The sample may be a biological sample. The invention may be carried out in vitro using a sample obtained from or extracted from any organism or microorganism. The organism or microorganism is typically archaeal, prokaryotic or eukaryotic and typically belongs to one of the five kingdoms: plantae, animalia, fungi, monera and protista. The invention may be carried out in vitro on a sample obtained from or extracted from any virus. The sample is preferably a fluid sample. The sample typically comprises a body fluid of the patient. The sample may be urine, lymph, saliva, mucus or amniotic fluid but is preferably blood, plasma or serum.

Typically, the sample is human in origin, but alternatively it may be from another mammal animal such as from commercially farmed animals such as horses, cattle, sheep, fish, chickens or pigs or may alternatively be pets such as cats or dogs. Alternatively, the sample may be of plant origin, such as a sample obtained from a commercial crop, such as a cereal, legume, fruit or vegetable, for example wheat, barley, oats, canola, maize, soya, rice, rhubarb, bananas, apples, tomatoes, potatoes, grapes, tobacco, beans, lentils, sugar cane, cocoa, cotton.

The sample may be a non-biological sample. The non-biological sample is preferably a fluid sample. Examples of non-biological samples include surgical fluids, water such as drinking water, sea water or river water, and reagents for laboratory tests.

The sample is typically processed prior to being used in the invention, for example by centrifugation or by passage through a membrane that filters out unwanted molecules or cells, such as red blood cells. The may be measured immediately upon being taken. The sample may also be typically stored prior to assay, preferably below −70° C.

### Characterisation

The method may involve measuring two, three, four or five or more characteristics of the polynucleotide. The one or more characteristics are preferably selected from (i) the length of the polynucleotide. (ii) the identity of the polynucleotide, (iii) the sequence of the polynucleotide, (iv) the secondary structure of the polynucleotide and (v) whether or not the polynucleotide is modified. Any combination of (i) to (v) may be measured in accordance with the invention, such as {i}, {ii}, {iii}, {iv}, {v}, {i,ii}, {i,iii}, {i,iv}, {i,v}, {ii,iii}, {ii,iv}, {ii,v}, {iii,iv}, {iii,v}, {iv,v}, {i,ii,iii}, {i,ii,iv}, {i,ii,v}, {i,iii,iv}, {i,iii,v}, {i,iv,v}, {ii,iii,iv}, {ii,iii,v}, {ii,iv,v}, {iii,iv,v}, {i,ii,iii,iv}, {i,ii,iii,v}, {i,ii,iv,v}, {i,iii,iv,v}, {i,iii,iv,v} or {i,ii,iii,iv,v}. Different combinations of (i) to (v) may be measured for the first polynucleotide compared with the second polynucleotide, including any of those combinations listed above.

For (i), the length of the polynucleotide may be measured for example by determining the number of interactions between the polynucleotide and the pore or the duration of interaction between the polynucleotide and the pore.

For (ii), the identity of the polynucleotide may be measured in a number of ways. The identity of the polynucleotide may be measured in conjunction with measurement of the sequence of the polynucleotide or without measurement of the sequence of the polynucleotide. The former is straightforward; the polynucleotide is sequenced and thereby identified The latter may be done in several ways. For instance, the presence of a particular motif In the polynucleotide may be measured (without measuring the remaining sequence of the polynucleotide). Alternatively, the measurement of a particular electrical and/or optical signal in the method may identify the polynucleotide as coming from a particular source.

For (ii), the sequence of the polynucleotide can be determined as described previously. Suitable sequencing methods, particularly those using electrical measurements, are described in Stoddart D et al., Proc Natl Acad Sci, 12; 106(19):7702-7, Lieberman K R et al, J Am Chem Soc. 2010; 132(50):17961-72, and International Application WO 2000/28312.

For (iv), the secondary structure may be measured in a variety of ways. For instance, if the method involves an electrical measurement, the secondary structure may be measured using a change in dwell time or a change in current flowing through the pore. This allows regions of single-stranded and double-stranded polynucleotide to be distinguished.

For (v), the presence or absence of any modification may be measured. The method preferably comprises determining whether or not the polynucleotide is modified by methylation, by oxidation, by damage, with one or more proteins or with one or more labels, tags or spacers. Specific modifications will result in specific interactions with the pore which can be measured using the methods described below. For instance, methylcyotsine may be distinguished from cytosine on the basis of the current flowing through the pore during its interaction with each nucleotide.

The target polynucleotide is contacted with a CsgG pore or mutant thereof, such as a pore of the invention. The pore is typically present in a membrane. Suitable membranes are discussed below. The method may be carried out using any apparatus that is suitable for investigating a membrane/pore system in which a pore is present in a membrane. The method may be carried out using any apparatus that is suitable for transmembrane pore sensing. For example, the apparatus comprises a chamber comprising an aqueous solution and a barrier that separates the chamber into two sections. The barrier typically has an aperture in which the membrane containing the pore is formed. Alternatively the barrier forms the membrane In which the pore is present.

The method may be carried out using the apparatus described in International Application No. PCT/GB08/000562 (WO 2008/102120).

A variety of different types of measurements may be made. This includes without limitation: electrical measurements and optical measurements. Possible electrical measurements include: current measurements, impedance measurements, tunnelling measurements (Ivanov A P et al., Nano Lett. 2011 Jan. 12; 11(1):279-85), and FET measurements (International Application WO 2005/124888). Optical measurements may be combined with electrical measurements (Soni G V et al., Rev Sci Instrum. 2010 January; 81(1):014301). The measurement may be a transmembrane current measurement such as measurement of ionic current flowing through the pore.

Electrical measurements may be made using standard single channel recording equipment as describe in Stoddart D et al., Proc Natl Acad Sci, 12; 106(19):7702-7, Lieberman K R et al, J Am Chem Soc. 2010; 132(50):17961-72, and International Application WO 2000/28312. Alternatively, electrical measurements may be made using a multi-channel system, for example as described in International Application WO 2009/077734 and International Application WO 2011/067559.

The method is preferably carried out with a potential applied across the membrane. The applied potential may be a voltage potential. Alternatively, the applied potential may be a chemical potential. An example of this is using a salt gradient across a membrane, such as an amphiphilic layer. A salt gradient is disclosed in Holden et al., J Am Chem Soc. 2007 Jul. 11; 129(27):8650-5. In some instances, the current passing through the pore as a polynucleotide moves with respect to the pore is used to estimate or determine the sequence of the polynucleotide This is strand sequencing.

The method may Involve measuring the current passing through the pore as the polynucleotide moves with respect to the pore. Therefore the apparatus used in the method may also comprise an electrical circuit capable of applying a potential and measuring an electrical signal across the membrane and pore. The methods may be carried out using a patch clamp or a voltage clamp. The methods preferably involve the use of a voltage clamp.

The method of the invention may involve the measuring of a current passing through the pore as the polynucleotide moves with respect to the pore. Suitable conditions for measuring ionic currents through transmembrane protein pores are known In the art and disclosed in the Example. The method is typically carried out with a voltage applied across the membrane and pore. The voltage used is typically from +5 V to −5 V, such as from +4 V to −4 V, +3 V to −3 V or +2 V to −2 V. The voltage used is typically from −600 mV to +600 mV or −400 mV to +400 mV. The voltage used is preferably in a range having a lower limit selected from −400 mV, −300 mV, −200 mV, −150 mV, −100 mV, −50 mV, −20 mV and 0 mV and an upper limit independently selected from +10 mV, +20 mV, +50 mV, +100 mV, +150 mV, +200 mV. +300 mV and +400 mV. The voltage used is more preferably in the range 100 mV to 240 mV and most preferably in the range of 120 mV to 220 mV. It is possible to increase discrimination between different nucleotides by a pore by using an increased applied potential.

The method is typically carried out in the presence of any charge carriers, such as metal salts, for example alkali metal salt, halide salts, for example chloride salts, such as alkali metal chloride salt. Charge carriers may include ionic liquids or organic salts, for example tetramethyl ammonium chloride, trimethylphenyl ammonium chloride, phenyltrimethyl ammonium chloride, or 1-ethyl-3-methyl imidazolium chloride. In the exemplary apparatus discussed above, the salt is present in the aqueous solution in the chamber. Potassium chloride (KCl), sodium chloride (NaCl), caesium chloride (CsCl) or a mixture of potassium ferrocyanide and potassium ferricyanide is typically used. KCl, NaCl and a mixture of potassium ferrocyanide and potassium ferricyanide are preferred. The charge carriers may be asymmetric across the membrane. For instance, the type and/or concentration of the charge carriers may be different on each side of the membrane.

The salt concentration may be at saturation. The salt concentration may be 3 M or lower and is typically from 0.1 to 2.5 M, from 0.3 to 1.9 M, from 0.5 to 1.8 M, from 0.7 to 1.7 M, from 0.9 to 1.6 M or from 1 M to 1.4 M. The salt concentration is preferably from 150 mM to 1 M. The method is preferably carried out using a salt concentration of at least 0.3 M, such as at least 0.4 M, at least 0.5 M, at least 0.6 M, at least 0.8 M, at least 1.0 M, at least 1.5 M, at least 2.0 M, at least 2.5 M or at least 3.0 M. High salt concentrations provide a high signal to noise ratio and allow for currents indicative of the presence of a nucleotide to be identified against the background of normal current fluctuations.

The method is typically carried out in the presence of a buffer. In the exemplary apparatus discussed above, the buffer is present in the aqueous solution in the chamber. Any buffer may be used in the method of the invention. Typically, the buffer is phosphate buffer. Other suitable buffers are HEPES and Tris-HCl buffer. The methods are typically carried out at a pH of from 4.0 to 12.0, from 4.5 to 10.0, from 5.0 to 9.0, from 5.5 to 8.8, from 6.0 to 8.7 or from 7.0 to 8.8 or 7.5 to 8.5. The pH used is preferably about 7.5.

The method may be carried out at from 0° C. to 100° C., from 15° C. to 95° C., from 16° C. to 90° C., from 17° C. to 85° C., from 18° C. to 80° C. 19° C. to 70° C., or from 20° C. to 60° C. The methods are typically carried out at room temperature. The methods are optionally carried out at a temperature that supports enzyme function, such as about 37° C.

### Polynucleotide Binding Protein

The strand characterisation method preferably comprises contacting the polynucleotide with a polynucleotide binding protein such that the protein controls the movement of the polynucleotide with respect to, such as through, the pore.

More preferably, the method comprises (a) contacting the polynucleotide with a CsgG pore or mutant thereof, such as a pore of the invention, and a polynucleotide binding protein such that the protein controls the movement of the polynucleotide with respect to, such as through, the pore and (b) taking one or more measurements as the polynucleotide moves with respect to the pore, wherein the measurements are indicative of one or more characteristics of the polynucleotide, and thereby characterising the polynucleotide.

More preferably, the method comprises (a) contacting the polynucleotide with a CsgG pore or mutant thereof, such as a pore of the invention, and a polynucleotide binding protein such that the protein controls the movement of the polynucleotide with respect to, such as through, the pore and (b) measuring the current through the pore as the polynucleotide moves with respect to the pore, wherein the current is indicative of one or more characteristics of the polynucleotide, and thereby characterising the polynucleotide.

The polynucleotide binding protein may be any protein that is capable of binding to the polynucleotide and controlling its movement through the pore. It is straightforward in the art to determine whether or not a protein binds to a polynucleotide. The protein typically Interacts with and modifies at least one property of the polynucleotide. The protein may modify the polynucleotide by cleaving it to form individual nucleotides or shorter chains of nucleotides, such as di- or trinucleotides. The protein may modify the polynucleotide by orienting it or moving it to a specific position, i.e. controlling its movement.

The polynucleotide binding protein is preferably derived from a polynucleotide handling enzyme. A polynucleotide handling enzyme is a polypeptide that is capable of interacting with and modifying at least one property of a polynucleotide. The enzyme may modify the polynucleotide by cleaving it to form individual nucleotides or shorter chains of nucleotides, such as di- or trinucleotides. The enzyme may modify the polynucleotide by orienting it or moving it to a specific position. The polynucleotide handling enzyme does not need to display enzymatic activity as long as it is capable of binding the polynucleotide and controlling its movement through the pore. For instance, the enzyme may be modified to remove its enzymatic activity or may be used under conditions which prevent it from acting as an enzyme. Such conditions are discussed in more detail below.

The polynucleotide handling enzyme is preferably derived from a nucleolytic enzyme. The polynucleotide handling enzyme used in the construct of the enzyme is more preferably derived from a member of any of the Enzyme Classification (EC) groups 3.1.11, 3.1.13, 3.1.14, 3.1.15, 3.1.16, 3.1.21, 3.1.22, 3.1.25, 3.1.26, 3.1.27, 3 1.30 and 3.1.31. The enzyme may be any of those disclosed in International Application No. PCT/GB10/000133 (published as WO 2010/086603).

Preferred enzymes are polymerases, exonucleases, helicases and topoisomerases, such as gyrases. Suitable enzymes include, but are not limited to, exonuclease I from E. coli (SEQ ID NO: 399), exonuclease III enzyme from E. coli (SEQ ID NO: 401), RecJ from T. thermophilus (SEQ ID NO: 403) and bacteriophage lambda exonuclease (SEQ ID NO: 405), TatD exonuclease and variants thereof. Three subunits comprising the sequence shown in SEQ ID NO: 403 or a variant thereof interact to form a trimer exonuclease. These exonucleases can also be used in the exonuclease method of the invention. The polymerase may be PyroPhage® 3173 DNA Polymerase (which is commercially available from Lucigen® Corporation), SD Polymerase (commercially available from Bioron®) or variants thereof. The enzyme is preferably Phi29 DNA polymerase (SEQ ID NO: 397) or a variant thereof. The topoisomerase is preferably a member of any of the Moiety Classification (EC) groups 5.99.1.2 and 5.99.1.3.

The enzyme is most preferably derived from a helicase, such as Hel308 Mbu (SEQ ID NO: 406), Hel308 Csy (SEQ ID NO: 407), Hel308 Tga (SEQ ID NO: 408), Hel308 Mhu (SEQ ID NO: 409), Tral Eco (SEQ ID NO: 410), XPD Mbu (SEQ ID NO: 411) or a variant thereof. Any helicase may be used in the invention. The helicase may be or be derived from a Hel308 helicase, a RecD helicase, such as Tral helicase or a TrwC helicase, a XPD helicase or a Dda helicase. The helicase may be any of the helicases, modified helicases or helicase constructs disclosed in International Application Nos. PCT/GB2012/052579 (published as WO 2013/057495); PCT/GB2012/053274 (published as WO 2013/098562); PCT/GB2012/053273 (published as WO2013098561); PCT/GB2013/051925 (published as WO 2014/013260); PCT/GB2013/051924 (published as WO 2014/013259); PCT/GB2013/051928 (published as WO 2014/013262) and PCT/GB2014/052736.

The helicase preferably comprises the sequence shown in SEQ ID NO: 413 (Trwc Cba) or as variant thereof, the sequence shown in SEQ ID NO: 406 (Hel308 Mbu) or a variant thereof or the sequence shown in SEQ ID NO: 412 (Dda) or a variant thereof. Variants may differ from the native sequences in any of the ways discussed below for transmembrane pores. A preferred variant of SEQ ID NO: 412 comprises (a) E94C and A360C or (b) E94C, A360C, C109A and C136A and then optionally (ΔM1)G1G2 (i.e. deletion of M1 and then addition G1 and G2).

Any number of helicases may be used in accordance with the invention. For instance, 1, 2, 3, 4, 5, 6, 7, 8, 9, 10 or more helicases may be used. In some embodiments, different numbers of helicases may be used.

The method of the invention preferably comprises contacting the polynucleotide with two or more helicases. The two or more helicases are typically the same helicase. The two or more helicases may be different helicases.

The two or more helicases may be any combination of the helicases mentioned above. The two or more helicases may be two or more Dda helicases. The two or more helicases may be one or more Dda helicases and one or more TrwC helicases. The two or more helicases may be different variants of the same helicase.

The two or more helicases are preferably attached to one another. The two or more helicases are more preferably covalently attached to one another. The helicases may be attached in any order and using any method. Preferred helicase constructs for use in the invention are described in International Application Nos. PCT/GB2013/051925 (published as WO 2014/013260); PCT/GB2013/051924 (published as WO 2014/013259); PCT/GB2013/051928 (published as WO 2014/013262) and PCT/GB2014/052736.

A variant of SEQ ID NOs: 397, 399, 401, 403, 405, 406, 407, 408, 409, 410, 411, 412 or 413 is an enzyme that has an amino acid sequence which varies from that of SEQ ID NO: 397, 399, 401, 403, 405, 406, 407, 408, 409, 410, 411, 412 or 413 and which retains polynucleotide binding ability. This can be measured using any method known in the art. For instance, the variant can be contacted with a polynucleotide and its ability to bind to and move along the polynucleotide can be measured. The variant may include modifications that facilitate binding of the polynucleotide and/or facilitate its activity at high salt concentrations and/or room temperature. Variants may be modified such that they bind polynucleotides (i.e. retain polynucleotide binding ability) but do not function as a helicase (i.e. do not move along polynucleotides when provided with all the necessary components to facilitate movement, e.g. ATP and Mg²⁺). Such modifications are known in the art. For instance, modification of the Mg²⁺ binding domain in helicases typically results in variants which do not function as helicases. These types of variants may act as molecular brakes (see below).

Over the entire length of the amino acid sequence of SEQ ID NO: 397, 399, 401, 403, 405, 406, 407, 408, 409, 410, 411, 412 or 413, a variant will preferably be at least 50% homologous to that sequence based on amino acid identity. More preferably, the variant polypeptide may be at least 55%, at least 60%, at least 65%, at least 70%, at least 75%, at least 80%, at least 85%, at least 90% and more preferably at least 95%, 97% or 99% homologous based on amino acid identity to the amino acid sequence of SEQ ID NO: 397, 399, 401, 403, 405, 406, 407, 408, 409, 410, 411, 412 or 413 over the entire sequence. There may be at least 80%, for example at least 85%, 90% or 95%, amino acid identity over a stretch of 200 or more, for example 230, 250, 270, 280, 300, 400, 500, 600, 700, 800, 900 or 1000 or more, contiguous amino acids (“hard homology”). Homology is determined as described above. The variant may differ from the wild-type sequence in any of the ways discussed above with reference to SEQ ID NO: 390 above. The enzyme may be covalently attached to the pore. Any method may be used to covalently attach the enzyme to the pore.

A preferred molecular brake is TrwC Cba-Q594A (SEQ ID NO: 413 with the mutation Q594A) This variant does not function as a helicase (i.e. binds polynucleotides but does not move along them when provided with all the necessary components to facilitate movement, e.g. ATP and Mg²⁺).

In strand sequencing, the polynucleotide is translocated through the pore either with or against an applied potential. Exonucleases that act progressively or processively on double stranded polynucleotides can be used on the cis side of the pore to feed the remaining single strand through under an applied potential or the trans side under a reverse potential. Likewise, a helicase that unwinds the double stranded DNA can also be used in a similar manner. A polymerase may also be used. There are also possibilities for sequencing applications that require strand translocation against an applied potential, but the DNA must be first “caught” by the enzyme under a reverse or no potential. With the potential then switched back following binding the strand will pass cis to trans through the pore and be held in an extended conformation by the current flow. The single strand DNA exonucleases or single strand DNA dependent polymerases can act as molecular motors to pull the recently translocated single strand back through the pore in a controlled stepwise manner, trans to cis, against the applied potential.

Any helicase may be used in the method. Helicases may work in two modes with respect to the pore. First, the method is preferably carried out using a helicase such that it moves the polynucleotide through the pore with the field resulting from the applied voltage. In this mode the 5′ end of the polynucleotide is first captured in the pore, and the helicase moves the polynucleotide into the pore such that it is passed through the pore with the field until it finally translocates through to the trans side of the membrane. Alternatively, the method is preferably carried out such that a helicase moves the polynucleotide through the pore against the field resulting from the applied voltage. In this mode the 3′ end of the polynucleotide is first captured in the pore, and the helicase moves the polynucleotide through the pore such that It is pulled out of the pore against the applied field until finally ejected back to the cis side of the membrane.

The method may also be carried out in the opposite direction. The 3′ end of the polynucleotide may be first captured in the pore and the helicase may move the polynucleotide into the pore such that it is passed through the pore with the field until it finally translocates through to the trans side of the membrane.

When the helicase is not provided with the necessary components to facilitate movement or is modified to hinder or prevent its movement, it can bind to the polynucleotide and act as a brake slowing the movement of the polynucleotide when it is pulled into the pore by the applied field. In the inactive mode, it does not matter whether the polynucleotide is captured either 3′ or 5′ down, it is the applied field which pulls the polynucleotide into the pore towards the trans side with the enzyme acting as a brake When in the inactive mode, the movement control of the polynucleotide by the helicase can be described in a number of ways including ratcheting, sliding and braking. Helicase variants which lack helicase activity can also be used in this way.

The polynucleotide may be contacted with the polynucleotide binding protein and the pore in any order. It is preferred that, when the polynucleotide is contacted with the polynucleotide binding protein, such as a helicase, and the pore, the polynucleotide firstly forms a complex with the protein. When the voltage is applied across the pore, the polynucleotide/protein complex then forms a complex with the pore and controls the movement of the polynucleotide through the pore.

Any steps in the method using a polynucleotide binding protein are typically carried out in the presence of free nucleotides or free nucleotide analogues and an enzyme cofactor that facilitates the action of the polynucleotide binding protein. The free nucleotides may be one or more of any of the individual nucleotides discussed above. The free nucleotides include, but are not limited to, adenosine monophosphate (AMP), adenosine diphosphate (ADP), adenosine triphosphate (ATP), guanosine monophosphate (GMP), guanosine diphosphate (GDP), guanosine triphosphate (GTP), thymidine monophosphate (TMP), thymidine diphosphate (TDP), thymidine triphosphate (TTP), uridine monophosphate (UMP), uridine diphosphate (UDP), uridine triphosphate (UTP), cytidine monophosphate (CMP), cytidine diphosphate (CDP), cytidine triphosphate (CTP), cyclic adenosine monophosphate (cAMP), cyclic guanosine monophosphate (cGMP), deoxyadenosine monophosphate (dAMP), deoxyadenosine diphosphate (dADP), deoxyadenosine triphosphate (dATP), deoxyguanosine monophosphate (dGMP), deoxyguanosine diphosphate (dGDP), deoxyguanosine triphosphate (dGTP), deoxythymidine monophosphate (dTMP), deoxythymidine diphosphate (dTDP), deoxythymidine triphosphate (dTTP), deoxyuridine monophosphate (dUMP), deoxyuridine diphosphate (dUDP), deoxyuridine triphosphate (dUTP), deoxycytidine monophosphate (dCMP), deoxycytidine diphosphate (dCDP) and deoxycytidine triphosphate (dCTP). The free nucleotides are preferably selected from AMP, TMP, GMP, CMP, UMP, dAMP, dTMP, dGMP or dCMP. The free nucleotides are preferably adenosine triphosphate (ATP). The enzyme cofactor is a factor that allows the construct to function. The enzyme cofactor is preferably a divalent metal cation. The divalent metal cation is preferably Mg²⁺, Mn²⁺, Ca²⁺ or Co²⁺. The enzyme cofactor is most preferably Mg²⁺.

### Helicase(s) and Molecular Brake(s)

In a preferred embodiment, the method comprises:
(a) providing the polynucleotide with one or more helicases and one or more molecular brakes attached to the polynucleotide;
(b) contacting the polynucleotide with a CsgG pore or mutant thereof, such as a pore of the invention, and applying a potential across the pore such that the one or more helicases and the one or more molecular brakes are brought together and both control the movement of the polynucleotide with respect to, such as through, the pore;
(c) taking one or more measurements as the polynucleotide moves with respect to the pore wherein the measurements are indicative of one or more characteristics of the polynucleotide and thereby characterising the polynucleotide.

This type of method is discussed in detail in the International Application PCT/GB2014/052737

The one or more helicases may be any of those discussed above. The one or more molecular brakes may be any compound or molecule which binds to the polynucleotide and slows the movement of the polynucleotide through the pore. The one or more molecular brakes preferably comprise one or more compounds which bind to the polynucleotide. The one or more compounds are preferably one or more macrocycles. Suitable macrocycles include, but are not limited to, cyclodextrins, calixarenes, cyclic peptides, crown ethers, cucurbiturils, pillararenes, derivatives thereof or a combination thereof. The cyclodextrin or derivative thereof may be any of those disclosed in Eliseev, A. V., and Schneider, H-J. (1994) J. Am. Chem. Soc. 116, 6081-6088. The agent is more preferably heptakis-6-amino-β-cyclodextrin (am₇-βCD), 6-monodeoxy-6-monoamino-β-cyclodextrin (am₁-□CD) or heptakis-(6-deoxy-& guanidino)-cyclodextrin (gu₇-βCD).

The one or more molecular brakes are preferably one or more single stranded binding proteins (SSB). The one or more molecular brakes are more preferably a single-stranded binding protein (SSB) comprising a carboxy-terminal (C-terminal) region which does not have a net negative charge or (ii) a modified SSB comprising one or more modifications in its C-terminal region which decreases the net negative charge of the C-terminal region. The one or more molecular brakes are most preferably one of the SSBs disclosed in International Application No. PCT/GB2013/051924 (published as WO 2014/013259).

The one or more molecular brakes are preferably one or more polynucleotide binding proteins. The polynucleotide binding protein may be any protein that is capable of binding to the polynucleotide and controlling its movement through the pore. It is straightforward in the art to determine whether or not a protein binds to a polynucleotide. The protein typically interacts with and modifies at least one property of the polynucleotide. The protein may modify the polynucleotide by cleaving it to form individual nucleotides or shorter chains of nucleotides, such as di- or trinucleotides. The moiety may modify the polynucleotide by orienting it or moving it to a specific position, i.e. controlling its movement.

The polynucleotide binding protein is preferably derived from a polynucleotide handling enzyme. The one or more molecular brakes may be derived from any of the polynucleotide handling enzymes discussed above. Modified versions of Phi29 polymerase (SEQ ID NO: 396) which act as molecular brakes are disclosed In U.S. Pat. No. 5,576,204. The one or more molecular brakes are preferably derived from a helicase.

Any number of molecular brakes derived from a helicase may be used. For instance, 1, 2, 3, 4, 5, 6, 7, 8, 9, 10 or more helicases may be used as molecular brakes. If two or more helicases are be used as molecular brakes, the two or more helicases are typically the same helicase. The two or more helicases may be different helicases.

The two or more helicases may be any combination of the helicases mentioned above. The two or more helicases may be two or more Dda helicases. The two or more helicases may be one or more Dda helicases and one or more TrwC helicases. The two or more helicases may be different variants of the same helicase.

The two or more helicases are preferably attached to one another. The two or more helicases are more preferably covalently attached to one another. The helicases may be attached in any order and using any method. The one or more molecular brakes derived from helicases are preferably modified to reduce the size of an opening in the polynucleotide binding domain through which in at least one conformational state the polynucleotide can unbind from the helicase. This is disclosed in WO 2014/013260.

Preferred helicase constructs for use in the invention are described in International Application Nos. PCT/GB2013/051925 (published as WO 2014/013260); PCT/GB2013/051924 (published as WO 2014/013259); PCT/GB2013/051928 (published as WO 2014/013262) and PCT/GB2014/052736.

If the one or more helicases are used in the active mode (i.e. when the one or more helicases are provided with all the necessary components to facilitate movement, e.g. ATP and Mg²⁺), the one or more molecular brakes are preferably (a) used In an inactive mode (i.e. are used in the absence of the necessary components to facilitate movement or are incapable of active movement), (b) used in an active mode where the one or more molecular brakes move in the opposite direction to the one or more helicases or (c) used in an active mode where the one or more molecular brakes move in the same direction as the one or more helicases and more slowly than the one or more helicases.

If the one or more helicases are used in the inactive mode (i.e. when the one or more helicases are not provided with all the necessary components to facilitate movement, e.g. ATP and Mg²⁺ or are Incapable of active movement), the one or more molecular brakes are preferably (a) used In an inactive mode (i.e. are used in the absence of the necessary components to facilitate movement or are incapable of active movement) or (b) used in an active mode where the one or more molecular brakes move along the polynucleotide in the same direction as the polynucleotide through the pore.

The one or more helicases and one or more molecular brakes may be attached to the polynucleotide at any positions so that they are brought together and both control the movement of the polynucleotide through the pore. The one or more helicases and one or more molecular brakes are at least one nucleotide apart, such as at least 5, at least 10, at least 50, at least 100, at least 500, at least 1000, at least 5000, at least 10,000, at least 50.000 nucleotides or more apart. If the method concerns characterising a double stranded polynucleotide provided with a Y adaptor at one end and a hairpin loop adaptor at the other end, the one or more helicases are preferably attached to the Y adaptor and the one or more molecular brakes are preferably attached to the hairpin loop adaptor. In this embodiment, the one or more molecular brakes are preferably one or more helicases that are modified such that they bind the polynucleotide but do not function as a helicase. The one or more helicases attached to the Y adaptor are preferably stalled at a spacer as discussed in more detail below. The one or more molecular brakes attach to the hairpin loop adaptor are preferably not stalled at a spacer. The one or more helicases and the one or more molecular brakes are preferably brought together when the one or more helicases reach the hairpin loop. The one or more helicases may be attached to the Y adaptor before the Y adaptor is attached to the polynucleotide or after the Y adaptor is attached to the polynucleotide. The one or more molecular brakes may be attached to the hairpin loop adaptor before the hairpin loop adaptor is attached to the polynucleotide or after the hairpin loop adaptor is attached to the polynucleotide.

The one or more helicases and the one or more molecular brakes are preferably not attached to one another. The one or more helicases and the one or more molecular brakes are more preferably not covalently attached to one another. The one or more helicases and the one or more molecular brakes are preferably not attached as described in International Application Nos. PCT/GB2013/051925 (published as WO 20141013260); PCT/GB20131051924 (published as WO 2014/013259); PCT/GB2013/051928 (published as WO 2014/013262) and PCT/GB2014/052736.

### Spacers

The one or more helicases may be stalled at one or more spacers as discussed in International Application No. PCT/GB20141050175. Any configuration of one or more helicases and one or more spacers disclosed in the International Application may be used in this invention.

When a part of the polynucleotide enters the pore and moves through the pore along the field resulting from the applied potential, the one or more helicases are moved past the spacer by the pore as the polynucleotide moves through the pore. This is because the polynucleotide (including the one or more spacers) moves through the pore and the one or more helicases remain on top of the pore.

The one or more spacers are preferably part of the polynucleotide, for instance they interrupt(s) the polynucleotide sequence. The one or more spacers are preferably not part of one or more blocking molecules, such as speed bumps, hybridised to the polynucleotide.

There may be any number of spacers in the polynucleotide, such as 1, 2, 3, 4, 5, 6, 7, 8, 9, 10 or more spacers. There are preferably two, four or six spacers in the polynucleotide. There may be one or more spacers in different regions of the polynucleotide, such as one or more spacers in the Y adaptor and/or hairpin loop adaptor.

The one or more spacers each provides an energy barrier which the one or more helicases cannot overcome even in the active mode. The one or more spacers may stall the one or more helicases by reducing the traction of the helicase (for instance by removing the bases from the nucleotides in the polynucleotide) or physically blocking movement of the one or more helicases (for instance using a bulky chemical group).

The one or more spacers may comprise any molecule or combination of molecules that stalls the one or more helicases. The one or more spacers may comprise any molecule or combination of molecules that prevents the one or more helicases from moving along the polynucleotide. It is straightforward to determine whether or not the one or more helicases are stalled at one or more spacers in the absence of a transmembrane pore and an applied potential. For instance, the ability of a helicase to move past a spacer and displace a complementary strand of DNA can be measured by PAGE.

The one or more spacers typically comprise a linear molecule, such as a polymer. The one or more spacers typically have a different structure from the polynucleotide. For instance, if the polynucleotide is DNA, the one or more spacers are typically not DNA. In particular, if the polynucleotide is deoxyribonucleic acid (DNA) or ribonucleic acid (RNA), the one or more spacers preferably comprise peptide nucleic acid (PNA), glycerol nucleic acid (GNA), threose nucleic acid (TNA), locked nucleic acid (LNA) or a synthetic polymer with nucleotide side chains. The one or more spacers may comprise one or more nucleotides in the opposite direction from the polynucleotide. For instance, the one or more spacers may comprise one or more nucleotides in the 3′ to 5′ direction when the polynucleotide is in the 5′ to 3′ direction. The nucleotides may be any of those discussed above.

The one or more spacers preferably comprises one or more nitroindoles, such as one or more 5-nitroindoles, one or more inosines, one or more acridines, one or more 2-aminopurines, one or more 2-6-diaminopurines, one or more 5-bromo-deoxyuridines, one or more Inverted thymidines (Inverted dTs), one or more inverted dideoxy-thymidines (ddTs), one or more dideoxy-cytidines (ddCs), one or more 5-methylcytidines, one or more 5-hydroxymethylcytidines, one or more 2′-O-Methyl RNA bases, one or more Iso-deoxycytidines (Iso-dCs), one or more Iso-deoxyguanosines (Iso-dGs), one or more iSpC3 groups (i.e. nucleotides which lack sugar and a base), one or more photo-cleavable (PC) groups, one or more hexandiol groups, one or more spacer 9 (iSp9) groups, one or more spacer 18 (iSp18) groups, a polymer or one or more thiol connections. The one or more spacers may comprise any combination of these groups. Many of these groups are commercially available from IDT® (Integrated DNA Technologies®).

The one or more spacers may contain any number of these groups. For instance, for 2-aminopurines, 2-6-diaminopurines, 5-bromo-deoxyuridines, inverted dTs, ddTs, ddCs, 5-methylcytidines, 5-hydroxymethylcytidines, 2′-O-Methyl RNA bases, Iso-dCs, Iso-dGs, ISpC3 groups, PC groups, hexandiol groups and thiol connections, the one or more spacers preferably comprise 2, 3, 4, 5, 6, 7, 8, 9, 10, 11, 12 or more. The one or more spacers preferably comprise 2, 3, 4, 5, 6, 7, 8 or more iSp9 groups. The one or more spacers preferably comprise 2, 3, 4, 5 or 6 or more iSp18 groups. The most preferred spacer is four iSp18 groups.

The polymer is preferably a polypeptide or a polyethylene glycol (PEG). The polypeptide preferably comprises 2, 3, 4, 5, 6, 7, 8, 9, 10, 11, 12 or more amino acids. The PEG preferably comprises 2, 3, 4, 5, 6, 7, 8, 9, 10, 11, 12 or more monomer units.

The one or more spacers preferably comprise one or more abasic nucleotides (i.e. nucleotides lacking a nucleobase), such as 2, 3, 4, 5, 6, 7, 8, 9, 10, 11, 12 or more abasic nucleotides. The nucleobase can be replaced by —H (idSp) or —OH in the abasic nucleotide. Abasic spacers can be inserted Into polynucleotides by removing the nucleobases from one or more adjacent nucleotides. For instance, polynucleotides may be modified to include 3-methyladenine, 7-methylguanine, 1,N6-ethenoadenine inosine or hypoxanthine and the nucleobases may be removed from these nucleotides using Human Alkyladenine DNA Glycosylase (hAAG). Alternatively, polynucleotides may be modified to include uracil and the nucleobases removed with Uracil-DNA Glycosylase (UDG). In one embodiment, the one or more spacers do not comprise any abasic nucleotides.

The one or more helicases may be stalled by (i.e. before) or on each linear molecule spacers. If linear molecule spacers are used, the polynucleotide is preferably provided with a double stranded region of polynucleotide adjacent to the end of each spacer past which the one or more helicases are to be moved. The double stranded region typically helps to stall the one or more helicases on the adjacent spacer. The presence of the double stranded region(s) is particularly preferred if the method is carried out at a salt concentration of about 100 mM or lower. Each double stranded region is typically at least 10, such as at least 12, nucleotides in length. If the polynucleotide used in the invention is single stranded, a double stranded region may be formed by hybridising a shorter polynucleotide to a region adjacent to a spacer. The shorter polynucleotide is typically formed from the same nucleotides as the polynucleotide, but may be formed from different nucleotides. For instance, the shorter polynucleotide may be formed from LNA.

If linear molecule spacers are used, the polynucleotide is preferably provided with a blocking molecule at the end of each spacer opposite to the end past which the one or more helicases are to be moved. This can help to ensure that the one or more helicases remain stalled on each spacer. It may also help retain the one or more helicases on the polynucleotide in the case that it/they diffuse(s) off in solution. The blocking molecule may be any of the chemical groups discussed below which physically cause the one or more helicases to stall. The blocking molecule may be a double stranded region of polynucleotide.

The one or more spacers preferably comprise one or more chemical groups which physically cause the one or more helicases to stall. The one or more chemical groups are preferably one or more pendant chemical groups. The one or more chemical groups may be attached to one or more nucleobases in the polynucleotide. The one or more chemical groups may be attached to the polynucleotide backbone. Any number of these chemical groups may be present, such as 2, 3, 4, 5, 6, 7, 8, 9, 10, 11, 12 or more. Suitable groups Include, but are not limited to, fluorophores, streptavidin and/or biotin, cholesterol, methylene blue, dinitrophenols (DNPs), digoxigenin and/or anti-digoxigenin and dibenzylcyclooctyne groups.

Different spacers in the polynucleotide may comprise different stalling molecules. For instance, one spacer may comprise one of the linear molecules discussed above and another spacer may comprise one or more chemical groups which physically cause the one or more helicases to stall. A spacer may comprise any of the linear molecules discussed above and one or more chemical groups which physically cause the one or more helicases to stall, such as one or more a basics and a fluorophore.

Suitable spacers can be designed depending on the type of polynucleotide and the conditions under which the method of the invention is carried out. Most helicases bind and move along DNA and so may be stalled using anything that is not DNA. Suitable molecules are discussed above.

The method of the invention is preferably carried out in the presence of free nucleotides and/or the presence of a helicase cofactor. This is discussed in more detail below. In the absence of the transmembrane pore and an applied potential, the one or more spacers are preferably capable of stalling the one or more helicases in the presence of free nucleotides and/or the presence of a helicase cofactor.

If the method of the invention is carried out in the presence of free nucleotides and a helicase cofactor as discussed below (such that the one of more helicases are in the active mode), one or more longer spacers are typically used to ensure that the one or more helicases are stalled on the polynucleotide before they are contacted with the transmembrane pore and a potential is applied. One or more shorter spacers may be used in the absence of free nucleotides and a helicase cofactor (such that the one or more helicases are in the inactive mode).

The salt concentration also affects the ability of the one or more spacers to stall the one or more helicases. In the absence of the transmembrane pore and an applied potential, the one or more spacers are preferably capable of stalling the one or more helicases at a salt concentration of about 100 mM or lower. The higher the salt concentration used in the method of the invention, the shorter the one or more spacers that are typically used and vice versa.

Preferred combinations of features are shown in Table 3 below.

TABLE 3

| | | Spacer length | | | | |
|---|---|---|---|---|---|---|
| | Spacer | (i.e. number | | | Free | Helicase |
| Polynucleotide | composition* | of *) | Salt [ ] | | nucleotides? | cofactor? |
| DNA | iSpC3 | 4 | 1M | | Yes | Yes |
| DNA | iSp18 | 4 | 100-1000 | mM | Yes | Yes |
| DNA | iSp18 | 6 | <100-1000 | mM | Yes | Yes |
| DNA | iSp18 | 2 | 1M | | Yes | Yes |
| DNA | iSpC3 | 12 | <100-1000 | mM | Yes | Yes |
| DNA | iSpC3 | 20 | <100-1000 | mM | Yes | Yes |
| DNA | iSp9 | 6 | 100-1000 | mM | Yes | Yes |

| | | | | | |
|---|---|---|---|---|---|
| DNA | idSp | 4 | 1M | Yes | Yes |

The method may concern moving two or more helicases past a spacer. In such instances, the length of the spacer is typically increased to prevent the trailing helicase from pushing the leading helicase past the spacer In the absence of the pore and applied potential. If the method concerns moving two or more helicases past one or more spacers, the spacer lengths discussed above may be increased at least 1.5 fold, such 2 fold, 2.5 fold or 3 fold. For instance, if the method concerns moving two or more helicases past one or more spacers, the spacer lengths in the third column of Table 3 above may be increased 1.5 fold, 2 fold, 2.5 fold or 3 fold.

### Membrane

The pore of the Invention may be present in a membrane. In the methods of the Invention, the polynucleotide is typically contacted with the CsgG pore or mutant thereof, such as a pore of the invention, in a membrane. Any membrane may be used in accordance with the invention. Suitable membranes are well-known in the art. The membrane is preferably an amphiphilic layer. An amphiphilic layer is a layer formed from amphiphilic molecules, such as phospholipids, which have both hydrophilic and lipophilic properties. The amphiphilic molecules may be synthetic or naturally occurring. Non-naturally occurring amphiphiles and amphiphiles which form a monolayer are known in the art and include, for example, block copolymers (Gonzalez-Perez et al., Langmuir, 2009, 25, 10447-10450). Block copolymers are polymeric materials in which two or more monomer sub-units that are polymerized together to create a single polymer chain. Block copolymers typically have properties that are contributed by each monomer sub-unit. However, a block copolymer may have unique properties that polymers formed from the individual sub-units do not possess. Block copolymers can be engineered such that one of the monomer sub-units is hydrophobic (i.e. lipophilic), whilst the other sub-unit(s) are hydrophilic whilst in aqueous media. In this case, the block copolymer may possess amphiphilic properties and may form a structure that mimics a biological membrane. The block copolymer may be a diblock (consisting of two monomer sub-units), but may also be constructed from more than two monomer sub-units to form more complex arrangements that behave as amphiphiles. The copolymer may be a triblock, tetrablock or pentablock copolymer. The membrane is preferably a triblock copolymer membrane.

Archaebacterial bipolar tetraether lipids are naturally occurring lipids that are constructed such that the lipid forms a monolayer membrane. These lipids are generally found in extremophiles that survive in harsh biological environments, thermophiles, halophiles and acidophiles. Their stability is believed to derive from the fused nature of the final bilayer. It is straightforward to construct block copolymer materials that mimic these biological entities by creating a triblock polymer that has the general motif hydrophilic-hydrophobic-hydrophilic. This material may form monomeric membranes that behave similarly to lipid bilayers and encompass a range of phase behaviours from vesicles through to laminar membranes. Membranes formed from these triblock copolymers hold several advantages over biological lipid membranes. Because the triblock copolymer is synthesised, the exact construction can be carefully controlled to provide the correct chain lengths and properties required to form membranes and to interact with pores and other proteins.

Block copolymers may also be constructed from sub-units that are not classed as lipid sub-materials, for example a hydrophobic polymer may be made from siloxane or other non-hydrocarbon based monomers. The hydrophilic sub-section of block copolymer can also possess low protein binding properties, which allows the creation of a membrane that is highly resistant when exposed to raw biological samples. This head group unit may also be derived from non-classical lipid head-groups.

Triblock copolymer membranes also have increased mechanical and environmental stability compared with biological lipid membranes, for example a much higher operational temperature or pH range. The synthetic nature of the block copolymers provides a platform to customise polymer based membranes for a wide range of applications The membrane is most preferably one of the membranes disclosed in International Application No. PCT/GB2013/052766 or PCT/GB2013/052767.

The amphiphilic molecules may be chemically-modified or functionalised to facilitate coupling of the polynucleotide.

The amphiphilic layer may be a monolayer or a bilayer. The amphiphilic layer is typically planar. The amphiphilic layer may be curved. The amphiphilic layer may be supported.

Amphiphilic membranes are typically naturally mobile, essentially acting as two dimensional fluids with lipid diffusion rates of approximately 10⁻⁸ cm s-1. This means that the pore and coupled polynucleotide can typically move within an amphiphilic membrane.

The membrane may be a lipid bilayer. Lipid bilayers are models of cell membranes and serve as excellent platforms for a range of experimental studies. For example, lipid bilayers can be used for in vitro investigation of membrane proteins by single-channel recording. Alternatively, lipid bilayers can be used as biosensors to detect the presence of a range of substances. The lipid bilayer may be any lipid bilayer. Suitable lipid bilayers include, but are not limited to, a planar lipid bilayer, a supported bilayer or a liposome. The lipid bilayer is preferably a planar lipid bilayer. Suitable lipid bilayers are disclosed in International Application No. PCT/GB08/000563 (published as WO 2008/102121), International Application No. PCT/GB08/004127 (published as WO 2009/077734) and International Application No. PCT/GB2006/001057 (published as WO 2006/100484).

Methods for forming lipid bilayers are known in the art. Lipid bilayers are commonly formed by the method of Montal and Mueller (Proc. Natl. Acad. Sci. USA., 1972; 69: 3561-3566), in which a lipid monolayer is carried on aqueous solution/air interface past either side of an aperture which is perpendicular to that interface. The lipid is normally added to the surface of an aqueous electrolyte solution by first dissolving it in an organic solvent and then allowing a drop of the solvent to evaporate on the surface of the aqueous solution on either side of the aperture. Once the organic solvent has evaporated, the solution/air interfaces on either side of the aperture are physically moved up and down past the aperture until a bilayer is formed. Planar lipid bilayers may be formed across an aperture in a membrane or across an opening into a recess.

The method of Montal & Mueller is popular because it is a cost-effective and relatively straightforward method of forming good quality lipid bilayers that are suitable for protein pore insertion. Other common methods of bilayer formation include tip-dipping, painting bilayers and patch-clamping of liposome bilayers.

Tip-dipping bilayer formation entails touching the aperture surface (for example, a pipette tip) onto the surface of a test solution that is carrying a monolayer of lipid. Again, the lipid monolayer is first generated at the solution/air interface by allowing a drop of lipid dissolved in organic solvent to evaporate at the solution surface. The bilayer is then formed by the Langmuir-Schaefer process and requires mechanical automation to move the aperture relative to the solution surface.

For painted bilayers, a drop of lipid dissolved in organic solvent is applied directly to the aperture, which is submerged in an aqueous test solution. The lipid solution is spread thinly over the aperture using a paintbrush or an equivalent. Thinning of the solvent results in formation of a lipid bilayer. However, complete removal of the solvent from the bilayer is difficult and consequently the bilayer formed by this method is less stable and more prone to noise during electrochemical measurement.

Patch-clamping is commonly used in the study of biological cell membranes. The cell membrane is clamped to the end of a pipette by suction and a patch of the membrane becomes attached over the aperture. The method has been adapted for producing lipid bilayers by clamping liposomes which then burst to leave a lipid bilayer sealing over the aperture of the pipette. The method requires stable, giant and unilamellar liposomes and the fabrication of small apertures in materials having a glass surface.

Liposomes can be formed by sonication, extrusion or the Mozafari method (Colas et al. (2007) Micron 38:841-847).

In a preferred embodiment, the lipid bilayer is formed as described in International Application No. PCT/GB08/004127 (published as WO 2009/077734). Advantageously in this method, the lipid bilayer is formed from dried lipids. In a most preferred embodiment, the lipid bilayer is formed across an opening as described in WO2009/077734 (PCT/GB08/004127).

A lipid bilayer is formed from two opposing layers of lipids. The two layers of lipids are arranged such that their hydrophobic tail groups face towards each other to form a hydrophobic interior. The hydrophilic head groups of the lipids face outwards towards the aqueous environment on each side of the bilayer. The bilayer may be present in a number of lipid phases including, but not limited to, the liquid disordered phase (fluid lamellar), liquid ordered phase, solid ordered phase (lamellar gel phase, interdigitated gel phase) and planar bilayer crystals (lamellar sub-gel phase, lamellar crystalline phase).

Any lipid composition that forms a lipid bilayer may be used. The lipid composition is chosen such that a lipid bilayer having the required properties, such surface charge, ability to support membrane proteins, packing density or mechanical properties, is formed. The lipid composition can comprise one or more different lipids. For instance, the lipid composition can contain up to 100 lipids. The lipid composition preferably contains 1 to 10 lipids. The lipid composition may comprise naturally-occurring lipids and/or artificial lipids.

The lipids typically comprise a head group, an interfacial moiety and two hydrophobic tail groups which may be the same or different. Suitable head groups include, but are not limited to, neutral head groups, such as diacylglycerides (DG) and ceramides (CM); zwitterionic head groups, such as phosphatidylcholine (PC), phosphatidylethanolamine (PE) and sphingomyelin (SM); negatively charged head groups, such as phosphatidylglycerol (PG); phosphatidylserine (PS), phosphatidylinositol (PI), phosphatic acid (PA) and cardiolipin (CA); and positively charged headgroups, such as trimethylammonium-Propane (TAP). Suitable interfacial moieties include, but are not limited to, naturally-occurring interfacial moieties, such as glycerol-based or ceramide-based moieties. Suitable hydrophobic tail groups include, but are not limited to, saturated hydrocarbon chains, such as lauric acid (n-Dodecanolic acid), myristic acid (n-Tetradecononic acid), palmitic acid (n-Hexadecanoic acid), stearic acid (n-Octadecanoic) and arachidic (n-Eicosanoic); unsaturated hydrocarbon chains, such as oleic acid (cis-9-Octadecanoic); and branched hydrocarbon chains, such as phytanoyl. The length of the chain and the position and number of the double bonds in the unsaturated hydrocarbon chains can vary. The length of the chains and the position and number of the branches, such as methyl groups, in the branched hydrocarbon chains can vary. The hydrophobic tail groups can be linked to the interfacial moiety as an ether or an ester. The lipids may be mycolic acid.

The lipids can also be chemically-modified. The head group or the tail group of the lipids may be chemically-modified. Suitable lipids whose head groups have been chemically-modified include, but are not limited to, PEG-modified lipids, such as 1,2-Diacyl-sn-Glycero-3-Phosphoethanolamine-N-[Methoxy(Polyethylene glycol)-2000]; functionalised PEG Lipids, such as 1,2-Distearoyl-sn-Glycero-3 Phosphoethanolamine-N-[Biotinyl(Polyethylene Glycol)2000]; and lipids modified for conjugation, such as 1,2-Dioleoyl-sn-Glycero-3-Phosphoethanolamine-N-(succinyl) and 1,2-Dipalmitoyl-sn-Glycero-3-Phosphoethanolamine-N-(Biotinyl). Suitable lipids whose tail groups have been chemically-modified include, but are not limited to, polymerisable lipids, such as 1,2-bis(10,12-tricosadiynoyl)-sn-Glycero-3-Phosphocholine; fluorinated lipids, such as 1-Palmitoyl-2-(16-Fluoropalmitoyl)-sn-Glycero-3-Phosphocholine; deuterated lipids, such as 1,2-Dipalmitoyl-D62-sn-Glycero-3-Phosphocholine; and ether linked lipids, such as 1,2-Di-O-phytanyl-sn-Glycero-3-Phosphocholine. The lipids may be chemically-modified or functionalised to facilitate coupling of the polynucleotide.

The amphiphilic layer, for example the lipid composition, typically comprises one or more additives that will affect the properties of the layer. Suitable additives include, but are not limited to, fatty acids, such as palmitic acid, myristic acid and oleic acid; fatty alcohols, such as palmitic alcohol, myristic alcohol and oleic alcohol; sterols, such as cholesterol, ergosterol, lanosterol, sitosterol and stigmasterol; lysophospholipids, such as 1-Acyl-2-Hydroxy-sn-Glycero-3-Phosphocholine; and ceramides.

In another preferred embodiment, the membrane comprises a solid state layer. Solid state layers can be formed from both organic and inorganic materials Including, but not limited to, microelectronic materials, insulating materials such as Si₃N₄, A1₂O₃, and SiO, organic and inorganic polymers such as polyamide, plastics such as Teflon® or elastomers such as two-component addition-cure silicone rubber, and glasses. The solid state layer may be formed from graphene. Suitable graphene layers are disclosed in International Application No. PCT/US2008/010637 (published as WO 2009/035647). If the membrane comprises a solid state layer, the pore is typically present in an amphiphilic membrane or layer contained within the solid state layer, for instance within a hole, well, gap, channel, trench or slit within the solid state layer. The skilled person can prepare suitable solid state/amphiphilic hybrid systems. Suitable systems are disclosed in WO 2009/020682 and WO 2012/005857. Any of the amphiphilic membranes or layers discussed above may be used.

The method is typically carried out using (i) an artificial amphiphilic layer comprising a pore, (ii) an isolated, naturally-occurring lipid bilayer comprising a pore, or (iii) a cell having a pore Inserted therein. The method is typically carried out using an artificial amphiphilic layer, such as an artificial triblock copolymer layer. The layer may comprise other transmembrane and/or intramembrane proteins as well as other molecules in addition to the pore. Suitable apparatus and conditions are discussed below. The method of the invention is typically carried out in vitro.

### Coupling

The polynucleotide is preferably coupled to the membrane comprising the pore. The method may comprise coupling the polynucleotide to the membrane comprising the pore. The polynucleotide is preferably coupled to the membrane using one or more anchors. The polynucleotide may be coupled to the membrane using any known method.

Each anchor comprises a group which couples (or binds) to the polynucleotide and a group which couples (or binds) to the membrane. Each anchor may covalently couple (or bind) to the polynucleotide and/or the membrane. If a Y adaptor and/or a hairpin loop adaptors are used, the polynucleotide is preferably coupled to the membrane using the adaptor(s).

The polynucleotide may be coupled to the membrane using any number of anchors, such as 2, 3, 4 or more anchors. For instance, a polynucleotide may be coupled to the membrane using two anchors each of which separately couples (or binds) to both the polynucleotide and membrane.

The one or more anchors may comprise the one or more helicases and/or the one or more molecular brakes.

If the membrane is an amphiphilic layer, such as a copolymer membrane or a lipid bilayer, the one or more anchors preferably comprise a polypeptide anchor present in the membrane and/or a hydrophobic anchor present In the membrane. The hydrophobic anchor is preferably a lipid, fatty acid, sterol, carbon nanotube, polypeptide, protein or amino acid, for example cholesterol, palmitate or tocopherol. In preferred embodiments, the one or more anchors are not the pore.

The components of the membrane, such as the amphiphilic molecules, copolymer or lipids, may be chemically-modified or functionalised to form the one or more anchors. Examples of suitable chemical modifications and suitable ways of functionalising the components of the membrane are discussed in more detail below. Any proportion of the membrane components may be functionalised, for example at least 0.01%, at least 0.1%, at least 1%, at least 10%, at least 25%, at least 50% or 100%.

The polynucleotide may be coupled directly to the membrane. The one or more anchors used to couple the polynucleotide to the membrane preferably comprise a linker. The one or more anchors may comprise one or more, such as 2, 3, 4 or more, linkers. One linker may be used couple more than one, such as 2, 3, 4 or more, polynucleotides to the membrane.

Preferred linkers include, but are not limited to, polymers, such as polynucleotides, polyethylene glycols (PEGs), polysaccharides and polypeptides. These linkers may be linear, branched or circular. For instance, the linker may be a circular polynucleotide. The polynucleotide may hybridise to a complementary sequence on the circular polynucleotide linker.

The one or more anchors or one or more linkers may comprise a component that can be cut to broken down, such as a restriction site or a photolabile group.

Functionalised linkers and the ways in which they can couple molecules are known in the art. For instance, linkers functionalised with maleimide groups will react with and attach to cysteine residues in proteins. In the context of this invention, the protein may be present in the membrane or may be used to couple (or bind) to the polynucleotide. This is discussed in more detail below.

Crosslinkage of polynucleotides can be avoided using a “lock and key” arrangement. Only one end of each linker may react together to form a longer linker and the other ends of the linker each react with the polynucleotide or membrane respectively. Such linkers are described In International Application No. PCT/GB10/000132 (published as WO 2010/086602).

The use of a linker is preferred in the sequencing embodiments discussed below. If a polynucleotide is permanently coupled directly to the membrane in the sense that it does not uncouple when interacting with the pore (i.e. does not uncouple in step (b) or (e)), then some sequence data will be lost as the sequencing run cannot continue to the end of the polynucleotide due to the distance between the membrane and the pore. If a linker is used, then the polynucleotide can be processed to completion.

The coupling may be permanent or stable. In other words, the coupling may be such that the polynucleotide remains coupled to the membrane when interacting with the pore.

The coupling may be transient. In other words, the coupling may be such that the polynucleotide may decouple from the membrane when interacting with the pore.

For certain applications, such as aptamer detection, the transient nature of the coupling is preferred. If a permanent or stable linker is attached directly to either the 5′ or 3′ end of a polynucleotide and the linker is shorter than the distance between the membrane and the transmembrane pore's channel, then some sequence data will be lost as the sequencing run cannot continue to the end of the polynucleotide. If the coupling is transient, then when the coupled end randomly becomes free of the membrane, then the polynucleotide can be processed to completion. Chemical groups that form permanent/stable or transient links are discussed in more detail below. The polynucleotide may be transiently coupled to an amphiphilic layer or triblock copolymer membrane using cholesterol or a fatty acyl chain. Any fatty acyl chain having a length of from 6 to 30 carbon atom, such as hexadecanoic acid, may be used.

In preferred embodiments, a polynucleotide, such as a nucleic acid, is coupled to an amphiphilic layer such as a triblock copolymer membrane or lipid bilayer. Coupling of nucleic acids to synthetic lipid bilayers has been carried out previously with various different tethering strategies. These are summarised in Table 4 below.

TABLE 4

| Anchor comprising | Type of coupling | Reference |
|---|---|---|
| Thiol | Stable | Yoshina-Ishii, C. and S. G. Boxer (2003). “Arrays of |
| | | mobile tethered vesicles on supported lipid |
| | | bilayers.” J Am Chem Soc 125(13): 3696-7. |
| Biotin | Stable | Nikolov, V., R. Lipowsky, et al. (2007). “Behavior of |
| | | giant vesicles with anchored DNA molecules.” |
| | | Biophys J 92(12): 4356-68 |
| Cholesterol | Transient | Pfeiffer, I. and F. Hook (2004). “Bivalent |
| | | cholesterol-based coupling of oligonucletides to |
| | | lipid membrane assemblies.” J Am Chem Soc |
| | | 126(33): 10224-5 |
| Surfactant (e.g. Lipid, | Stable | van Lengerich, B., R. J. Rawle, et al. “Covalent |
| Palmitate, etc) | | attachment of lipid vesicles to a fluid-supported |
| | | bilayer allows observation of DNA-mediated vesicle |
| | | interactions.” Langmuir 26(11): 8666-72 |

Synthetic polynucleotides and/or linkers may be functionalised using a modified phosphoramidite in the synthesis reaction, which is easily compatible for the direct addition of suitable anchoring groups, such as cholesterol, tocopherol, palmitate, thiol, lipid and biotin groups. These different attachment chemistries give a suite of options for attachment to polynucleotides. Each different modification group couples the polynucleotide in a slightly different way and coupling is not always permanent so giving different dwell times for the polynucleotide to the membrane. The advantages of transient coupling are discussed above.

Coupling of polynucleotides to a linker or to a functionalised membrane can also be achieved by a number of other means provided that a complementary reactive group or an anchoring group can be added to the polynucleotide. The addition of reactive groups to either end of a polynucleotide has been reported previously. A thiol group can be added to the 5′ of ssDNA or dsDNA using T4 polynucleotide kinase and ATPγS (Grant, G. P. and P. Z. Qin (2007). “A facile method for attaching nitroxide spin labels at the 5′ terminus of nucleic acids.” Nucleic Acids Res 35(10): e77). An azide group can be added to the 5′-phosphate of ssDNA or dsDNA using T4 polynucleotide kinase and γ-[2-Azidoethyl]-ATP or γ-[6-Azidohexyl]-ATP. Using thiol or Click chemistry a tether, containing either a thiol, iodoacetamide OPSS or maleimide group (reactive to thiols) or a DIBO (dibenzocyclooxtyne) or alkyne group (reactive to azides), can be covalently attached to the polynucleotide. A more diverse selection of chemical groups, such as biotin, thiols and fluorophores, can be added using terminal transferase to Incorporate modified oligonucleotides to the 3′ of ssDNA (Kumar, A., P. Tchen, et al. (1988). “Nonradioactive labeling of synthetic oligonucleotide probes with terminal deoxynucleotidyl transferase.” Anal Biochem 169(2): 376-82). Streptavidin/biotin and/or streptavidin/desthiobiotin coupling may be used for any other polynucleotide. The Examples below describes how a polynucleotide can be coupled to a membrane using streptavidin/biotin and streptavidin/desthiobiotin. It may also be possible that anchors may be directly added to polynucleotides using terminal transferase with suitably modified nucleotides (e.g. cholesterol or palmitate).

The one or more anchors preferably couple the polynucleotide to the membrane via hybridisation. Hybridisation in the one or more anchors allows coupling in a transient manner as discussed above. The hybridisation may be present in any part of the one or more anchors, such as between the one or more anchors and the polynucleotide, within the one or more anchors or between the one or more anchors and the membrane. For instance, a linker may comprise two or more polynucleotides, such as 3, 4 or 5 polynucleotides, hybridised together. The one or more anchors may hybridise to the polynucleotide. The one or more anchors may hybridise directly to the polynucleotide or directly to a Y adaptor and/or leader sequence attached to the polynucleotide or directly to a hairpin loop adaptor attached to the polynucleotide (as discussed below). Alternatively, the one or more anchors may be hybridised to one or more, such as 2 or 3, intermediate polynucleotides (or “splints”) which are hybridised to the polynucleotide, to a Y adaptor and/or leader sequence attached to the polynucleotide or to a hairpin loop adaptor attached to the polynucleotide (as discussed below).

The one or more anchors may comprise a single stranded or double stranded polynucleotide. One part of the anchor may be ligated to a single stranded or double stranded polynucleotide. Ligation of short pieces of ssDNA have been reported using T4 RNA ligase I (Troutt, A. B., M. G. McHeyzer-Williams, et al. (1992). “Ligation-anchored PCR: a simple amplification technique with single-sided specificity.” Proc Natl Aced Sd USA 89(20): 9823-5). Alternatively, either a single stranded or double stranded polynucleotide can be ligated to a double stranded polynucleotide and then the two strands separated by thermal or chemical denaturation. To a double stranded polynucleotide, it is possible to add either a piece of single stranded polynucleotide to one or both of the ends of the duplex, or a double stranded polynucleotide to one or both ends. For addition of single stranded polynucleotides to the a double stranded polynucleotide, this can be achieved using T4 RNA ligase I as for ligation to other regions of single stranded polynucleotides. For addition of double stranded polynucleotides to a double stranded polynucleotide then ligation can be “blunt-ended”, with complementary 3 dA/dT tails on the polynucleotide and added polynucleotide respectively (as is routinely done for many sample prep applications to prevent concatemer or dimer formation) or using “sticky-ends” generated by restriction digestion of the polynucleotide and ligation of compatible adapters. Then, when the duplex is melted, each single strand will have either a 5′ or 3 modification If a single stranded polynucleotide was used for ligation or a modification at the 5′ end, the 3′ end or both if a double stranded polynucleotide was used for ligation.

If the polynucleotide is a synthetic strand, the one or more anchors can be incorporated during the chemical synthesis of the polynucleotide. For Instance, the polynucleotide can be synthesised using a primer having a reactive group attached to it.

Adenylated polynucleotides are intermediates in ligation reactions, where an adenosine-monophosphate is attached to the 5′-phosphate of the polynucleotide. Various kits are available for generation of this intermediate, such as the 5′ DNA Adenylation Kit from NEB. By substituting ATP in the reaction for a modified nucleotide triphosphate, then addition of reactive groups (such as thiols, amines, biotin, azides, etc) to the 5′ of a polynucleotide can be possible. It may also be possible that anchors could be directly added to polynucleotides using a 5′ DNA adenylation kit with suitably modified nucleotides (e.g. cholesterol or palmitate).

A common technique for the amplification of sections of genomic DNA is using polymerase chain reaction (PCR). Here, using two synthetic oligonucleotide primers, a number of copies of the same section of DNA can be generated, where for each copy the 5′ of each strand in the duplex will be a synthetic polynucleotide. Single or multiple nucleotides can be added to 3′ end of single or double stranded DNA by employing a polymerase. Examples of polymerases which could be used include, but are not limited to, Terminal Transferase. Klenow and E. coli Poly(A) polymerase). By substituting ATP in the reaction for a modified nucleotide triphosphate then anchors, such as a cholesterol, thiol, amine, azide, biotin or lipid, can be incorporated into double stranded polynucleotides. Therefore, each copy of the amplified polynucleotide will contain an anchor.

Ideally, the polynucleotide is coupled to the membrane without having to functionalise the polynucleotide. This can be achieved by coupling the one or more anchors, such as a polynucleotide binding protein or a chemical group, to the membrane and allowing the one or more anchors to interact with the polynucleotide or by functionalising the membrane. The one or more anchors may be coupled to the membrane by any of the methods described herein. In particular, the one or more anchors may comprise one or more linkers, such as maleimide functionalised linkers.

In this embodiment, the polynucleotide is typically RNA, DNA, PNA, TNA or LNA and may be double or single stranded. This embodiment is particularly suited to genomic DNA polynucleotides.

The one or more anchors can comprise any group that couples to, binds to or interacts with single or double stranded polynucleotides, specific nucleotide sequences within the polynucleotide or patterns of modified nucleotides within the polynucleotide, or any other ligand that is present on the polynucleotide.

Suitable binding proteins for use in anchors include, but are not limited to, E. coli single stranded binding protein, P5 single stranded binding protein, T4 gp32 single stranded binding protein, the TOPO V dsDNA binding region, human histone proteins, E. coli HU DNA binding protein and other archaeal, prokaryotic or eukaryotic single stranded or double stranded polynucleotide (or nucleic acid) binding proteins, including those listed below.

The specific nucleotide sequences could be sequences recognised by transcription factors, ribosomes, endonucleases, topoisomerases or replication initiation factors. The patterns of modified nucleotides could be patterns of methylation or damage.

The one or more anchors can comprise any group which couples to, binds to, intercalates with or interacts with a polynucleotide. The group may intercalate or interact with the polynucleotide via electrostatic, hydrogen bonding or Van der Waals interactions. Such groups include a lysine monomer, poly-lysine (which will interact with ssDNA or dsDNA), ethidium bromide (which will intercalate with dsDNA), universal bases or universal nucleotides (which can hybridise with any polynucleotide) and osmium complexes (which can react to methylated bases). A polynucleotide may therefore be coupled to the membrane using one or more universal nucleotides attached to the membrane. Each universal nucleotide may be coupled to the membrane using one or more linkers. The universal nucleotide preferably comprises one of the following nucleobases: hypoxanthine, 4-nitroindole, 5-nitroindole, 6-nitroindole, formylindole, 3-nitropyrrole, nitroimidazole, 4-nitropyrazole, 4-nitrobenzimidazole, 5-nitroindazole, 4-aminobenzimidazole or phenyl (C6-aromatic ring). The universal nucleotide more preferably comprises one of the following nucleosides: 2′-deoxyinosine, inosine, 7-deaza-2′-deoxyinosine, 7-deaza-inosine, 2-aza-deoxyinosine, 2-aza-inosine, 2-O′-methylinosine, 4-nitroindole 2′-deoxyribonucleoside, 4-nitroindole ribonucleoside, 5-nitroindole 2′-deoxyribonucleoside, 5-nitroindole ribonucleoside, 6-nitroindole 2′-deoxyribonucleoside, 6-nitroindole ribonucleoside, 3-nitropyrrole 2′-deoxyribonucleoside, 3-nitropyrrole ribonucleoside, an acyclic sugar analogue of hypoxanthine, nitroimidazole 2′-deoxyribonucleoside, nitroimidazole ribonucleoside, 4-nitropyrazole 2′-deoxyribonucleoside, 4-nitropyrazole ribonucleoside, 4-nitrobenzimidazole 2′-deoxyribonucleoside, 4-nitrobenzimidazole ribonucleoside, 5-nitroindazole 2′-deoxyribonucleoside, 5-nitroindazole ribonucleoside, 4-aminobenzimidazole 2′-deoxyribonucleoside, 4-aminobenzimidazole ribonucleoside, phenyl C-ribonucleoside, phenyl C-2′-deoxyribosyl nucleoside, 2′-deoxynebularine, 2′-deoxyisoguanosine, K-2′-deoxyribose, P-2′-deoxyribose and pyrrolidine. The universal nucleotide more preferably comprises 2′-deoxyinosine. The universal nucleotide is more preferably IMP or dIMP. The universal nucleotide is most preferably dPMP (2′-Deoxy-P-nucleoside monophosphate) or dKMP (N6-methoxy-2,6-diaminopurine monophosphate).

The one or more anchors may couple to (or bind to) the polynucleotide via Hoogsteen hydrogen bonds (where two nucleobases are held together by hydrogen bonds) or reversed Hoogsteen hydrogen bonds (where one nucleobase is rotated through 180° with respect to the other nucleobase). For instance, the one or more anchors may comprise one or more nucleotides, one or more oligonucleotides or one or more polynucleotides which form Hoogsteen hydrogen bonds or reversed Hoogsteen hydrogen bonds with the polynucleotide. These types of hydrogen bonds allow a third polynucleotide strand to wind around a double stranded helix and form a triplex. The one or more anchors may couple to (or bind to) a double stranded polynucleotide by forming a triplex with the double stranded duplex.

In this embodiment at least 1%, at least 10%, at least 25%, at least 50% or 100% of the membrane components may be functionalised.

Where the one or more anchors comprise a protein, they may be able to anchor directly into the membrane without further functionalisation, for example if it already has an external hydrophobic region which is compatible with the membrane. Examples of such proteins include, but are not limited to, transmembrane proteins, intramembrane proteins and membrane proteins. Alternatively the protein may be expressed with a genetically fused hydrophobic region which is compatible with the membrane. Such hydrophobic protein regions are known in the art.

The one or more anchors are preferably mixed with the polynucleotide before contacting with the membrane, but the one or more anchors may be contacted with the membrane and subsequently contacted with the polynucleotide.

In another aspect the polynucleotide may be functionalised, using methods described above, so that it can be recognised by a specific binding group. Specifically the polynucleotide may be functionalised with a ligand such as biotin (for binding to streptavidin), amylose (for binding to maltose binding protein or a fusion protein), Ni-NTA (for binding to poly-histidine or poly-histidine tagged proteins) or a peptides (such as an antigen).

According to a preferred embodiment, the one or more anchors may be used to couple a polynucleotide to the membrane when the polynucleotide is attached to a leader sequence which preferentially threads into the pore. Leader sequences are discussed in more detail below. Preferably, the polynucleotide is attached (such as ligated) to a leader sequence which preferentially threads into the pore. Such a leader sequence may comprise a homopolymeric polynucleotide or an abasic region. The leader sequence is typically designed to hybridise to the one or more anchors either directly or via one or more intermediate polynucleotides (or splints). In such instances, the one or more anchors typically comprise a polynucleotide sequence which is complementary to a sequence in the leader sequence or a sequence in the one or more intermediate polynucleotides (or splints). In such instances, the one or more splints typically comprise a polynucleotide sequence which is complementary to a sequence in the leader sequence.

An example of a molecule used in chemical attachment is EDC (1-ethyl-3-[3-dimethylaminopropyl]carbodiimide hydrochloride). Reactive groups can also be added to the 5′ of polynucleotides using commercially available kits (Thermo Pierce, Part No. 22980). Suitable methods include, but are not limited to, transient affinity attachment using histidine residues and Ni-NTA, as well as more robust covalent attachment by reactive cysteines, lysines or non natural amino acids.

### Double Stranded Polynucleotide

The polynucleotide may be double stranded. If the polynucleotide is double stranded, the method preferably further comprises before the contacting step ligating a bridging moiety, such as a hairpin loop, to one end of the polynucleotide. The two strands of the polynucleotide may then be separated as or before the polynucleotide is contacted with the pore in accordance with the invention The two strands may be separated as the polynucleotide movement through the pore is controlled by a polynucleotide binding protein, such as a helicase, or molecular brake.

Linking and interrogating both strands on a double stranded construct in this way increases the efficiency and accuracy of characterisation.

The bridging moiety is capable of linking the two strands of the target polynucleotide. The bridging moiety typically covalently links the two strands of the target polynucleotide. The bridging moiety can be anything that is capable of linking the two strands of the target polynucleotide, provided that the bridging moiety does not interfere with movement of the single stranded polynucleotide through the transmembrane pore.

The bridging moiety may be linked to the target polynucleotide by any suitable means known in the art. The bridging moiety may be synthesised separately and chemically attached or enzymatically ligated to the target polynucleotide. Alternatively, the bridging moiety may be generated in the processing of the target polynucleotide.

The bridging moiety is linked to the target polynucleotide at or near one end of the target polynucleotide. The bridging moiety is preferably linked to the target polynucleotide within 10 nucleotides of the end of the target polynucleotide

Suitable bridging moieties include, but are not limited to a polymeric linker, a chemical linker, a polynucleotide or a polypeptide. Preferably, the bridging moiety comprises DNA, RNA, modified DNA (such as abasic DNA), RNA, PNA, LNA or PEG. The bridging moiety is more preferably DNA or RNA.

The bridging moiety is most preferably a hairpin loop or a hairpin loop adaptor. Suitable hairpin adaptors can be designed using methods known in the art. The hairpin loop may be any length. The hairpin loop is typically 110 or fewer nucleotides, such as 100 or fewer nucleotides, 90 or fewer nucleotides, 80 or fewer nucleotides, 70 or fewer nucleotides, 60 or fewer nucleotides, 50 or fewer nucleotides, 40 or fewer nucleotides, 30 or fewer nucleotides, 20 or fewer nucleotides or 10 or fewer nucleotides, in length. The hairpin loop is preferably from about 1 to 110, from 2 to 100, from 5 to 80 or from 6 to 50 nucleotides in length. Longer lengths of the hairpin loop, such as from 50 to 110 nucleotides, are preferred if the loop is involved in the differential selectability of the adaptor. Similarly, shorter lengths of the hairpin loop, such as from 1 to 5 nucleotides, are preferred if the loop is not Involved In the selectable binding as discussed below.

The hairpin adaptor may be ligated to either end of the first and/or second polynucleotide, i.e. the 5′ or the 3′ end. The hairpin adaptor may be ligated to the first and/or second polynucleotide using any method known in the art. The hairpin adaptor may be ligated using a ligase, such as T4 DNA ligase, E. coli DNA ligase, Taq DNA ligase, Tma DNA ligase and 9° N DNA ligase.

The two strands of the polynucleotide may be separated using any method known in the art. For instance, they may be separated by a polynucleotide binding protein or using conditions which favour dehybridisation (examples of conditions which favour dehybridisation include, but are not limited to, high temperature, high pH and the addition of agents that can disrupt hydrogen bonding or base pairing, such as formamide and urea).

The hairpin adaptor preferably comprises a selectable binding moiety. This allows the first and/or second polynucleotide to be purified or Isolated. A selectable binding moiety is a moiety that can be selected on the basis of its binding properties. Hence, a selectable binding moiety is preferably a moiety that specifically binds to a surface. A selectable binding moiety specifically binds to a surface if it binds to the surface to a much greater degree than any other moiety used in the invention. In preferred embodiments, the moiety binds to a surface to which no other moiety used in the invention binds.

Suitable selective binding moieties are known in the art. Preferred selective binding moieties include, but are not limited to, biotin, a polynucleotide sequence, antibodies, antibody fragments, such as Fab and ScSv, antigens, polynucleotide binding proteins, poly histidine tails and GST tags. The most preferred selective binding moieties are biotin and a selectable polynucleotide sequence. Biotin specifically binds to a surface coated with avidins. Selectable polynucleotide sequences specifically bind (i.e. hybridise) to a surface coated with homologus sequences Alternatively, selectable polynucleotide sequences specifically bind to a surface coated with polynucleotide binding proteins.

The hairpin adaptor and/or the selectable binding moiety may comprise a region that can be cut, nicked, cleaved or hydrolysed. Such a region can be designed to allow the first and/or second polynucleotide to be removed from the surface to which it is bound following purification or isolation. Suitable regions are known in the art. Suitable regions include, but are not limited to, an RNA region, a region comprising desthiobiotin and streptavidin, a disulphide bond and a photocleavable region.

The double stranded target polynucleotide preferably comprises a leader sequence at the opposite end of the bridging moiety, such as a hairpin loop or hairpin loop adaptor. Leader sequences are discussed in more detail below.

### Round the Corner Sequencing

In a preferred embodiment, a target double stranded polynucleotide is provided with a bridging moiety, such as a hairpin loop or hairpin loop adaptor, at one end and the method comprises contacting the polynucleotide with the pore such that both strands of the polynucleotide move through the pore and taking one or more measurements as the both strands of the polynucleotide move with respect to the pore wherein the measurements are indicative of one or more characteristics of the strands of the polynucleotide and thereby characterising the target double stranded polynucleotide. In another preferred embodiment, a target double stranded polynucleotide is provided with a bridging moiety, such as a hairpin loop or hairpin loop adaptor, at one end and the method comprises contacting the polynucleotide with the pore and exonuclease such that both strands of the polynucleotide are digested to form individual nucleotides. Any of the embodiments discussed above equally apply to this embodiment.

### Leader Sequence

Before the contacting step in the strand characterisation/sequencing method, the method preferably comprises attaching to the polynucleotide a leader sequence which preferentially threads into the pore. The leader sequence facilitates the method of the invention. The leader sequence is designed to preferentially thread into the pore and thereby facilitate the movement of polynucleotide through the pore. The leader sequence can also be used to link the polynucleotide to the one or more anchors as discussed above.

The leader sequence typically comprises a polymer. The polymer is preferably negatively charged. The polymer is preferably a polynucleotide, such as DNA or RNA, a modified polynucleotide (such as abasic DNA), PNA, LNA, polyethylene glycol (PEG) or a polypeptide. The leader preferably comprises a polynucleotide and more preferably comprises a single stranded polynucleotide. The leader sequence can comprise any of the polynucleotides discussed above. The single stranded leader sequence most preferably comprises a single strand of DNA, such as a poly dT section. The leader sequence preferably comprises the one or more spacers.

The leader sequence can be any length, but is typically 10 to 150 nucleotides in length, such as from 20 to 150 nucleotides in length. The length of the leader typically depends on the transmembrane pore used in the method.

The leader sequence is preferably part of a Y adaptor as defined below.

### Double Coupling

The method of the invention may involve double coupling of a double stranded polynucleotide.

In a preferred embodiment, the method of the invention comprises:
(a) providing the double stranded polynucleotide with a Y adaptor at one end and a bridging moiety adaptor, such as a hairpin loop adaptor, at the other end, wherein the Y adaptor comprises one or more first anchors for coupling the polynucleotide to the membrane, wherein the bridging moiety adaptor comprises one or more second anchors for coupling the polynucleotide to the membrane and wherein the strength of coupling of the bridging moiety adaptor to the membrane is greater than the strength of coupling of the Y adaptor to the membrane;
(b) contacting the polynucleotide provided In step (a) with a CsgG pore or mutant thereof, such as a pore the invention, such that the polynucleotide moves with respect to, such as through, the pore; and
(c) taking one or more measurements as the polynucleotide moves with respect to the pore, wherein the measurements are indicative of one or more characteristics of the polynucleotide, and thereby characterising the target polynucleotide.

This type of method is discussed in detail in the UK Application No. 1406147.7.

The double stranded polynucleotide is provided with a Y adaptor at one end and a bridging moiety adaptor at the other end. The Y adaptor and/or the bridging moiety adaptor are typically polynucleotide adaptors. They may be formed from any of the polynucleotides discussed above.

The Y adaptor typically comprises (a) a double stranded region and (b) a single stranded region or a region that is not complementary at the other end. The Y adaptor may be described as having an overhang if it comprises a single stranded region. The presence of a non-complementary region in the Y adaptor gives the adaptor its Y shape since the two strands typically do not hybridise to each other unlike the double stranded portion. The Y adaptor comprises the one or more first anchors. Anchors are discussed in more detail above.

The Y adaptor preferably comprises a leader sequence which preferentially threads into the pore. This is discussed above.

The bridging moiety adaptor preferably comprises a selectable binding moiety as discussed above. The bridging moiety adaptor and/or the selectable binding moiety may compose a region that can be cut, nicked, cleaved or hydrolysed as discussed above.

If one or more helicases and one or more molecular brakes are used as discussed above, the Y adaptor preferably comprises the one or more helicases and the bridging moiety adaptor preferably comprises the one or more molecular brakes.

The Y adaptor and/or the bridging moiety adaptor may be ligated to the polynucleotide using any method known in the art. One or both of the adaptors may be ligated using a ligase, such as T4 DNA ligase, E. coli DNA ligase, Taq DNA ligase, Tma DNA ligase and 9° N DNA ligase. Alternatively, the adaptors may be added to the polynucleotide using the methods of the invention discussed below.

In a preferred embodiment, step a) of the method comprises modifying the double stranded polynucleotide so that it comprises the Y adaptor at one end and the bridging moiety adaptor at the other end. Any manner of modification can be used. The method preferably comprises modifying the double stranded polynucleotide in accordance with the invention. This is discussed in more detail below. The methods of modification and characterisation may be combined in any way.

The strength of coupling (or binding) of the bridging moiety adaptor to the membrane is greater than the strength of coupling (or binding) of the Y adaptor to the membrane. This can be measured in any way. A suitable method for measuring the strength of coupling (or binding) is disclosed in the Examples of the UK Application No. 1406147.7.

The strength of coupling (or binding) of the bridging moiety adaptor is preferably at least 1.5 times the strength of coupling (or binding) of the Y adaptor, such as at least twice, at least three times, at least four times, at least five or at least ten times the strength of coupling (or binding) of the anchor adaptor. The affinity constant (Kd) of the bridging moiety adaptor for the membrane is preferably at least 1.5 times the affinity constant of the Y adaptor, such as at least twice, at least three times, at least four times, at least five or at least ten times the strength of coupling of the Y adaptor.

There are several ways in which the bridging moiety adaptor couples (or binds) more strongly to the membrane than the Y adaptor. For instance, the bridging moiety adaptor may comprise more anchors than the Y adaptor. For instance, the bridging moiety adaptor may comprise 2, 3 or more second anchors whereas the Y adaptor may comprise one first anchor.

The strength of coupling (or binding) of the one or more second anchors to the membrane may be greater than the strength of coupling (or binding) of the one or more first anchors to the membrane. The strength of coupling (or binding) of the one or more second anchors to the bridging moiety adaptor may be greater than the strength of coupling (or binding) of the one or more first anchors to the Y adaptor. The one or more first anchors and the one or more second anchors may be attached to their respective adaptors via hybridisation and the strength of hybridisation is greater in the one or more second anchors than in the one or more first anchors. Any combination of these embodiments may also be used in the invention. Strength of coupling (or binding) may be measure using known techniques in the art.

The one or more second anchors preferably comprise one or more groups which couple(s) (or bind(s)) to the membrane with a greater strength than the one or more groups In the one or more first anchors which couple(s) (or bind(s)) to the membrane. In preferred embodiments, the bridging moiety adaptor/one or more second anchors couple (or bind) to the membrane using cholesterol and the Y adaptor/one or more first anchors couple (or bind) to the membrane using palmitate. Cholesterol binds to triblock copolymer membranes and lipid membranes more strongly than palmitate. In an alternative embodiment, the bridging moiety adaptor/one or more second anchors couple (or bind) to the membrane using a mono-acyl species, such as palmitate, and the Y adaptor/one or more first anchors couple (or bind) to the membrane using a diacyl species, such as dipalmitoylphosphatidylcholine.

### Adding Hairpin Loops and Leader Sequences

Before provision, a double stranded polynucleotide may be contacted with a MuA transposase and a population of double stranded MuA substrates, wherein a proportion of the substrates in the population are Y adaptors comprising the leader sequence and wherein a proportion of the substrates in the population are hairpin loop adaptors. The transposase fragments the double stranded polynucleotide analyte and ligates MuA substrates to one or both ends of the fragments. This produces a plurality of modified double stranded polynucleotides comprising the leader sequence at one end and the hairpin loop at the other. The modified double stranded polynucleotides may then be investigated using the method of the invention.

Each substrate in the population preferably comprises at least one overhang of universal nucleotides such that the transposase fragments the template polynucleotide and ligates a substrate to one or both ends of the double stranded fragments and thereby produces a plurality of fragment/substrate constructs and wherein the method further comprises ligating the overhangs to the fragments in the constructs and thereby producing a plurality of modified double stranded polynucleotides. Suitable universal nucleotides are discussed above. The overhang is preferably five nucleotides in length.

Alternatively, each substrate in population preferably comprises (i) at least one overhang and (ii) at least one nucleotide in the same strand as the at least one overhang which comprises a nucleoside that is not present in the template polynucleotide such that the transposase fragments the template polynucleotide and ligates a substrate to one or both ends of the double stranded fragments and thereby produces a plurality of fragment/substrate constructs, and wherein the method further comprises (a) removing the overhangs from the constructs by selectively removing the at least one nucleotide and thereby producing a plurality of double stranded constructs comprising single stranded gaps and (b) repairing the single stranded gaps in the constructs and thereby producing a plurality of modified double stranded polynucleotides. The polynucleotide typically comprises the nucleosides deoxyadenosine (dA), deoxyuridine (dU) and/or thymidine (dT), deoxyguanosine (dG) and deoxycytidine (dC). The nucleoside that is not present in the polynucleotide is preferably abasic, adenosine (A), uridine (U), 5-methyluridine (m⁵U), cytidine (C) or guanosine (G) or comprises urea, 5,6 dihydroxythymine, thymine glycol, 5-hydroxy-5 methylhydanton, uracil glycol, 6-hydroxy-5,6-dihdrothimine, methyltartronylurea, 7,8-dihydro-8-oxoguanine (8-oxoguanine), 8-oxoadenine, fapy-guanine, methy-fapy-guanine, fapy-adenine, aflatoxin B1-fapy-guanine, 5-hydroxy-cytosine, 5-hydroxy-uracil, 3-methyladenine, 7-methylguanine, 1,N6-ethenoadenine, hypoxanthine, 5-hydroxyuracil, 5-hydroxymethyluracil, 5-formyluracil or a cis-syn-cyclobutane pyrimidine dimer. The at least one nucleotide preferably is 10 nucleotides or fewer from the overhang. The at least one nucleotide is the first nucleotide in the overhang. All of the nucleotides in the overhang preferably comprise a nucleoside that is not present in the template polynucleotide.

These MuA based methods are disclosed in International Application No. PCT/GB2014/052505. They are also discussed in detail in the UK Application No. 1406147.7.

One or more helicases may be attached to the MuA substrate Y adaptors before they are contacted with the double stranded polynucleotide and MuA transposase. Alternatively, one or more helicases may be attached to the MuA substrate Y adaptors before they are contacted with the double stranded polynucleotide and MuA transposase.

One or more molecular brakes may be attached to the MuA substrate hairpin loop adaptors before they are contacted with the double stranded polynucleotide and MuA transposase. Alternatively, one or more molecular brakes may be attached to the MuA substrate hairpin loop adaptors before they are contacted with the double stranded polynucleotide and MuA transposase.

### Uncoupling

The method of the invention may involve characterising multiple target polynucleotides and uncoupling of the at least the first target polynucleotide.

In a preferred embodiment, the Invention involves characterising two or more target polynucleotides. The method comprises:
(a) providing a first polynucleotide in a first sample;
(b) providing a second polynucleotide in a second sample;
(c) coupling the first polynucleotide in the first sample to a membrane using one or more anchors;
(d) contacting the first polynucleotide with CsgG pore or mutant thereof, such as a pore of the invention, such that the polynucleotide moves with respect to, such as through, the pore;
(e) taking one or more measurements as the first polynucleotide moves with respect to the pore wherein the measurements are indicative of one or more characteristics of the first polynucleotide and thereby characterising the first polynucleotide;
(f) uncoupling the first polynucleotide from the membrane;
(g) coupling the second polynucleotide in the second sample to the membrane using one or more anchors;
(h) contacting the second polynucleotide with the CsgG pore or mutant thereof, such as a pore of the invention, such that the second polynucleotide moves with respect to, such as through, the pore; and
(i) taking one or more measurements as the second polynucleotide moves with respect to the pore wherein the measurements are Indicative of one or more characteristics of the second polynucleotide and thereby characterising the second polynucleotide.

This type of method is discussed in detail in the UK Application No. 1406155.0.

Step (f) (i.e. uncoupling of the first polynucleotide) may be performed before step (g) (i.e. before coupling the second polynucleotide to the membrane). Step (g) may be performed before step (f). If the second polynucleotide is coupled to the membrane before the first polynucleotide is uncoupled, step (f) preferably comprises selectively uncoupling the first polynucleotide from the membrane (i.e. uncoupling the first polynucleotide but not the second polynucleotide from the membrane). A skilled person can design a system in which selective uncoupling is achieved. Steps (f) and (g) may be performed at the same time. This is discussed in more detail below.

In step (f), at least 10% of the first polynucleotide is preferably uncoupled from the membrane. For instance, at least 20%, at least 30%, at least 40%, at least 50%, at least 60%, at least 70%, at least 80%, at least 90% or at least 95% of the first polynucleotide may be uncoupled from the membrane. Preferably, all of the first polynucleotide is uncoupled from the membrane. The amount of the first polynucleotide uncoupled from the membrane can be determined using the pore. This is disclosed in the Examples.

The first polynucleotide and second polynucleotide may be different from one another. Alternatively, the first and second polynucleotides may be different polynucleotides. In such instances, there may be no need to remove at least part of the first sample before adding the second polynucleotide. This is discussed in more detail below. If the method concerns investigating three or more polynucleotides, they may all be different from one another or some of them may be different from one another.

The first polynucleotide and the second polynucleotide may be two Instances of the same polynucleotide. The first polynucleotide may be identical to the second polynucleotide. This allows proof reading. If the method concerns investigating three or more polynucleotides, they may all be three or more instances of the same polynucleotide or some of them may be separate instances of the same polynucleotide.

The first sample and second sample may be different from one another. For instance, the first sample may be derived from a human and the second sample may be derived from a virus. If the first and second samples are different from one another, they may contain or be suspected of containing the same first and second polynucleotides. If the method concerns investigating three or more samples, they may all be different from one another or some of them may be different from one another.

The first sample and the second sample are preferably two instances of the same sample. The first sample is preferably Identical to the second sample. This allows proof reading. If the method concerns investigating three or more samples, they may all be three or more instances of the same sample or some of them may be separate instances of the same sample.

Any number of polynucleotides can be investigated. For instance, the method of the invention may concern characterising 3, 4, 5, 6, 7, 8, 9, 10, 20, 30, 50, 100 or more polynucleotides. If three or more polynucleotides are investigated using the method of the invention, the second polynucleotide is also uncoupled from the membrane and the requisite number of steps are added for the third polynucleotide. The same is true for four or more polynucleotides.

The method of the invention involves uncoupling the first polynucleotide from the membrane. The method of the invention may involve uncoupling the second polynucleotide from the membrane if three or more polynucleotides are being investigated.

The first polynucleotide can be uncoupled from the membrane using any known method. The first polynucleotide is preferably not uncoupled from the membrane in step (f) using the transmembrane pore. The first polynucleotide is preferably not uncoupled from the membrane using a voltage or an applied potential.

Step (f) preferably comprises uncoupling the first polynucleotide from the membrane by removing the one or more anchors from the membrane. If the anchors are removed, the second polynucleotide is coupled to the membrane using other (or separate) anchors. The anchors used to couple the second polynucleotide may be the same type of anchors used to couple the first polynucleotide or different type of anchors.

Step (f) more preferably comprises contacting the one or more anchors with an agent which has a higher affinity for the one or more anchors than the anchors have for the membrane. A variety of protocols for competitive binding or immunoradiometric assays to determine the specific binding capability of molecules are well known in the art (see for example Maddox et al, J. Exp. Med. 1, 1211-1226, 1993). The agent removes the anchor(s) from the membrane and thereby uncouples the first polynucleotide. The agent is preferably a sugar. Any sugar which binds to the one or more anchors with a higher affinity than the one or more anchors have for the membrane may be used. The sugar may be a cyclodextrin or derivative thereof as discussed below.

If one or more anchors comprise a hydrophobic anchor, such as cholesterol, the agent is preferably a cyclodextrin or a derivative thereof or a lipid. The cyclodextrin or derivative thereof may be any of those disclosed in Eliseev, A. V., and Schneider, H-J. (1994) J. Am. Chem. Soc. 116, 6081-6088. The agent is more preferably heptakis-6-amino-β-cyclodextrin (am₇-βCD), 6-monodeoxy-6-monoamino-β-cyclodextrin (am₁-□CD) or heptakis-(6-deoxy-6-guanidino)-cyclodextrin (gu₇-βCD). Any of the lipids disclosed herein may be used.

If an anchor comprise(s) streptavidin, biotin or desthiobiotin, the agent is preferably biotin, desthiobiotin or streptavidin. Both biotin and desthiobiotin bind to streptavidin with a higher affinity than streptavidin binds to the membrane and vice versa. Biotin has a stronger affinity for streptavidin than desthiobiotin. An anchor comprising streptavidin may therefore be removed from the membrane using biotin or streptavidin and vice versa.

If an anchor comprises a protein, the agent is preferably an antibody or fragment thereof which specifically binds to the protein. An antibody specifically binds to a protein if it binds to the protein with preferential or high affinity, but does not bind or binds with only low affinity to other or different proteins. An antibody binds with preferential or high affinity if it binds with a Kd of 1×10⁻⁶ M or less, more preferably 1×10⁻⁷ M or less, 5×10⁻⁸ M or less, more preferably 1×10⁻⁸ M or less or more preferably 5×10⁻⁹ M or less. An antibody binds with low affinity if it binds with a Kd of 1×10⁻⁶ M or more, more preferably 1×10⁻⁵ M or more, more preferably 1×10⁻⁴ M or more, more preferably 1×10⁻³ M or more, even more preferably 1×10⁻² M or more. Any method may be used to detect binding or specific binding. Methods of quantitatively measuring the binding of an antibody to a protein are well known in the art. The antibody may be a monoclonal antibody or a polyclonal antibody. Suitable fragments of antibodies include, but are not limited to, Fv, F(ab′) and F(ab′)₂ fragments, as well as single chain antibodies. Furthermore, the antibody or fragment thereof may be a chimeric antibody or fragment thereof, a CDR-grafted antibody or fragment thereof or a humanised antibody or fragment thereof.

Step (f) preferably comprises contacting the one or more anchors with an agent which reduces ability of the one or more anchors to couple to the membrane. For instance, the agent could interfere with the structure and/or hydrophobicity of the one or more anchors and thereby reduce their ability to couple to the membrane. If an anchor comprises cholesterol, the agent is preferably cholesterol dehydrogenase. If an anchor comprises a lipid, the agent is preferably a phospholipase. If an anchor comprises a protein, the agent is preferably a proteinase or urea. Other combination of suitable anchors and agents will be clear to a person skilled in the art.

Step (f) preferably comprises uncoupling the first polynucleotide from the membrane by separating the first polynucleotide from the one or more anchors. This can be done in any manner. For instance, the linker could be cut in an anchor comprising a linker This embodiment is particularly applicable to anchors which involve linkage via hybridisation. Such anchors are discussed above.

Step (f) more preferably comprises uncoupling the first polynucleotide from the membrane by contacting the first polynucleotide and the one or more anchors with an agent which competes with the first polynucleotide for binding to one or more anchors. Methods for determining and measuring competitive binding are known in the art. The agent is preferably a polynucleotide which competes with the first polynucleotide for hybridisation to the one or more anchors. For instance, if the first polynucleotide is coupled to the membrane using one or more anchors which involve hybridisation, the polynucleotide can be uncoupled by contacting the one or more anchors with a polynucleotide which also hybridises to the site of hybridisation. The polynucleotide agent is typically added at a concentration that is higher than the concentration of the first polynucleotide and one or more anchors. Alternatively, the polynucleotide agent may hybridise more strongly to the one or more anchors than the first polynucleotide.

Step (f) more preferably comprises (i) contacting the first polynucleotide and the one or more anchors with urea, tris(2-carboxyethyl)phosphine (TCEP), dithiothreitol (DTT), streptavidin or biotin, UV light, an enzyme or a binding agent; (ii) heating the first polynucleotide and the one or more anchors; or (iii) altering the pH. Urea, tris(2-carboxyethyl)phosphine (TCEP) or dithiothreitol (DTT) are capable of disrupting anchors and separating the first polynucleotide from the membrane. If an anchor comprises a streptavidin-biotin link, then a streptavidin agent will compete for binding to the biotin. If an anchor comprises a streptavidin-desthiobiotin link, then a biotin agent will compete for binding to the streptavidin. UV light can be used to breakdown photolabile groups. Enzymes and binding agents can be used to cut, breakdown or unravel the anchor. Preferred enzymes include, but are not limited to, an exonuclease, an endonuclease or a helicase. Preferred binding agents include, but are not limited to, an enzyme, an antibody or a fragment thereof or a single-stranded binding protein (SSB). Any of the enzymes discussed below or antibodies discussed above may be used. Heat and pH can be used to disrupt hybridisation and other linkages.

If the first polynucleotide is uncoupled from the membrane by separating the first polynucleotide from the one or more anchors, the one or more anchors will remain in the membrane. Step (g) preferably comprises coupling the second polynucleotide to the membrane using the one or more anchors that was separated from the first polynucleotide. For instance, the second polynucleotide may also be provided with one or more polynucleotides which hybridise(s) to the one or more anchors that remain in the membrane. Alternatively, step (g) preferably comprises coupling the second polynucleotide to the membrane using one or more separate anchors from the ones separated from the first polynucleotide (i.e. one or more other anchors). The one or more separate anchors may be the same type of anchors used to couple the first polynucleotide to the membrane or may be different types of anchors. Step (g) preferably comprises coupling the second polynucleotide to the membrane using one or more different anchors from the one or more anchors separated from the first polynucleotide.

In a preferred embodiment, steps (f) and (g) comprise uncoupling the first polynucleotide from the membrane by contacting the membrane with the second polynucleotide such that the second polynucleotide competes with the first polynucleotide for binding to the one or more anchors and replaces the first polynucleotide. For instance, if the first polynucleotide is coupled to the membrane using one or more anchors which involve hybridisation, the first polynucleotide can be uncoupled by contacting the anchors with the second polynucleotide attached to polynucleotides which also hybridise to the sites of hybridisation in the one or more anchors. The second polynucleotide is typically added at a concentration that is higher than the concentration of the first polynucleotide and the one or more anchors. Alternatively, the second polynucleotide may hybridise more strongly to the one or more anchors than the first polynucleotide.

### Removal or Washing

Although the first polynucleotide is uncoupled from the membrane in step (f), it is not necessarily removed or washed away. If the second polynucleotide can be easily distinguished from the first polynucleotide, there is no need to remove the first polynucleotide.

Between steps (f) and (g), the method preferably further comprises removing at least some of the first sample from the membrane. At least 10% of the first sample may be removed, such as at least 20%, at least 30%, at least 40%, at least 50%, at least 60%, at least 70%, at least 80% or at least 90% of the first sample may be removed.

The method more preferably further comprises removing all of the first sample from the membrane. This can be done in any way. For instance, the membrane can be washed with a buffer after the first polynucleotide has been uncoupled. Suitable buffers are discussed below.

### Modified Polynucleotides

Before characterisation, a target polynucleotide may be modified by contacting the polynucleotide with a polymerase and a population of free nucleotides under conditions in which the polymerase forms a modified polynucleotide using the target polynucleotide as a template, wherein the polymerase replaces one or more of the nucleotide species in the target polynucleotide with a different nucleotide species when forming the modified polynucleotide. The modified polynucleotide may then be provided with one or more helicases attached to the polynucleotide and one or more molecular brakes attached to the polynucleotide. This type of modification is described in UK Application No. 1403096.9. Any of the polymerases discussed above may be used. The polymerase is preferably Klenow or 9° North.

The template polynucleotide is contacted with the polymerase under conditions in which the polymerase forms a modified polynucleotide using the template polynucleotide as a template. Such conditions are known in the art. For instance, the polynucleotide is typically contacted with the polymerase in commercially available polymerase buffer, such as buffer from New England Biolabs®. The temperature is preferably from 20 to 37° C. for Klenow or from 60 to 75° C. for 9° North. A primer or a 3′ hairpin is typically used as the nucleation point for polymerase extension.

Characterisation, such as sequencing, of a polynucleotide using a transmembrane pore typically involves analyzing polymer units made up of k nucleotides where k is a positive integer (i.e. ‘k-mers’). This is discussed in International Application No. PCT/GB2012/052343 (published as WO 2013/041878). While it is desirable to have clear separation between current measurements for different k-mers, it is common for some of these measurements to overlap. Especially with high numbers of polymer units in the k-mer, i.e. high values of k, it can become difficult to resolve the measurements produced by different k-mers, to the detriment of deriving information about the polynucleotide, for example an estimate of the underlying sequence of the polynucleotide.

By replacing one or more nucleotide species in the target polynucleotide with different nucleotide species in the modified polynucleotide, the modified polynucleotide contains k-mers which differ from those in the target polynucleotide. The different k-mers In the modified polynucleotide are capable of producing different current measurements from the k-mers in the target polynucleotide and so the modified polynucleotide provides different information from the target polynucleotide. The additional information from the modified polynucleotide can make it easier to characterise the target polynucleotide. In some instances, the modified polynucleotide itself may be easier to characterise. For instance, the modified polynucleotide may be designed to include k-mers with an increased separation or a clear separation between their current measurements or k-mers which have a decreased noise.

The polymerase preferably replaces two or more of the nucleotide species in the target polynucleotide with different nucleotide species when forming the modified polynucleotide. The polymerase may replace each of the two or more nucleotide species in the target polynucleotide with a distinct nucleotide species. The polymerase may replace each of the two or more nucleotide species in the target polynucleotide with the same nucleotide species.

If the target polynucleotide is DNA, the different nucleotide species in the modified typically comprises a nucleobase which differs from adenine, guanine, thymine, cytosine or methylcytosine and/or comprises a nucleoside which differs from deoxyadenosine, deoxyguanosine, thymidine, deoxycytidine or deoxymethylcytidine. If the target polynucleotide is RNA, the different nucleotide species in the modified polynucleotide typically comprises a nucleobase which differs from adenine, guanine, uracil, cytosine or methylcytosine and/or comprises a nucleoside which differs from adenosine, guanosine, uridine, cytidine or methylcytidine. The different nucleotide species may be any of the universal nucleotides discussed above.

The polymerase may replace the one or more nucleotide species with a different nucleotide species which comprises a chemical group or atom absent from the one or more nucleotide species. The chemical group may be a propynyl group, a thio group, an oxo group, a methyl group, a hydroxymethyl group, a formyl group, a carboxy group, a carbonyl group, a benzyl group, a propargyl group or a propargylamine group.

The polymerase may replace the one or more nucleotide species with a different nucleotide species which lacks a chemical group or atom present in the one or more nucleotide species. The polymerase may replace the one or more of the nucleotide species with a different nucleotide species having an altered electronegativity. The different nucleotide species having an altered electronegativity preferably comprises a halogen atom.

The method preferably further comprises selectively removing the nucleobases from the one or more different nucleotides species in the modified polynucleotide.

### Analyte Delivery

The target analyte is preferably attached to a microparticle which delivers the analyte towards the membrane. This type of delivery is disclosed in UK Application No. 1418469.1.

Any type of microparticle and attachment method may be used.

### Other Characterisation Method

In another embodiment, a polynucleotide is characterised by detecting labelled species that are added to the target polynucleotide by a polymerase and then released. The polymerase uses the polynucleotide as a template. Each labelled species is specific for each nucleotide. The polynucleotide is contacted with a CsgG pore or mutant thereof, such as a pore of the invention, a polymerase and labelled nucleotides such that phosphate labelled species are sequentially added to the polynucleotide by the polymerase, wherein the phosphate species contain a label specific for each nucleotide. The labelled species may be detected using the pore before they are released from the nucleotides (i.e. as they are added to the target polynucleotide) or after they are released from the nucleotides.

The polymerase may be any of those discussed above. The phosphate labelled species are detected using the pore and thereby characterising the polynucleotide. This type of method is disclosed in European Application No. 13187149.3 (published as EP 2682460). Any of the embodiments discussed above equally apply to this method.

Examples of labelled species include, but are not limited to, polymers, polyethylene gycols, sugars, cyclodextrins, fluorophores, drugs, metabolites, peptides. A non-limiting example of such tags can be found in the work of Kumar et al. Sci Rep. 2012; 2:684. Epub 2012 Sep. 21.

### Methods of Forming Sensors

The invention also provides a method of forming a sensor for characterising a target polynucleotide. The method comprises forming a complex between a CsgG pore or mutant thereof, such as a pore of the invention, and a polynucleotide binding protein, such as a helicase or an exonuclease. The complex may be formed by contacting the pore and the protein in the presence of the target polynucleotide and then applying a potential across the pore. The applied potential may be a chemical potential or a voltage potential as described above. Alternatively, the complex may be formed by covalently attaching the pore to the protein. Methods for covalent attachment are known in the art and disclosed, for example, in International Application Nos. PCT/GB09/001679 (published as WO 2010/004265) and PCT/GB10/000133 (published as WO 2010/086603). The complex is a sensor for characterising the target polynucleotide. The method preferably comprises forming a complex between a CsgG pore or mutant thereof, such as a pore of the invention, and a helicase. Any of the embodiments discussed above equally apply to this method.

The invention also provides a sensor for characterising a target polynucleotide. The sensor comprises a complex between a CsgG pore or mutant thereof, such as a pore of the invention, and a polynucleotide binding protein. Any of the embodiments discussed above equally apply to the sensor of the invention.

### Kits

The present invention also provides a kit for characterising a target polynucleotide. The kit comprises a CsgG pore or mutant thereof, such as a pore of the invention, and the components of a membrane. The membrane is preferably formed from the components. The pore is preferably present in the membrane. The kit may comprise components of any of the membranes disclosed above, such as an amphiphilic layer or a triblock copolymer membrane.

The kit may further comprise a polynucleotide binding protein. Any of the polynucleotide binding proteins discussed above may be used.

The kit may further comprise one or more anchors for coupling the polynucleotide to the membrane.

The kit is preferably for characterising a double stranded polynucleotide and preferably comprises a Y adaptor and a hairpin loop adaptor. The Y adaptor preferably has one or more helicases attached and the hairpin loop adaptor preferably has one or more molecular brakes attached. The Y adaptor preferably comprises one or more first anchors for coupling the polynucleotide to the membrane, the hairpin loop adaptor preferably comprises one or more second anchors for coupling the polynucleotide to the membrane and the strength of coupling of the hairpin loop adaptor to the membrane is preferably greater than the strength of coupling of the Y adaptor to the membrane.

The kit of the Invention may additionally comprise one or more other reagents or instruments which enable any of the embodiments mentioned above to be carried out. Such reagents or instruments include one or more of the following: suitable buffer(s) (aqueous solutions), means to obtain a sample from a subject (such as a vessel or an instrument comprising a needle), means to amplify and/or express polynucleotides or voltage or patch clamp apparatus. Reagents may be present in the kit in a dry state such that a fluid sample resuspends the reagents. The kit may also, optionally, comprise instructions to enable the kit to be used in the method of the invention or details regarding for which organism the method may be used.

### Apparatus

The invention also provides an apparatus for characterising a target analyte, such as a target polynucleotide. The apparatus comprises a plurality of CsgG pores or mutants thereof and a plurality of membranes. The plurality of pores are preferably present in the plurality of membranes. The number of pores and membranes is preferably equal. Preferably, a single pore is present in each membrane.

The apparatus preferably further comprises instructions for carrying out the method of the invention. The apparatus may be any conventional apparatus for analyte analysis, such as an array or a chip. Any of the embodiments discussed above with reference to the methods of the invention are equally applicable to the apparatus of the invention. The apparatus may further comprise any of the features present in the kit of the Invention.

The apparatus is preferably set up to carry out the method of the invention.

The apparatus preferably comprises:
a sensor device that is capable of supporting the plurality of pores and membranes and being operable to perform analyte characterisation using the pores and membranes; and
at least one port for delivery of the material for performing the characterisation.

Alternatively, the apparatus preferably comprises:
a sensor device that is capable of supporting the plurality of pores and membranes being operable to perform analyte characterisation using the pores and membranes; and
at least one reservoir for holding material for performing the characterisation.

The apparatus more preferably comprises:
a sensor device that is capable of supporting the membrane and plurality of pores and membranes and being operable to perform analyte characterising using the pores and membranes;
at least one reservoir for holding material for performing the characterising;
a fluidics system configured to controllably supply material from the at least one reservoir to the sensor device; and
one or more containers for receiving respective samples, the fluidics system being configured to supply the samples selectively from one or more containers to the sensor device.

The apparatus may be any of those described in International Application No. PCT/GB08/004127 (published as WO 2009/077734), PCT/GB10/000789 (published as WO 2010/122293), International Application No. PCT/GB10/002206 (published as WO 2011/067559) or International Application No. PCT/US99/25679 (published as WO 00/28312).

The following Examples Illustrate the Invention.

### EXAMPLES

### Example 1: Cloning and Strains of CsgG

Expression constructs for the production of outer membrane localized C-terminally StrepII-tagged CsgG (pPG1) and periplasmic C-terminally StrepII-tagged CsgG_{C1S} (pPG2) have been described (Goyal. P. et al., Acta Crystallogr. F. Struct. Biol. Cryst. Commun. 2013, 69, 1349-1353). For selenomethionine labelling, StrepII-tagged CsgG_{C1S} was expressed in the cytoplasm because of increased yields. Therefore, pPG2 was altered to remove the N-terminal signal peptide using inverse PCR with primers 5′-TCT TTA AC CGC CCC GCC TAA AG-3′ (forward) (SEQ ID NO: 437) and 5′-CAT TTT TTG CCC TCG TTA TC-3′ (reverse) (pPG3) (SEQ ID NO: 438). For phenotypic assays, a csgG deletion mutant of E. coli BW25141 (E. coli NVG2) was constructed by the method described [Datsenko, K. A. et al, Proc. Natl Acad. Sci. USA 97, 6640-6645 (2000)](with primers 5′-AAT AAC TCA ACC GAT TTT TAA GCC CCA GCT TCA TAA GGA AAA TAA TCG TGT AGG CTG GAG CTG CTT C-3′ (SEQ ID NO: 439) and 5′-CGC TTA AAC AGT AAA ATG CCG GAT GAT AAT TCC GGC TTT TTT ATC TGC ATA TGA ATA TCC TCC TTA G-3′ (SEQ ID NO: 440)). The various CsgG substitution mutants used for Cys accessibility assays and for phenotypic probing of the channel constriction were constructed by site-directed mutagenesis (QuikChange protocol; Stratagene) starting from pMC2, a pTRC99a vector containing csgG under control of the trc promoter (Robinson. L. S., et al, Mol. Microbiol., 2006, 59, 870-881).

### Example 2: Protein Expression and Purification

CsgG and CsgG_{C1S} were expressed and purified as described (Robinson, L. S., et al, Mol. Microbiol., 2006, 59, 870-881). In brief, CsgG was recombinantly produced In E. coli BL 21 (DE3) transformed with pPG1 and extracted from isolated outer membranes with the use of 1% n-dodecyl-β-D-maltoside (DDM) in buffer A (50 mM Tris-HCl pH 8.0, 500 mM NaCl, 1 mM EDTA, 1 mM dithiothreitol (DTT)). StrepII-tagged CsgG was loaded onto a 5 ml Strep-Tactin Sepharose column (Iba GmbH) and detergent-exchanged by washing with 20 column volumes of buffer A supplemented with 0.5% tetraethylene glycol monooctyl ether (C8E4; Affymetrix) and 4 mM lauryldimethylamine-N-oxide (LDAO; Affymetrix). The protein was eluted by the addition of 2.5 mM D-desthiobiotin and concentrated to 5 mg ml⁻¹ for crystallization experiments. For selenomethionine labelling, CsgG_{C1S} was produced In the Met auxotrophic strain B834 (DE3) transformed with pPG3 and grown on M9 minimal medium supplemented with 40 mg l⁻¹ L-selenomethionine. Cell pellets were resuspended in 50 mM Tris-HCl pH 8.0, 150 mM NaCl, 1 mM EDTA, 5 mM DTT, supplemented with cOmplete Protease Inhibitor Cocktail (Roche) and disrupted by passage through a TS series cell disruptor (Constant Systems Ltd) operated at 20×10³ lb in⁻². Labelled CsgG_{C1S} was purified as described (Robinson. L. S., et al, Mol. Microbiol., 2006, 59, 870-881). DTT (5 mM) was added throughout the purification procedure to avoid oxidation of selenomethionine.

CsgE was produced in E. coli NEBC2566 cells harbouring pNH27 (Nenninger, A. A. et al., Mol. Microbiol. 2011, 81, 486-499). Cell lysates in 25 mM Tris-HCl pH 8.0, 150 mM NaCl, 25 mM imidazole, 5% (v/v) glycerol were loaded on a HisTrap FF (GE Healthcare). CsgE-his was eluted with a linear gradient to 500 mM imidazole in 20 mM Tris-HCl pH 8.0, 150 mM NaCl, 5% (v/v) glycerol buffer Fractions containing CsgE were supplemented with 250 mM (NH₄)₂SO₄ and applied to a 5 ml HiTrap Phenyl HP column (GE Healthcare) equilibrated with 20 mM Tris-HCl pH 8.0, 100 mM NaCl, 250 mM (NH₄)₂SO₄, 5% (v/v) glycerol. A linear gradient to 20 mM Tris-HCl pH 8.0, 10 mM NaCl, 5% (v/v) glycerol was applied for elution. CsgE containing fractions were loaded onto a Superose 6 Prep Grade 10/600 (GE Healthcare) column equilibrated in 20 mM Tris-HCl pH 8.0, 100 mM NaCl, 5% (v/v) glycerol.

### Example 3: In-Solution Oligomeric State Assessment

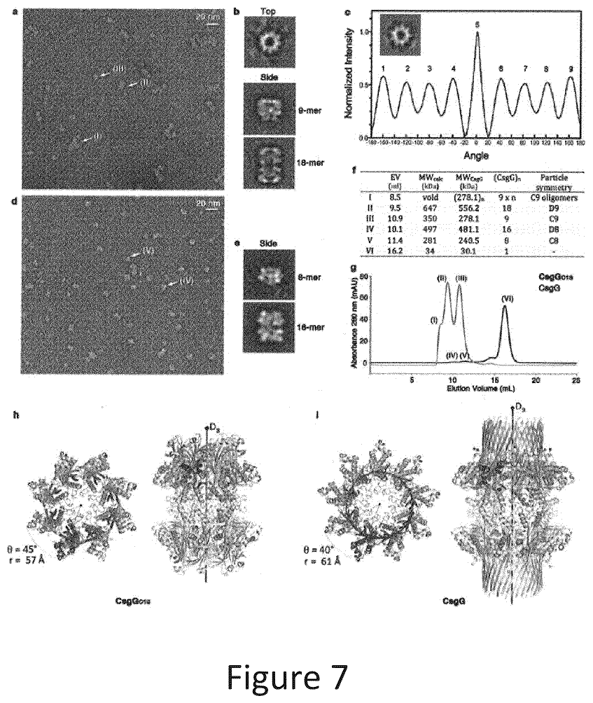

About 0.5 mg each of detergent-solubilized CsgG (0.5% C8E4, 4 mM LDAO) and CsgG_{C1S} were applied to a Superdex 200 10/300 GL analytical gel filtration column (GE Healthcare) equilibrated with 25 mM Tris-HCl pH 8.0, 500 mM NaCl, 1 mM DTT, 4 mM LDAO and 0.5% C8E4 (CsgG) or with 25 mM Tris-HCl pH 8.0, 200 mM NaCl (CsgG_{C1S}), and run at 0.7 ml min⁻¹. The column elution volumes were calibrated with bovine thyroglobulin, bovine γ-globulin, chicken ovalbumin, horse myoglobulin and vitamin B₁₂ (Bio-Rad) (FIG. 7). Membrane-extracted CsgG. 20 μg of the detergent-solubilized protein was also run on 3-10% blue native PAGE using the procedure described in Swamy, M., et al., Sci. STKE 2006, pl 4, [http://dx.doi.org/10.1126/stke.3452006pl4(2006)](FIG. 7). NativeMark (Life Technologies) unstained protein standard (7 μl) was used for molecular mass estimation. Mature CsgG is predominantly found as discrete nonameric poreforming particles with C9 symmetry, as well as tail-to-tail dimers of nonameric pores (i.e. octadecamers with D9 symmetry). For the purpose of nanopore sensing applications, a preferred state of the proteins is a single nonameric pore. The population of nonameric versus D9 octadecameric pores can be increased by heating samples prior to size exclusion chromatography and/or insertion in a lipid bilayer for nanopore sensing applications.

### Example 4: Crystallization, Data Collection and Structure Determination

Selenomethionine-labelled CsgG_{C1S} was concentrated to 3.8 mg ml⁻¹ and crystallized by sitting-drop vapour diffusion against a solution containing 100 mM sodium acetate pH 4.2, 8% PEG 4000 and 100 mM sodium malonate pH 7.0. Crystals were Incubated In crystallization buffer supplemented with 15% glycerol and flash-frozen in liquid nitrogen. Detergent-solubilized CsgG was concentrated to 5 mg ml⁻¹ and crystallized by hanging-drop vapour diffusion against a solution containing 100 mM Tris-HCl pH 8.0, 8% PEG 4000, 100 mM NaCl and 500 mM MgCl₂. Crystals were flash-frozen in liquid nitrogen and cryoprotected by the detergent present in the crystallization solution. For optimization of crystal conditions and screening for crystals with good diffraction quality, crystals were analysed on beamlines Proxima-1 and Proxima-2a (Soleil, France), PX-I (Swiss Light Source, Switzerland), I02, I03, I04 and I24 (Diamond Light Source, UK) and ID14eh2; ID23eh1 and ID23eh2 (ESRF, France). Final diffraction data used for structure determination of CsgG_{C1S} and CsgG were collected at beamlines I04 and I03, respectively (Table 5).

TABLE 5
Data collection and refinement statistics

| | |
|---|---|
| CsgG_{C1S} | CsgG |

Data collection

| | | |
|---|---|---|
| Space group | P1 | C2 |
| Cell dimensions | | |
| a, b, c (Å) | 101.3, 103.6, 141.7 | 161.9, 372.8, 161.9 |
| α, β, γ (°) | 111.3, 90.5, 118.2 | 90.0, 92.9, 90.0 |

| | | | | |
|---|---|---|---|---|
| Resolution (Å)* | 30-2.8 | (2.9-2.8) | 30-3.6 | (3.7-3.6) |

30 −3.6 (a*), −3.7

| | | | (b*), −3.8 (c*)† | |
|---|---|---|---|---|
| Rmeas* | 15.1 | (81.8) | 16.2 | (90.6)† |
| I/σI* | 9.82 | (2.03) | 6.80 | (1.89)† |
| Completeness (%)* | 98.7 | (98.3) | 91.57 | (27.26) |

| | |
|---|---|
| 99.9 | (99.1)† |

| | | | | |
|---|---|---|---|---|
| Redundancy* | 11.2 | (7.0) | 4.4 | (4.3) |

| | | |
|---|---|---|
| Wilson B (Å²) | 46.7 | 101.0 |

Refinement

| | | | | |
|---|---|---|---|---|
| Resolution (Å)* | 30-2.8 | (2.9-2.8) | 30-3.6 | (3.7-3.6) |

| | |
|---|---|
| | 30 −3.6 (a*), −3.7 |
| Z | (b*), −3.8 (c*)† |

| | | | | |
|---|---|---|---|---|
| No. reflections* | 112419 | (11159) | 102130 | (11094) |

| | | |
|---|---|---|
| R_{work}/R_{free} | 0.1881/0.2337 | 0.3024/0.3542 |
| No. atoms | | |

| | | |
|---|---|---|
| Protein | 28853 | 34165 |
| Ligand/ion | 0 | 0 |
| Water | 0 | 0 |

B-factors (Å²)

| | | |
|---|---|---|
| Protein | 57.3 | 116.7 |

Ligand/ion
Water
R.m.s deviations

| | | |
|---|---|---|
| Bond lengths (Å) | 0.01 | 0.03 |
| Bond angles (°) | 1.31 | 1.87 |

Data statistics for CsgG_{C1S} and membrane-extracted CsgG, collected from a single crystal each.
*Highest resolution shell is shown in parenthesis.
†Values corrected for anisotropic truncation along reciprocal directions a*, b* and c*.

Diffraction data for CsgG_{C1S} were processed using Xia2 and the XDS package (Winter, G., J. Appl. Cryst., 2010, 43, 186-190; Kabsch, W., Acta Crystallogr. D Biol. Crystallogr. 2010, 66, 125-132). Crystals of CsgG_{C1S} belonged to space group P1 with unit cell dimensions of a=101.3 Å, b=103.6 Å, c=141.7 Å, α=111.3°, β=90.5°, γ=118.2°, containing 16 protein copies in the asymmetric unit. For structure determination and refinement, data collected at 0.9795 Å wavelength were truncated at 2.8 Å on the basis of an IIσI cutoff of 2 in the highest-resolution shell. The structure was solved using experimental phases calculated from a single anomalous dispersion (SAD) experiment. A total of 92 selenium sites were located in the asymmetric unit by using ShelxC and ShelxD (Sheldrick, G. M., Acta Crystallogr. D Biol. Crystallogr. 2010, 66, 479-485), and were refined and used for phase calculation with Sharp (Bricogne, G., Acta Crystallogr. D Biol. Crystallogr. 2003, 59, 2023-2030) (phasing power 0.79, Figure of merit (FOM) 0.25). Experimental phases were density modified and averaged by non-crystallographic symmetry (NCS) using Parrot (Cowtan, K., Ada Crystallogr. D Biol. Crystallogr. 2010, 66, 470-478 (FIG. 7; FOM 0.85). An initial model was built with Buccaneer (Cowtan, K., Ada Crystallogr. D Biol. Crystallogr. 2006, 62, 1002-1011) and refined by iterative rounds of maximum-likelihood refinement with Phenix refine (Adams, P. D. et al. Acta Crystallogr. D Biol. Crystallogr. 2010, 66, 213-221) and manual inspection and model (re)building in Coot (Emsley, P. et al., Ada Crystallogr. D Biol. Crystallogr. 2010, 66, 486-501). The final structure contained 28,853 atoms in 3,700 residues belonging to 16 CsgG_{C1S} chains (FIG. 7), with a molprobity (Davis, I. W. et al., Nucleic Acids Res. 35 (Suppl 2), W375-W383) score of 1.34; 98% of the residues lay in favoured regions of the Ramachandran plot (99.7% in allowed regions). Electron density maps showed no unambiguous density corresponding to possible solvent molecules, and no water molecules or ions were therefore built in. Sixteenfold NCS averaging was maintained throughout refinement, using strict and local NCS restraints in early and late stages of refinement, respectively.

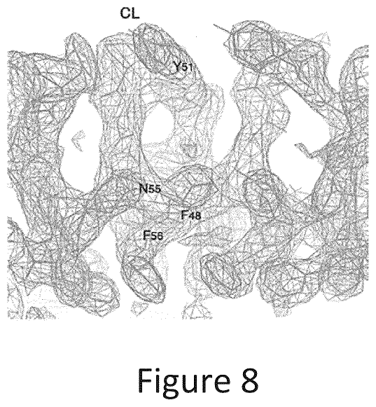

Diffraction data for CsgG were collected from a single crystal at 0.9763 Å wavelength and were Indexed and scaled, using the XDS package (Winter, G., J. Appl. Cryst. 2010, 43, 186-190; Kabsch, W., Ada Crystallogr. D Biol. Crystallogr. 2010, 66, 125-132), in space group C2 with unit-cell dimensions a=161.7 Å, b=372.3 Å, c=161.8 Å and β=92.9°, encompassing 18 CsgG copies in the asymmetric unit and a 72% solvent content. Diffraction data for structure determination and refinement were elliptically truncated to resolution limits of 3.6 Å, 3.7 Å and 3.8 Å along reciprocal cell directions a*, b* and c* and scaled anisotropically with the Diffraction Anisotropy Server (Strong, M et al., Proc. Natl Acad. Sci. USA 2006, 103, 8060-8065). Molecular replacement using the CsgG_{C1S} monomer proved unsuccessful. Analysis of the self rotation function revealed D) symmetry in the asymmetric unit (not shown). On the basis of on the CsgG_{C1S} structure, a nonameric search model was generated in the assumption that after going from a C₈ to C₉ oligomer, the interprotomer arc at the particle circumference would stay approximately the same as the interprotomer angle changed from 45° to 40°, giving a calculated increase in radius of about 4 Å. Using the calculated nonamer as search model, a molecular replacement solution containing two copies was found with Phaser (McCoy, A. J. et al., J. Appl. Cryst. 2007, 40, 658-674). Inspection of density-modified and NCS-averaged electron density maps (Parrot [Cowtan, K., Acta Crystallogr. D Biol. Crystallogr. 2010, 66, 470-478]; FIG. 8) allowed manual building of the TM1 and TM2 and remodelling of adjacent residues in the protein core, as well as the building of residues 2-18, which were missing from the CsgG_{C1S} model and linked the α1 helix to the N-terminal lipid anchor. Refinement of the CsgG model was performed with Buster-TNT (Smart, O. S. et al., Acta Crystallogr. D Biol. Crystallogr. 2012, 68, 368-380) and Refmac5 (Murshudov, G. N. et al., Acta Crystallogr. D Biol. Crystallogr., 2011, 67, 355-367) for initial and final refinement rounds, respectively. Eighteenfold local NCS restraints were applied throughout refinement, and Refmac5 was run employing a jelly-body refinement with sigma 0.01 and hydrogen-bond restraints generated by Prosmart (Nicholls, R. A., Long, F. & Murshudov, G. N., Acta Crystallogr. D Biol. Crystallogr. 2011, 68, 404-417). The final structure contained 34,165 atoms in 4,451 residues belonging to 18 CsgG chains (FIG. 7), with a molprobity score of 2.79; 93.0% of the residues lay in favoured regions of the Ramachandran plot (99.3% in allowed regions). No unambiguous electron density corresponding the N-terminal lipid anchor could be discerned.

### Example 5: Congo Red Assay

For analysis of Congo red binding, a bacterial overnight culture grown at 37° C. in Lysogeny Broth (LB) was diluted in LB medium until a D₆₀₀ of 0.5 was reached. A 5 μl sample was spotted on LB agar plates supplemented with ampicillin (100 mg l⁻¹), Congo red (100 mg l⁻¹) and 0.1% (w/v) isopropyl β-D-thiogalactoside (IPTG). Plates were incubated at room temperature (20 to 22° C.) for 48 h to induce curli expression. The development of the colony morphology and dye binding were observed at 48 h.

### Example 6: Cysteine Accessibility Assays

Cysteine mutants were generated In pMC2 using site-directed mutagenesis and expressed in E. coli LSR12 (Chapman, M. R. et al., Science, 2002, 295, 851-855). Bacterial cultures grown overnight were spotted onto LB agar plates containing 1 mM IPTG and 100 mg l⁻¹ ampicillin. Plates were incubated at room temperature and cells were scraped after 48 h, resuspended in 1 ml of PBS and normalized using D m. The cells were lysed by sonication and centrifuged for 20 s at 3,000 g at 4° C. to remove unbroken cells from cell lysate and suspended membranes. Proteins in the supernatant were labelled with 15 mM methoxypolyethylene glycol-maleimide (MAL-PEG 5 kDa) for 1 h at room temperature. The reaction was stopped with 100 mM DTT and centrifuged at 40.000 r.p.m. (˜100.000 g) in a 50.4 Ti rotor for 20 min at 4° C. to pellet total membranes. The pellet was washed with 1% sodium lauroyl sarcosinate to solubilize cytoplasmic membranes and centrifuged again. The resulting outer membranes were resuspended and solubilized using PBS containing 1% DDM. Metal-affinity pulldowns with nickel beads were used for SDS-PAGE and anti-His western blots. E. coli LSR12 cells with empty pMC2 vector were used as negative control.

### Example 7: ATR-FTIR Spectroscopy

ATR-FTIR measurements were performed on an Equinox 55 infrared spectrophotometer (Bruker), continuously purged with dried air, equipped with a liquid-nitrogen-refrigerated mercury cadmium telluride detector and a Golden Gate reflectance accessory (Specac). The internal reflection element was a diamond crystal (2 mm×2 mm) and the beam incidence angle was 45°. Each purified protein sample (1 μl) was spread at the surface of the crystal and dried under a gaseous nitrogen flow to form a film. Each spectrum, recorded at 2 cm⁻¹ resolution, was an average of 128 accumulations for improved signal-to-noise ratio. All the spectra were treated with water vapour contribution subtraction, smoothed at a final resolution of 4 cmi by apodization and normalized on the area of the Amide I band (1,700-1,600 cm⁻¹) to allow their comparison (Goormaghtigh, E.; Ruysschaert, J. M., Spectrochim. Acta, 1994, 50A, 2137-2144).

### Example 8: Negative Stain EM and Symmetry Determination

Negative stain EM was used to monitor in-solution oligomerization states of CsgG, CsgG_{C1S} and CsgE. CsgE, CsgG_{C1S} and amphipol-bound CsgG were adjusted to a concentration of 0.05 mg ml⁻¹ and applied to glow-discharged carbon-coated copper grids (CF-400: Electron Microscopy Sciences). After 1 min incubation, samples were blotted, then washed and stained in 2% uranyl acetate. Images were collected on a Tecnai T12 BioTWIN LaB6 microscope operating at a voltage of 120 kV, at a magnification of ×49,000 and defocus between 800 and 2,000 nm. Contrast transfer function (CTF), phase flipping and particle selection were performed as described for cryo-EM. For membrane-extracted CsgG, octadecameric particles (1,780 in all) were analysed separately from nonamers and top views. For purified CsgE a total of 2,452 particles were analysed. Three-dimensional models were obtained as described for the CsgG-CsgE cryo-EM analysis below and refined by several rounds of multi-reference alignment (MRA), multi-statistical analysis (MSA) and anchor set refinement. In all cases, after normalization and centring, images were classified using IMAGIC-4D as described in the cryo-EM section. The best classes corresponding to characteristic views were selected for each set of particles. Symmetry determination of CsgG top views was performed using the best class averages with roughly images per class. The rotational autocorrelation function was calculated using IMAGIC and plotted.

### Example 9: Negative Stain EM and Symmetry Determination

Negative stain EM was used to monitor in-solution oligomerization states of CsgG, CsgGC1S and CsgE. CsgE, CsgGC1S and amphipol-bound CsgG were adjusted to a concentration of 0.05 mg ml-1 and applied to glow-discharged carbon-coated copper grids (CF-400; Electron Microscopy Sciences). After 1 min incubation, samples were blotted, then washed and stained in 2% uranyl acetate. Images were collected on a Tecnai T12 BioTWIN LaB6 microscope operating at a voltage of 120 kV, at a magnification of ×49,000 and defocus between 800 and 2,000 nm. Contrast transfer function (CTF), phase flipping and particle selection were performed as described for cryo-EM. For membrane-extracted CsgG, octadecameric particles (1,780 in all) were analysed separately from nonamers and top views. For purified CsgE a total of 2,452 particles were analysed. Three-dimensional models were obtained as described for the CsgG-CsgE cryo-EM analysis below and refined by several rounds of multi-reference alignment (MRA), multi-statistical analysis (MSA) and anchor set refinement. In all cases, after normalization and centring, images were classified using IMAGIC-4D as described in the cryo-EM section. The best classes corresponding to characteristic views were selected for each set of particles. Symmetry determination of CsgG top views was performed using the best class averages with roughly 20 images per class. The rotational autocorrelation function was calculated using IMAGIC and plotted.

### Example 10: CsgG-CsgE Complex Formation

For CsgG-CsgE complex formation, the solubilizing detergents in purified CsgG were exchanged for Amphipols A8-35 (Anatrace) by adding 120 ml of CsgG (24 mg ml⁻¹ protein in 0.5% C8E4, 4 mM LDAO, 25 mM Tris-HCl pH 8.0, 500 mM NaCl, 1 mM DTT) to 300 ml of detergent-destabilized liposomes (1 mg ml⁻¹ 1,2-dimyristoyl-sn-glycero-3-phosphocholine (DMPC) and 0.4% LDAO) and incubating for 5 min on Ice before the addition of 90 ml of A8-35 amphipols at 100 mg ml⁻¹ stock. After an additional 15 min incubation on ice, the sample was loaded on a Superose 6 10/300 GL (GE Healthcare) column and gel filtration was performed in 200 mM NaCl, 2.5% xylitol, 25 mM Tris-HCl pH 8, 0.2 mM DTT. An equal volume of purified monomeric CsgE in 200 mM NaCl, 2.5% xylitol, 25 mM Tris-HCl pH 8, 0.2 mM DTT was added to the amphipol-solubilized CsgG at final protein concentrations of 15 and 5 mM for CsgE and CsgG, respectively, and the sample was run at 125 V at 18° C. on a 4.5% native PAGE in 0.5×TBE buffer. For the second, denaturing dimension, the band corresponding to the CsgG-CsgE complex was cut out of unstained lanes run in parallel on the same gel, boiled for 5 min in Laemmli buffer (60 mM Tris-HCl pH 6.8, 2% SDS, 10% glycerol, 5% 2-mercaptoethanol, 0.01% bromophenol blue) and run on 4-20% SDS-PAGE. Purified CsgE and CsgG were run alongside the complex as control samples. Gels were stained with InstantBlue Coomassie for visual inspection or SYPRO orange for stoichiometry assessment of the CsgG-CsgE complex by fluorescence detection (Typhoon FLA 9000) of the CsgE and CsgG bands on SDS-PAGE, yielding a CsgG/CsgE ratio of 0.97.

### Example 11: CsgG-CsgE Cryo-EM

Cryo-electron microscopy was used to determine the in-solution structure of the C₉ CsgG-CsgE complex. CsgG-CsgE complex prepared as described above was bound and eluted from a HisTrap FF (GE Healthcare) to remove unbound CsgG, and on elution it was Immediately applied to Quantifoil R2/2 carbon coated grids (Quantifoil Micro Tools GmbH) that had been glow-discharged at 20 mA for 30 s. Samples were plunge-frozen in liquid nitrogen using an automated system (Leica) and observed under a FEI F20 microscope operating at a voltage of 200 kV, a nominal magnification of ×50,000 under low-dose conditions and a defocus range of 1.4-3 mm. Image frames were recorded on a Falcon II detector. The pixel size at the specimen level was 1.9 Å per pixel. The CTF parameters were assessed using CTFFIND3 (Mindell, J. A. & Grigorieff, N., J. Struct. Biol. 2003, 142, 334-347), and the phase flipping was done in SPIDER (Shaikh, T. R. et al., Nature Protocols, 2008, 3, 1941-1974). Particles were automatically selected from CTF-corrected micrographs using BOXER (EMAN2; Tang, G. et al., J. Struct. Biol., 2007, 157, 38-46). Images with an astigmatism of more than 10% were discarded. A total of 1,221 particles were selected from 75 micrographs and windowed into 128-pixel×128-pixel boxes. Images were normalized to the same mean and standard deviation and high-pass filtered at a low-resolution cut-off of ˜200 Å. They were centred and then subjected to a first round of MSA. An initial reference set was obtained using reference free classification In IMAGIC-4D (Image Science Software). The best classes corresponding to characteristic side views of the C₉ cylindrical particles were used as references for the MRA. For CsgG-CsgE complex, the first three-dimensional model was calculated from the best 125 characteristic views (with good contrast and well-defined features) encompassing 1,221 particles of the complex with orientations determined by angular reconstitution (Image Science Software). The three-dimensional map was refined by iterative rounds of MRA. MSA and anchor set refinement. The resolution was estimated to be 24 Å by Fourier shell correlation (FSC) according to the 0.5 criteria level (FIG. 5). Visualization of the map and Figures was performed in UCSF Chimera (Pettersen, E. F. et al., J. Comput. Chem., 2004, 25, 1605-1612).

### Example 12: Single-Channel Current Analysis of CsgG and CsgG+CsgE Pores

Under negative field potential, CsgG pores show two conductance states. FIG. 5 shows representative single-channel current traces of, respectively, the normal (measured at +50, 0 and −50 mV) and the low-conductance forms (measured at 0, +50 and −50 mV). No conversions between both states were observed during the total observation time (n=22), indicating that the conductance states have long lifetimes (second to minute timescale). The lower left panel shows a current histogram for the normal and low-conductance forms of CsgG pores acquired at +50 and −50 mV (n=33). I-V curves for CsgG pores with regular and low conductance are shown in the lower right panel. The data represent averages and standard deviations from at least four independent recordings. The nature or physiological existence of the low-conductance form is unknown.

FIG. 6 shows the results of electrophysiology of CsgG channels titrated with the accessory factor CsgE. The plots display the fraction of open, intermediate and closed channels as a function of CsgE concentration. Open and closed states of CsgG are illustrated in FIG. 6 (0 nM and 100 nM CsgE respectively). Increasing the concentration of CsgE to more than 10 nM leads to the closure of CsgG pores. The effect occurs at +50 mV (left) and −50 mV (right), ruling out the possibility that the pore blockade is caused by electrophoresis of CsgE (calculated pl 4.7) into the CsgG pore. An infrequent (<5%) intermediate state has roughly half the conductance of the open channel. It may represent CsgE-induced incomplete closures of the CsgG channel; alternatively, it could represent the temporary formation of a CsgG dimer caused by the binding of residual CsgG monomer from the electrolyte solution to the membrane-embedded pore. The fraction for the three states was obtained from all-point histogram analysis of single-channel current traces. The histograms yielded peak areas for up to three states, and the fraction for a given state was obtained by dividing the corresponding peak area by the sum of all other states in the recording. Under negative field potential, two open conductance states are discerned, similar to the observations for CsgG (see a). Because both open channel variations were blocked by higher CsgE concentrations, the ‘open’ traces in FIG. 6 (0 nM) combine both conductance forms. The data in the plot represent averages and standard deviations from three independent recordings.

The crystal structure, size-exclusion chromatography and EM show that detergent extracted CsgG pores form non-native tail-to-tail stacked dimers (for example, two nonamers as D9 particle, FIG. 7) at higher protein concentration. These dimers can also be observed in single-channel recordings. The upper panel shows the single-channel current trace of a stacked CsgG pore at +50, 0 and −50 mV (left to right). The lower left panel shows a current histogram of dimeric CsgG pores recorded at +50 and −50 mV. The experimental conductances of +16.2±1.8 and −16.0±3.0 pA (n=15) at +50 and −50 mV, respectively, are near the theoretically calculated value of 23 pA. The lower right panel shows an I-V curve for the stacked CsgG pores. The data represent averages and standard deviations from six independent recordings.

The ability of CsgE to bind and block stacked CsgG pores was tested by electrophysiology. Shown are single-channel current traces of stacked CsgG pore in the presence of 10 or 100 nM CsgE at +50 mV (upper) and −50 mV (lower) are shown. The current traces indicate that otherwise saturating concentrations of CsgE do not lead to pore closure for stacked CsgG dimers. These observations are in good agreement with the mapping of the CsgG-CsgE contact zone to helix 2 and the mouth of the CsgG periplasmic cavity as discerned by EM and site-directed mutagenesis (FIGS. 5 and 6).

### Example 13: Bile Salt Toxicity Assay

Outer-membrane permeability was investigated by decreased growth on agar plates containing bile salts. Tenfold serial dilutions of E. coli LSR12 (Chapman, M. R. et al., Science, 2002, 295, 851-855) cells (5 ml) harbouring both pLR42 (Nenninger, A. A. et al., Mol. Microbiol., 2011, 81, 486-499) and pMC2 (Robinson, L. S. et al., Mol. Microbiol., 2006, 59, 870-881) (or derived helix 2 mutants) were spotted on McConkey agar plates containing 100 mg l⁻¹ ampicillin, 25 mg l⁻¹ chloramphenicol, 1 mM IPTG with or without 0.2% (w/v) L-arabinose. After incubation overnight at 37° C., colony growth was examined.

### Example 14: Single-Channel Current Recordings

Single-channel current recordings were performed using parallel high-resolution electrical recording with the Orbit 16 kit from Nanion. In brief, horizontal bilayers of 1,2-diphytanoyl-sn-glycero-3-phosphocholine (Avanti Polar Lipids) were formed over microcavities (of subpicolitre volume) in a 16-channel multielectrode cavity array (MECA) chip (Ionera) (del Rio Martinez, J. M., ACS NanoSmall, 2014, 5, 8080-8088). Both the cis and trans cavities above and below the bilayer contained 1.0 M KCl, 25 mM Tris-HCl pH 8.0. To insert channels into the membrane, CsgG dissolved in 25 mM Tris-HCl pH 8.0, 500 mM NaCl, 1 mM DTT, 0.5% C8E4. 5 mM LDAO was added to the cis compartment to a final concentration of 90-300 nM. To test the interaction of the CsgG channel with CsgE, a solution of the latter protein dissolved in 25 mM Tris-HCl pH 8.0, 150 mM NaCl was added to the cis compartment to final concentrations of 0.1, 1, 10 and 100 nM. Transmembrane currents were recorded at a holding potential of +50 mV and −50 mV (with the cis side grounded) using a Tecella Triton 16-channel amplifier at a low-pass filtering frequency of 3 kHz and a sampling frequency of 10 kHz. Current traces were analysed using the Clampfit of the pClamp suite (Molecular Devices). Plots were generated using Origin 8.6 (Microcal) (Movileanu, L., Nature Biotechnol., 2000, 18, 1091-1095).

Measured currents were compared with those calculated based on the pore dimensions of the CsgG X-ray structure, modelled to be composed of three segments: the transmembrane section, the periplasmic vestibule, and the inner channel constriction connecting the two. The first two segments were modeled to be of conical shape while the constriction was represented as a cylinder. The corresponding resistances R₁, R₂ and R₃, respectively, were calculated as

R₁=L₁/(πD₁d₁κ)

R₂=L₂/(πD₂d₂κ)

R₃=L₃/(πd₁d₂κ)
where L₁, L₂ and L₃ are the axial lengths of the segments, measuring 3.5, 4.0 and 2.0 nm, respectively, and D₁, d₁, D₂ and d₂ are the maximum and minimum diameters of segments 1 and 2, measuring 4.0, 0.8, 3.4 and 0.8 nm, respectively. The conductivity K has a value of 10.6 S m⁻¹. The current was calculated by inserting R₁, R₂ and R₃ and voltage V=50 mV into

I=V/(R₁+R₂+R₃)

Access resistance was not found to alter the predicted current significantly.

Single channel current recordings such as those described above may be made in the presence of analytes, thereby allowing the channel to assume the role of a biosensor.

### Example 15: Molecular Dynamics Simulation of CsgG Constriction with Model Polyalanine Chain

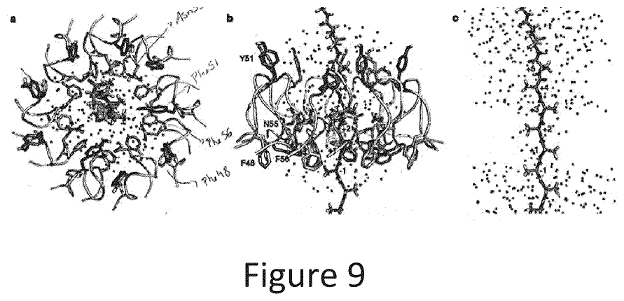

The CsgG constriction has been modelled with a polyalanine chain threaded through the channel in an extended conformation, shown in FIG. 9 in a C-terminal to N-terminal direction. Substrate passage through the CsgG transporter is itself not sequence specific (Nenninger, A. A. et al, Mol. Microbiol., 2011, 81, 486-499; Van Gerven, N. et al., Mol. Microbiol. 2014, 91, 1022-1035 2014). For clarity, a polyalanine chain was used for modelling the putative interactions of a passing polypeptide chain. The modelled area is composed of nine concentric CsgG C-loops, each comprising residues 47-58. Side chains lining the constriction are shown in stick representation with Asn 55 and Phe 56 marked. Solvent molecules (water) within 10 Å of the polyalanine residues inside the constriction (residues labelled+1 to +5) are shown as dots. FIG. 9c shows the modelled solvation of the polyalanine chain, positioned as shown in FIG. 9b and with C-loops removed for clarity (shown solvent molecules are those within 10 Å of the full polyalanine chain). At the height of ring of Asn 55 and Phe 56, the solvation of the polyalanine chain is reduced to a single water shell that bridges the peptide backbone and amide-clamp side chains. Most side chains in the Tyr 51 ring have rotated towards the solvent in comparison with their inward, centre-pointing position observed in the CsgG (and the CsgG_{C1S}) X-ray structure. The model is the result of a 40 ns all-atom explicit solvent molecular dynamics simulation with GROMACS (Pronk, S. et al., Bioinformatics, 2013, 29, 845-854) using the AMBER99SB-ILDN (Lindorff-Larsen, K. et al., Proteins 2010, 78, 1950-1958) force field and with the Ca atoms of the residues at the extremity of the C-loop (Gln 47 and Thr 58) positionally restricted.

### Example 16: Use of the CsgG Nanopore for Nucleic Acid Sequencing

The Phi29 DNA polymerase (DNAP) may be used as a molecular motor with a mutant or wild type CsgG nanopore located within a membrane to allow controlled movement of an oligomeric probe DNA strand through the pore. A voltage may be applied across the pore and a current generated from the movement of ions in a salt solution on either side of the nanopore. As the probe DNA moves through the pore, the ionic flow through the pore changes with respect to the DNA. This information has been shown to be sequence dependent and allows for the sequence of the probe to be read with accuracy from current measurements such as those described above in Example 14.

### Example 17

This Example describes the simulations which were run to investigate DNA behaviour within CsgG.

### Materials and Methods

Steered molecular dynamics simulations were performed to investigate the magnitude of the energetic barrier of CsgG-Eco and various mutants to DNA translocation. Simulations were performed using the GROMACS package version 4.0.5, with the GROMOS 53a6 forcefield and the SPC water model. The structure of CsgG-Eco (SEQ ID NO: 390) was taken from the protein data bank, accession code 4UV3. In order to make models of the CsgG-Eco mutants, the wild-type protein structure was mutated using PyMOL. The mutants studied were CsgG-Eco-(F56A) (SEQ ID NO: 390 with mutation F56A), CsgG-Eco-(F56A-N55S) (SEQ ID NO: 390 with mutations F56A/N55S) and CsgG-Eco-(F56A-N55S-Y51A) (SEQ ID NO: 390 with mutations F56A/N55S/Y51A).

DNA was then placed into the pores. Two different systems were set up:
i. A single guanine nucleotide was placed into the pore, just above the constriction region (approximately 5-10 Angstroms above the residue 56 ring)
ii. A single strand of DNA (ssDNA) was placed along the pore axis, with the 5′ end towards the beta-barrel side of the pore. In this set up, the ssDNA was pre-threaded through the entire length of the pore.

The simulation box was then solvated and then energy minimised using the steepest descents algorithm.

Each system was simulated in the NPT ensemble, using the Berendsen thermostat and Berendsen barostat to 300 K. Throughout the simulation, restraints were applied to the backbone of the pore.

In order to pull the DNA through the pore, a pulling force was applied to the phosphorus atom in the single guanine simulations. In the ssDNA simulations the pulling force was applied to the phosphorus atom at the 5′ end of the strand. The pulling force was applied at a constant velocity by connecting a spring between the DNA phosphorus atom mentioned above and an Imaginary point travelling at a constant velocity parallel to the pore axis. Note that the spring does not have any shape nor does it undergo any hydrodynamic drag. The spring constant was equal to 5 kJmol⁻¹ Å⁻².

### Results

### Single G Translocation

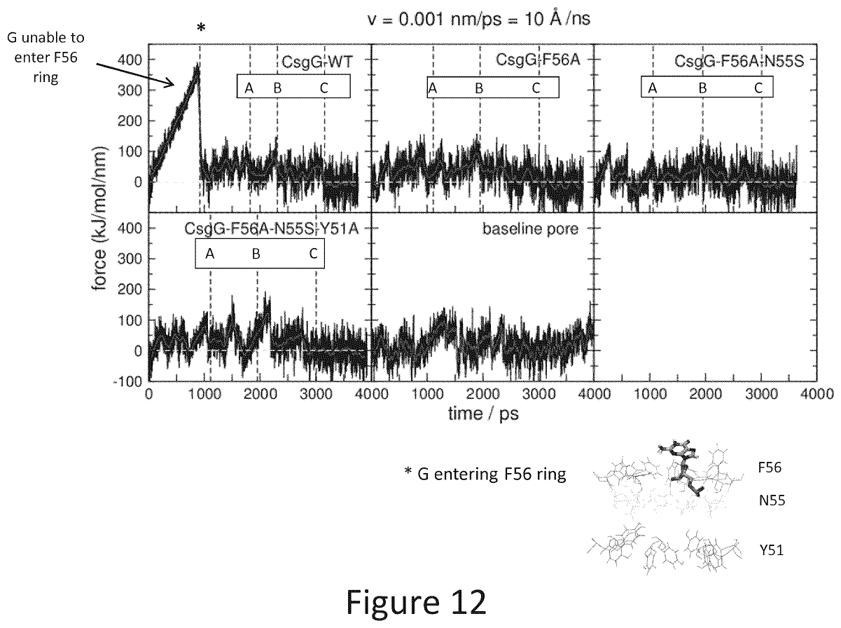

As shown in FIG. 12, a plot of the pulling force versus time shows that there is a large barrier for nucleotide entry into the ring of phenylalanine residues F56 in the wild type CsgG-Eco pore. There was no significant barrier to guanine translocation observed for the CsgG-Eco mutants studied.

ssDNA Translocation

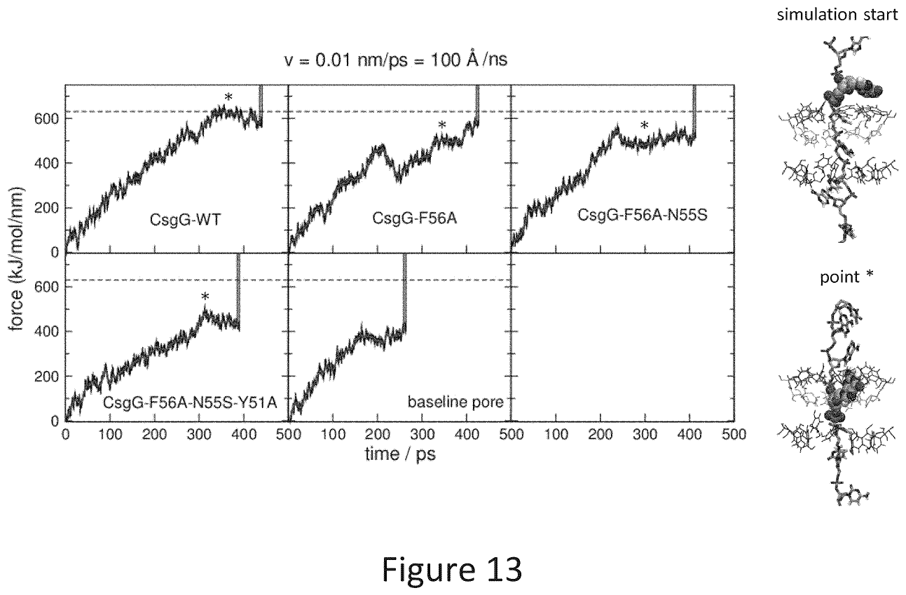

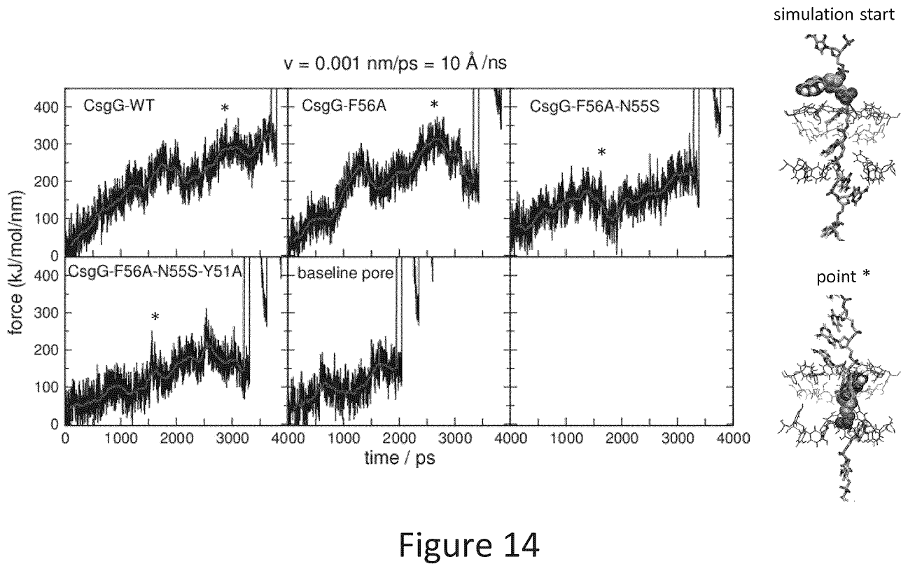

For ssDNA translocation, two simulations were run per pore with each run having a different applied pulling velocity (100 Å/ns and 10 Å/ns). As shown in FIG. 13, which illustrates the faster pulling velocity simulations, the CsgG wild-type pore required the largest pulling force to enable ssDNA translocation. As shown in FIG. 14, which illustrates the slower pulling velocity simulations, both the CsgG-Eco (wild-type, SEQ ID NO: 390) and CsgG-Eco-(F56A) pores required the largest applied force to enable ssDNA translocation. Comparisons between the pulling force required for ssDNA translocation through CsgG and MspA baseline pore, suggest that mutation of the CsgG pore is required to allow a similar level of ssDNA translocation.

### Example 18

This Example describes the characterisation of several CsgG mutants.

### Materials and Methods

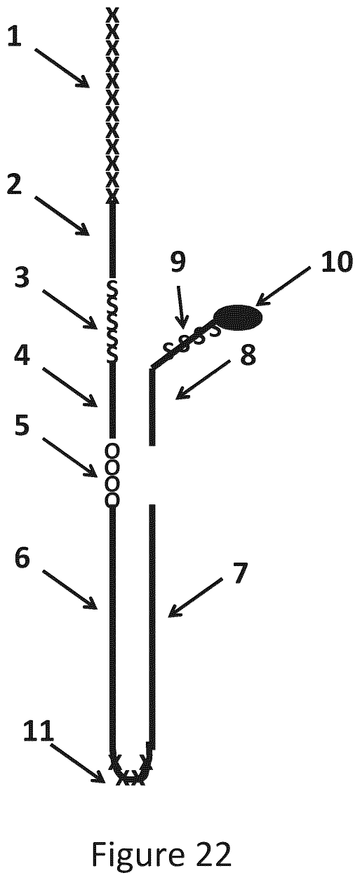

Prior to setting up the experiment, DNA construct X (final concentration 0.1 nM, see FIG. 22 for cartoon representation of construct X and description) was pre-incubated at room temperature for five minutes with T4 Dda—E94C/C109A/C136A/A360C (SEQ ID NO: 412 with mutations E94C/C109A/C136A/A360C, final concentration added to the nanopore system 10 nM, which was provided in buffer (151.5 mM KCl, 25 mM potassium phosphate, 5% glycerol, pH 7.0, 1 mM EDTA)). After five minutes. TMAD (100 μM) was added to the pre-mix and the mixture incubated for a further 5 minutes. Finally, MgCl2 (1.5 mM final concentration added to the nanopore system), ATP (1.5 mM final concentration added to the nanopore system), KCl (500 mM final concentration added to the nanopore system) and potassium phosphate buffer (25 mM final concentration added to the nanopore system) were added to the pre-mix.

Electrical measurements were acquired from a variety of single CsgG nanopores inserted in block co-polymer in buffer (25 mM K Phosphate buffer, 150 mM Potassium Ferrocyanide (II), 150 mM Potassium Ferricyanide (III), pH 8.0). After achieving a single pore inserted in the block co-polymer, then buffer (2 mL, 25 mM K Phosphate buffer, 150 mM Potassium Ferrocyanide (II), 150 mM Potassium Ferricyanide (III), pH 8.0) was flowed through the system to remove any excess CsgG nanopores. 150 uL of 500 mM KCl, 25 mM K Phosphate, 1.5 mM MgCl2, 1.5 mM ATP, pH8.0 was then flowed through the system. After 10 minutes a 150 uL of 500 mM KCl, 25 mM potassium phosphate, 1.5 mM MgCl2, 1.5 mM ATP, pH8.0 was flowed through the system and then the enzyme (T4 Dda—E94C/C109A/C136A/A360C, 10 nM final concentration), DNA construct X (0.1 nM final concentration), fuel (MgCl2 1.5 mM final concentration, ATP 1.5 mM final concentration) pre-mix (150 μL total) was then flowed into the single nanopore experimental system. The experiment was run at −120 mV and helicase-controlled DNA movement monitored.

### Results

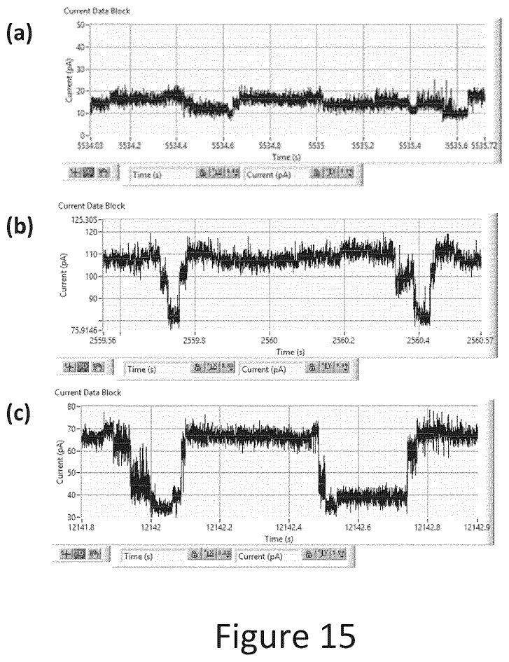

### Pones Showing Increased Range (FIGS. 15 to 17, and 27 to 39)

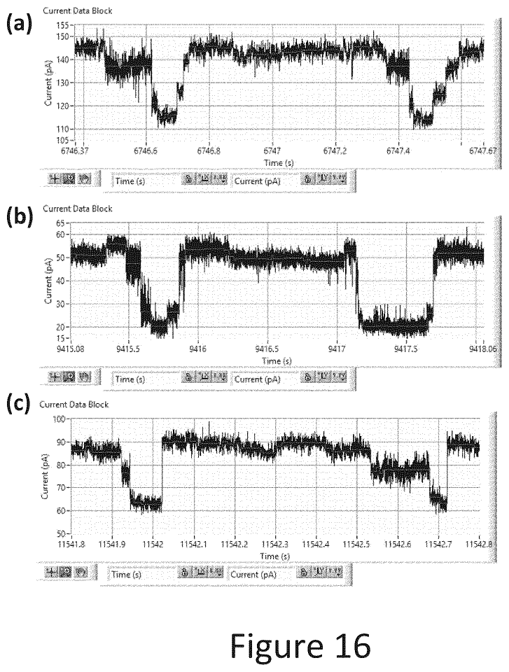

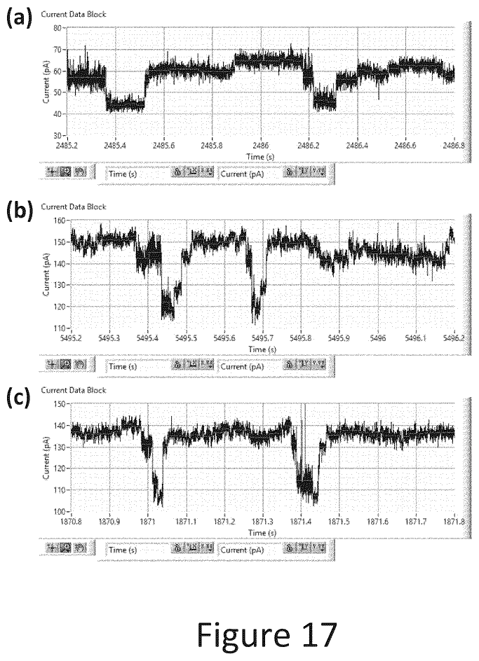

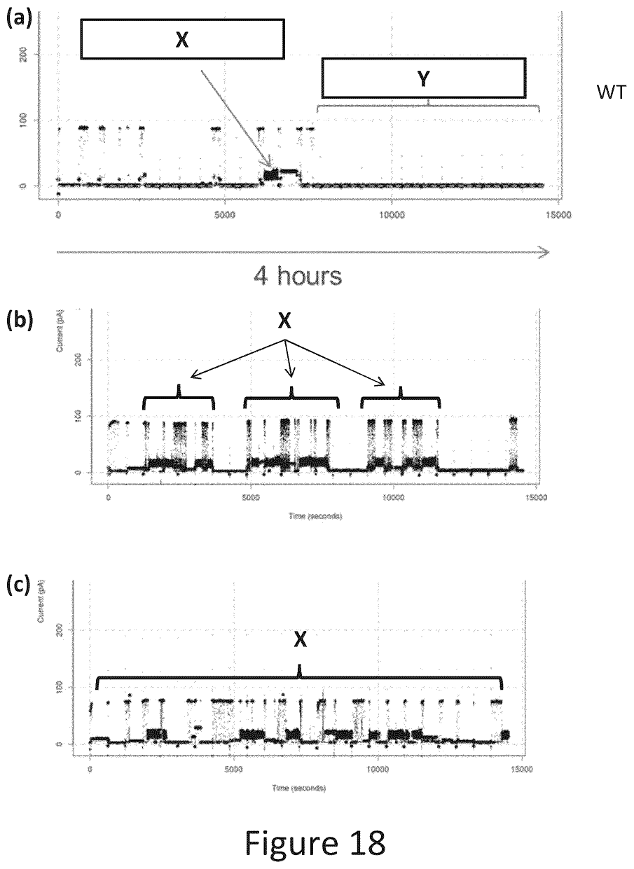

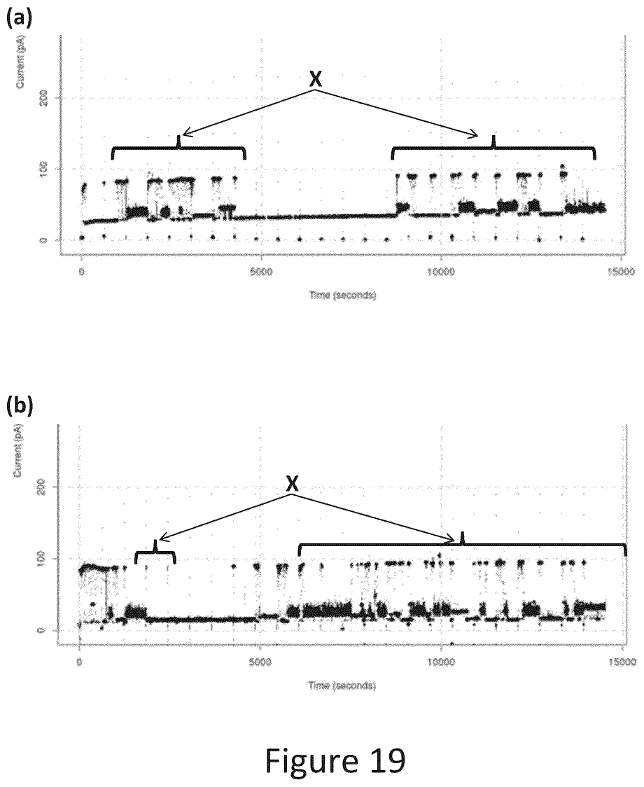

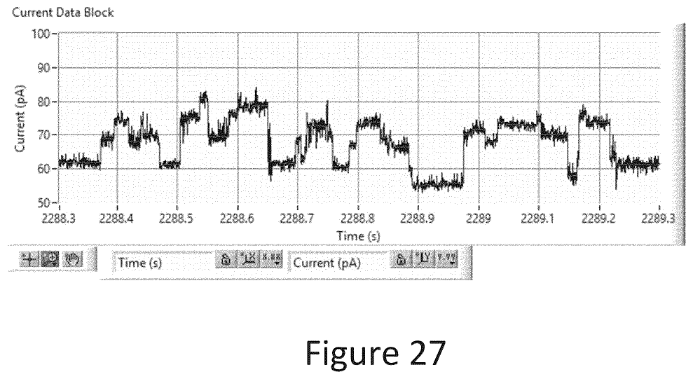

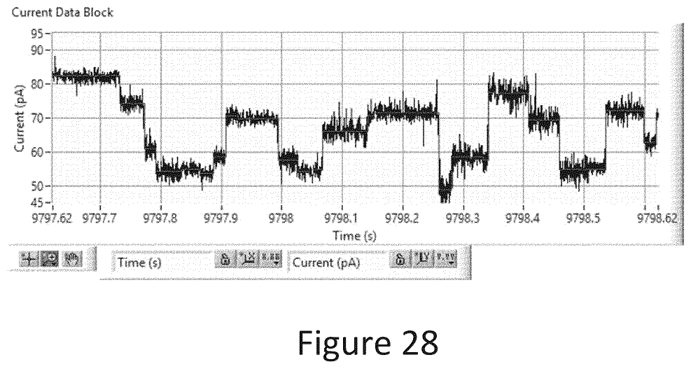

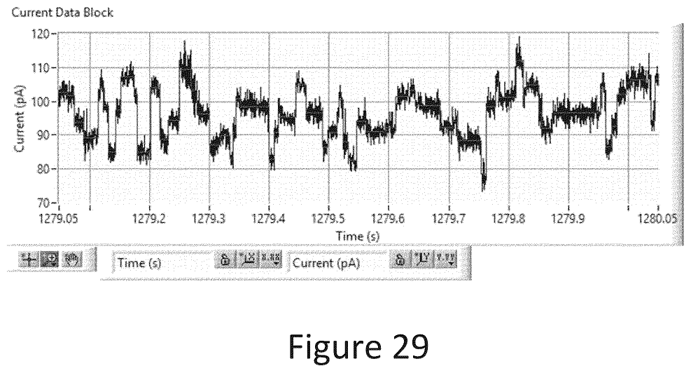

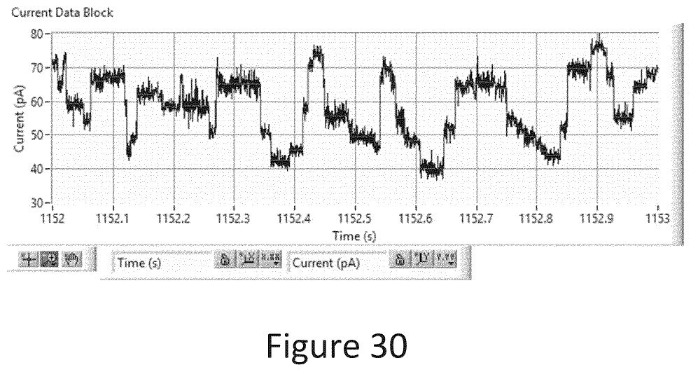

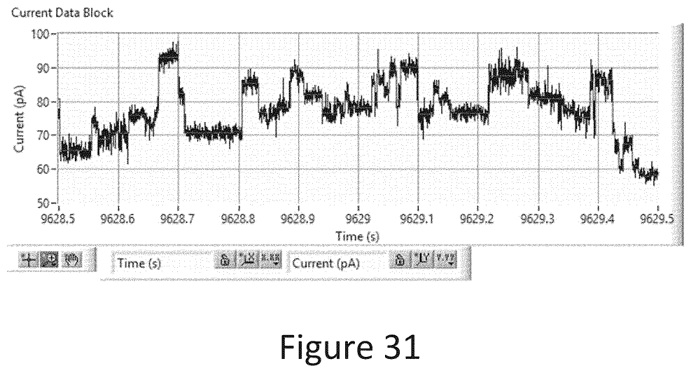

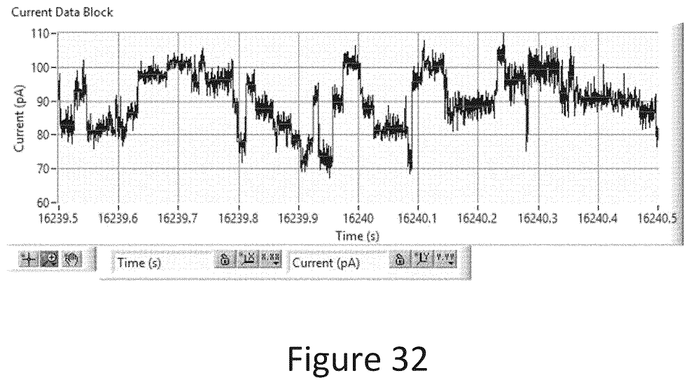

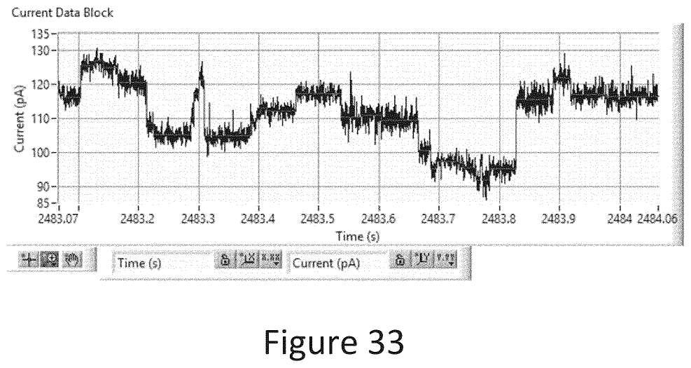

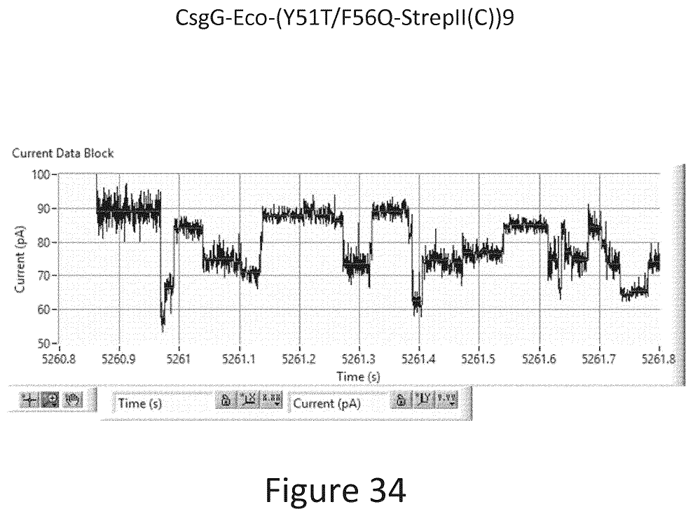

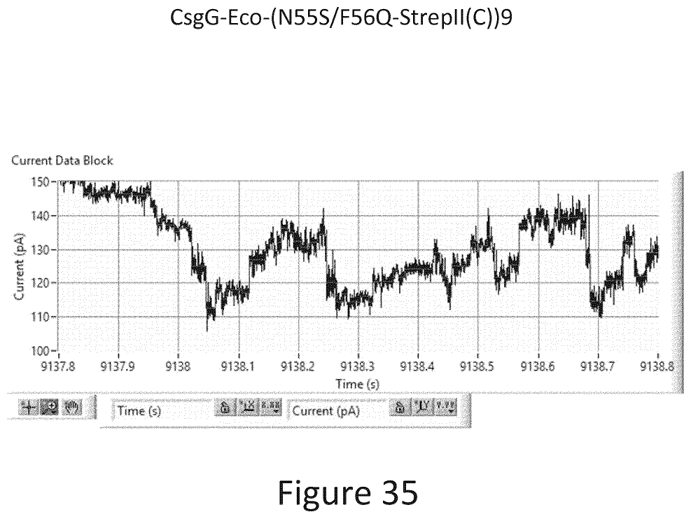

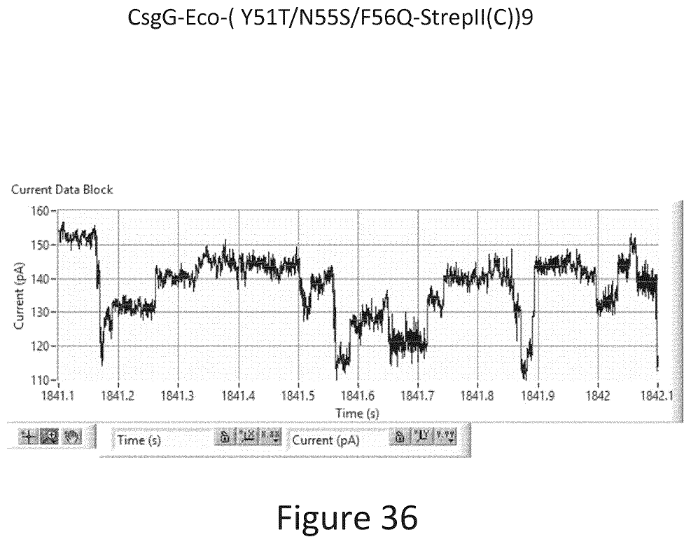

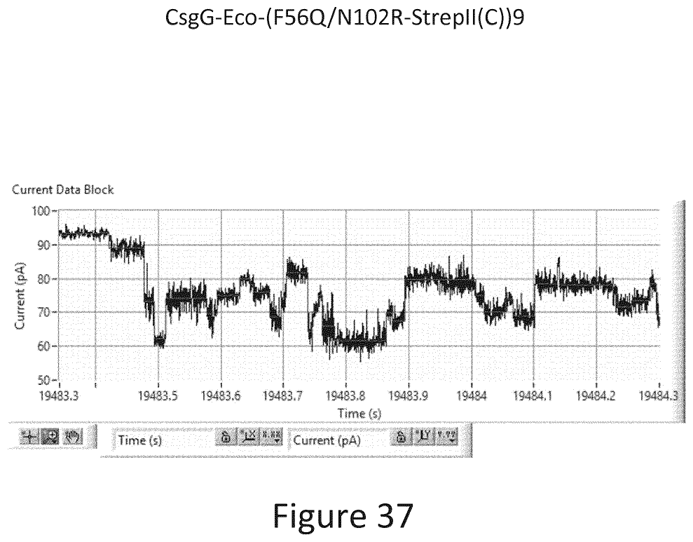

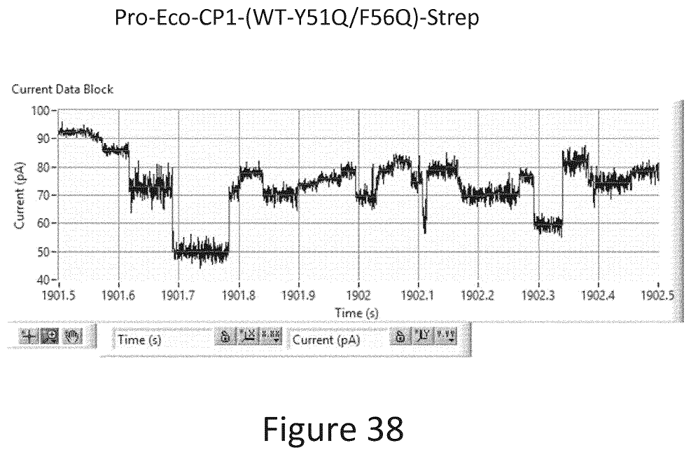

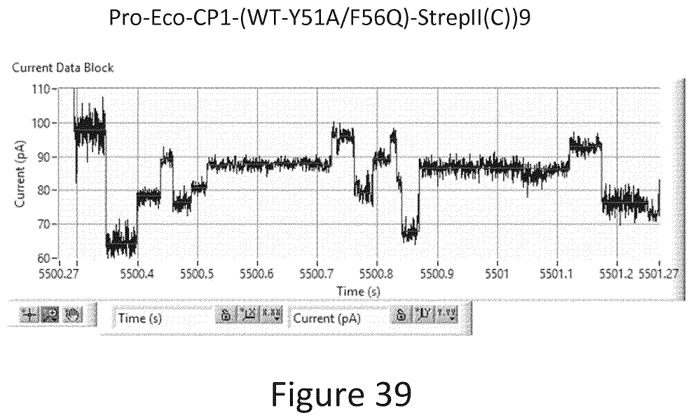

CsgG-Eco-(StrepII(C)) (SEQ ID NO: 390 where StepII(C) is SEQ ID NO: 435 and is attached at the C-terminus) has a range of ˜10 pA (see FIG. 15(a)) whereas the CsgG-Eco pore mutants below exhibited an increased current range—
1—CsgG-Eco-(Y51N-F56A-D149N-E185R-E201N-E203N-StrepII(C))9 (SEQ ID NO: 390 with mutations Y51N/F56A/D149N/E185R/E201N/E203N where StepII(C) is SEQ ID NO: 435 and is attached at the C-terminus) exhibited a range of ˜30 pA (See FIG. 15(b)).
2—CsgG-Eco-(N55A-StrepII(C))9 (SEQ ID NO: 390 with mutation N55A where StepII(C) is has SEQ ID NO: 435 and is attached at the C-terminus) exhibited a range of ˜35 pA (see FIG. 15(c)).
3—CsgG-Eco-(N55S-StrepII(C))9 (SEQ ID NO: 390 with mutations N55S where StepII(C) is SEQ ID NO: 435 and is attached at the C-terminus) exhibited a range of ˜40 pA (see FIG. 16(a)).
4—CsgG-Eco-(Y51N-StrepII(C))9 (SEQ ID NO: 390 with mutation Y51N where StepII(C) is SEQ ID NO: 435 and is attached at the C-terminus) exhibited a range of ˜40 pA (see FIG. 16(b)).
5—CsgG-Eco-(Y51A-F56A-StrepII(C))9 (SEQ ID NO: 390 with mutations Y51A/F56A where StepII(C) is SEQ ID NO: 435 and is attached at the C-terminus) exhibited a range of ˜30 pA (see FIG. 16(c)).
6—CsgG-Eco-(Y51A-F56N-StrepII(C))9 (SEQ ID NO: 390 with mutations Y51A/F56N where StepII(C) is SEQ ID NO: 435 and is attached at the C-terminus) exhibited a range of ˜20 pA (see FIG. 17(a)).
7—CsgG-Eco-(Y51A-N55S-F56A-StrepII(C))9 (SEQ ID NO: 390 with mutations Y51A/N55S/F56A where StepII(C) is SEQ ID NO: 435 and is attached at the C-terminus) exhibited a range of ˜30 pA (see FIG. 17(b)).
8—CsgG-Eco-(Y51A-N55S-F56N-StrepII(C))9 (SEQ ID NO: 390 with mutations Y51A/N55S/F56N where StepII(C) is SEQ ID NO: 435 and is attached at the C-terminus) exhibited a range of ˜30 pA (see FIG. 17(c)).
13—CsgG-Eco-(F56H-StrepII(C))9 (SEQ ID NO: 390 with mutation F56H where StepII(C) is SEQ ID NO: 435 and is attached at the C-terminus) exhibited a range of ˜35 pA (see FIG. 27).
14—CsgG-Eco-(F56Q-StrepII(C))9 (SEQ ID NO: 390 with mutation F56Q where StepII(C) is SEQ ID NO: 435 and is attached at the C-terminus) exhibited a range of ˜40 pA (see FIG. 28).
15—CsgG-Eco-(F56T-StrepII(C))9 (SEQ ID NO: 390 with mutation F56T where StepII(C) is SEQ ID NO: 435 and is attached at the C-terminus) exhibited a range of ˜35 pA (see FIG. 29).
16—CsgG-Eco-(S54P/F56A-StrepII(C))9 (SEQ ID NO: 390 with mutation S54P/F56A where StepII(C) is SEQ ID NO: 435 and is attached at the C-terminus) exhibited a range of ˜35 pA (see FIG. 30).
17—CsgG-Eco-(Y51T/F56A-StrepII(C))9 (SEQ ID NO: 390 with mutation Y51T/F56A where StepII(C) is SEQ ID NO: 435 and is attached at the C-terminus) exhibited a range of ˜30 pA (see FIG. 31).
18—CsgG-Eco-(F56P-StrepII(C))9 (SEQ ID NO: 390 with mutation F56P where StepII(C) is SEQ ID NO: 435 and is attached at the C-terminus) exhibited a range of ˜30 pA (see FIG. 32).
19—CsgG-Eco-(F56A-StrepII(C))9 (SEQ ID NO: 390 with mutation F56A where StepII(C) is SEQ ID NO: 435 and is attached at the C-terminus) exhibited a range of ˜40 pA (see FIG. 33).
20—CsgG-Eco-(Y51T/F56Q-StrepII(C))9 (SEQ ID NO: 390 with mutations Y51T/F56Q where StepII(C) is SEQ ID NO: 435 and is attached at the C-terminus) exhibited a range of ˜30 pA (see FIG. 34).
21—CsgG-Eco-(N55S/F56Q-StrepII(C))9 (SEQ ID NO: 390 with mutations N55S/F56Q where StepII(C) is SEQ ID NO: 435 and is attached at the C-terminus) exhibited a range of ˜35 pA (see FIG. 35).
22—CsgG-Eco-(Y51T/N55S/F56Q-StrepII(C))9 (SEQ ID NO: 390 with mutations Y51T/N55S/F56Q where StepII(C) is SEQ ID NO: 435 and is attached at the C-terminus) exhibited a range of ˜35 pA (see FIG. 36).
23—CsgG-Eco-(F56Q/N102R-StrepII(C))9 (SEQ ID NO: 390 with mutations F56Q/N102R where StepII(C) is SEQ ID NO: 435 and is attached at the C-terminus) exhibited a range of ˜30 pA (see FIG. 37).
24—CsgG-Eco-(Y51Q/F56Q-StrepII(C))9 (SEQ ID NO: 390 with mutations Y51Q/F56Q where StepII(C) is SEQ ID NO: 435 and is attached at the C-terminus) exhibited a range of ˜40 pA (see FIG. 38).
25—CsgG-Eco-(Y51A/F56Q-StrepII(C))9 (SEQ ID NO: 390 with mutations Y51A/F56Q where StepII(C) is SEQ ID NO: 435 and is attached at the C-terminus) exhibited a range of ˜35 pA (see FIG. 39).
   Pores showing Increased throughput (FIGS. 18 and 19)

As can be seen from FIGS. 18 and 19, the following mutant pores (9-12 below) exhibited multiple helicase controlled DNA movements (Labelled as X in FIGS. 18 and 19) per channel in 4 hours, whereas CsgG-Eco-(StrepII(C)) (SEQ ID NO: 390 where StepII(C) is SEQ ID NO: 435 and is attached at the C-terminus) shown in FIG. 18(a) frequently exhibited only 1 or 2 helicase controlled DNA movements (labelled as X in FIG. 18(a)) per channel in 4 hours and instead exhibited prolonged block regions (labelled as Y in FIG. 18(a)).
9—CsgG-Eco-(D149N-E185N-E203N-StrepII(C))9 (SEQ ID NO: 390 with mutations D149N/E185N1E203N where StepII(C) is SEQ ID NO: 435 and is attached at the C-terminus) (FIG. 18(b))
10—CsgG-Eco-(D149N-E185N-E201N-E203N-StrepII(C))9 (SEQ ID NO: 390 with mutations D149N/E185N/E201N/E203N where StepII(C) is SEQ ID NO: 435 and is attached at the C-terminus) (FIG. 18(c))
11—CsgG-Eco-(D149N-E185R-D195N-E201N-E203N)-StrepII(C))9 (SEQ ID NO 390 with mutations D149N/E185R/D195N/E201N/E203N where StepII(C) is SEQ ID NO: 435 and is attached at the C-terminus) (FIG. 19(a))
12—CsgG-Eco-(D149N-E185R-D195N-E201R-E203N)-StrepII(C))9 (SEQ ID NO: 390 with mutations D149N/E185R/D195N/E201R/E203N where StepII(C) is SEQ ID NO: 435 and is attached at the C-terminus) (FIG. 19(b))

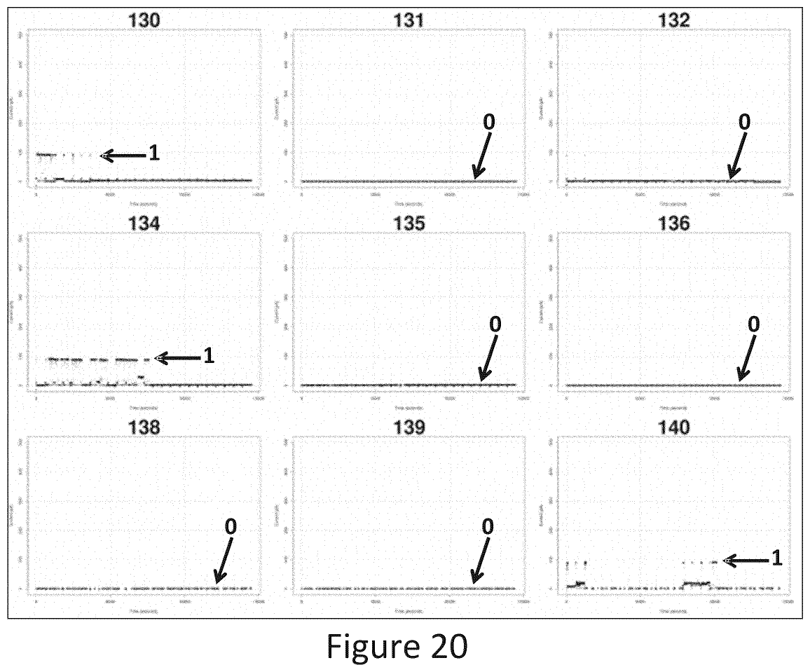

### Pore Showing Increased Insertion (FIGS. 20 and 211

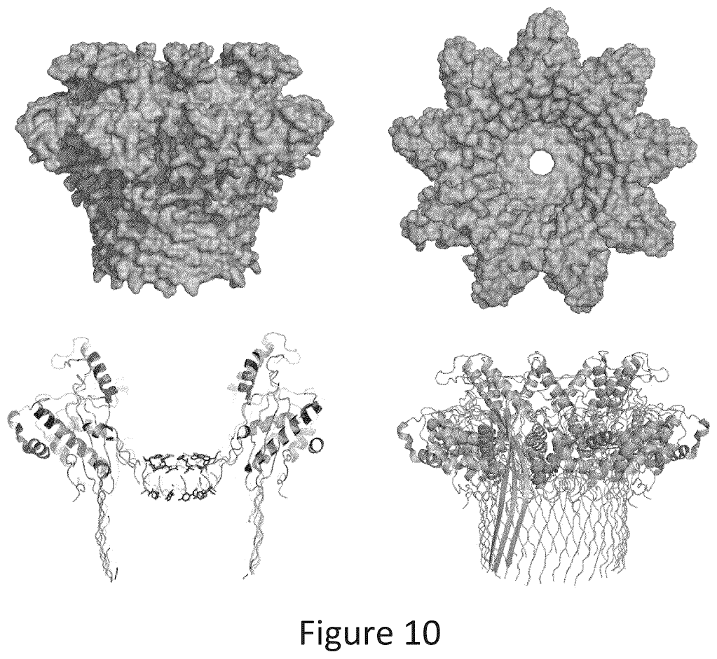

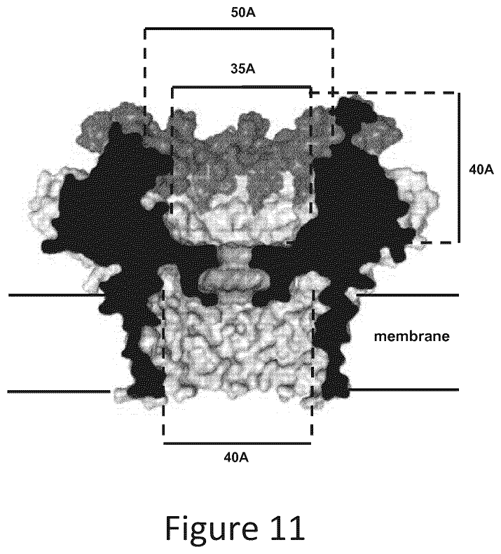

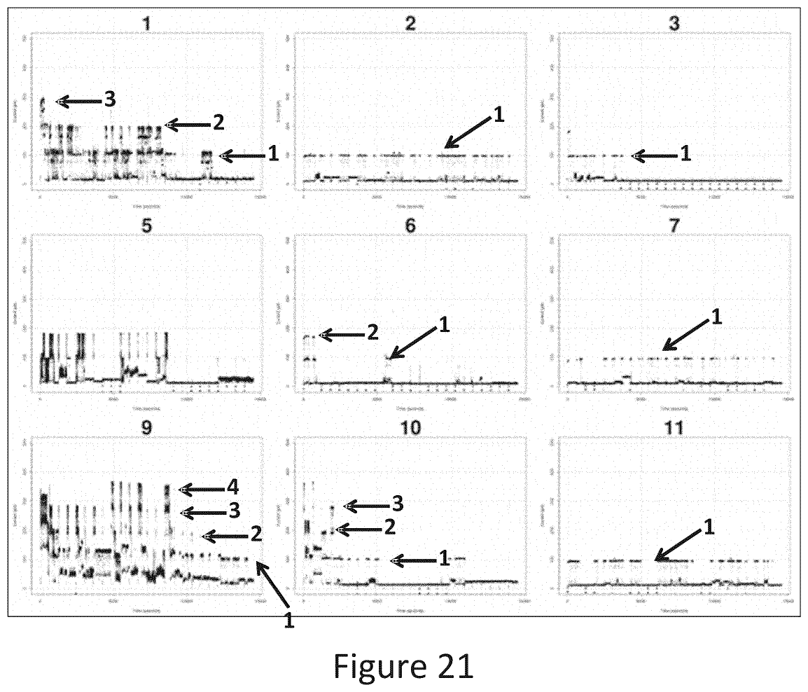

As can be seen by comparing FIGS. 20 and 21, the mutant pore CsgG-Eco-(T150I-StrepII(C))9 (SEQ ID NO: 390 with mutations T150I where StepII(C) is SEQ ID NO: 435 and is attached at the C-terminus) shown in FIG. 21, was present in the membrane in increased pore numbers (˜4-5 fold) compared with the CsgG-Eco-(StrepII(C)) (SEQ ID NO: 390 where StepII(C) is SEQ ID NO: 435 and is attached at the C-terminus) pore (shown in FIG. 20). Arrows in FIGS. 20 and 21 illustrated the number of CsgG-Eco nanopores which inserted into the block co-polymer in a 4 hour experiment (130-140 in FIG. 20 and 1-11 in FIG. 21 each corresponded to a separate nanopore experiment). For CsgG-Eco-(StrepII(C)) (SEQ ID NO: 390 where StepII(C) is SEQ ID NO: 435 and is attached at the C-terminus) three experiments showed insertion of one nanopore, whereas for the mutant pore (CsgG-Eco-(T150I-StrepII(C))9) each experiment showed insertion of at least one nanopore and several experiments showed multiple pore insertions.

### Example 19

This example described an E. coli purification method developed to purify the CsgG pore.

### Materials and Methods

DNA encoding the polypeptide Pro-CsgG-Eco-(StrepII(C)) (SEQ ID NO: 390 where StepII(C) is SEQ ID NO: 435 and is attached at the C-terminus and where Pro is SEQ ID NO: 436 and is attached at the N-terminus) was synthesised in GenScript USA Inc. and cloned into a pT7 vector containing ampicillin resistance gene. Protein expression of the pT7 vector was induced by Isopropyl β-D-1-thiogalactopyranoside (IPTG). The concentration of the DNA solution was adjusted to 400 ng/uL. DNA (1 μl) was used to transform Lemo21(DE3) competent E. coli cells (50 μl, NEB, catalogue number C2528H). Prior to transformation, the CsgG gene was knocked out from Lemo21(DE3) cells (Gene Bridges GmbH, Germany). The cells were then plated out on LB agar containing ampicillin (0.1 mg/mL) and incubated for approx 16 hours at 37° C.

Bacterial colonies grown on LB plates, containing ampicillin, incorporated the CsgG plasmid. One such colony was used to inoculate a starter culture of LB media (100 mL) containing carbenicillin (0.1 mg/mL). The starter culture was grown at 37° C. with agitation until OD600 was reached to 1.0-1.2. The starter culture was used to inoculate a fresh 500 mL of LB media containing carbenicillin (0.1 mg/mL) and Rhamnose (500 μM) to an O.D. 600 of 0.1. The culture was grown at 37° C. with agitation until OD600 reached 0.6. The temperature of the culture was then adjusted to 18° C. and induction was initiated by the addition of IPTG (0.2 mM final concentration). Induction was carried out for approximately 18 hours with agitation at 18° C.

Following induction, the culture was pelleted by centrifugation at 6,000 g for 30 minutes. The pellet was resuspended in 50 mM Tris, 300 mM NaCl, containing protease inhibitors (Merck Millipore 539138), benzonase nuclease (Sigma E1014) and 1× bugbuster (Merck Millipore 70921) pH8.0 (approximately 10 mL of buffer per gram of pellet). Suspension was mixed well until it was fully homogeneous, the sample was then transferred to roller mixer at 4° c. for approx 5 hours. Lysate was pelleted by centrifugation at 20,000 g for 45 minutes and the supernatant was filtered through 0.22 μM PES syringe filter. Supernatant which contained CsgG (known as sample 1) was taken forward for purification by column chromatography.

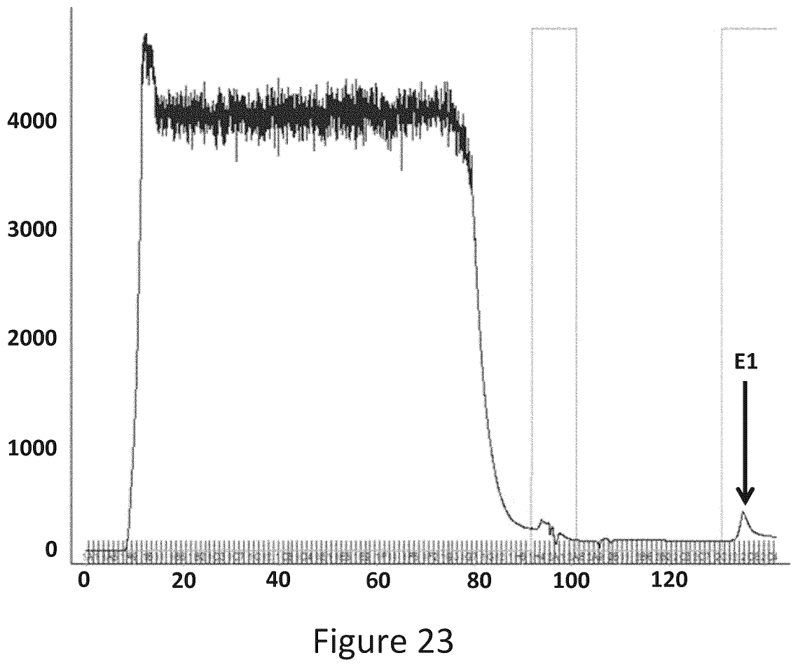

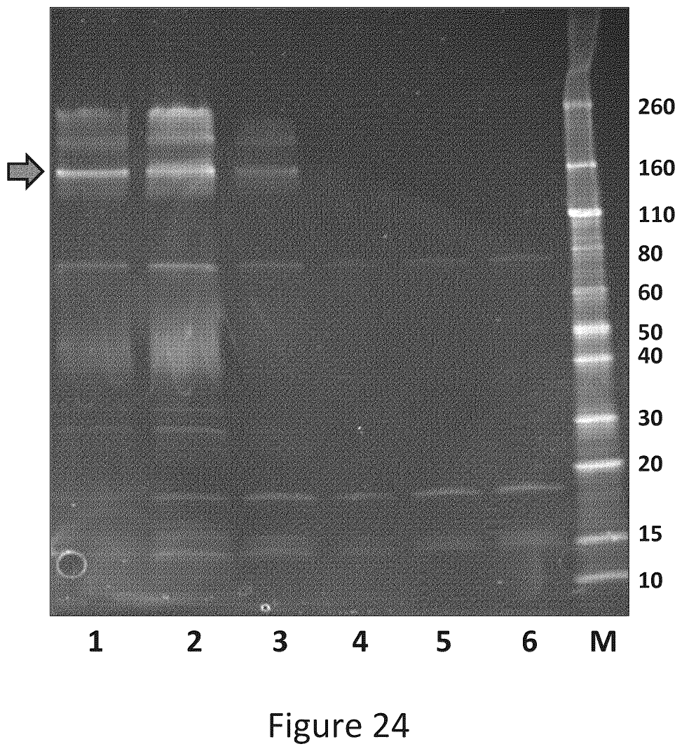

Sample 1 was applied to a 5 mL Strep Trap column (GE Healthcare). The column was washed with 25 mM Tris, 150 mM NaCl, 2 mM EDTA, 0.01% DDM pH8 until a stable baseline of 10 column volumes was maintained. The column was then washed with 25 mM Tris, 2M NaCl, 2 mM EDTA, 0.01% DDM pH8 before being returned to the 150 mM buffer. Elution was carried out with 10 mM desthiobiotin. An example of a chromatography trace of Strep trap (GE Healthcare) purification of a CsgG protein is shown in FIG. 23. The elution peak is labelled E1. FIG. 24 shows an example of a typical SDS-PAGE visualization of CsgG-Eco protein after the initial Strep purification. Lanes 1-3 shows the main elution peak (labelled E1 in FIG. 23) which contained CsgG protein as indicated by the arrow. Lanes 4-6 corresponded to elution fractions of the tail of the main elution peak (labelled E1 in FIG. 23) which contained contaminants.

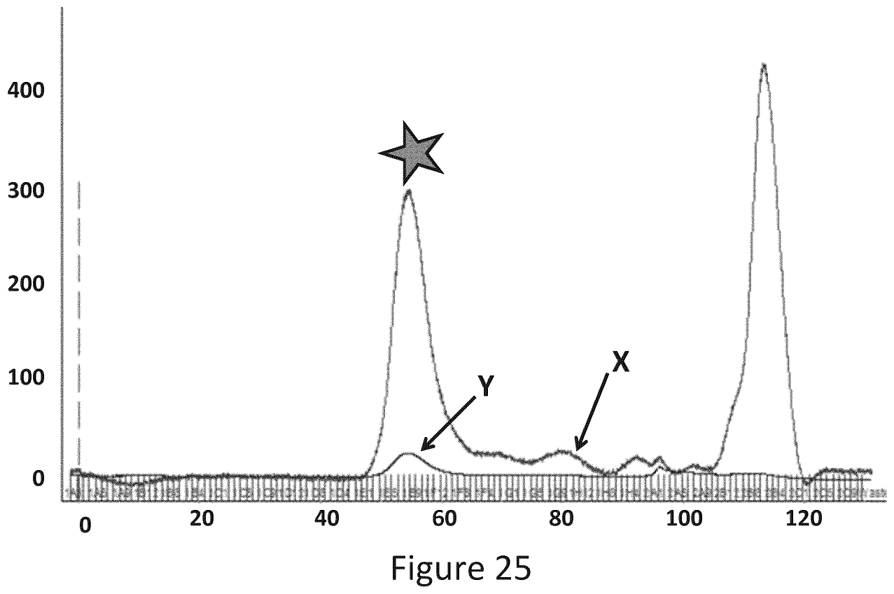

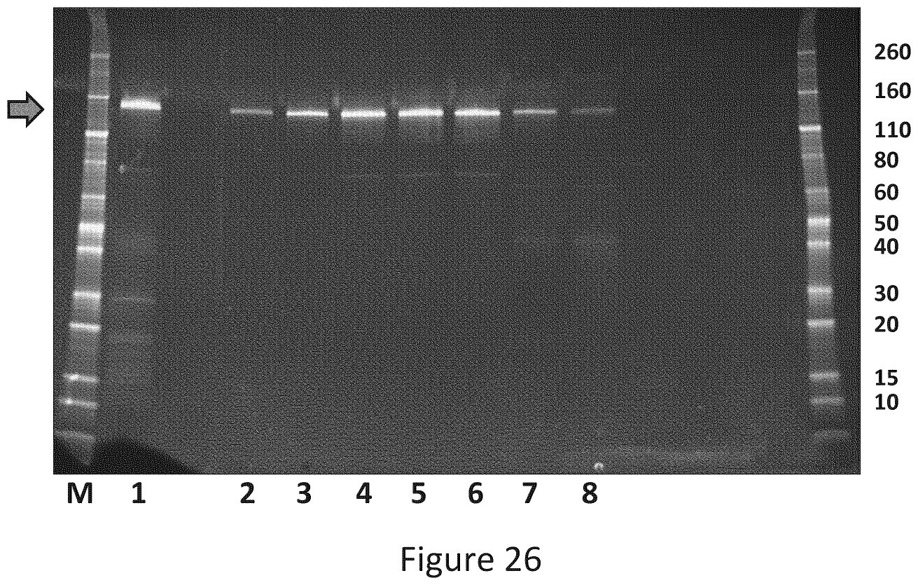

The elution peak was pooled and heated to 65′C for 15 minutes to remove heat unstable contaminated proteins. The heated solution was subjected to centrifugation at 20,000 g for 10 minutes and the pellet was discarded. The supernatant was subjected to gel filtration on a 120 mL Sephadex S200 column (GE Healthcare) in 25 mM Tris, 150 mM NaCl, 2 mM EDTA, 0.01% DDM, 0.1% SDS pH8. Monitoring was carried out at 220 nM due to low Tryptophan component of protein. The sample was eluted at approximately 55 mL volume (FIG. 25 shows the size exclusion column trace with the 55 mL sample peak labeled with a star). The elution peak was run on a 4-20% TGX (see FIG. 26, Bio Rad) to confirm the presence of the pore of interest CsgG-Eco-(StrepII(C)) (SEQ ID NO: 390 where StepII(C) is SEQ ID NO: 435 and is attached at the C-terminus). Identified fractions were pooled and concentrated by 50 kD Amicon spin column.

### Example 20

This example describes the simulations which were run to investigate the interaction between CsgG-Eco-(Y51T/F56Q)-StrepII(C))9 (SEQ ID NO: 390 with mutations Y51T/F56Q where StepII(C) is SEQ ID NO: 435 and is attached at the C-terminus pore mutant No. 20) with T4 Dda—(E94C/C109A/C136A/A360C) (SEQ ID NO: 412 with mutations E94C/C109A/C136A/A360C and then (AM 1)G1 G2).

### Simulation Methods

Simulations were performed using the GROMACS package version 4.0.5, with the GROMOS 53a6 forcefield and the SPC water model.

The CsgG-Eco-(Y51T/F56Q)-StrepII(C))9 (SEQ ID NO: 390 with mutations Y51T/F56Q where StepII(C) is SEQ ID NO: 435 and is attached at the C-terminus pore mutant No. 20) model was based on the crystal structures of CsgG found in the protein data bank, accession codes 4UV3 and 4Q79. The relevant mutations were made using PyMOL. The resultant pore model was then energy minimised using the steepest descents algorithm. The T4 Dda—(E94C/C109A/C136A/A360C) (SEQ ID NO: 412 with mutations E94C/C109A/C136A/A360C and then (ΔM1)G1G2) model was based on the Dda1993 structure found in the protein data bank, accession code 3UPU. Again, relevant mutations were made using PyMOL, and the model was energy minimised using the steepest descents algorithm.

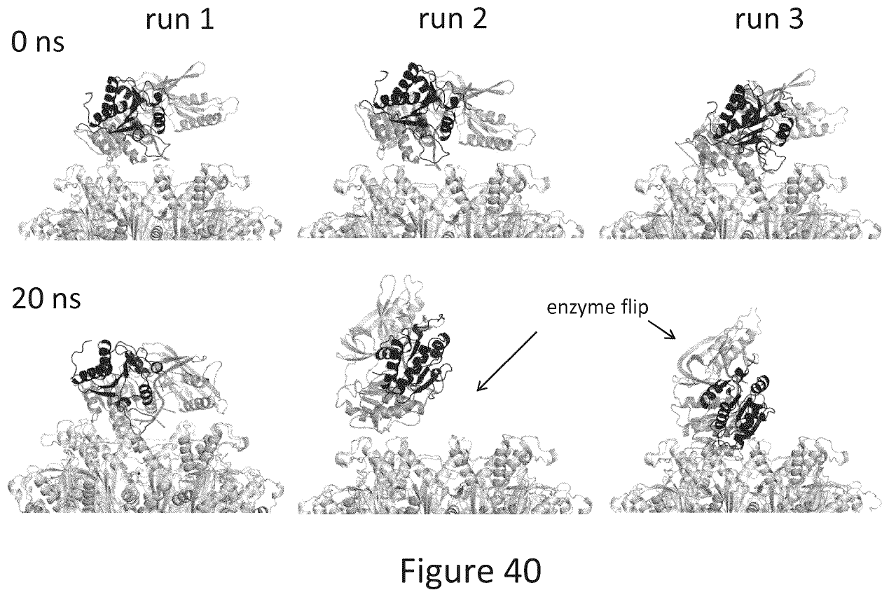

The T4 Dda—(E94C/C109A/C136A/A360C) (SEQ ID NO: 412 with mutations E94C/C109A/C136A/A360C and then (ΔM1)G1G2) model was then placed above CsgG-Eco-(Y51T/F56Q)-StrepII(C))9 (SEQ ID NO: 390 with mutations Y51T(F56Q where StepII(C) is SEQ ID NO: 435 and is attached at the C-terminus pore mutant No. 20). Three simulations were performed with a different initial enzyme conformation (Runs 1 to 3 (0 ns), see FIG. 40):

In all enzyme conformations, the enzyme was oriented such that the 5′ end of the DNA was pointing towards the pore, and the enzyme was unrestrained throughout the simulation. The pore backbone was restrained and the simulation box was solvated. The system was simulated in the NPT ensemble for 40 ns, using the Berendsen thermostat and Berendsen barostat to 300 K.

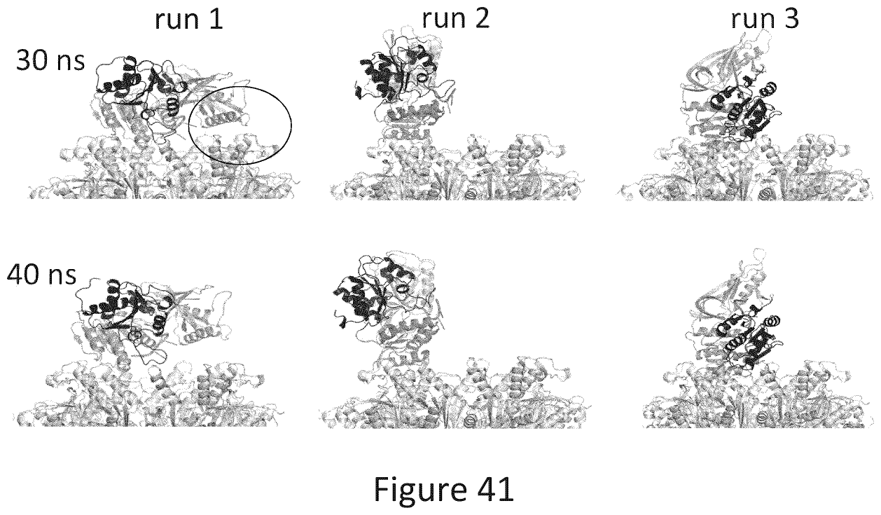

The contacts between the enzyme and pore were analysed using both GROMACS analysis software and also locally written code. The tables below show the number of contacts observed for both pore and enzyme amino acids. Tables 6-8 show the amino acid contact points on pore which interact with the amino acid contact points on the enzyme. In two out of the three simulations the enzyme tilts on top of the pore (see run 2 and 3 (20, 30 and 40 ns), FIGS. 40 and 41). Run 1 shows that the enzyme has not tilted and so points that are shown to have high interaction in table 6 can be optimised in order to increase enzyme stability on the pore cap.

TABLE 6
run 1 enzyme and pore contact interactions

| Pore | Enzyme | # contacts |
|---|---|---|
| ASN 102 | ASP 198 | 8200 |
| ASN 102 | TYR 438 | 8130 |
| GLN 100 | ASP 212 | 7369 |
| GLU 101 | TRP 195 | 5979 |
| ARG 97 | TYR 350 | 4873 |
| GLU 101 | LEU 215 | 4851 |
| ASN 102 | TRP 195 | 3988 |
| ARG 97 | TYR 415 | 3798 |
| GLU 101 | TYR 350 | 3759 |
| LEU 113 | ASP 212 | 3718 |
| ASN 102 | LYS 358 | 3124 |
| ARG 97 | GLY 211 | 2765 |
| GLU 101 | CYS 412 | 2715 |
| ARG 97 | GLY 193 | 2708 |
| ASN 102 | ILE 196 | 2342 |
| GLU 101 | TYR 415 | 2268 |
| GLU 101 | ARG 216 | 2158 |
| ARG 110 | THR 213 | 2094 |
| ARG 110 | ASP 212 | 2066 |
| GLY 103 | ARG 216 | 1456 |
| GLU 101 | TYR 318 | 1333 |
| ASN 102 | GLU 347 | 1316 |
| GLU 101 | LYS 194 | 1310 |
| ARG 97 | PRO 411 | 1203 |
| GLU 101 | LYS 358 | 1161 |
| ASN 102 | ARG 216 | 1132 |
| ARG 97 | TRP 195 | 888 |
| LYS 94 | TYR 415 | 793 |
| ASN 102 | PRO 315 | 696 |
| ASN 102 | LYS 247 | 541 |
| GLU 101 | ALA 214 | 449 |
| ASN 102 | ASP 346 | 440 |
| ARG 97 | ALA 214 | 366 |
| ARG 97 | LYS 194 | 336 |
| GLU 101 | ASP 212 | 302 |
| ARG 97 | VAL 439 | 267 |
| ARG 110 | THR 210 | 263 |
| ARG 97 | THR 210 | 259 |
| ARG 97 | GLN 422 | 257 |
| GLU 101 | TYR 409 | 228 |
| ALA 98 | TRP 195 | 207 |
| GLU 101 | LYS 247 | 201 |
| ASN 102 | GLU 317 | 179 |
| ARG 110 | ARG 216 | 147 |
| ARG 97 | ASP 212 | 108 |
| ASN 102 | VAL 314 | 87 |
| GLU 101 | THR 213 | 72 |
| ASN 102 | LYS 255 | 70 |
| VAL 105 | ARG 216 | 69 |
| ASN 102 | LEU 215 | 59 |
| ASN 102 | THR 210 | 55 |
| ILE 111 | ASP 212 | 48 |
| ARG 97 | HIS 414 | 48 |
| THR 104 | ARG 216 | 36 |
| ASN 102 | TYR 197 | 32 |
| GLN 100 | THR 213 | 30 |
| ASN 102 | GLU 361 | 28 |
| ARG 97 | VAL 418 | 28 |
| ALA 98 | TYR 415 | 27 |
| GLU 101 | LEU 354 | 17 |
| GLU 101 | TYR 197 | 16 |
| ASN 102 | GLY 316 | 16 |
| ARG 97 | GLU 361 | 16 |
| ARG 97 | GLU 347 | 14 |
| ILE 107 | ARG 216 | 12 |
| ASN 102 | GLY 208 | 12 |
| ARG 97 | TYR 409 | 11 |
| ARG 97 | LYS 247 | 11 |
| GLU 101 | LYS 364 | 8 |
| ARG 97 | PHE 209 | 7 |
| LYS 94 | GLU 419 | 6 |
| GLU 101 | PRO 411 | 5 |
| GLU 101 | GLU 317 | 5 |
| ASN 102 | ILE 251 | 5 |
| ARG 97 | LEU 354 | 5 |
| LYS 94 | VAL 418 | 3 |
| ASN 102 | ARG 321 | 3 |
| ARG 97 | LYS 243 | 3 |
| LYS 94 | CYS 412 | 2 |
| LEU 113 | THR 210 | 2 |
| GLY 103 | GLU 317 | 2 |
| GLU 101 | LYS 351 | 2 |
| ASN 102 | TYR 318 | 2 |
| ASN 102 | MET 219 | 2 |
| ASN 102 | LYS 194 | 2 |
| ARG 97 | VAL 314 | 2 |
| ARG 97 | LYS 364 | 2 |
| THR 104 | PRO 315 | 1 |
| GLY 103 | THR 213 | 1 |
| GLU 101 | PRO 315 | 1 |

TABLE 7
run 2 enzyme and pore contact interactions

| Pore | Enzyme | # contacts |
|---|---|---|
| GLU 101 | THR 210 | 14155 |
| SER 115 | ASP 202 | 9477 |
| ARG 97 | THR 210 | 9064 |
| ASN 102 | VAL 200 | 5323 |
| THR 104 | ASP 202 | 4476 |
| ASN 102 | ASN 221 | 3422 |
| GLU 101 | PHE 437 | 3171 |
| ARG 97 | ASP 217 | 2698 |
| GLU 101 | ARG 216 | 2198 |
| ARG 97 | GLY 208 | 1730 |
| GLU 101 | LYS 199 | 1710 |
| SER 115 | SER 224 | 1440 |
| ASN 102 | LYS 199 | 1351 |
| ASN 102 | ASP 212 | 1298 |
| ASN 102 | ARG 405 | 1219 |
| GLU 101 | ARG 207 | 1180 |
| ASN 102 | SER 224 | 1150 |
| ASN 102 | LYS 255 | 1114 |
| ARG 97 | ASP 198 | 946 |
| GLU 101 | PHE 209 | 931 |
| ARG 97 | THR 213 | 791 |
| ARG 97 | ARG 216 | 599 |
| ASN 102 | THR 210 | 589 |
| GLN 114 | ASP 202 | 530 |
| ASN 102 | ASP 202 | 492 |
| ARG 97 | ASP 212 | 490 |
| GLY 103 | ARG 405 | 474 |
| THR 104 | SER 224 | 451 |
| GLU 101 | LYS 255 | 429 |
| ASN 102 | ASP 198 | 405 |
| ASN 102 | PHE 209 | 400 |
| ASN 102 | ARG 178 | 316 |
| ARG 110 | GLU 258 | 309 |
| ASN 102 | ASN 180 | 257 |
| GLN 100 | PHE 223 | 256 |
| GLU 101 | TYR 197 | 220 |
| GLN 114 | SER 228 | 212 |
| LEU 113 | PHE 223 | 210 |
| ASN 102 | ILE 225 | 204 |
| GLN 114 | LYS 227 | 194 |
| GLU 101 | GLY 211 | 189 |
| GLU 101 | ASP 212 | 174 |
| LEU 113 | SER 224 | 159 |
| LEU 113 | GLY 203 | 145 |
| ARG 97 | VAL 220 | 134 |
| GLU 101 | THR 213 | 133 |
| THR 104 | SER 228 | 125 |
| ARG 97 | TYR 197 | 123 |
| LYS 94 | ASP 212 | 118 |
| ASN 102 | ARG 216 | 110 |
| ASN 102 | ASN 235 | 108 |
| ASN 102 | GLY 211 | 104 |
| GLU 101 | ARG 405 | 79 |
| GLN 114 | SER 224 | 69 |
| ASN 102 | VAL 220 | 63 |
| LEU 113 | LYS 227 | 49 |
| ASN 102 | VAL 201 | 42 |
| ARG 97 | PHE 209 | 42 |
| GLU 101 | ASN 180 | 40 |
| ARG 97 | TYR 438 | 38 |
| ARG 97 | ARG 207 | 32 |
| ASN 102 | PHE 407 | 28 |
| SER 115 | ASN 221 | 23 |
| ARG 110 | HIS 204 | 22 |
| GLU 101 | PHE 223 | 21 |
| ARG 97 | ASP 189 | 19 |
| ARG 110 | PHE 223 | 16 |
| THR 104 | ILE 225 | 13 |
| GLY 103 | ASN 180 | 11 |
| ARG 97 | LYS 194 | 11 |
| GLU 101 | PHE 407 | 10 |
| ARG 97 | MET 219 | 9 |
| THR 104 | ASN 235 | 8 |
| ARG 110 | ARG 405 | 8 |
| ARG 97 | TRP 195 | 7 |
| ILE 111 | PHE 223 | 6 |
| GLU 101 | GLY 208 | 6 |
| LEU 113 | ASP 202 | 5 |
| GLU 101 | ARG 178 | 5 |
| ASN 102 | THR 213 | 5 |
| ALA 98 | ARG 216 | 5 |
| ASN 102 | ASP 217 | 4 |
| ARG 97 | LYS 199 | 4 |
| THR 104 | LEU 229 | 3 |
| THR 104 | ARG 405 | 3 |
| GLU 101 | VAL 201 | 3 |
| GLU 101 | MET 219 | 3 |
| ARG 110 | ASP 202 | 3 |
| ARG 110 | ARG 207 | 2 |
| THR 104 | VAL 201 | 1 |
| GLY 103 | SER 224 | 1 |
| GLY 103 | LYS 255 | 1 |
| GLY 103 | GLU 258 | 1 |
| GLY 103 | ASN 235 | 1 |
| GLU 101 | ASP 198 | 1 |
| ASN 102 | PHE 437 | 1 |
| ARG 97 | PHE 437 | 1 |
| ARG 110 | LYS 227 | 1 |

TABLE 8
run 3 enzyme and pore contact interactions

| Pore | Enzyme | # contacts |
|---|---|---|
| ARG 97 | THR 174 | 15557 |
| GLN 100 | ASP 5 | 10353 |
| GLU 101 | LYS 177 | 9238 |
| ARG 97 | SER 179 | 6630 |
| LEU 116 | ASP 202 | 6545 |
| GLU 101 | TYR 434 | 6524 |
| SER 115 | ASP 202 | 5693 |
| GLU 101 | HIS 204 | 5457 |
| ARG 97 | GLN 10 | 5106 |
| ARG 93 | ASP 202 | 4646 |
| ARG 93 | GLU 8 | 4446 |
| SER 115 | LYS 11 | 4342 |
| LEU 113 | ASP 5 | 3871 |
| ASN 102 | SER 224 | 3605 |
| GLU 101 | ASN 12 | 3344 |
| GLU 101 | GLN 10 | 3327 |
| ARG 97 | GLU 175 | 3096 |
| GLU 101 | SER 224 | 3028 |
| LEU 116 | GLU 8 | 2936 |
| LYS 94 | ASP 185 | 2708 |
| ARG 97 | ASN 180 | 2700 |
| GLU 101 | PHE 3 | 2500 |
| THR 104 | LYS 11 | 2352 |
| SER 115 | GLU 8 | 2323 |
| ARG 93 | ASN 180 | 1912 |
| ASN 102 | LYS 177 | 1838 |
| LYS 94 | ASP 198 | 1828 |
| ARG 110 | ASP 5 | 1714 |
| ALA 98 | GLY 203 | 1701 |
| ASN 102 | ASN 12 | 1695 |
| GLU 101 | TYR 169 | 1691 |
| ARG 97 | THR 7 | 1593 |
| ARG 110 | ASP 4 | 1404 |
| ARG 97 | ASP 212 | 1381 |
| ASN 102 | HIS 204 | 1226 |
| ASN 102 | ASN 15 | 1173 |
| ARG 97 | VAL 176 | 1096 |
| ALA 98 | HIS 204 | 998 |
| ARG 97 | ASP 202 | 875 |
| ASN 102 | TYR 434 | 850 |
| ALA 98 | ASN 12 | 716 |
| GLU 101 | THR 213 | 702 |
| GLU 101 | ARG 178 | 642 |
| GLU 101 | ASN 221 | 600 |
| ASN 102 | LYS 11 | 588 |
| ARG 97 | ASP 217 | 585 |
| ARG 97 | ARG 207 | 537 |
| GLU 101 | ARG 207 | 525 |
| ARG 97 | PHE 437 | 511 |
| GLU 101 | ARG 216 | 510 |
| ASN 102 | LYS 19 | 482 |
| ARG 97 | HIS 204 | 473 |
| LEU 113 | LYS 11 | 409 |
| ARG 97 | THR 213 | 358 |
| ARG 93 | ASP 212 | 354 |
| ARG 97 | TYR 169 | 316 |
| ARG 97 | GLY 203 | 308 |
| ARG 97 | ASP 435 | 300 |
| GLN 87 | LYS 199 | 249 |
| THR 104 | ASN 15 | 221 |
| ARG 97 | ALA 181 | 220 |
| ASN 102 | LYS 227 | 198 |
| LYS 94 | ARG 178 | 184 |
| ASN 102 | GLU 8 | 183 |
| LEU 113 | LEU 6 | 182 |
| ARG 93 | SER 179 | 179 |
| LEU 90 | ASN 180 | 172 |
| LEU 90 | ASP 202 | 144 |
| ARG 97 | ILE 225 | 138 |
| GLU 101 | ASN 15 | 135 |
| GLU 101 | LYS 19 | 113 |
| LYS 94 | ASN 180 | 109 |
| LYS 94 | GLU 175 | 105 |
| ARG 93 | THR 7 | 81 |
| LYS 94 | ARG 207 | 77 |
| GLN 100 | PHE 3 | 72 |
| ASN 102 | ARG 216 | 66 |
| ARG 97 | LYS 177 | 62 |
| GLU 101 | THR 210 | 59 |
| ARG 97 | ARG 178 | 56 |
| LYS 94 | ASP 212 | 55 |
| ARG 97 | GLU 172 | 53 |
| GLU 101 | VAL 176 | 51 |
| ALA 98 | ARG 207 | 49 |
| ARG 110 | PHE 3 | 48 |
| ALA 98 | ASP 202 | 47 |
| ARG 97 | VAL 200 | 40 |
| ALA 98 | VAL 201 | 36 |
| LYS 94 | THR 210 | 35 |
| ILE 111 | ASP 5 | 32 |
| ARG 97 | ARG 405 | 27 |
| LEU 90 | VAL 200 | 26 |
| ARG 97 | THR 210 | 26 |
| GLY 103 | PHE 3 | 25 |
| GLU 101 | PHE 209 | 25 |
| ARG 97 | ARG 216 | 22 |
| ASN 102 | VAL 220 | 21 |
| LYS 94 | GLY 211 | 19 |
| ARG 97 | PHE 209 | 17 |
| GLU 101 | LYS 227 | 15 |
| GLN 114 | LYS 11 | 15 |
| GLY 103 | LYS 19 | 13 |
| ARG 97 | PHE 3 | 13 |
| GLU 101 | THR 2 | 12 |
| GLU 101 | ILE 225 | 12 |
| ARG 97 | ILE 184 | 12 |
| ALA 98 | GLU 8 | 12 |
| ALA 98 | ARG 178 | 12 |
| ASN 102 | ILE 225 | 11 |
| LYS 94 | LYS 199 | 10 |
| GLU 101 | ARG 433 | 8 |
| ARG 97 | ASN 221 | 8 |
| LYS 94 | VAL 200 | 7 |
| ASN 102 | ASP 202 | 7 |
| ASN 102 | ASN 221 | 7 |
| ARG 97 | LEU 173 | 7 |
| SER 115 | HIS 204 | 6 |
| ASN 102 | GLY 203 | 6 |
| GLU 101 | CYS 171 | 5 |
| ARG 97 | ASN 12 | 5 |
| ASN 102 | PHE 223 | 4 |
| ASN 102 | LYS 166 | 4 |
| ARG 97 | GLY 211 | 4 |
| ARG 97 | GLN 170 | 4 |
| GLU 101 | ARG 405 | 3 |
| ASN 102 | PHE 3 | 3 |
| GLU 101 | GLU 175 | 2 |
| ARG 97 | VAL 220 | 2 |
| ARG 93 | GLY 203 | 2 |
| LYS 94 | THR 174 | 1 |
| LEU 90 | LYS 199 | 1 |
| LEU 116 | ASN 180 | 1 |
| LEU 113 | ASP 212 | 1 |
| LEU 113 | ASP 202 | 1 |
| GLY 103 | ASN 15 | 1 |
| GLU 101 | THR 7 | 1 |
| GLU 101 | PHE 437 | 1 |
| GLN 114 | ASP 202 | 1 |
| ASN 102 | ARG 405 | 1 |
| ARG 97 | TYR 434 | 1 |
| ARG 97 | PRO 182 | 1 |
| ARG 97 | GLY 9 | 1 |
| ARG 97 | GLU 8 | 1 |
| ALA 99 | ASP 202 | 1 |

### Example 21: Ability of the CsgG Nanopore to the Capture Nucleic Acids in the Channel Constriction

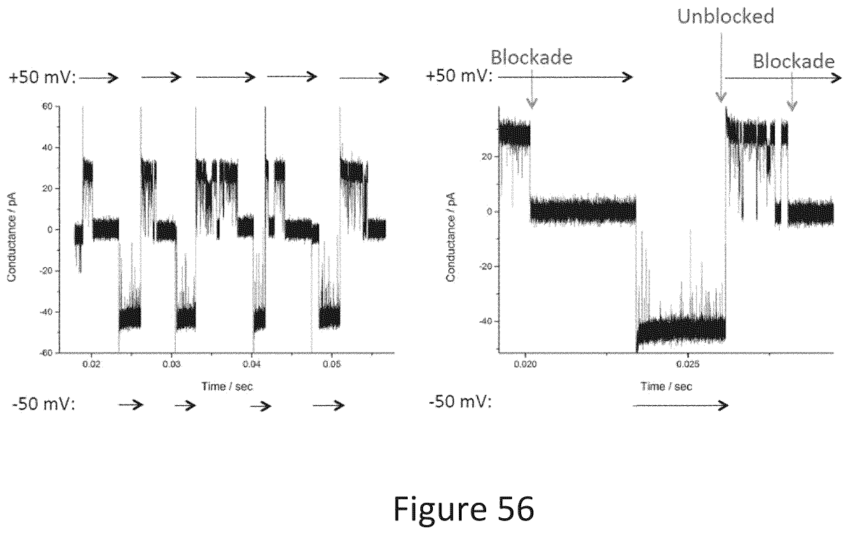

The use of nanopores for nucleic acid sequencing requires the capture and threading of single stranded DNA by the nanopore. In this example, single channel current traces of a CsgG WT protein were followed in presence of a DNA hairpin carrying a single-stranded DNA overhang. The example trace presented in FIG. 56 shows the current which alters in response to the potential measured at +50 mV or −50 mV intervals (indicated by arrows). The downward current blockades in the last +50 mV segment represent the threading of the single-stranded hairpin end into inner pore construction leading to an almost complete current blockade. Reversal of the electrical field to −50 mV results In the electrophoretic unblocking of the pore. A new +50 mV episode results again in DNA hairpin binding and pore blockage. On the +50 mV segments, unfolding of the hairpin structure can lead to the termination of the current blockade indicated by the reversal of the current blockade. The hairpin with the sequence 3′ GCGGGGA GCGTATTAGAGTTGGATCGGATGCAGCTGGCTACTGACGTCATGACGTCAGTAGCCAGCATGCATC CGATC-5′ (SEQ ID NO: 441) was added to the cis side of the chamber at a final concentration of 10 nM.

### Example 22. This Example Describes the Generation of a Mutant CsgG Pore with an Altered Inner Constriction

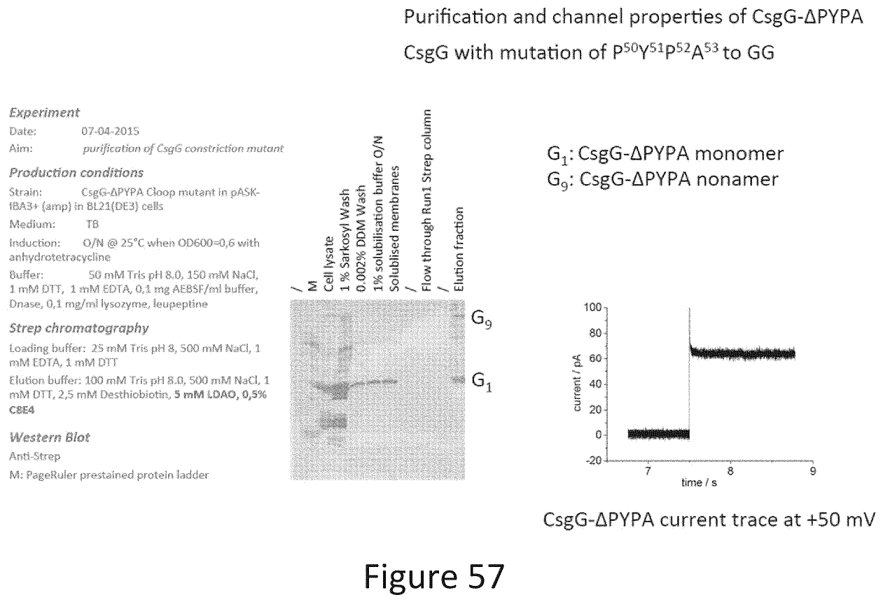

In this example, the stability and channel properties are demonstrated for CsgG-ΔPYPA, a mutant CsgG pore where the sequence PYPA (residues 50-53) in the constriction loop is replaced by GG. For this mutant, the constriction motif from position 38 to 63 corresponds to SEQ ID NO: 354. This mutation foresees in the removal of Y51 from the pore constriction and the shortening of the constriction loop in order to reduce complexity in the pore reading head, i.e. the narrowest part of the pore where the conductivity measured during sensing applications is most sensitive to the nature of the substrate binding or threading the pore. Replacement of the PYPA sequence retains stability of the CsgG nonamer and results in a pore with increase conductivity as shown in FIG. 57.

The CsgG-ΔPYPA pore mutant was analysed with single-channel current recordings using parallel high-resolution electrical recording with the Orbit 16 kit from Nanion (Munich. Germany). Briefly, horizontal bilayers of 1,2-diphytanoyl-sn-glycero-3-phosphocholine (Avanti Polar Lipids) were formed over microcavities (of sub-picoliter volume) in a 16-channel multielectrode cavity array (MECA) chip (Ionera, Freiburg, Germany) 51. Both the cis and trans cavities above and below the bilayer contained 1.0 M KCl, 25 mM Tris-HCl, pH 8.0. To Insert channels into the membrane, CsgG dissolved In 25 mM Tris pH 8.0, 500 mM NaCl, 1 mM DTT, 0.5% C8E4, 5 mM LDAO was added to the cis compartment to a final concentration of 100 nM. HTransmembrane currents were recorded at a holding potential of +50 mV and −50 mV (with the cis side grounded) using a Tecella Triton 16 channel amplifier at a low-pass filtering frequency of 3 kHz and a sampling frequency of 10 kHz. Current traces were analyzed using the Clampfit of the pClamp suite (Molecular Devices, USA).

### Example 23

This example describes the structural and mechanistic insights into the bacterial amyloid secretion channel CsgG.

Curli are functional amyloid fibres that constitute the major protein component of the extracellular matrix in pellicle biofilms formed by Bacteroidetes and Proteobacteria (predominantly of the a and c classes)¹⁻³. They provide a fitness advantage in pathogenic strains and induce a strong pro-inflammatory response during bacteraemia^{1,4,5}. Curli formation requires a dedicated protein secretion machinery comprising the outer membrane lipoprotein CsgG and two soluble accessory proteins, CsgE and CsgF^{6,7}. Here we report the X-ray structure of Escherichia coli CsgG in a non-lipidated, soluble form as well as in its Native membrane-extracted conformation. CsgG forms an oligomeric transport complex composed of nine anticodon-binding-domain-like units that give rise to a 36-stranded β-barrel that traverses the bilayer and is connected to a cage-like vestibule in the periplasm. The transmembrane and periplasmic domains are separated by a 0.9-nm channel constriction composed of three stacked concentric phenylalanine, asparagine and tyrosine rings that may guide the extended polypeptide substrate through the secretion pore. The specificity factor CsgE forms a nonameric adaptor that binds and closes off the periplasmic face of the secretion channel, creating a 24,000 Å⁻³ pre-constriction chamber. Our structural, functional and electrophysiological analyses imply that CsgG is an ungated, non-selective protein secretion channel that is expected to employ a diffusion-based, entropy-driven transport mechanism.

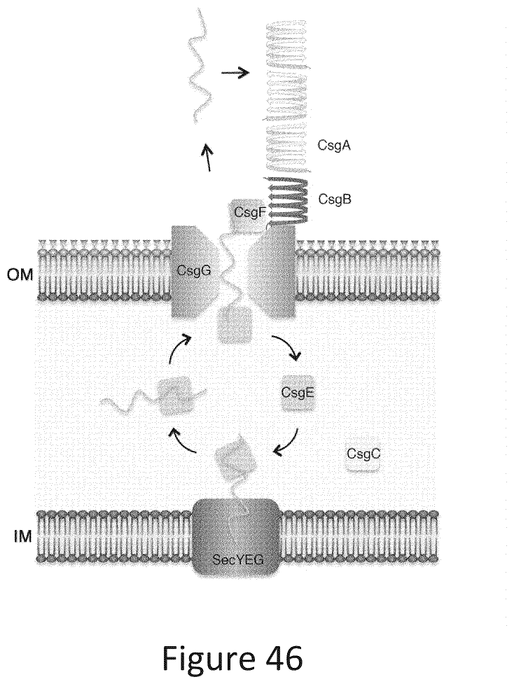

Curli are bacterial surface appendages that have structural and physical characteristics of amyloid fibrils, best known from human degenerative diseases⁷⁻⁹. However, the role of bacterial amyloids such as curli are to facilitate biofilmformation^{4,10}. Unlike pathogenic amyloids, which are the product of protein misfolding, curli formation is coordinated by proteins encoded in two dedicated operons, csgBAC (curli specific genes BAC) and csgDEFG in Escherichia coli (FIG. 46)^{6,7}. After secretion, CsgB nucleates CsgA subunits into curli fibres^{7,11,12}. Secretion and extracellular deposition of CsgA and CsgB are dependent on two soluble accessory factors, respectively CsgE and CsgF, as well as CsgG, a 262-residue lipoprotein located in the outer membrane¹³⁻¹⁶. Because of the lack of hydrolysable energy sources or ion gradients at the outer membrane, CsgG falls into a specialized class of protein translocators that must operate through an alternatively energized transport mechanism. In the absence of a structural model, the dynamic workings of how CsgG promotes the secretion and assembly of highly stable amyloid like fibres in a regulated fashion across a biological membrane has so far remained enigmatic.

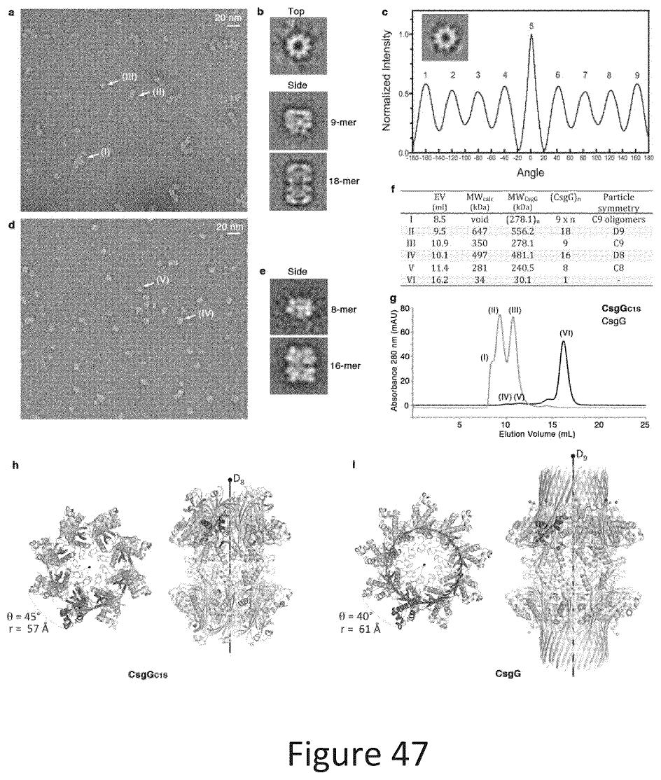

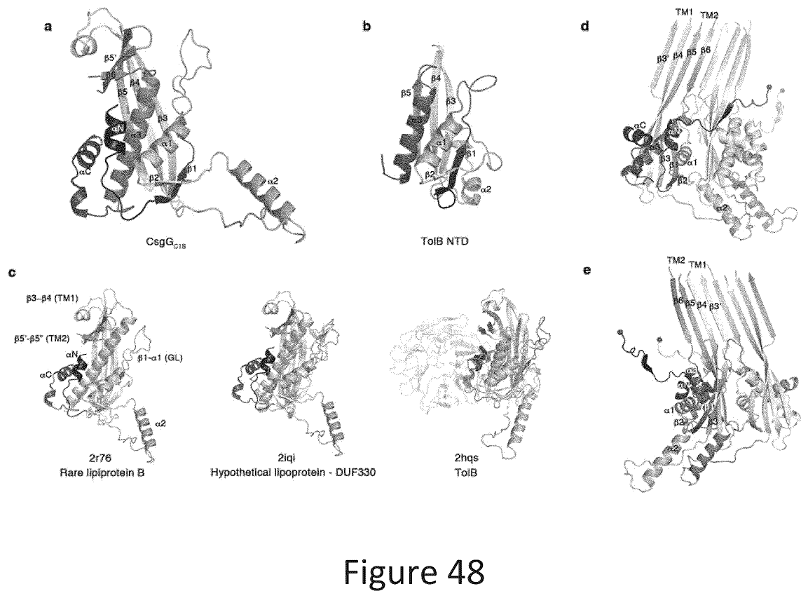

Before insertion into the outer membrane, lipoproteins are piloted across the periplasm by means of the lipoprotein localization (Lol) pathway¹⁷. We observed that non-lipidated CsgG (CsgG_{C1S}) could be isolated as a soluble periplasmic intermediate, analogous to the pre-pore forms observed in pore-forming proteins and toxins¹⁸. CsgG_{C1S} was found predominantly as monomers, in addition to a minor fraction of discrete oligomeric complexes (FIG. 47)¹⁹. The soluble CsgG_{C1S} oligomers were crystallized and their structure was determined to 2.8 Å, revealing a hexadecameric particle with eight-fold dihedral symmetry (D8), consisting of two ring-shaped octameric complexes (C8) that are joined in a tail-to-tail interaction (FIG. 47 and FIG. 46).The CsgG_{C1S} protomer shows an anticodon-binding domain (ABD)-like fold that is extended with two α-helices at the amino and carboxy termini (αN and αC, respectively; FIG. 42 and FIG. 48a-c). Additional CsgG-specific elements are an extended loop linking β1 and α1, two Insertions in the loops connecting β3-β4 and β5-α3 and an extended α2 helix that is Implicated in CsgG oligomerization by packing between adjacent monomers (FIG. 42b). Further inter-protomer contacts are formed between the back of the β3-β5 sheet and the extended β1-α1 loop (FIG. 48d, e).

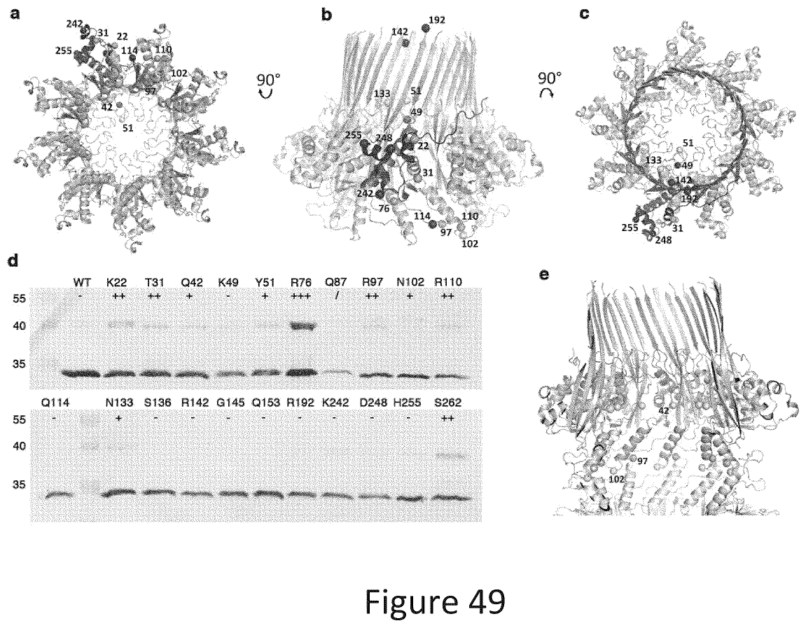

In the CsgG_{C1S} structure, residues 1-17, which would link β1 to the N-terminal lipid anchor, are disordered and no obvious transmembrane (TM) domain can be discerned (FIG. 42). Attenuated total reflection Fourier transform infrared spectroscopy (ATR-FTIR) of CsgG_{C1S} and native, membrane-extracted CsgG revealed that the latter has a higher absorption In the β-sheet region (1,625-1,630 cm⁻¹) and a concomitant reduction in the random coil and α-helical regions (1,645-1,650 cm⁻¹ and 1,656 cm⁻¹, respectively; FIG. 43a), suggesting that membrane-associated CsgG contains a β-barrel domain. Candidate sequence stretches for β-strand formation are found in the poorly ordered, extended loops connecting β3-β4 (residues 134-154) and β5-α3 (residues 184-204); deletion of these resulted in the loss of curli formation (FIG. 43b).The crystal structure of detergent-extracted CsgG confirmed a conformational rearrangement of both regions into two adjacent β-hairpins, extending the β-sheet formed by β3-β4 (TM1) and β5-α3 (TM2) (FIG. 43c). Their juxtaposition in the CsgG oligomer gave rise to a composite 36-stranded β-barrel (FIG. 43d). Whereas the crystallized CsgG_{C1S} oligomers showed a D8 symmetry, the CsgG structure showed D9 symmetry, with CsgG protomers retaining equivalent interprotomer contacts, except for a 5° rotation relative to the central axis and a 4 Å translation along the radial axes FIG. 47). This observation is reconciled In the in-solution oligomeric states revealed by single-particle electron microscopy, which exclusively found C9 and D9 symmetries for membrane-extracted CsgG (FIG. 47). The predominant presence of monomers in the non-lipidated sample and the symmetry mismatch with the membrane-bound protein argue that before membrane insertion, CsgG is targeted to the outer membrane in a monomeric, LolA-bound form and that the C8 and D8 particles are an artefact of highly concentrated solutions of CsgG_{C1S}. Furthermore, we show that the C9 nonamer rather than the D9 complex forms the physiologically relevant particle, because in isolated E. coli outer membranes, cysteine substitutions in residues enclosed by the observed tail-to-tail dimerization are accessible to labelling with maleimidepolyethylene glycol (PEG, 5 kDa; FIG. 49).

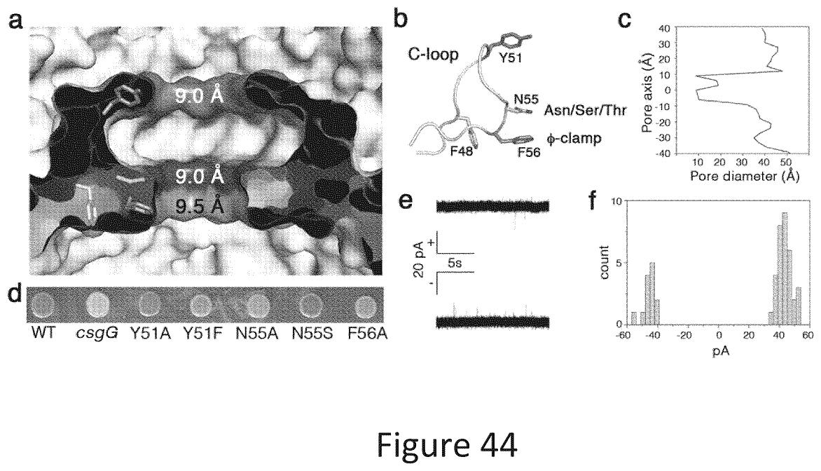

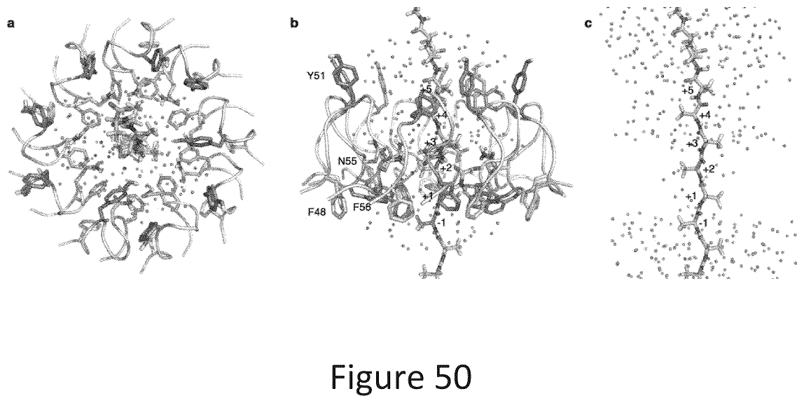

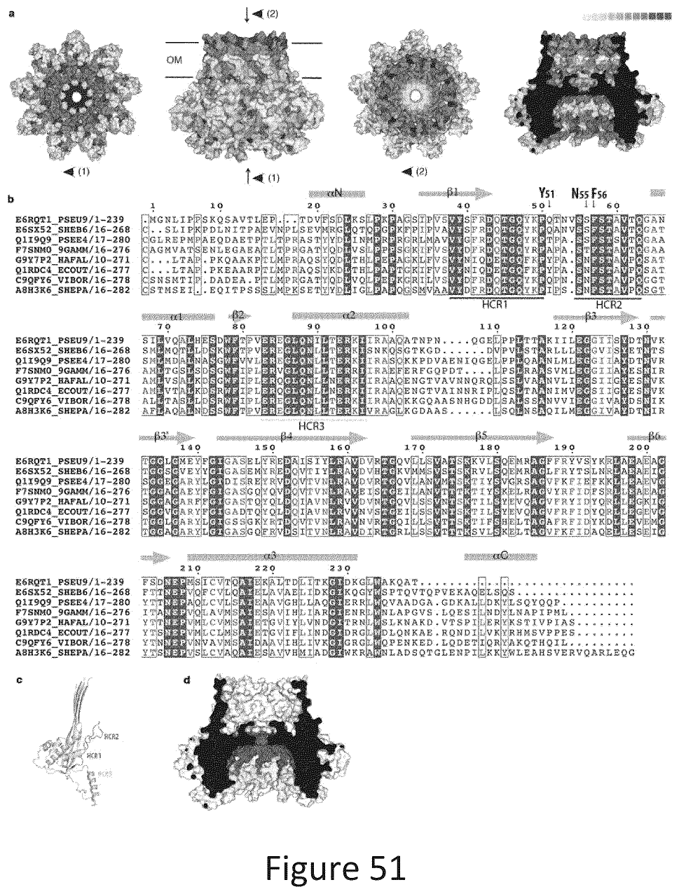

Thus, CsgG forms a nonameric transport complex 120 Å in width and 85 Å in height. The complex traverses the outer membrane through a 36-stranded β-barrel with an Inner diameter of 40 Å (FIG. 43e). The N-terminal lipid anchor is separated from the core domain by an 18-residue linker that wraps over the adjacent protomer (FIG. 48d). The diacylglycerol- and amide-linked acyl chain on the N-terminal Cys are not resolved in the electron density maps, but on the basis of the location of Leu 2 the lipid anchor is expected to flank the outer wall of the 0-barrel. On the periplasmic side, the transporter forms a large solvent—accessible cavity with an inner diameter of 35 Å and a height of 40 Å that opens to the periplasmin a 50 Å mouth formed by helix 2 (FIG. 43e). At its apex, this periplasmic vestibule is separated from the TM channel by a conserved 12-residue loop connecting β1 to α1 (C-loop; FIGS. 43e and 44a, b), which constricts the secretion conduit to a solvent-excluded diameter of 9.0 Å (FIG. 44a, c). These pore dimensions would be compatible with the residence of one or two (for example a looped structure) extended polypeptide segments, with five residues spanning the height of the constriction (FIG. 50).The luminal lining of the constriction is composed of three stacked concentric rings formed by the side chains of residues Tyr 51, Asn 55 and Phe 56 (FIG. 44a, b). In the anthrax PA63 toxin, a topologically equivalent concentric Phe ring (referred to as a Φ-clamp) lines the entry of the translocation channel and catalyses polypeptide capture and passage²⁰⁻²². Multiple sequence alignment of CsgG-like translocators shows the absolute conservation of Phe 56 and the conservative variation of Asn 55 to Ser or Thr (FIG. 51). Mutation of Phe 56 or Asn 55 to Ala leads to a near loss of curli production (FIG. 44d), whereas a Asn 55→Ser substitution retains wild-type secretion levels, together alluding to the requirement of the stacked configuration of a 0-clamp followed by a hydrogen-bond donor/acceptor in the CsgG constriction (FIG. 44b and FIG. 51).

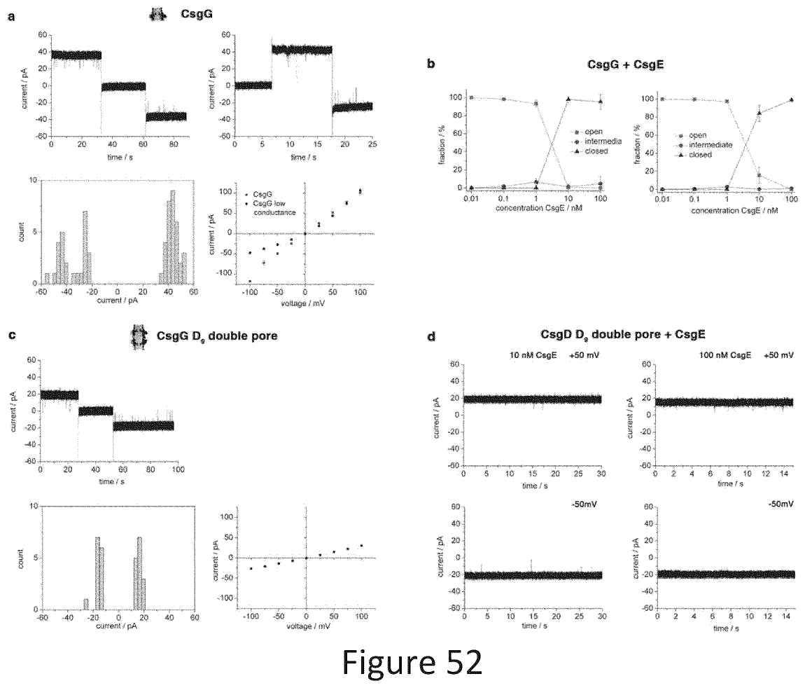

Single-channel current recordings of CsgG reconstituted in planar phospholipid bilayers led to a steady current of 43.1±4.5 pA (n=33) or −45.1±4.0 pA (n=13) using standard electrolyte conditions and a potential of 450 mV or −50 mV, respectively (FIG. 44e, f and FIG. 52). The observed current was in good agreement with the predicted value of 46.6 pA calculated on the basis of a simple three-segment pore model and the dimensions of the central constriction seen in the X-ray structure (FIG. 44c). A second, low-conductance conformation can also be observed under negative electrical field potential (−26.2±3.6 pA (n=13); FIG. 52). It is unclear, however, whether this species is present under physiological conditions.

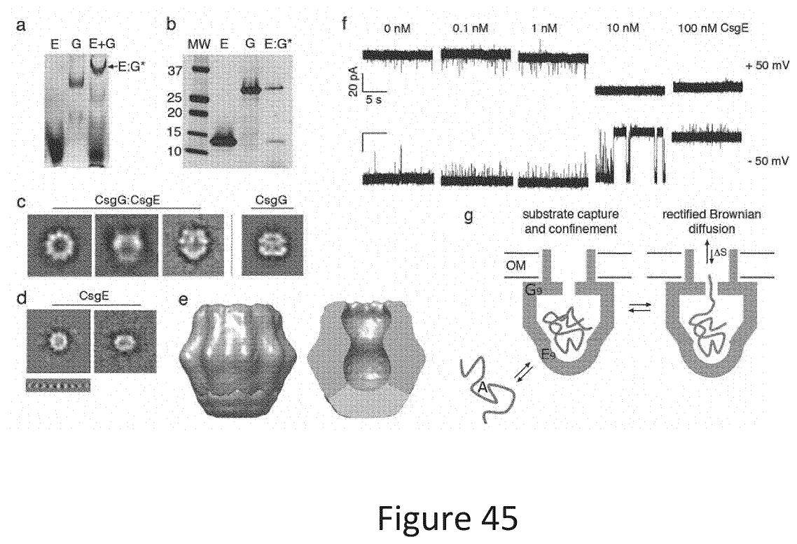

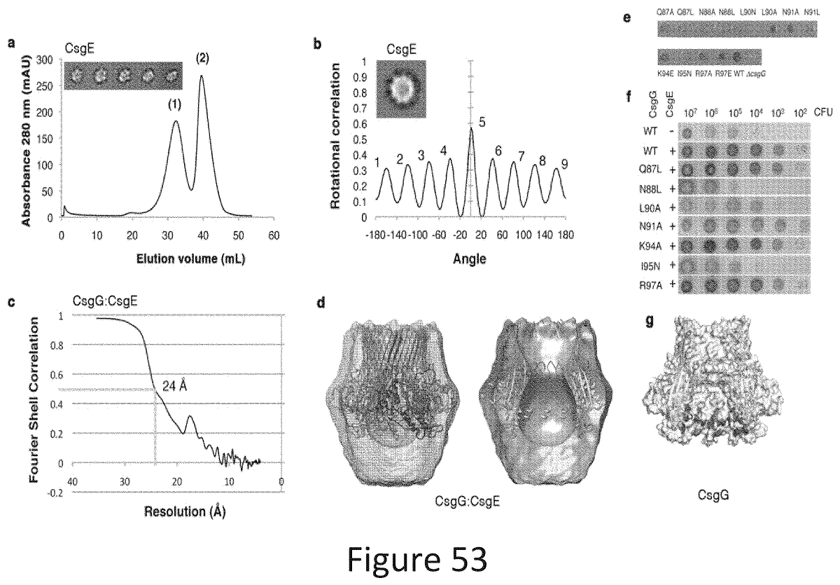

Our structural data and single-channel recordings imply that CsgG forms an ungated peptide diffusion channel. In PA₆₃, a model peptide diffusion channel, polypeptide passage depends on a ΔpH-driven Brownian ratchet that rectifies the diffusive steps in the translocation channel²⁰⁻²². However, such proton gradients are not present at the outer membrane, requiring an alternative driving force. Whereas at elevated concentrations CsgG facilitates a non-selective diffusive leakage of periplasmic polypeptides, secretion is specific for CsgA under native conditions and requires the periplasmic factor CsgE^{16,23}. In the presence of excess CsgE, purified CsgG forms a more slowly migrating species on native PAGE (FIG. 45a).SDS-PAGE analysis shows this new species consists of a CsgG-CsgE complex that is present in a 1:1 stoichiometry (FIG. 45b). Cryoelectron microscopy (cryo-EM) visualization of CsgG-CsgE isolated by pull-down affinity purification revealed a nine-fold symmetrical particle corresponding to the CsgG nonamer and an additional capping density at the entrance to the periplasmic vestibule, similar in size and shape to a C9 CsgE oligomer also observed by single-particle EM and size-exclusion chromatography (FIG. 45c-e and FIG. 53). The location of the observed CsgG-CsgE contact interface was corroborated by blocking point mutations in CsgG helix 2 (FIG. 53). In agreement with a capping function, single-channel recordings showed that CsgE binding to the translocator led to the specific silencing of its ion conductance (FIG. 45f and FIG. 52). This CsgE capping of the channel seemed to be an all-or-none response in function of CsgE nomamerbinding. At saturation. CsgE binding induced full blockage of the channel, whereas at about 10 nM, an equilibrium between CsgE binding and dissociation events resulted in an intermittently blocked or fully open translocator. At 1 nM or below, transient (<1 ms) partial blockage events may have stemmed from short-lived encounters with monomeric CsgE.

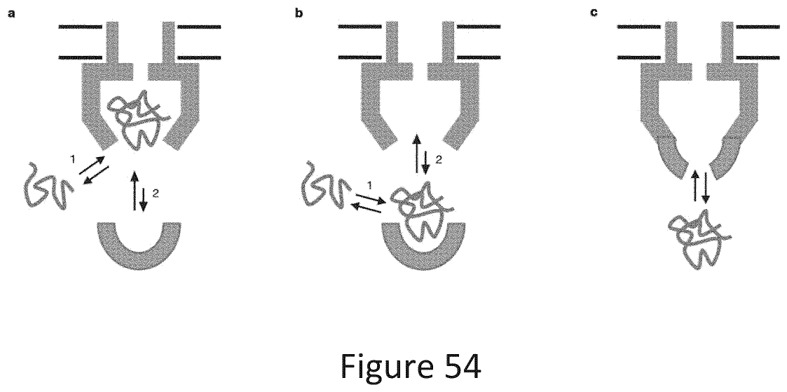

Thus. CsgG and CsgE seem to form an encaging complex enclosing a central cavity of ˜24,000 Å³, reminiscent in appearance to the substrate binding cavity and encapsulating lid structure seen in the GroEL chaperonin and GroES co-chaperonin²⁴. The CsgG-CsgE enclosure would be compatible with the full or partial entrapment of the 129-residue CsgA. The caging of a translocation substrate has recently been observed in ABC toxins²⁵. Spatial confinement of an unfolded polypeptide leads to a decrease in its conformational space, creating an entropic potential that has been shown to favour polypeptide folding in the case of chaperonins^{24,26}. Similarly, we speculate that in curli transport the local high concentration and conformational confinement of curli subunits in the CsgG vestibule would generate an entropic free-energy gradient aver the translocation channel (FIG. 45g).On capture into the constriction, the polypeptide chain is then expected to move progressively outwards by Brownian diffusion, rectified by the entropic potential generated from the CsgE-mediated confinement and/or substrate trapping near the secretion channel. For full confinement in the pre-constriction cavity, the escape of an unfolded 129-residue polypeptide to the bulk solvent would correspond to an entropic free-energy release of up to ˜80 kcal mol⁻¹ (about 340 kJ mol⁻¹; ref. 27). The initial entropic cost of substrate docking and confinement are likely to be at least partly compensated for by binding energy released during assembly of the CsgG-CsgE-CsgA complex and an already lowered CsgA entropy in the periplasm. On theoretical grounds, three potential routes of CsgA recruitment to the secretion complex can be envisaged (FIG. 54).

Curli-induced biofilms form a fitness and virulence factor in pathogenic Enterobacteriaceae^{4,5}. Their unique secretion and assembly properties are also rapidly gaining interest for (bio)technological application^{23,28,29}, Our structural characterization and biochemical study of two key secretion components provide a tentative model of an iterative mechanism for the membrane translocation of unfolded protein substrates in the absence of a hydrolysable energy source, a membrane potential or an ion gradient (FIG. 45e and FIG. 54). The full validation and deconstruction of the contributing factors in the proposed secretion model will require the in vitro reconstitution of the translocator to allow transport kinetics to be followed accurately at the single-molecule level.

### Methods

Cloning and strains. Expression constructs for the production of outer membrane localized C-terminally StrepII-tagged CsgG (pPG1) and periplasmic C-terminally StrepII-tagged CsgG_{C1S} (pPG2) have been described in ref. 19. For selenomethionine labelling, StrepII-tagged CsgG_{C1S} was expressed in the cytoplasm because of increased yields. Therefore, ₚPG2 was altered to remove the N-terminal signal peptide using inverse PCR with primers 5′-TCTTTAACCGCCCCGCCTAAAG-3′ (forward) and 5′-CATTTTTTGCCCTCGTTATC-3′ (reverse) (pPG3). For phenotypic assays, a csgG deletion mutant of E. coli BW25141 (E. coli NVG2) was constructed by the method described in ref. 30 (with primers 5′-AATAACTCAACC GAT TTT TAA GCC CCA GCT TCA TAA GGA AAA TAA TCG TGT AGG CTG GAG CTG CTT C-3′ and 5′-CGC TTA AAC AGT AAA ATG CCG GAT GAT AAT TCC GGC TTT TTT ATC TGC ATA TGA ATA TCC TCC TTA G-3′). The various CsgG substitution mutants used for Cys accessibility assays and for phenotypic probing of the channel constriction were constructed by site-directed mutagenesis (QuikChange protocol; Stratagene) starting from pMC2, a pTRC99a vector containing csgG under control of the trc promoter¹⁴.

Protein expression and purification CsgG and CsgG_{C1S} were expressed and purified as described¹⁹. In brief, CsgG was recombinantly produced in E. coli BL 21 (DE3) transformed with pPG1 and extracted from isolated outer membranes with the use of 1% n-dodecyl-b-D-maltoside (DDM) in buffer A (50 mM Tris-HCl pH 8.0, 500 mM NaCl, 1 mM EDTA, 1 mM dithiothreitol (DTT)). StrepII-tagged CsgG was loaded onto a 5 ml Strep-Tactin Sepharose column (Iba GmbH) and detergent exchanged by washing with 20 column volumes of buffer A supplemented with 0.5% tetraethylene glycol monooctyl ether (C8E4; Affymetrix) and 4 mM lauryldimethylamine-N-oxide (LDAO; Affymetrix). The protein was eluted by the addition of 2.5 mM D-desthiobiotin and concentrated to 5 mg ml⁻¹ for crystallization experiments. For selenomethionine labelling, CsgG_{C1S} was produced in the Met auxotrophic strain B834 (DE3) transformed with pPG3 and grown on M9 minimal medium supplemented with 40 mg l⁻¹ L-selenomethionine. Cell pellets were resuspended in 50 mM Tris-HCl pH 8.0, 150 mM NaCl, 1 mM EDTA, 5 mM DTT, supplemented with cOmplete Protease Inhibitor Cocktail (Roche) and disrupted by passage through a TS series cell disruptor (Constant Systems Ltd) operated at 20×10³ lb in⁻². Labelled CsgG_{C1S} was purified as described¹⁹. DTT (5 mM) was added throughout the purification procedure to avoid oxidation of selenomethionine.

CsgE was produced in E. coli NEBC2566 cells harbouring pNH27 (ref. 16). Cell lysates in 25 mM Tris-HCl pH 8.0, 150 mM NaCl, 25 mM imidazole, 5% (v/v) glycerol were loaded on a HisTrap FF (GE Healthcare). CsgE-his was eluted with a linear gradient to 500 mM imidazole in 20 mM Tris-HCl pH 8.0, 150 mM NaCl, 5% (v/v) glycerol buffer. Fractions containing CsgE were supplemented with 250 mM (NH₄)₂SO₄ and applied to a 5 ml HiTrap Phenyl HP column (GE Healthcare) equilibrated with 20 mM Tris-HCl pH 8.0, 100 mM NaCl, 250 mM (NH₄)₂SO₄, 5% (v/v) glycerol. A linear gradient to 20 mM Tris-HCl pH 8.0, 10 mM NaCl, 5% (v/v) glycerol was applied for elution. CsgE containing fractions were loaded onto a Superose 6 Prep Grade 10/600 (GE Healthcare) column equilibrated in 20 mM Tris-HCl pH 8.0, 100 mM NaCl, 5% (v/v) glycerol.

In-solution oligomeric state assessment. About 0.5 mg each of detergent-solubilized CsgG (0.5% C8E4, 4 mM LDAO) and CsgG_{C1S} were applied to a Superdex 200 10/300 GL analytical gel filtration column (GE Healthcare) equilibrated with 25 mM Tris-HCl pH 8.0, 500 mM NaCl, 1 mM DTT, 4 mM LDAO and 0.5% C8E4 (CsgG) or with 25 mM Tris-HCl pH 8.0, 200 mM NaCl (CsgG_{C1S}), and run at 0.7 ml min⁻¹. The column elution volumes were calibrated with bovine thyroglobulin, bovine γ-globulin, chicken ovalbumin, horse myoglobulin and vitamin B12 (Bio-Rad) (FIG. 47). Membrane-extracted CsgG, 20 mg of the detergent-solubilized protein was also run on 3-10% blue native PAGE using the procedure described in ref. 31 (FIG. 47). NativeMark (Life Technologies) unstained protein standard (7 ml) was used for molecular mass estimation

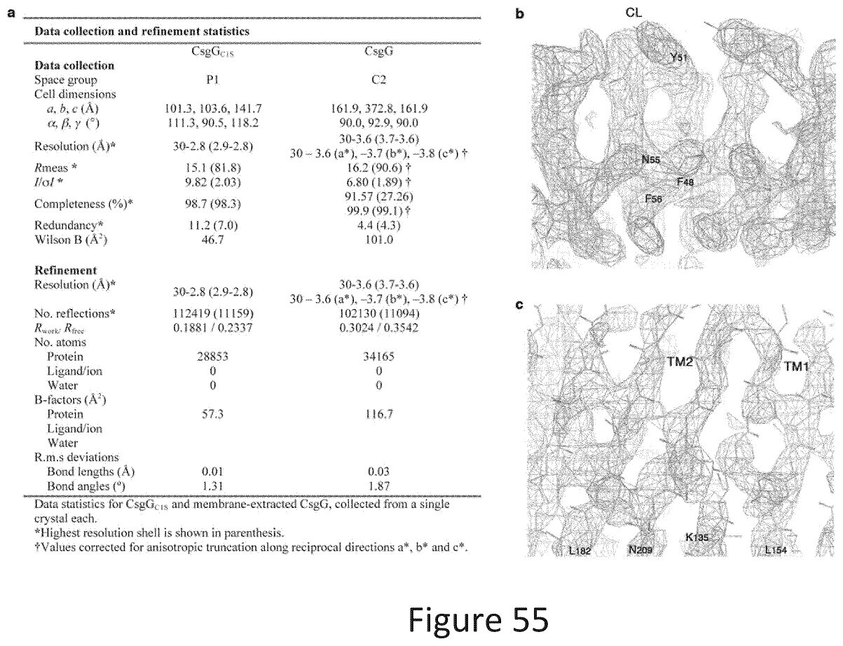

Crystallization, data collection and structure determination. Selenomethionine-labelled CsgG_{C1S} was concentrated to 3.8 mg ml¹ and crystallized by sitting-drop vapour diffusion against a solution containing 100 mM sodium acetate pH 4.2, 8% PEG 4000 and 100 mM sodium malonate pH 7.0. Crystals were incubated in crystallization buffer supplemented with 15% glycerol and flash-frozen in liquid nitrogen. Detergent-solubilized CsgG was concentrated to 5 mg ml¹ and crystallized by hanging-drop vapour diffusion against a solution containing 100 mM Tris-HCl pH 8.0, 8% PEG 4000, 100 mM NaCl and 500 mM MgCl₂. Crystals were flash-frozen in liquid nitrogen and cryoprotected by the detergent present in the crystallization solution. For optimization of crystal conditions and screening for crystals with good diffraction quality, crystals were analysed on beamlines Proxima-1 and Proxima-2a (Soleil, France), PX-I (Swiss Light Source. Switzerland), 102, 103, 104 and 124 (Diamond Light Source, UK) and ID14eh2, ID23eh1 and ID23eh2 (ESRF, France). Final diffraction data used for structure determination of CsgG_{C1S} and CsgG were collected at beamlines 104 and 103, respectively (see FIG. 55a for data collection and refinement statistics). Diffraction data for CsgG_{C1S} were processed using Xia2 and the XDS package^{32,33}. Crystals of CsgG_{C1S} belonged to space group P1 with unit cell dimensions of a=101.3 Å, b=103.6 Å, c=141.7 Å, α=111.3°, β=90.5, γ=118.2°, containing 16 protein copies in the asymmetric unit. For structure determination and refinement, data collected at 0.9795 Å wavelength were truncated at 2.8 Å on the basis of an I/δI cutoff of 2 in the highest-resolution shell. The structure was solved using experimental phases calculated from a single anomalous dispersion (SAD) experiment. A total of 92 selenium sites were located in the asymmetric unit by using ShelxC and ShelxD³⁴, and were refined and used for phase calculation with Sharp³⁵ (phasing power 0.79, figure of merit (FOM) 0.25). Experimental phases were density modified and averaged by non-crystallographic symmetry (NCS) using Parrot³⁶ (FIG. 55; FOM 0.85). An Initial model was built with Buccaneer³⁷ and refined by Iterative rounds of maximum-likelihood refinement with Phenix refine³⁸ and manual inspection and model (re)building in Coot³⁹. The final structure contained 28.853 atoms in 3,700 residues belonging to 16 CsgG_{C1S} chains (FIG. 47), with a molprobily⁴⁰ score of 1.34; 98% of the residues lay in favoured regions of the Ramachandran plot (99.7% in allowed regions). Electron density maps showed no unambiguous density corresponding to possible solvent molecules, and no water molecules or ions were therefore built in. Sixteenfold NCS averaging was maintained throughout refinement, using strict and local NCS restraints In early and late stages of refinement, respectively.

Diffraction data for CsgG were collected from a single crystal at 0.9763 Å wavelength and were indexed and scaled, using the XDS package^{32,33}, in space group C2 with unit-cell dimensions a=161.7 Å, b=372.3 Å, c=161.8 Å and β=92.9°, encompassing 18 CsgG copies in the asymmetric unit and a 72% solvent content. Diffraction data for structure determination and refinement were elliptically truncated to resolution limits of 3.6 Å, 3.7 Å and 3.8 Å along reciprocal cell directions a*, b* and c* and scaled anisotropically with the Diffraction Anisotropy Server⁴¹. Molecular replacement using the CsgG_{C1S} monomer proved unsuccessful. Analysis of the self rotation function revealed D_{g} symmetry in the asymmetric unit (not shown). On the basis of on the CsgG_{C1S} structure, a nonameric search model was generated in the assumption that after going from a C₈ to C₉ oligomer, the interprotomer arc at the particle circumference would stay approximately the same as the Interprotomer angle changed from 45° to 40°, giving a calculated increase in radius of about 4 Å. Using the calculated nonamer as search model, a molecular replacement solution containing two copies was found with Phaser⁴². Inspection of density-modified and NCS-averaged electron density maps (Parrot³⁶; FIG. 55) allowed manual building of the TM1 and TM2 and remodeling of adjacent residues in the protein core, as well as the building of residues 2-18, which were missing from the CsgG_{C1S} model and linked the α1 helix to the N-terminal lipid anchor. Refinement of the CsgG mode I was performed withBuster-TNT⁴³ and Refmac5 (ref. 44) for initial and final refinement rounds, respectively. Eighteenfold local NCS restraints were applied throughout refinement, and Refmac5 was run employing a jelly-body refinement with sigma 0.01 and hydrogen-bond restraints generated by Prosmart⁴⁵. The final structure contained 34,165 atoms in 4,451 residues belonging to 18 CsgG chains (FIG. 47), with a molprobity score of 2.79; 93.0% of the residues lay in favoured regions of the Ramachandran plot (99.3% In allowed regions). No unambiguous electron density corresponding the N-terminal lipid anchor could be discerned.

Congo red assay. For analysis of Congo red binding, a bacterial overnight culture grown at 37° C. in Lysogeny Broth (LB) was diluted in LB medium until a D₆₀₀ of 0.5 was reached. A 5 μl sample was spotted on LB agar plates supplemented with ampillicin (100 mg l⁻¹). Congo red (100 mg l⁻¹) and 0.1% (w/v) isopropyl β-D-thiogalactoside (IPTG) Plates were incubated at room temperature (20-22° C.) for 48 h to induce curli expression. The development of the colony morphology and dye binding were observed at 48 h.

Cysteine accessibility assays. Cysteine mutants were generated in pMC2 using site-directed mutagenesis and expressed in E. coli LSR12 (ref. 7). Bacterial cultures grown overnight were spotted onto LB agar plates containing 1 mM IPTG and 100 mg l⁻¹ ampicillin. Plates were incubated at room temperature and cells were scraped after 48 h, resuspended in 1 ml of PBS and normalized using D₆₀₀. The cells were lysed by sonication and centrifuged for 20 s at 3,000 g at 4° C. to remove unbroken cells from cell lysate and suspended membranes. Proteins in the supernatant were labelled with 15 mM methoxypolyethylene glycol-maleimide (MAL-PEG 5 kDa) for 1 h at room temperature. The reaction was stopped with 100 mM DTT and centrifuged at 40,000 r.p.m. (˜100,000 g) in a 50.4 Ti rotor for 20 min at 4° C. to pellet total membranes. The pellet was washed with 1% sodiumlauroyl sarcosinate to solubilize cytoplasmic membranes and centrifuged again. The resulting outer membranes were resuspended and solubilized using PBS containing 1% DDM. Metal affinity pull downs with nickel beads were used for SDS-PAGE and anti-His western blots. E. coli LSR12 cells with empty pMC2 vector were used as negative control.

ATR-FTIR spectroscopy. ATR-FTIR measurements were performed on an Equinox 55 infrared spectrophotometer (Bruker), continuously purged with dried air, equipped with a liquid-nitrogen-refrigerated mercury cadmium telluride detector and a Golden Gate reflectance accessory (Specac). The internal reflection element was a diamond crystal (2 mm×2 mm) and the beam incidence angle was 45°. Each purified protein sample (1 μl) was spread at the surface of the crystal and dried under a gaseous nitrogen flow to form a film. Each spectrum, recorded at 2 cm⁻¹ resolution, was an average of 128 accumulations for improved signal-to-noise ratio. All the spectra were treated with water vapour contribution subtraction, smoothed at a final resolution of 4 cm⁻¹ by apodization and normalized on the area of the Amide I band (1,700-1,600 cm⁻¹) to allow their comparison⁴⁶.

Negative stain EM and symmetry determination. Negative stain EM was used to monitor in-solution oligomerization states of CsgG, CsgG_{C1S} and CsgE. CsgE, CsgG_{C1S} and amphipol-bound CsgG were adjusted to a concentration of 0.05 mg ml⁻¹ and applied to glow-discharged carbon-coated copper grids (CF-400; Electron Microscopy Sciences). After 1 min incubation, samples were blotted, then washed and stained in 2% uranyl acetate. Images were collected on a Tecnai T12 BioTWIN LaB6 microscope operating at a voltage of 120 kV, at a magnification of ×49,000 and defocus between 800 and 2,000 nm. Contrast transfer function (CTF), phase flipping and particle selection were performed as described for cryo-EM. For membrane extracted CsgG, octadecameric particles (1,780 in all) were analysed separately from nonamers and top views. For purified CsgE a total of 2,452 particles were analysed. Three-dimensional models were obtained as described for the CsgG-CsgE cryo-EManalysis below and refined by several rounds of multi-reference alignment (MRA), multi-statistical analysis (MSA) and anchor set refinement. In all cases, after normalization and centring, images were classified using IMAGIC-4D as described in the cryo-EM section. The best classes corresponding to characteristic views were selected for each set of particles. Symmetry determination of CsgG top views was performed using the best class averages with roughly 20 images per class. The rotational autocorrelation function was calculated using IMAGIC and plotted.

CsgG-CsgE complex formation. For CsgG-CsgE complex formation, the solubilizing detergents in purified CsgG were exchanged for Amphipols A8-35 (Anatrace) by adding 120 μl of CsgG (24 mg ml⁻¹ protein in 0.5% C8E4, 4 mM LDAO, 25 mM Tris-HCl pH 8.0, 500 mM NaCl. 1 mM DTT) to 300 μl of detergent-destabilized liposomes (1 mg ml⁻¹ 1,2-dimyristoyl-sn-glycero-3-phosphocholine (DMPC) and 0.4% LDAO) and incubating for 5 min on ice before the addition of 90 ml of A8-35 amphipols at 100 mg ml⁻¹ stock. After an additional 15 min incubation on ice, the sample was loaded on a Superose 6 10/300 GL (GE Healthcare) column and gel filtration was performed in 200 mM NaCl, 2.5% xylitol, 25 mM Tris-HCl pH 8, 0.2 mM DTT. An equal volume of purified monomeric CsgE in 200 mM NaCl, 2.5% xylitol, 25 mM Tris-HCl pH 8, 0.2 mM DTT was added to the amphipol-solubilized CsgG at final protein concentrations of 15 and 5 μM for CsgE and CsgG, respectively, and the sample was run at 125V at 18° C. on a 4.5% native PAGE in 0.5×TBE buffer. For the second, denaturing dimension, the band corresponding to the CsgG-CsgE complex was cut out of unstained lanes run in parallel on the same gel, boiled for 5 min in Laemmli buffer (60 mM Tris-HCl pH 6.8, 2% SDS, 10% glycerol, 5% 2-mercaptoethanol, 0.01% bromophenol blue) and run on 4-20% SDS-PAGE. Purified CsgE and CsgG were run alongside the complex as control samples. Geis were stained with InstantBlue Coomassie for visual inspection or SYPRO orange for stoichiometry assessment of the CsgG-CsgE complex by fluorescence detection (Typhoon FLA 9000) of the CsgE and CsgG bands on SDS-PAGE, yielding a CsgG/CsgE ratio of 0.97.

CsgG-CsgE Cryo-EM. Cryo-electron microscopy was used to determine the in-solution structure of the C, CsgG-CsgE complex. CsgG-CsgE complex prepared as described above was bound and eluted from a HisTrap FF (GE Healthcare) to remove unbound CsgG, and on elution it was immediately applied to Quantifoil R212 carbon coated grids (Quantifoil Micro Tools GmbH) that had been glow-discharged at mA for 30 s. Samples were plunge-frozen in liquid nitrogen using an automated system (Leica) and observed under a FEI F20 microscope operating at a voltage of 200 kV, a nominal magnification of ×50,000 under low-dose conditions and a defocus range of 1.4-3 pm. Image frames were recorded on a Falcon II detector. The pixel size at the specimen level was 1.9 Å per pixel. The CTF parameters were assessed using CTFFIND3 (ref. 47), and the phase flipping was done in SPIDER⁴⁶. Particles were automatically selected from CTF-corrected micrographs using BOXER (EMAN2; ref. 49). Images with an astigmatism of more than 10% were discarded. A total of 1,221 particles were selected from 75 micrographs and windowed into 128-pixel X 128-pixel boxes. Images were normalized to the same mean and standard deviation and high-pass filtered at a low-resolution cut-off of ˜200 Å. They were centred and then subjected to a first round of MSA. An initial reference set was obtained using reference free classification in IMAGIC-4D (Image Science Software). The best classes corresponding to characteristic side views of the C₉ cylindrical particles were used as references for the MRA. For CsgG-CsgE complex, the first three-dimensional model was calculated from the best 125 characteristic views (with good contrast and well-defined features) encompassing 1,221 particles of the complex with orientations determined by angular reconstitution (Image Science Software). The three-dimensional map was refined by iterative rounds of MRA, MSA and anchor set refinement. The resolution was estimated to be 24 Å by Fourier shell correlation (FSC) according to the 0.5 criteria level FIG. 52). Visualization of the map and figures was performed in UCSF Chimera⁵⁰.

Bile salt toxicity assay. Outer-membrane permeability was investigated by decreased growth on agar plates containing bile salts. Tenfold serial dilutions of E. coli LSR12 (ref. 7) cells (5 μl) harbouring both pLR42 (ref. 16) and pMC2 (ref. 14) (or derived helix 2 mutants) were spotted on McConkey agar plates containing 100 μg l⁻¹ ampicillin, 25 μg l⁻¹ chloramphenicol, 1 mM IPTG with or without 0.2% (w/v) L-arabinose. After Incubation overnight at 37° C., colony growth was examined.

Single-channel current recordings. Single-channel current recordings were performed using parallel high-resolution electrical recording with the Orbit 16 kit from Nanion. In brief, horizontal bilayers of 1,2-diphytanoyl-sn-glycero-3-phosphocholine (Avanti Polar Lipids) were formed over microcavities (of subpicolitre volume) in a 16-channel multielectrode cavity array (MECA) chip (Ionera)⁵¹. Both the cis and trans cavities above and below the bilayer contained 1.0 M KCl, 25 mM Tris-HCl pH 8.0. To insert channels into the membrane, CsgG dissolved in 25 mM Tris-HCl pH 8.0, 500 mM NaCl, 1 mM DTT, 0.5% C8E4, 5 mM LDAO was added to the cis compartment to a final concentration of 90-300 nM. To test the interaction of the CsgG channel with CsgE, a solution of the latter protein dissolved in 25 mM Tris-HCl pH 8.0, 150 mM NaCl was added to the cis compartment to final concentrations of 0.1, 1, 10 and 100 nM. Transmembrane currents were recorded at a holding potential of +50 mV and −50 mV (with the cis side grounded) using a Tecella Triton 16-channel amplifier at a low-pass filtering frequency of 3 kHz and a sampling frequency of 10 kHz. Current traces were analysed using the Clampfit of the pClamp suite (Molecular Devices). Plots were generated using Origin 8.6 (Microcal)⁵².

Measured currents were compared with those calculated based on the pore dimensions of the CsgGX-ray structure, modelled to be composed of three segments: the transmembrane section, the periplasmic vestibule, and the inner channel constriction connecting the two. The first two segments were modelled to be of conical shape; the constriction was represented as a cylinder. The corresponding resistances R1, R2 and R3, respectively, were calculated as

$$\begin{matrix}R 1 = L 1 / ( π {D}_{1} {d}_{1} κ ) \\ R 2 = L 2 / ( π {D}_{2} {d}_{2} κ ) \\ R 3 = L 3 / ( π {D}_{1} {d}_{2} κ )\end{matrix}$$

where L1, L2 and L3 are the axial lengths of the segments, measuring 3.5, 4.0 and 2.0 nm, respectively, and D1, d1, D2 and d2 are the maximum and minimum diameters of segments 1 and 2, measuring 4.0, 0.8, 3.4 and 0.8 nm, respectively. The conductivity K has a value of 10.6 Sm⁻¹. The current was calculated by inserting R1, R2 and R3 and voltage V=50 mV into

$$I = V / ( R 1 + R 2 + R 3 )$$

Access resistance was not found to alter the predicted current significantly.

### REFERENCES

1. Olsen, A., Jonsson, A. & Normark, S. Fibronectin binding mediated by a novel class of surface organelles on Escherichia coli. Nature 338, 652-655 (1989).
2. Collinson, S. K et al. Thin, aggregative fimbriae mediate binding of Salmonella enteritidis to fibronectin. J. Bacteriol. 175, 12-18 (1993).
3. Dueholm, M. S., Albertsen, M., Otzen, D. & Nielsen, P. H. Curli functional amyloid systems are phylogenetically widespread and display large diversity in operon and protein structure. PLoS ONE 7, e51274 (2012).
4. Cegelski, L. et al. Small-molecule inhibitors target Escherichia coli amyloid biogenesis and biofilm formation. Nature Chem. Biol. 5, 913-919 (2009).
5. Herwald, H. et al. Activation of the contact-phase system on bacterial surfaces—a clue to serious complications in infectious diseases. Nature Med. 4, 298-302 (1998).
6. Hammar, M., Arnqvist, A., Bian, Z., Olsen, A. & Normark, S. Expression of two csg operons is required for production of fibronectin- and Congo red-binding curli polymers in Escherichia coli K-12. Mol. Microbiol. 18, 661-670 (1995).
7. Chapman, M. R. et al. Role of Escherichia coli curli operons in directing amyloid fiber formation. Science 295, 851-855 (2002).
8. Wang, X., Smith. D. R., Jones. J. W. & Chapman, M. R. In vitro polymerization of a functional Escherichia coli amyloid protein. J. Biol. Chem. 282, 3713-3719 (2007).
9. Dueholm, M. S. et al. Fibrillation of the major curli subunit CsgA under a wide range of conditions implies a robust design of aggregation. Biochemistry 50, 8281-8290 (2011).
10. Hung, C. et al. Escherichia coli biofilms have an organized and complex extracellular matrix structure. MBio 4, e00645-13 (2013).
11. Hammar, M., Bian, Z.& Normark, S. Nucleator-dependent intercellular assembly of adhesive curli organelles in Escherichia coli. Proc. Natl Acad. Sci. USA 93, 6562-6566 (1996).
12. Bian, Z. & Normark, S. Nucleator function of CsgB for the assembly of adhesive surface organelles in Escherichia coli. EMBO J. 16, 5827-5836 (1997).
13. Loferer, H., Hammar, M.& Normark, S. Availability of the fibre subunit CsgA and the nucleator protein CsgB during assembly of fibronectin-binding curli is limited by the intracellular concentration of the novel lipoprotein CsgG. Mol. Microbiol. 26, 11-23 (1997).
14. Robinson, L. S., Ashman. E. M., Hultgren, S. J. & Chapman, M. R. Secretion of curli fibre subunits is mediated by the outer membrane-localized CsgG protein. Mol. Microbiol. 59, 870-881 (2006)
15. Nenninger, A. A., Robinson, L. S. & Hultgren, S. J. Localized and efficient curli nucleation requires the chaperone-like amyloid assembly protein CsgF. Proc. Natl Aced. Sci. USA 106, 900-905 (2009).
16. Nenninger, A. A. et al. CsgE is a curli secretion specificity factor that prevents amyloid fibre aggregation. Mol. Microbiol. 81, 486-499 (2011).
17. Okuda, S. & Tokuda, H. Lipoprotein sorting in bacteria. Annu. Rev. Microbiol. 65, 239-259 (2011).
18. Iacovache, I., Bischofberger, M. & van der Goot, F. G. Structure and assembly of pore-forming proteins. Curr. Opin. Struct. Biol. 20, 241-246 (2010).
19. Goyal, P., Van Gerven, N., Jonckheere, W. & Remaut, H. Crystallization and preliminary X-ray crystallographic analysis of the curli transporter CsgG. Acta Crystallogr. F Struct. Biol. Cryst. Commun. 69, 1349-1353 (2013).
20. Krantz, B. A. et al. A phenylalanine clamp catalyzes protein translocation through the anthrax toxin pore. Science 309, 777-781 (2005).
21. Janowiak, B. E., Fischer, A. & Collier, R. J. Effects of introducing a single charged residue into the phenylalanine clamp of multimeric anthrax protective antigen. J. Biol. Chem. 285, 8130-8137 (2010).
22. Feld, G. K., Brown, M. J. & Krantz, B. A. Ratcheting up protein translocation with anthrax toxin. Protein Sci. 21, 606-624 (2012).
23. Van Gerven, N. et al. Secretion and functional display of fusion proteins through the curli biogenesis pathway. Mol. Microbiol. 91, 1022-1035 (2014).
24. Brinker, A. et al. Dual function of protein confinement in chaperonin-assisted protein folding. Cell 107, 223-233 (2001).
25. Busby, J. N., Panjikar, S., Landsberg, M. J., Hurst, M. R. & Lott, J. S. The BC component of ABC toxins is an RHS-repeat-containing protein encapsulation device. Nature 501, 547-550 (2013).
26. Takagi, F., Koga, N. & Takada, S. How protein thermodynamics and folding mechanisms are altered by the chaperonin cage:molecular simulations. Proc. Natl Acad. Sci. USA 100, 11367-11372 (2003).
27. Zhou, H. X. Protein folding in confined and crowded environments. Arch. Biochem. Biophys. 469, 76-82 (2008).
28. Chen, A. Y. et al. Synthesis and patterning of tunable multiscale materials with engineered cells. Nature Mater. 13, 515-523 (2014).
29. Sivanathan, V. & Hochschild. A. A bacterial export system for generating extracellular amyloid aggregates. Nature Protocols 8, 1381-1390 (2013).
30. Datsenko, K. A. & Wanner, B. L. One-step inactivation of chromosomal genes in Escherichia coli K-12 using PCR products. Proc. Natl Acad. Sci. USA 97, 6640-6645 (2000).
31. Swamy, M., Siegers, G. M., Minguet, S., Wollscheid, B. & Schamel, W. W. A. Blue native polyacrylamide gel electrophoresis (BN-PAGE) for the identification and analysis of multiprotein complexes. Sci. STKE 2006, pl 4, http://dx.doi.org/10.1126/stke.3452006pl4 (2006).
32. Winter, G. xia2: an expert system for macromolecular crystallography data reduction. J. Appl. Cryst. 43, 186-190 (2010).
33. Kabsch, W. Xds. Acta Crystallogr. D Biol. Crystallogr. 66, 125-132 (2010).
34. Sheldrick, G. M. Experimental phasing with SHELXC/D/E: combining chain tracing with density modification. Acta Crystallogr. D Biol. Crystallogr. 66, 479-485 (2010).
35. Bricogne, G., Vonrhein. C., Flensburg, C., Schilitz, M. & Paciorek, W. Generation, representation and flow of phase information in structure determination: recent developments in and around SHARP 2.0. Acta Crystallogr. D Biol. Crystallogr. 59, 2023-2030 (2003).
36. Cowtan, K. Recent developments in classical density modification. Acta Crystallogr. D Biol. Crystallogr. 66, 470-478 (2010).
37. Cowtan, K. The Buccaneer software for automated model building. 1. Tracing protein chains. Acta Crystallogr. D Biol. Crystallogr. 62, 1002-1011 (2006).
38. Adams. P. D. et al. PHENIX: a comprehensive Python-based system for macromolecular structure solution. Acta Crystallogr. D Biol. Crystallogr. 66.213-221 (2010).
39. Emsley, P., Lohkamp. B., Scott. W. G. & Cowtan, K. Features and development of Coot. Acta Crystallogr. D Biol. Crystallogr. 66, 486-501 (2010).
40. Davis. I. W. et al. MolProbity: all-atom contacts and structure validation for proteins and nucleic acids. Nucleic Acids Res. 35 (Suppl 2), W375-W383 (2007).
41. Strong, M. et al. Toward the structural genomics of complexes: crystal structure of a PE/PPE protein complex from Mycobacterium tuberculosis. Proc. Natl Acad. Sci. USA 103, 8060-8065 (2006).
42. McCoy, A. J. et al. Phaser crystallographic software. J. Appl. Cryst. 40, 658-874 (2007).
43. Smart, O. S. et al. Exploiting structure similarity in refinement: automated NCS and target-structure restraints in BUSTER. Acta Crystallogr. D Biol. Crystallogr. 68, 368-380 (2012).
44. Murshudov, G. N et al. REFMAC5 for the refinement of macromolecular crystal structures. Acta Crystallogr. D Biol. Crystallogr. 67, 355-367 (2011).
45. Nicholls, R. A., Long, F. & Murshudov, G. N. Low-resolution refinement tools in REFMAC5. Acta Crystallogr. D Biol. Crystallogr. 68, 404-417 (2012).
46. Goormaghtigh, E. & Ruysschaert, J. M. Subtraction of atmospheric water contribution in Fourier transform infrared spectroscopy of biological membranes and proteins. Spectrochim. Acta 50A, 2137-2144 (1994).
47. Mindell, J. A.& Grigorieff, N. Accurate determination of local defocus and specimen tilt in electron microscopy. J. Struct. Biol. 142, 334-347 (2003).
48. Shaikh, T. R. et al. SPIDER Image processing for single-particle reconstruction of biological macromolecules from electron micrographs. Nature Protocols 3, 1941-1974 (2008).
49. Tang, G. et al. EMAN2: an extensible image processing suite for electron microscopy. J. Struct. Biol. 157, 38-46 (2007).
50. Pettersen, E. F. et al. UCSF Chimera—a visualization system for exploratory research and analysis. J. Comput. Chem. 25, 1605-1612 (2004).
51. Del Rio Martinez, J. M., Zaitseva, E., Petersen. S., Baaken, G. & Behrends, J. C. Automated formation of lipid membrane microarrays for ionic single molecule sensing with protein nanopores. Small http://dx.doi.org/10.1002/smil.201402016 (13 Aug. 2014).
52. Movileanu, L., Howorka, S., Braha, O. & Bayley, H. Detecting protein analytes that modulate transmembrane movement of a polymer chain within a single protein pore. Nature Biotechnol. 18, 1091-1095 (2000).
53. Pronk, S. et al. GROMACS 4.5: a high-throughput and highly parallel open source molecular simulation toolkit. Bioinformatics 29, 8454854 (2013).
54. Lindorff-Larsen, K. et al. Improved side-chain torsion potentials for the Amber ff99SB protein force field. Proteins 78, 1950-1958 (2010).
55. Capra, J. A. & Singh, M. Predicting functionally important residues from sequence conservation. Bioinformatics 23, 1875-1882 (2007).

1.-76. (canceled)

77. A method for molecular sensing comprising:
a) providing a CsgG pore inserted into a membrane, wherein the CsgG pore comprises at least one CsgG monomer;
b) contacting the CsgG pore with a test substrate such that the test substrate interacts with the CsgG pore; and
c) taking one or more measurements during the interaction.

78. The method of claim 77, wherein the test substrate is a nucleic acid, a protein, a peptide, or a small molecule.

79. The method of claim 78, wherein the small molecule is a pharmaceutical, a toxin, or a cytokine.

80. The method of claim 77, wherein the CsgG monomer comprises amino acid mutations at two or more positions corresponding to Y51, N53, and F56 of the amino acid sequence of SEQ ID NO: 390.

81. The method of claim 77, wherein the CsgG monomer comprises amino acid mutations corresponding to Y51/N55, Y51/F56, N55/F56 or Y51/N55/F56.

82. The method of claim 80, wherein the amino acid mutations comprise amino acid deletions or substitutions.

83. The method of claim 80, wherein the at least one CsgG monomer comprises mutations corresponding to F56N/N55Q, F56N/N55R, F56N/N55K, F56N/N55S, F56N/N55G, F56N/N55A, F56N/N55T, F56Q/N55Q, F56Q/N55R, F56Q/N55K, F56Q/N55S, F56Q/N55G, F56Q/N55A, F56Q/N55T, F56R/N55Q, F56R/N55R, F56R/N55K, F56R/N55S, F56R/N55G, F56R/N55A, F56R/N55T, F56S/N55Q, F56S/N55R, F56S/N55K, F56S/N55S, F56S/N55G, F56S/N55A, F56S/N55T, F56G/N55Q, F56G/N55R, F56G/N55K, F56G/N55S, F56G/N55G, F56G/N55A, F56G/N55T, F56A/N55Q, F56A/N55R, F56A/N55K, F56A/N55S, F56A/N55G, F56A/N55A, F56A/N55T, F56K/N55Q, F56K/N55R, F56K/N55K, F56K/N55S, F56K/N55G, F56K/N55A, F56K/N55T, F56N/Y51L, F56N/Y51V, F56N/Y51A, F56N/Y51N, F56N/Y51Q, F56N/Y51S, F56N/Y51G, F56Q/Y51L, F56Q/Y51V, F56Q/Y51A, F56Q/Y51N, F56Q/Y51Q, F56Q/Y51S, F56Q/Y51G, F56R/Y51L, F56R/Y51V, F56R/Y51A, F56R/Y51N, F56R/Y51Q, F56R/Y51S, F56R/Y51G, F56S/Y51L, F56S/Y51V, F56S/Y51A, F56S/Y51N, F56S/Y51Q, F56S/Y51S, F56S/Y51G, F56G/Y51L, F56G/Y51V, F56G/Y51A, F56G/Y51N, F56G/Y51Q, F56G/Y51S, F56G/Y51G, F56A/Y51L, F56A/Y51V, F56A/Y51A, F56A/Y51N, F56A/Y51Q, F56A/Y51S, F56A/Y51G, F56K/Y51L, F56K/Y51V, F56K/Y51A, F56K/Y51N, F56K/Y51Q, F56K/Y51S, F56K/Y51G, N55Q/Y51L, N55Q/Y51V, N55Q/Y51A, N55Q/Y51N, N55Q/Y51Q, N55Q/Y51S, N55Q/Y51G, N55R/Y51L, N55R/Y51V, N55R/Y51A, N55R/Y51N, N55R/Y51Q, N55R/Y51S, N55R/Y51G, N55K/Y51L, N55K/Y51V, N55K/Y51A, N55K/Y51N, N55K/Y51Q, N55K/Y51S, N55K/Y51G, N55S/Y51L, N55S/Y51V, N55S/Y51A, N55S/Y51N, N55S/Y51Q, N55S/Y51S, N55S/Y51G, N55G/Y51L, N55G/Y51V, N55G/Y51A, N55G/Y51N, N55G/Y51Q, N55G/Y51S, N55G/Y51G, N55A/Y51L, N55A/Y51V, N55A/Y51A, N55A/Y51N, N55A/Y51Q, N55A/Y51S, N55A/Y51G, N55T/Y51L, N55T/Y51V, N55T/Y51A, N55T/Y51N, N55T/Y51Q, N55T/Y51S, N55T/Y51G, F56N/N55Q/Y51L, F56N/N55Q/Y51V, F56N/N55Q/Y51A, F56N/N55Q/Y51N, F56N/N55Q/Y51Q, F56N/N55Q/Y51S, F56N/N55Q/Y51G, F56N/N55R/Y51L, F56N/N55R/Y51V, F56N/N55R/Y51A, F56N/N55R/Y51N, F56N/N55R/Y51Q, F56N/N55R/Y51S, F56N/N55R/Y51G, F56N/N55K/Y51L, F56N/N55K/Y51V, F56N/N55K/Y51A, F56N/N55K/Y51N, F56N/N55K/Y51Q, F56N/N55K/Y51S, F56N/N55K/Y51G, F56N/N55S/Y51L, F56N/N55S/Y51V, F56N/N55S/Y51A, F56N/N55S/Y51N, F56N/N55S/Y51Q, F56N/N55S/Y51S, F56N/N55S/Y51G, F56N/N55G/Y51L, F56N/N55G/Y51V, F56N/N55G/Y51A, F56N/N55G/Y51N, F56N/N55G/Y51Q, F56N/N55G/Y51S, F56N/N55G/Y51G, F56N/N55A/Y51L, F56N/N55A/Y51V, F56N/N55A/Y51A, F56N/N55A/Y51N, F56N/N55A/Y51Q, F56N/N55A/Y51S, F56N/N55A/Y51G, F56N/N55T/Y51L, F56N/N55T/Y51V, F56N/N55T/Y51A, F56N/N55T/Y51N, F56N/N55T/Y51Q, F56N/N55T/Y51S, F56N/N55T/Y51G, F56Q/N55Q/Y51L, F56Q/N55Q/Y51V, F56Q/N55Q/Y51A, F56Q/N55Q/Y51N, F56Q/N55Q/Y51Q, F56Q/N55Q/Y51S, F56Q/N55Q/Y51G, F56Q/N55R/Y51L, F56Q/N55R/Y51V, F56Q/N55R/Y51A, F56Q/N55R/Y51N, F56Q/N55R/Y51Q, F56Q/N55R/Y51S, F56Q/N55R/Y51G, F56Q/N55K/Y51L, F56Q/N55K/Y51V, F56Q/N55K/Y51A, F56Q/N55K/Y51N, F56Q/N55K/Y51Q, F56Q/N55K/Y51S, F56Q/N55K/Y51G, F56Q/N55S/Y51L, F56Q/N55S/Y51V, F56Q/N55S/Y51A, F56Q/N55S/Y51N, F56Q/N55S/Y51Q, F56Q/N55S/Y51S, F56Q/N55S/Y51G, F56Q/N55G/Y51L, F56Q/N55G/Y51V, F56Q/N55G/Y51A, F56Q/N55G/Y51N, F56Q/N55G/Y51Q, F56Q/N55G/Y51S, F56Q/N55G/Y51G, F56Q/N55A/Y51L, F56Q/N55A/Y51V, F56Q/N55A/Y51A, F56Q/N55A/Y51N, F56Q/N55A/Y51Q, F56Q/N55A/Y51S, F56Q/N55A/Y51G, F56Q/N55T/Y51L, F56Q/N55T/Y51V, F56Q/N55T/Y51A, F56Q/N55T/Y51N, F56Q/N55T/Y51Q, F56Q/N55T/Y51S, F56Q/N55T/Y51G, F56R/N55Q/Y51L, F56R/N55Q/Y51V, F56R/N55Q/Y51A, F56R/N55Q/Y51N, F56R/N55Q/Y51Q, F56R/N55Q/Y51S, F56R/N55Q/Y51G, F56R/N55R/Y51L, F56R/N55R/Y51V, F56R/N55R/Y51A, F56R/N55R/Y51N, F56R/N55R/Y51Q, F56R/N55R/Y51S, F56R/N55R/Y51G, F56R/N55K/Y51L, F56R/N55K/Y51V, F56R/N55K/Y51A, F56R/N55K/Y51N, F56R/N55K/Y51Q, F56R/N55K/Y51S, F56R/N55K/Y51G, F56R/N55S/Y51L, F56R/N55S/Y51V, F56R/N55S/Y51A, F56R/N55S/Y51N, F56R/N55S/Y51Q, F56R/N55S/Y51S, F56R/N55S/Y51G, F56R/N55G/Y51L, F56R/N55G/Y51V, F56R/N55G/Y51A, F56R/N55G/Y51N, F56R/N55G/Y51Q, F56R/N55G/Y51S, F56R/N55G/Y51G, F56R/N55A/Y51L, F56R/N55A/Y51V, F56R/N55A/Y51A, F56R/N55A/Y51N, F56R/N55A/Y51Q, F56R/N55A/Y51S, F56R/N55A/Y51G, F56R/N55T/Y51L, F56R/N55T/Y51V, F56R/N55T/Y51A, F56R/N55T/Y51N, F56R/N55T/Y51Q, F56R/N55T/Y51S, F56R/N55T/Y51G, F56S/N55Q/Y51L, F56S/N55Q/Y51V, F56S/N55Q/Y51A, F56S/N55Q/Y51N, F56S/N55Q/Y51Q, F56S/N55Q/Y51S, F56S/N55Q/Y51G, F56S/N55R/Y51L, F56S/N55R/Y51V, F56S/N55R/Y51A, F56S/N55R/Y51N, F56S/N55R/Y51Q, F56S/N55R/Y51S, F56S/N55R/Y51G, F56S/N55K/Y51L, F56S/N55K/Y51V, F56S/N55K/Y51A, F56S/N55K/Y51N, F56S/N55K/Y51Q, F56S/N55K/Y51S, F56S/N55K/Y51G, F56S/N55S/Y51L, F56S/N55S/Y51V, F56S/N55S/Y51A, F56S/N55S/Y51N, F56S/N55S/Y51Q, F56S/N55S/Y51S, F56S/N55S/Y51G, F56S/N55G/Y51L, F56S/N55G/Y51V, F56S/N55G/Y51A, F56S/N55G/Y51N, F56S/N55G/Y51Q, F56S/N55G/Y51S, F56S/N55G/Y51G, F56S/N55A/Y51L, F56S/N55A/Y51V, F56S/N55A/Y51A, F56S/N55A/Y51N, F56S/N55A/Y51Q, F56S/N55A/Y51S, F56S/N55A/Y51G, F56S/N55T/Y51L, F56S/N55T/Y51V, F56S/N55T/Y51A, F56S/N55T/Y51N, F56S/N55T/Y51Q, F56S/N55T/Y51S, F56S/N55T/Y51G, F56G/N55Q/Y51L, F56G/N55Q/Y51V, F56G/N55Q/Y51A, F56G/N55Q/Y51N, F56G/N55Q/Y51Q, F56G/N55Q/Y51S, F56G/N55Q/Y51G, F56G/N55R/Y51L, F56G/N55R/Y51V, F56G/N55R/Y51A, F56G/N55R/Y51N, F56G/N55R/Y51Q, F56G/N55R/Y51S, F56G/N55R/Y51G, F56G/N55K/Y51L, F56G/N55K/Y51V, F56G/N55K/Y51A, F56G/N55K/Y51N, F56G/N55K/Y51Q, F56G/N55K/Y51S, F56G/N55K/Y51G, F56G/N55S/Y51L, F56G/N55S/Y51V, F56G/N55S/Y51A, F56G/N55S/Y51N, F56G/N55S/Y51Q, F56G/N55S/Y51S, F56G/N55S/Y51G, F56G/N55G/Y51L, F56G/N55G/Y51V, F56G/N55G/Y51A, F56G/N55G/Y51N, F56G/N55G/Y51Q, F56G/N55G/Y51S, F56G/N55G/Y51G, F56G/N55A/Y51L, F56G/N55A/Y51V, F56G/N55A/Y51A, F56G/N55A/Y51N, F56G/N55A/Y51Q, F56G/N55A/Y51S, F56G/N55A/Y51G, F56G/N55T/Y51L, F56G/N55T/Y51V, F56G/N55T/Y51A, F56G/N55T/Y51N, F56G/N55T/Y51Q, F56G/N55T/Y51S, F56G/N55T/Y51G, F56A/N55Q/Y51L, F56A/N55Q/Y51V, F56A/N55Q/Y51A, F56A/N55Q/Y51N, F56A/N55Q/Y51Q, F56A/N55Q/Y51S, F56A/N55Q/Y51G, F56A/N55R/Y51L, F56A/N55R/Y51V, F56A/N55R/Y51A, F56A/N55R/Y51N, F56A/N55R/Y51Q, F56A/N55R/Y51S, F56A/N55R/Y51G, F56A/N55K/Y51L, F56A/N55K/Y51V, F56A/N55K/Y51A, F56A/N55K/Y51N, F56A/N55K/Y51Q, F56A/N55K/Y51S, F56A/N55K/Y51G, F56A/N55S/Y51L, F56A/N55S51Y5V, F56A/N55S/Y51A, F56A/N55S/Y51N, F56A/N55S/Y51Q, F56A/N55S/Y51S, F56A/N55S/Y51G, F56A/N55G/Y51L, F56A/N55G/Y51V, F56A/N55G/Y51A, F56A/N55G/Y51N, F56A/N55G/Y51Q, F56A/N55G/Y51S, F56A/N55G/Y51G, F56A/N55A/Y51L, F56A/N55A/Y51V, F56A/N55A/Y51A, F56A/N55A/Y51N, F56A/N55A/Y51Q, F56A/N55A/Y51S, F56A/N55A/Y51G, F56A/N55T/Y51L, F56A/N55T/Y51V, F56A/N55T/Y51A, F56A/N55T/Y51N, F56A/N55T/Y51Q, F56A/N55T/Y51S, F56A/N55T/Y51G, F56K/N55Q/Y51L, F56K/N55Q/Y51V, F56K/N55Q/Y51A, F56K/N55Q/Y51N, F56K/N55Q/Y51Q, F56K/N55Q/Y51S, F56K/N55Q/Y51G, F56K/N55R/Y51L, F56K/N55R/Y51V, F56K/N55R/Y51A, F56K/N55R/Y51N, F56K/N55R/Y51Q, F56K/N55R/Y51S, F56K/N55R/Y51G, F56K/N55K/Y51L, F56K/N55K/Y51V, F56K/N55K/Y51A, F56K/N55K/Y51N, F56K/N55K/Y51Q, F56K/N55K/Y51S, F56K/N55K/Y51G, F56K/N55S/Y51L, F56K/N55S/Y51V, F56K/N55S/Y51A, F56K/N55S/Y51N, F56K/N55S/Y51Q, F56K/N55S/Y51S, F56K/N55S/Y51G, F56K/N55G/Y51L, F56K/N55G/Y51V, F56K/N55G/Y51A, F56K/N55G/Y51N, F56K/N55G/Y51Q, F56K/N55G/Y51S, F56K/N55G/Y51G, F56K/N55A/Y51L, F56K/N55A/Y51V, F56K/N55A/Y51A, F56K/N55A/Y51N, F56K/N55A/Y51Q, F56K/N55A/Y51S, F56K/N55A/Y51G, F56K/N55T/Y51L, F56K/N55T/Y51V, F56K/N55T/Y51A, F56K/N55T/Y51N, F56K/N55T/Y51Q, F56K/N55T/Y51S, F56K/N55T/Y51G, F56E/N55R, F56E/N55K, F56D/N55R, F56D/N55K, F56R/N55E, F56R/N55D, F56K/N55E or F56K/N55D.

84. The method of claim 77, wherein the CsgG pore is a hetero-oligomeric pore.

85. The method of claim 84, wherein the hetero-oligomeric pore comprises nine CsgG monomers.

86. The method of claim 85, wherein at least one of the nine CsgG monomers is different from the other CsgG monomers.

87. The method of claim 77, wherein the CsgG pore is a homo-oligomeric pore.

88. The method of claim 87, wherein the homo-oligomeric pore consists of nine identical CsgG monomers.

89. A kit comprising:
(i) a CsgG pore that comprises at least one CsgG monomer, wherein the CsgG monomer comprises amino acid mutations at two or more of positions corresponding to Y51, N55 and F56 of the amino acid sequence of SEQ ID NO: 390; and
(ii) components of a membrane.
